SEQUENCE LISTING (19 sequences)

SEQ ID NO: 1 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 2 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 3 - protein, 7 aa, Simian virus 40
PKKKRKV

SEQ ID NO: 4 - protein, 10 aa, Simian virus 40
PKKKRKVEAS

SEQ ID NO: 5 - protein, 16 aa, Simian virus 40
KRPAATKKAGQAKKKK

SEQ ID NO: 6 - protein, 9 aa, Homo sapiens
PAAKRVKLD

SEQ ID NO: 7 - protein, 11 aa, Homo sapiens
RQRRNELKRSP

SEQ ID NO: 8 - protein, 38 aa, Homo sapiens
NQSSNFGPMKGGNFGGRSSGPYGGGGQYFAKPRNQGGY

SEQ ID NO: 9 - protein, 42 aa, Homo sapiens
RMRIZFKNKGKDTAELRRRRVEVSVELRKAKKDEQILKRRNV

SEQ ID NO: 10 - protein, 8 aa, Homo sapiens
VSRKRPRP

SEQ ID NO: 11 - protein, 8 aa, Homo sapiens
PPKKARED

SEQ ID NO: 12 - protein, 8 aa, Homo sapiens
PQPKKKPL

SEQ ID NO: 13 - protein, 12 aa, Mus sp.
SALIKKKKKMAP

SEQ ID NO: 14 - protein, 5 aa, Influenza virus
DRLRR

SEQ ID NO: 15 - protein, 7 aa, Influenza virus
PKQKKRK

SEQ ID NO: 16 - protein, 10 aa, Hepatitis virus
RKLKKKIKKL

SEQ ID NO: 17 - protein, 10 aa, Mus sp.
REKKKFLKRR

SEQ ID NO: 18 - protein, 20 aa, Homo sapiens
KRKGDEVDGVDEVAKKKSKK

SEQ ID NO: 19 - protein, 17 aa, Homo sapiens
RKCLQAGMNLEARKTKK

(19) United States
(12) Patent Application Publication
(10) Pub. No.: US 2022/0249701 A1
(43) Pub. Date: Aug. 11, 2022

(54) COMPOSITIONS AND METHODS FOR TARGETING MULTINUCLEATED CELLS

(71) Applicants: The Broad Institute, Inc., Cambridge, MA (US); Massachusetts Institute of Technology, Cambridge, MA (US)

(72) Inventors: Aviv Regev, Cambridge, MA (US); Eugene Drokhlyansky, Cambridge, MA (US); Orit Rozenblatt-Rosen, Cambridge, MA (US); Gökcen Eraslan, Cambridge, MA (US)

(21) Appl. No.: 17/610,942

(22) PCT Filed: May 14, 2020
(86) PCT No.: PCT/US2020/032933

§ 371 Date: Nov. 12, 2021

Related U.S. Application Data
(60) Provisional application No. 62/847,668, filed on May 14, 2019.

(51) Int. Cl. A61K 48/00; C12N 9/22; C12N 15/11; C12N 15/62; C12N 15/86; C12N 15/90; C12N 9/78; A61K 35/76; A61K 38/46; A61K 31/7088; A61P 21/00

(52) U.S. Cl. CPC A61K 48/0058; C12N 2830/008; C12N 15/11; C12N 15/62; C12N 15/86; C12N 15/907; C12N 9/78; C12Y 305/04004; C12Y 305/04005; A61K 35/76; A61K 38/465; A61K 31/7088; A61P 21/00; C12N 2310/20; C12N 2800/80; C12N 2750/14143; C12N 9/22

(57) ABSTRACT

The present invention provides for methods and compositions for specifically targeting a nuclei type in a multinucleated cell or population of multinucleated cells, wherein the composition comprises one or more nucleotide sequences operably linked to one or more regulatory sequences specifically active in the nuclei.

### CROSS-REFERENCE TO RELATED APPLICATIONS

This application claims the benefit of U.S. Provisional Application No. 62/847,668, filed May 14, 2019. The entire contents of the above-identified application are hereby fully incorporated herein by reference.

### REFERENCE TO AN ELECTRONIC SEQUENCE LISTING

The contents of the electronic sequence listing (BROD-4180WP_ST25.txt”; Size is 8 Kilobytes and it was created on May 5, 2020) is herein incorporated by reference in its entirety.

### TECHNICAL FIELD

The subject matter disclosed herein is generally directed to gene therapies that target specific types of nuclei in multinucleated cells.

### BACKGROUND

Various gene therapy approaches have been used for treating diseases resulting from genetic mutations. However, the current gene therapies may not be suitable for treating diseases related to multinucleated cells, e.g., muscular dystrophy related to muscle cells. It is presently not known whether all nuclei in a multinucleated cell express the same genes and whether current gene therapy approaches targeting multinucleated cells can effectively treat the diseases.

### SUMMARY

In one aspect, the present invention provides for a composition for specifically targeting a nuclei type in a multinucleated cell or population of multinucleated cells, wherein the composition comprises one or more nucleotide sequences operably linked to one or more regulatory sequences specifically active in the nuclei. In certain embodiments, the multinucleated cell is selected from the group consisting of a skeletal muscle cell, cardiac muscle cell, liver cell and placenta cell. In certain embodiments, the one or more regulatory sequences comprises a promoter active in the nuclei. In certain embodiments, the one or more regulatory sequences comprises an enhancer active in the nuclei.

In certain embodiments, the composition further comprises one or more viral vectors selected from the group consisting of adeno-associated virus (AAV) vectors, lentivirus vectors, and adenovirus vectors. In certain embodiments, the viral vector is a recombinant AAV (rAAV) vector. In certain embodiments, the rAAV is selected from the group consisting of rAAV6, rAAV1, rAAV7, rAAV8, and rAAV 9. In certain embodiments, the rAAV is rAAV6. In certain embodiments, the composition is a nucleoprotein complex.

In certain embodiments, the one or more nucleotide sequences encodes one or more components of a CRISPR system, a zinc finger nuclease system, a TALE system, a TALEN system, a meganuclease, antisense oligonucleotide, or RNAi system. In certain embodiments, the composition further comprises a homologous donor template. In certain embodiments, the CRISPR system is a Class 1 or Class 2 CRISPR system. In certain embodiments, the Class 2 system comprises a Type II Cas polypeptide. In certain embodiments, the Type II Cas is a Cas9. In certain embodiments, the Class 2 system comprises a Type V Cas polypeptide. In certain embodiments, the Type V Cas is Cas12a, Cas12b, Cas12c, Cas12d (CasY), Cas12e(CasX), or Cas14. In certain embodiments, the Class 2 system comprises a Type VI Cas polypeptide. In certain embodiments, the Type VI Cas is Cas13a, Cas13b, Cas13c or Cas13d. In certain embodiments, the CRISPR system comprises an inactivated CRISPR enzyme (dCas) fused or otherwise linked to a nucleotide deaminase. In certain embodiments, the nucleotide deaminase is a cytidine deaminase or an adenosine deaminase. In certain embodiments, the dCas is a dCas9, dCas12, or dCas13. In certain embodiments, the nucleotide deaminase is capable of DNA editing. In certain embodiments, the nucleotide deaminase is capable of altering mRNA splicing by editing mRNA. In certain embodiments, the composition further comprises an AAV replication system capable of generating antisense DNA molecules or homologous donor templates.

In certain embodiments, the one or more regulatory sequences are derived from endogenous regulatory sequences that control expression of one or more myocyte nuclei specific genes selected from the group consisting of: PDK4, PDE4D, DMD, NEB, TTN, SVIL, RP11-434D9.1, MLIP, PDLIM5 and EYA4; or TTN, NEB, MLIP, SVIL, DMD, NEXN, RP11-362A9.3, PDE4D, FBXO32 and TRDN; or PDE4D, NEB, TRIM63, MLIP, EYA4, FBXO32, TBC1D8, TTN, GBE1 and DMD; or RP11-434D9.1, ART3, EMC10, SVIL, NEXN, BCL6, DMD, MYO18B, SESN1 and TTN; or GBE1, PDE4D, DMD, MLIP, EYA4, NEAT1, ACACB, UBE2E2, TBC1D8 and KIAA1217; or NEAT1, MALAT1, BCL6, NEXN, DMD, NEDD4, TNNT3, ART3, SESN1 and SVIL; or MLIP, PDE4D, DMD, SVIL, RP11-362A9.3, NEB, TTN, EYA4, NEXN and FBXO32; or genes in Table 1A cluster 2; or genes in Table 1A cluster 0; or genes in Table 2A cluster 2; or genes in Table 2A cluster 0; or genes in Table 3A cluster 2; or genes in Table 3A cluster 0; or genes in Table 4A cluster 0, whereby the composition is specific to nuclei expressing the one or more markers. In certain embodiments, the one or more regulatory sequences comprises a promoter specific to the one or more genes. In certain embodiments, the one or more regulatory sequences comprises an enhancer specific to the one or more genes.

In certain embodiments, the composition is for use in treating muscular dystrophy in a subject in need thereof, wherein the composition is capable of altering expression of dystrophin in the specific nuclei. In certain embodiments, the muscular dystrophy is Duchenne's muscular dystrophy (DMD). In certain embodiments, the muscular dystrophy is Becker muscular dystrophy (BMD). In certain embodiments, the nucleotide sequence encodes an antisense oligonucleotide or antisense nucleotide system capable of inducing exon skipping in dystrophin mRNA. In certain embodiments, the antisense oligonucleotide or antisense nucleotide system induces exon skipping at exon 43, 44, 50, 51, 52 or 55. In certain embodiments, the nucleotide sequence encodes an exogenous dystrophin gene or functional fragment. In certain embodiments, the CRISPR system, zinc finger nuclease system, or TALE system edits a splice site in dystrophin mRNA. In certain embodiments, the splice site is within 50 bases from exon 43, 44, 50, 51, 52, or 55 in dystrophin mRNA. In certain embodiments, the CRISPR system, zinc finger nuclease system, TALEN system, or a meganuclease specifically cuts the endogenous dystrophin gene. In certain embodiments, the composition further comprises a means for generating a homologous donor template specific for repairing the endogenous dystrophin gene.

In another aspect, the present invention provides for a method of treating muscular dystrophy in a subject in need thereof comprising administering the composition to the subject. In certain embodiments, the composition according to any embodiment herein is administered by intramuscular or intravascular injection.

In another aspect, the present invention provides for a composition according to any embodiment herein for use in treating a monogenic disease associated with multinucleated cells in a subject in need thereof, wherein the composition is capable of altering expression of the gene causing the disease in the specific nuclei where the gene is normally expressed in a healthy subject. In certain embodiments, the disease is autosomal dominant, autosomal recessive, X-linked recessive, or X-linked dominant.

In another aspect, the present invention provides for a method of treating a monogenic disease associated with multinucleated cells in a subject in need thereof comprising administering the composition to the subject. In certain embodiments, the composition is administered by intramuscular or intravascular injection.

In certain embodiments, the composition according to any embodiment herein alters the gene exclusively in nuclei where it is expressed in a normal subject. In certain embodiments, the regulatory sequence of the composition according to any embodiment herein does not comprise the CMV, CK8, MHCK7, MCK, MB, ACTA1, TPM2, TPM3 or CKM promoter.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Plots on expression of example genes in clusters of CST isolated nuclei from skeletal muscle.

FIG. 2—shows clusters of cells identified in skeletal muscle nuclei by Uniform Manifold Approximation and Projection (UMAP). Nuclei were isolated from tissues using the following lysis buffers: CST, TZ, NST, and TST.

FIG. 3A-FIG. 3E—UMAP clusters from nuclei extracted with different lysis buffers where the expression of the indicated genes is projected on the clusters. FIG. 3A—UMAP plots from combined four nuclei extractions across three individuals per extraction. FIG. 3B—UMAP plots from EZ nuclei extraction and across three individuals. FIG. 3C—UMAP plots from NST nuclei extraction and across three individuals. FIG. 3D—UMAP plots from CST nuclei extraction and across three individuals. FIG. 3E—UMAP plots from TST nuclei extraction and across three individuals.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

The present disclosure provides for compositions and methods for manipulating genes in specific nuclei in multinucleated cells. Using single nuclei sequencing, Applicants have discovered that nuclei in a multinucleated cell have different gene expression programs. Applicants have identified that genes with mutations related to genetic diseases are expressed in different nuclei in the multinucleated cell. Thus, Applicants have identified that a challenge of using gene therapy for treating a disease related to multinucleated cells is the selective delivery of therapeutic reagents to specific nuclei that express the mutated genes. As used herein, “expression” may refer to transcription of a gene in the nucleus to obtain mRNA. Targeting the correct nuclei can be critical to ensure the efficacy and safety of the treatment. For example, if a mutated gene is corrected in the wrong nuclei there will be no therapeutic effect. Furthermore, off target effects caused by the gene therapy can be limited if the mutation is corrected in only nuclei where the mutated gene is transcribed. Thus, there is a need for compositions and methods for targeted delivery of gene therapy in specific nuclei of multinucleated cells.

The compositions and methods may be used for treating diseases related to multinucleated cells, such as muscular dystrophy. In general, the approach takes advantage of regulatory sequences that are active in specific nuclei. A nucleotide sequence may be operably linked with such regulatory sequence(s). With the control of the regulatory sequence(s), the nucleotide sequence may only be active (expressed) in the specific nuclei in a multinucleated cell.

In an aspect, the present disclosure includes a composition (e.g., a plasmid, vector) comprising one or more nucleotide sequences operably linked to one or more regulatory sequences specifically active in selected nuclei. The nucleotide sequences may manipulate the sequences, expression, and/or activity of genes in the selected nuclei. For example, the nucleotide sequences may be or encode guide RNA in a CRISPR-Cas system or interference RNA.

In another aspect, the disclosure provides methods for treating a disease. The method may comprise delivering the composition to multinucleated cells (e.g., muscle cells) where it specifically manipulates genes in only selected nuclei in the cells.

### Compositions for Targeting Nuclei Subtypes

The present disclosure includes compositions for targeting specific nuclei in a multinucleated cell. In general, the composition may comprise one or more nucleotide sequences operably linked to one or more regulatory sequences. In some embodiments, the composition is capable of specifically targeting a type of nuclei in a multinucleated cell or population of multinucleated cells. Such composition may comprise one or more nucleotide sequences operably linked to one or more regulatory sequences specifically active in the nuclei.

In certain embodiments, the compositions are therapeutic compositions or agents. The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

### Nucleotide Sequences

Nucleotide sequences herein may be nucleic acids or polynucleotides. A nucleotide sequence may be DNA, RNA, or a hybrid thereof, including without limitation, cDNA, mRNA, genomic DNA, mitochondrial DNA, guide sequence, sgRNA, siRNA, shRNA, miRNA, tRNA, rRNA, snRNA, lncRNA, and synthetic (such as chemically synthesized) DNA or RNA or hybrids thereof, or fragments thereof. The nucleotide sequence may be double-stranded or single-stranded. Where single-stranded, the nucleic acid may be the sense strand or the antisense strand. The nucleotide sequences may include natural nucleotides (such as A, T/U, C, and G), modified nucleotides, analogs of natural nucleotides, such as labeled nucleotides, or any combination thereof.

### Modified Nucleotides

In some cases, the nucleotide sequences may include modified nucleotides. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example as described in U.S. Pat. No. 5,866,336 to Nazarenko et al.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others.

Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

### Labels

The nucleotide sequences may comprise one or more labels such as detectable moieties. In some examples, compositions with such nucleotide sequences may be used for labelling specific nuclei in a multinucleated cell. The labels may be used for visualizing, tracking, identifying, isolating, and/or analyzing the specific nuclei. In some examples, the labels may be detectable by spectroscopic, photochemical, biochemical, immunochemical, electrical, optical or chemical means. Such labeled nucleotide sequence may be used for diagnosis of diseases related to multinucleated cells.

Examples of the labels include fluorophores, fluorescent dyes, enzymes, coenzymes, chemiluminescent substances, and radioactive substances. Specific examples include radioisotopes (e.g., ³²P, ¹⁴C, ¹²⁵I, ³H, and ¹³¹I), fluorescein, rhodamine, dansyl chloride, umbelliferone, luciferase, peroxidase, alkaline phosphatase, 0-galactosidase, p-glucosidase, horseradish peroxidase, glucoamylase, lysozyme, saccharide oxidase, microperoxidase, biotin, and ruthenium. In the case where biotin is employed as a label, after addition of a biotin-labeled antibody, streptavidin bound to an enzyme (e.g., peroxidase) is further added. In some examples, the label may be a fluorescent label. Examples of fluorescent labels include, but are not limited to, Atto dyes, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′ tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolta Blue; phthalo cyanine; and naphthalo cyanine. A fluorescent label may be a fluorescent protein, such as blue fluorescent protein, cyan fluorescent protein, green fluorescent protein, red fluorescent protein, yellow fluorescent protein or any photoconvertible protein. The labels may be colorimetric labels, bioluminescent labeling and/or chemiluminescent labeling. Labeling further may include energy transfer between molecules in the hybridization complex by perturbation analysis, quenching, or electron transport between donor and acceptor molecules, the latter of which may be facilitated by double stranded match hybridization complexes. The fluorescent label may be a perylene or a terrylen. In the alternative, the fluorescent label may be a fluorescent bar code. In some examples, the label may be light sensitive, wherein the label may be light-activated and/or light cleaves the one or more linkers to release the molecular cargo. The light-activated molecular cargo may be a major light-harvesting complex (LHCII). In another embodiment, the fluorescent label may induce free radical formation. In some embodiments, the detectable moieties may be quantum dots. A nucleotide sequence herein may comprise one label. In certain cases, a nucleotide sequence herein may comprise multiple labels.

### Regulatory Sequences

The nucleotide sequence may be operably linked to one or more regulatory sequences. The term “operably linked” as used herein refers to linkage of a regulatory sequence to a DNA sequence, such that the regulatory sequence regulates the mediates transcription of the DNA sequence. Regulatory sequences include transcription control sequences, e.g., sequences which control the initiation, elongation and termination of transcription. In some cases, regulatory sequences include those transcription control sequences. Examples of such regulatory sequences include promoters, enhancers, operators, repressor, transcription terminator sequences.

### Promoters

In some examples, the regulatory sequences are promoters. The regulatory sequences may be promoters that are active in specific types of nuclei. A promoter refers to a nucleic acid sequence that directs the transcription of an operably linked sequence into mRNA. The promoter or promoter region may provide a recognition site for RNA polymerase and the other factors necessary for proper initiation of transcription when a sequence operably linked to a promoter is controlled or driven by the promoter. A promoter may include at least the Core promoter, e.g., a sequence for initiating transcription. The promoter may further at least the Proximal promoter, e.g., a proximal sequence upstream of the gene that tends to contain primary regulatory elements. The promoter may also include the Distal promoter, e.g., the distal sequence upstream of the gene that may contain additional regulatory elements.

The promoters may be from about 50 to about 2000 base pairs (bp), from about 100 to about 1000, from about 50 to about 150, from about 100 to about 200, from about 150 to about 250, from about 200 to about 300, from about 250 to about 350, from about 300 to about 400, from about 350 to about 450, from about 400 to about 500, from about 450 to about 550, from about 500 to about 600, from about 550 to about 650, from about 600 to about 700, from about 650 to about 750, from about 700 to about 800, from about 750 to about 850, from about 800 to about 900, from about 850 to about 950, from about 900 to about 1000, from about 950 to about 1050, from about 1000 to about 1100 in length.

The promoters may include sequences that bind to regulatory proteins. In some examples, the regulatory sequences may be sequences that bind to transcription activators. In certain examples, the regulatory sequences may be sequences that bind to transcription repressors.

In some cases, the promoter may be a constitutive promoter, e.g., U6 and H1 promoters, retroviral Rous sarcoma virus (RSV) LTR promoter, cytomegalovirus (CMV) promoter, SV40 promoter, dihydrofolate reductase promoter, β-actin promoter, phosphoglycerol kinase (PGK) promoter, ubiquitin C, U5 snRNA, U7 snRNA, tRNA promoters or EF1α promoter. In certain cases, the promoter may be a tissue-specific promoter may direct expression primarily in a desired tissue of interest, such as muscle, neuron, bone, skin, blood, specific organs (e.g. liver, pancreas), or particular cell types (e.g. lymphocytes). Examples of tissue-specific promoters include Ick, myogenin, or thy1 promoters. In some embodiments, the promoter may direct expression in a temporal-dependent manner, such as in a cell-cycle dependent or developmental stage-dependent manner, which may or may not also be tissue or cell-type specific. In certain cases, the promoter may be an inducible promoter, e.g., can be activated by a chemical such as doxycycline.

In some cases, a promoter is specific to one or more genes. For examples, the promoter may only regulate (e.g., activates) transcription of the one or more genes, not other genes.

In some cases, the promoters may be cell-specific, tissue-specific, or organ-specific promoters. Example of cell-specific, tissue-specific, or organ-specific promoters include promoter for creatine kinase (for expression in muscle and cardiac tissue), immunoglobulin heavy or light chain promoters (for expression in B cells), smooth muscle alpha-actin promoter. Exemplary tissue-specific promoters for the liver include HMG-COA reductase promoter, sterol regulatory element 1, phosphoenol pyruvate carboxy kinase (PEPCK) promoter, human C-reactive protein (CRP) promoter, human glucokinase promoter, cholesterol 7-alpha hydroylase (CYP-7) promoter, beta-galactosidase alpha-2,6 sialyltransferase promoter, insulin-like growth factor binding protein (IGFBP-1) promoter, aldolase B promoter, human transferrin promoter, and collagen type I promoter. Exemplary tissue-specific promoters for the prostate include the prostatic acid phosphatase (PAP) promoter, prostatic secretory protein of 94 (PSP 94) promoter, prostate specific antigen complex promoter, and human glandular kallikrein gene promoter (hgt-1). Exemplary tissue-specific promoters for gastric tissue include H+/K+-ATPase alpha subunit promoter. Exemplary tissue-specific expression elements for the pancreas include pancreatitis associated protein promoter (PAP), elastase 1 transcriptional enhancer, pancreas specific amylase and elastase enhancer promoter, and pancreatic cholesterol esterase gene promoter. Exemplary tissue-specific promoters for the endometrium include, the uteroglobin promoter. Exemplary tissue-specific promoters for adrenal cells include cholesterol side-chain cleavage (SCC) promoter. Exemplary tissue-specific promoters for the general nervous system include gamma-gamma enolase (neuron-specific enolase, NSE) promoter. Exemplary tissue-specific promoters for the brain include the neurofilament heavy chain (NF-H) promoter. Exemplary tissue-specific promoters for lymphocytes include the human CGL-1/granzyme B promoter, the terminal deoxy transferase (TdT), lambda 5, VpreB, and 1ck (lymphocyte specific tyrosine protein kinase p561ck) promoter, the humans CD2 promoter and its 3′transcriptional enhancer, and the human NK and T cell specific activation (NKG5) promoter. Exemplary tissue-specific promoters for the colon include pp60c-src tyrosine kinase promoter, organ-specific neoantigens (OSNs) promoter, and colon specific antigen-P promoter. Exemplary tissue-specific promoters for breast cells include the human alpha-lactalbumin promoter. Exemplary tissue-specific promoters for the lung include the cystic fibrosis transmembrane conductance regulator (CFTR) gene promoter.

Examples of cell-specific, tissue-specific, or organ-specific promoters may also include those used for expressing the nucleotide sequences within a particular plant tissue (See e.g., WO 2001/098480A2, “Promoters for regulation of plant gene expression”). Examples of such promoters include the lectin (Vodkin, Prog. Clinc. Biol. Res., 138:87-98 (1983); and Lindstrom et al., Dev. Genet., 11:160-167 (1990)), corn alcohol dehydrogenase 1 (Dennis et al., Nucleic Acids Res., 12:3983-4000 (1984)), corn light harvesting complex (Becker, Plant Mol Biol., 20(1): 49-60 (1992); and Bansal et al., Proc. Natl. Acad. Sci. U.S.A., 89:3654-3658 (1992)), corn heat shock protein (Odell et al., Nature (1985) 313:810-812; and Marrs et al., Dev. Genet., 14(1):27-41 (1993)), small subunit RuBP carboxylase (Waksman et al., Nucleic Acids Res., 15(17):7181 (1987); and Berry-Lowe et al., J. Mol. Appl. Genet., 1(6):483-498 (1982)), Ti plasmid mannopine synthase (Ni et al., Plant Mol. Biol., 30(1):77-96 (1996)), Ti plasmid nopaline synthase (Bevan, Nucleic Acids Res., 11(2):369-385 (1983)), petunia chalcone isomerase (Van Tunen et al., EMBO J., 7:1257-1263 (1988)), bean glycine rich protein 1 (Keller et al., Genes Dev., 3:1639-1646 (1989)), truncated CaMV 35s (Odell et al., Nature (1985) 313:810-812), potato patatin (Wenzler et al., Plant Mol. Biol., 13:347-354 (1989)), root cell (Yamamoto et al., Nucleic Acids Res., 18:7449 (1990)), maize zein (Reina et al., Nucleic Acids Res., 18:6425 (1990); Kriz et al., Mol. Gen. Genet., 207:90-98 1987; Wandelt and Feix, Nucleic Acids Res., 17:2354 (1989); Langridge and Feix, Cell, 34:1015-1022 (1983); and Reina et al., Nucleic Acids Res., 18:7449 (1990)), globulin-1 (Belanger et al., Genetics, 129:863-872 (1991)), α-tubulin, cab (Sullivan et al., Mol. Gen. Genet., 215:431-440 (1989)), PEPCase (Cushman et al., Plant Cell, 1(7):715-25 (1989)), R gene complex-associated promoters (Chandler et al., Plant Cell, 1: 1175-1183 (1989)), and chalcone synthase promoters (Franken et al., EMBO J., 10:2605-2612, 1991)). Examples of tissue-specific promoters also include those described in the following references: Yamamoto et al., Plant J (1997) 12(2):255-265; Kawamata et al., Plant Cell Physiol. (1997) 38(7):792-803; Hansen et al., Mol. Gen Genet. (1997) 254(3):337); Russell et al., Transgenic Res. (1997) 6(2):157-168; Rinehart et al., Plant Physiol. (1996) 112(3):1331; Van Camp et al., Plant Physiol. (1996) 112(2):525-535; Canevascini et al., Plant Physiol. (1996) 112(2):513-524; Yamamoto et al., Plant Cell Pkysiol. (1994) 35(5):773-778; Lam, Results Probl. Cell Differ. (1994) 20:181-196; Orozco et al., Plant Mol. Biol. (1993) 23(6):1129-1138; Matsuoka et al., Proc Natl. Acad. Sci. USA (1993) 90(20):9586-9590; and Guevara-Garcia et al., Plant J. (1993) 4(3):495-505; maize phosphoenol carboxylase (PEPC) has been described by Hudspeth & Grula (Plant Molec Biol 12: 579-589 (1989)); leaf-specific promoters such as those described in Yamamoto et al., Plant J. (1997) 12(2):255-265; Kwon et al., Plant Physiol. (1994) 105:357-367; Yamamoto et al., Plant Cell Physiol. (1994) 35(5):773-778; Gotor et al., Plant J. (1993) 3:509-518; Orozco et al., Plant Mol. Biol. (1993) 23(6):1129-1138; and Matsuoka et al., Proc. Natl. Acad. Sci. USA (1993) 90(20):9586-9590.

### Enhancers

In some embodiments, the regulatory sequences may be enhancers. The regulatory sequences may be enhancers active in specific types of nuclei. An enhancer refers to a region of DNA that functions to increase the transcription of one or more genes. More specifically, the term “enhancer”, as used herein, is a DNA regulatory element that enhances, augments, improves, or ameliorates expression of a gene irrespective of its location and orientation vis-à-vis the gene to be expressed, and may be enhancing, augmenting, improving, or ameliorating expression of more than one promoter. In some cases, the enhancer enhances expression from more than one promoter simultaneously. A functional expression enhancing fragment of an enhancer is a shortened or truncated enhancer sequence retaining the enhancing activity.

In some cases, an enhancer may be active in specific types of cells or nuclei (e.g., BCL11A1 enhancer; Canver, et al., Nature. 2015 Nov. 12; 527(7577):192-7). In certain embodiments, an enhancer specific for a type of nuclei may be used in combination with a promoter (e.g., a core promoter).

### Example Regulatory Sequences

In some embodiments, the regulatory sequences comprise endogenous regulatory sequences (or fragments thereof) that control expression of genes in specific types of nuclei. In some cases, the regulatory sequences are derived from endogenous regulatory sequences that control expression of genes in specific types of nuclei. For example, a regulatory sequence may comprise an endogenous regulatory sequence, or a fragment thereof that retains its regulatory activity. In certain examples, a regulatory sequence may comprise a variant of an endogenous regulatory sequence (e.g., mutations) that has substantially the same, higher, or lower regulatory activity compared to the wildtype endogenous regulatory sequence. The activity or strength of a regulatory sequence (e.g., promoter) may be measured by measuring the production (e.g., quantity) of RNA from a reporter gene under control of the regulatory sequence comparing to the production of RNA from the same reporter gene that is under control of a reference regulatory sequence.

In some embodiments, the one or more regulatory sequences are derived from endogenous regulatory sequences that control expression of one or more myocyte nuclei specific genes. In some cases, compositions comprising nucleotide sequences with such regulatory sequences may be specific to nuclei expressing the one or more markers.

In some cases, the nucleic specific genes may be PDK4, PDE4D, DMD, NEB, TTN, SVIL, RP11-434D9.1, MLIP, PDLIM5, EYA4, or any combination thereof. In some cases, the nucleic specific genes may be TTN, NEB, MLIP, SVIL, DMD, NEXN, RP11-362A9.3, PDE4D, FBXO32, TRDN, or any combination thereof. In some cases, the nucleic specific genes may be PDE4D, NEB, TRIM63, MLIP, EYA4, FBXO32, TBC1D8, TTN, GBE1, DMD, or any combination thereof. In some cases, the nucleic specific genes may be RP11-434D9.1, ART3, EMC10, SVIL, NEXN, BCL6, DMD, MYO18B, SESN1, TTN, or any combination thereof. In some cases, the nucleic specific genes may be GBE1, PDE4D, DMD, MLIP, EYA4, NEAT1, ACACB, UBE2E2, TBC1D8, KIAA1217 or any combination thereof. In some cases, the nucleic specific genes may be NEAT1, MALAT1, BCL6, NEXN, DMD, NEDD4, TNNT3, ART3, SESN1, SVIL or any combination thereof. In some cases, the nucleic specific genes may be MLIP, PDE4D, DMD, SVIL, RP11-362A9.3, NEB, TTN, EYA4, NEXN, FBXO32 or any combination thereof.

In some cases, the nuclei specific genes may be one or more from Table 1A. In some cases, the nucleic specific genes may be one or more from cluster 0 of Table 1A. In some cases, the nucleic specific genes may be one or more from cluster 2 of Table 1A. In some cases, the nucleic specific genes may be one or more from cluster 6 of Table 1A.

TABLE 1A
Single Nuclei TST Preparation (Myocyte clusters)

| | | | | | | | |
|---|---|---|---|---|---|---|---|
| cluster | gene | tstat | log2FC | pval | pval_adj | percent_exp | mean_exp |

Cluster 0 Myocyte

| | | | | | | | |
|---|---|---|---|---|---|---|---|
| 0 | PDK4 | 107.4918594 | 4.874361515 | 0 | 0 | 0.963989442 | 3.928916693 |
| 0 | PDE4D | 81.22364807 | 3.759793043 | 0 | 0 | 0.953996983 | 4.191121101 |
| 0 | DMD | 74.39215851 | 3.19527483 | 0 | 0 | 0.962292609 | 3.920099974 |
| 0 | NEB | 73.99095917 | 3.440121174 | 0 | 0 | 0.925904977 | 3.625823736 |
| 0 | TTN | 72.93958282 | 3.171783447 | 0 | 0 | 0.954751131 | 3.639686108 |
| 0 | SVIL | 72.05633545 | 3.19019866 | 0 | 0 | 0.914969834 | 3.439188719 |
| 0 | RP11-434D9.1 | 67.86113739 | 3.645878553 | 0 | 0 | 0.788084465 | 3.496703625 |
| 0 | MLIP | 67.6209259 | 3.131190777 | 0 | 0 | 0.860105581 | 3.163345098 |
| 0 | PDLIM5 | 65.51317596 | 2.941664219 | 0 | 0 | 0.892722474 | 3.329166174 |
| 0 | EYA4 | 65.12088776 | 3.119018555 | 0 | 0 | 0.821455505 | 3.067982674 |
| 0 | EMC10 | 64.1467514 | 3.553575277 | 0 | 0 | 0.750942685 | 3.328134537 |
| 0 | RP11-362A9.3 | 63.44179916 | 3.229053736 | 0 | 0 | 0.808069382 | 3.235378027 |
| 0 | PDLIM3 | 62.69000244 | 3.344083071 | 0 | 0 | 0.809577677 | 3.470199347 |
| 0 | ACACB | 62.46204376 | 2.9269135 | 0 | 0 | 0.829751131 | 3.067731857 |
| 0 | RYR1 | 60.51604462 | 2.990078211 | 0 | 0 | 0.757918552 | 2.851527929 |
| 0 | TTN-AS1 | 60.27650452 | 2.773089409 | 0 | 0 | 0.820324284 | 2.967164278 |
| 0 | MYPN | 60.18128204 | 3.018295527 | 0 | 0 | 0.755467572 | 2.87583971 |
| 0 | CAP2 | 59.11434174 | 2.941689014 | 0 | 0 | 0.772624434 | 2.979908466 |
| 0 | NEXN | 59.09638596 | 2.900067806 | 0 | 0 | 0.814102564 | 3.197187662 |
| 0 | TRIM63 | 58.35753632 | 2.949579716 | 0 | 0 | 0.793363499 | 3.161252737 |
| 0 | MYO18B | 57.91911697 | 3.073394775 | 0 | 0 | 0.708710407 | 2.91880846 |
| 0 | TBC1D8 | 56.47376251 | 2.703377962 | 0 | 0 | 0.801847662 | 3.02310276 |
| 0 | FBXO32 | 54.8805275 | 2.840924025 | 0 | 0 | 0.80015083 | 3.286741495 |
| 0 | NEAT1 | 53.12233353 | 1.952271342 | 0 | 0 | 0.987179487 | 4.761385441 |
| 0 | WDPCP | 51.72141647 | 2.764147282 | 0 | 0 | 0.720211161 | 3.137215853 |
| 0 | TRDN | 51.42260361 | 2.701838255 | 0 | 0 | 0.756221719 | 3.117156029 |
| 0 | MYOM1 | 50.28709412 | 2.832439899 | 0 | 0 | 0.648190045 | 2.972721815 |
| 0 | SLC16A10 | 49.67194366 | 2.754383564 | 0 | 0 | 0.630656109 | 2.726503849 |
| 0 | BACH1 | 49.47677994 | 2.503251791 | 0 | 0 | 0.718891403 | 2.805686474 |
| 0 | RBFOX1 | 49.39567947 | 2.583550215 | 0 | 0 | 0.706636501 | 2.8932724 |
| 0 | ABLIM2 | 49.31342316 | 2.560685635 | 0 | 0 | 0.686651584 | 2.730609179 |
| 0 | GPATCH8 | 49.17394257 | 2.269079924 | 0 | 0 | 0.805241327 | 2.974346638 |
| 0 | FAM184B | 48.97390366 | 2.606703997 | 0 | 0 | 0.667043741 | 2.766336203 |
| 0 | SNX1 | 48.29244995 | 2.665385246 | 0 | 0 | 0.614064857 | 2.665727615 |
| 0 | MYBPC1 | 48.13316727 | 2.894086123 | 0 | 0 | 0.733597285 | 3.719248056 |
| 0 | TP63 | 47.90929794 | 2.746870279 | 0 | 0 | 0.618778281 | 2.891594887 |
| 0 | PDE4DIP | 47.37867737 | 2.159953117 | 0 | 0 | 0.823152338 | 2.994854689 |
| 0 | KCNQ5 | 46.7166481 | 2.642562389 | 0 | 0 | 0.658559578 | 3.065823317 |
| 0 | CMYA5 | 46.30514145 | 2.448080778 | 0 | 0 | 0.654788839 | 2.685562611 |
| 0 | CLIP1 | 45.83179855 | 2.290398359 | 0 | 0 | 0.704939668 | 2.734212637 |
| 0 | KIF1B | 45.29418945 | 2.240287781 | 0 | 0 | 0.729449472 | 2.852203608 |
| 0 | OBSCN | 42.29396057 | 2.333571911 | 0 | 0 | 0.6076546 | 2.681447029 |
| 0 | MPV17 | 41.82595825 | 2.386232376 | 0 | 0 | 0.561840121 | 2.555136919 |
| 0 | PDE7A | 41.3157692 | 2.216132402 | 0 | 0 | 0.614630468 | 2.616008282 |
| 0 | MYOT | 41.29111099 | 2.456134081 | 0 | 0 | 0.529788839 | 2.567605972 |
| 0 | RP11-381K20.2 | 41.11019135 | 2.586491823 | 0 | 0 | 0.483220211 | 2.529435396 |
| 0 | GBE1 | 41.02796173 | 2.146986246 | 0 | 0 | 0.722473605 | 3.133983374 |
| 0 | LPIN1 | 40.9535141 | 2.227391481 | 0 | 0 | 0.621040724 | 2.750242949 |
| 0 | ARPP21 | 40.91569901 | 2.530961275 | 0 | 0 | 0.504901961 | 2.658741236 |
| 0 | MYOM3 | 40.26186371 | 2.606243372 | 0 | 0 | 0.466063348 | 2.59230566 |
| 0 | GRIK1 | 39.97338104 | 2.42224741 | 0 | 0 | 0.528846154 | 2.706529379 |
| 0 | PCNT | 39.95726776 | 2.275577545 | 0 | 0 | 0.553921569 | 2.554341555 |
| 0 | BCL6 | 39.67106247 | 1.858748794 | 0 | 0 | 0.815045249 | 3.212130308 |
| 0 | HDAC4 | 39.60694504 | 2.148341894 | 0 | 0 | 0.601244344 | 2.651978493 |
| 0 | GTF2IRD1 | 39.47352982 | 2.342724085 | 0 | 0 | 0.517533937 | 2.537209749 |
| 0 | CPED1 | 39.3083992 | 2.353335857 | 0 | 0 | 0.547511312 | 2.829708576 |
| 0 | UBE2E2 | 39.2369194 | 1.891581893 | 0 | 0 | 0.745852187 | 2.867790222 |
| 0 | ALK | 38.9556427 | 2.387090921 | 2.6789E−308 | 7.4946E−306 | 0.511689291 | 2.669386148 |
| 0 | ASB5 | 38.62573624 | 2.205428839 | 1.1852E−304 | 3.2143E−302 | 0.564102564 | 2.621860266 |
| 0 | RP11-64D24.2 | 38.40656662 | 2.014400244 | 4.9552E−302 | 1.3039E−299 | 0.659125189 | 2.839048386 |
| 0 | RP11-532N4.2 | 38.19285202 | 2.421304941 | 1.8926E−297 | 4.8366E−295 | 0.512443439 | 2.966953039 |
| 0 | SESN1 | 37.92779541 | 1.934857845 | 3.4352E−295 | 8.6951E−293 | 0.683446456 | 2.794881582 |
| 0 | ASB8 | 37.89464951 | 2.341087341 | 1.0803E−292 | 2.7087E−290 | 0.483597285 | 2.53686285 |
| 0 | SPDYE2 | 37.64619827 | 1.950896263 | 5.4748E−291 | 1.3598E−288 | 0.657616893 | 2.82236743 |
| 0 | ERMP1 | 37.6241188 | 2.477949858 | 8.3621E−288 | 2.0578E−285 | 0.437217195 | 2.505386591 |
| 0 | FAM134B | 37.13576889 | 2.215013027 | 5.0145E−283 | 1.2006E−280 | 0.545625943 | 2.738149643 |
| 0 | NEDD4 | 37.00656128 | 2.110789537 | 2.2341E−281 | 5.2546E−279 | 0.576168929 | 2.779803991 |
| 0 | DEPTOR | 36.89151382 | 2.147603989 | 2.125E−279 | 4.911E−277 | 0.533748115 | 2.620849848 |
| 0 | CTNNA3 | 36.8684082 | 2.116257906 | 4.0096E−279 | 9.1864E−277 | 0.542420814 | 2.617680311 |
| 0 | RIF1 | 36.80512238 | 2.052874565 | 1.4604E−278 | 3.3173E−276 | 0.577677225 | 2.676970243 |
| 0 | MAST2 | 36.17778778 | 2.037740707 | 1.5537E−269 | 3.4126E−267 | 0.547511312 | 2.570504904 |
| 0 | ACTN2 | 36.1384964 | 2.214659214 | 2.0828E−268 | 4.5373E−266 | 0.490007541 | 2.569849491 |
| 0 | PPP1R27 | 35.44884491 | 2.254201651 | 1.0896E−258 | 2.2623E−256 | 0.472473605 | 2.678123713 |
| 0 | ART3 | 35.37556839 | 2.56644845 | 6.779E−256 | 1.3753E−253 | 0.380279035 | 2.546901464 |
| 0 | TACC2 | 35.30202866 | 2.013589144 | 8.5426E−258 | 1.76E−255 | 0.556372549 | 2.61273098 |
| 0 | PEX5L | 35.18170929 | 2.163415194 | 8.4609E−256 | 1.7035E−253 | 0.523944193 | 2.813352108 |
| 0 | ATP1A2 | 34.9576149 | 2.110675335 | 2.892E−252 | 5.7791E−250 | 0.478129713 | 2.467801809 |
| 0 | TTC19 | 34.74345779 | 1.840337515 | 2.8966E−250 | 5.7025E−248 | 0.597285068 | 2.589784861 |
| 0 | CLTCL1 | 34.71992493 | 2.149417639 | 7.5242E−249 | 1.4704E−246 | 0.468702866 | 2.576246262 |
| 0 | DPF3 | 34.65425873 | 2.343105078 | 3.8781E−247 | 7.4687E−245 | 0.402714932 | 2.437114239 |
| 0 | SF3A3 | 34.43700409 | 2.34385705 | 2.5829E−244 | 4.8004E−242 | 0.405165913 | 2.541976452 |
| 0 | SLC20A2 | 34.33453751 | 1.970783234 | 1.7747E−244 | 3.3216E−242 | 0.535633484 | 2.629187107 |
| 0 | RP11-768F21.1 | 33.71094513 | 2.140252829 | 1.6277E−235 | 2.9428E−233 | 0.448529412 | 2.552348614 |
| 0 | ACSL1 | 33.69581985 | 1.93145144 | 5.2939E−236 | 9.6367E−234 | 0.527526395 | 2.554764032 |
| 0 | LRRFIP2 | 33.32804489 | 1.80058074 | 2.0101E−231 | 3.5379E−229 | 0.575414781 | 2.57872963 |
| 0 | CAPN7 | 33.14011765 | 1.942094088 | 1.8278E−228 | 3.1544E−226 | 0.496040724 | 2.54025507 |
| 0 | DAPK2 | 32.89918518 | 2.353608847 | 6.5695E−224 | 1.0912E−221 | 0.363310709 | 2.429645061 |
| 0 | MAP4K1 | 32.87556458 | 2.403526545 | 1.6848E−223 | 2.7811E−221 | 0.351998492 | 2.402886868 |
| 0 | RP3-355L5.4 | 32.6476593 | 2.325052261 | 8.5627E−221 | 1.3709E−218 | 0.361802413 | 2.372825384 |
| 0 | CTC-431G16.2 | 32.09917068 | 2.361427546 | 1.2667E−213 | 1.902E−211 | 0.344645551 | 2.414256811 |
| 0 | FKBP5 | 31.99011993 | 1.203566432 | 1.9359E−213 | 2.8905E−211 | 0.957579186 | 3.873175383 |
| 0 | CACNA2D1 | 31.94462395 | 1.612888217 | 7.2998E−214 | 1.1023E−211 | 0.671568627 | 2.765884638 |
| 0 | AKAP6 | 31.59602928 | 1.909113407 | 5.8914E−209 | 8.6987E−207 | 0.484162896 | 2.581261396 |
| 0 | USP54 | 31.52794456 | 1.763552308 | 2.4205E−208 | 3.5542E−206 | 0.540158371 | 2.567929506 |
| 0 | SYT2 | 31.38165474 | 2.125917912 | 1.8219E−205 | 2.6316E−203 | 0.385935143 | 2.466381311 |
| 0 | NFE2L3 | 31.27213097 | 2.128283501 | 3.7251E−204 | 5.2942E−202 | 0.386500754 | 2.439472437 |
| 0 | RP11-266N13.2 | 31.02531433 | 2.224796057 | 1.0161E−200 | 1.3992E−198 | 0.352375566 | 2.435158253 |
| 0 | SGCD | 30.83654022 | 1.940677881 | 3.1206E−199 | 4.275E−197 | 0.436840121 | 2.534911394 |
| 0 | KCNMA1 | 30.77889061 | 1.963105798 | 1.5716E−198 | 2.142E−196 | 0.440422323 | 2.615980625 |
| 0 | CD70 | 30.7460022 | 2.562033892 | 2.0985E−196 | 2.8026E−194 | 0.288650075 | 2.442364216 |
| 0 | RP11-144F15.1 | 30.73619461 | 2.380492926 | 1.0045E−196 | 1.3552E−194 | 0.315233786 | 2.422985077 |
| 0 | UBAC1 | 30.62685966 | 2.00123477 | 1.9633E−196 | 2.6353E−194 | 0.397624434 | 2.402034283 |
| 0 | ATG14 | 30.56244469 | 1.989482403 | 9.7577E−196 | 1.2966E−193 | 0.406297134 | 2.443701744 |
| 0 | ASB2 | 30.40594482 | 1.885886312 | 4.4027E−194 | 5.7926E−192 | 0.432126697 | 2.392113686 |
| 0 | LRRTM3 | 30.21966934 | 2.118291378 | 2.9936E−191 | 3.881E−189 | 0.367269985 | 2.550768375 |
| 0 | FOXO1 | 30.20650482 | 1.478382587 | 2.3621E−192 | 3.0925E−190 | 0.731711916 | 2.9133358 |
| 0 | SLC7A2 | 30.05247116 | 2.197748184 | 4.6208E−189 | 5.876E−187 | 0.339177979 | 2.406337261 |
| 0 | OSBPL6 | 30.012743 | 1.988159418 | 4.0837E−189 | 5.2179E−187 | 0.397435897 | 2.491722345 |
| 0 | DENND2C | 29.95227051 | 2.057172775 | 2.969E−188 | 3.722E−186 | 0.378205128 | 2.54251647 |
| 0 | EPS15L1 | 29.88773346 | 1.704733253 | 3.1211E−188 | 3.8944E−186 | 0.506975867 | 2.487792015 |
| 0 | SLC22A4 | 29.88521957 | 2.195148706 | 5.2049E−187 | 6.434E−185 | 0.332390649 | 2.398044348 |
| 0 | OSBPL9 | 29.76552391 | 1.541874886 | 5.2587E−187 | 6.4704E−185 | 0.625942685 | 2.714361429 |
| 0 | PPP2R3A | 29.67979431 | 1.836186886 | 2.0077E−185 | 2.4476E−183 | 0.434012066 | 2.433994532 |
| 0 | RP11-309L24.2 | 29.65971184 | 2.362791777 | 3.954E−184 | 4.755E−182 | 0.30260181 | 2.458113194 |
| 0 | MTFR1 | 29.64041328 | 1.892938733 | 8.105E−185 | 9.7912E−183 | 0.406485671 | 2.368260622 |
| 0 | SPRTN | 29.43842697 | 1.972465396 | 2.1591E−182 | 2.5617E−180 | 0.39046003 | 2.492576838 |
| 0 | IL12RB2 | 29.40408516 | 1.967996478 | 5.8388E−182 | 6.8968E−180 | 0.386877828 | 2.443862915 |
| 0 | CEP128 | 29.29460526 | 2.128109932 | 3.1992E−180 | 3.7129E−178 | 0.361802413 | 2.907363415 |
| 0 | HADHB | 29.17480469 | 1.785540104 | 1.5705E−179 | 1.8147E−177 | 0.443250377 | 2.510764837 |
| 0 | FAM166B | 29.12709427 | 2.603777647 | 3.1029E−177 | 3.5699E−175 | 0.259803922 | 2.572217464 |
| 0 | MLIP-AS1 | 29.05377579 | 2.243587494 | 3.8199E−177 | 4.3385E−175 | 0.303167421 | 2.311602592 |
| 0 | TMEM233 | 29.01055145 | 2.017206669 | 3.1361E−177 | 3.5926E−175 | 0.363122172 | 2.462609291 |
| 0 | HSPB7 | 28.99982643 | 1.958457708 | 3.5377E−177 | 4.0352E−175 | 0.376131222 | 2.425681829 |
| 0 | CH17-189H20.1 | 28.92128944 | 1.559741259 | 5.6391E−177 | 6.3775E−175 | 0.560143288 | 2.54373312 |
| 0 | SOX6 | 28.80459595 | 1.779551864 | 2.8768E−175 | 3.1857E−173 | 0.439856712 | 2.531128645 |
| 0 | CUX1 | 28.7573452 | 1.460805416 | 3.6415E−175 | 4.0158E−173 | 0.634615385 | 2.634729624 |
| 0 | RP11-701H24.9 | 28.75137138 | 1.971463323 | 3.56E−174 | 3.8461E−172 | 0.358408748 | 2.386003971 |
| 0 | ANKRD2 | 28.611269 | 2.06455493 | 1.259E−172 | 1.3331E−170 | 0.377262443 | 3.035745621 |
| 0 | CKMT2 | 28.58091354 | 2.052670002 | 4.2715E−172 | 4.4694E−170 | 0.33974359 | 2.459234953 |
| 0 | ZCCHC11 | 28.4266777 | 1.621710062 | 4.286E−171 | 4.4496E−169 | 0.501319759 | 2.538511992 |
| 0 | MIR133A1HG | 28.41670036 | 2.405048847 | 1.3791E−169 | 1.4043E−167 | 0.273190045 | 2.568895578 |
| 0 | PDXDC1 | 28.28449059 | 1.262985349 | 1.1146E−169 | 1.1437E−167 | 0.789215686 | 2.892272234 |
| 0 | AC016831.7 | 28.28355217 | 1.334462881 | 1.1587E−169 | 1.1845E−167 | 0.789215686 | 3.060251951 |
| 0 | TPD52L1 | 28.26714897 | 1.726905942 | 3.1721E−169 | 3.2055E−167 | 0.459841629 | 2.582608461 |
| 0 | LDB3 | 28.12636375 | 1.893344998 | 2.3394E−167 | 2.2943E−165 | 0.389894419 | 2.573708057 |
| 0 | TMEM161B-AS1 | 28.1040535 | 1.892350078 | 5.5351E−167 | 5.4083E−165 | 0.37254902 | 2.440244913 |
| 0 | SPPL2A | 28.05758667 | 1.651353717 | 7.6339E−167 | 7.4317E−165 | 0.468137255 | 2.474246264 |
| 0 | PHKG1 | 27.98149681 | 1.973104835 | 2.251E−165 | 2.1676E−163 | 0.340120664 | 2.396347761 |
| 0 | ADAMTS17 | 27.92046738 | 1.963477015 | 9.7806E−165 | 9.3168E−163 | 0.348039216 | 2.471639872 |
| 0 | RP11-428F8.2 | 27.75865555 | 2.101187706 | 1.0781E−162 | 1.0089E−160 | 0.308257919 | 2.380795479 |
| 0 | AHCYL1 | 27.7516346 | 1.959301233 | 9.8281E−163 | 9.2298E−161 | 0.342383107 | 2.497029781 |
| 0 | ZNF565 | 27.73575211 | 1.562548995 | 2.3655E−163 | 2.2373E−161 | 0.509992459 | 2.491806507 |
| 0 | IDNK | 27.5756321 | 2.217142582 | 1.8144E−160 | 1.6628E−158 | 0.282616893 | 2.360355616 |
| 0 | SRL | 27.55795479 | 1.989269495 | 1.27E−160 | 1.1679E−158 | 0.326168929 | 2.349868298 |
| 0 | FASTKD1 | 27.52350616 | 1.948456168 | 2.4792E−160 | 2.2643E−158 | 0.339555053 | 2.391572237 |
| 0 | ERAP1 | 27.5077877 | 1.660863996 | 1.1498E−160 | 1.061E−158 | 0.463800905 | 2.587401152 |
| 0 | KIAA1217 | 27.49358177 | 1.379873037 | 8.6233E−161 | 7.9854E−159 | 0.72775264 | 3.001834869 |
| 0 | ZBTB43 | 27.48158836 | 1.798199415 | 4.378E−160 | 3.9711E−158 | 0.378770739 | 2.364684343 |
| 0 | SMG1 | 27.47232437 | 1.646218061 | 3.0785E−160 | 2.802E−158 | 0.443438914 | 2.411998987 |
| 0 | THUMPD3-AS1 | 27.39427757 | 1.614851713 | 2.0619E−159 | 1.845E−157 | 0.459841629 | 2.459041834 |
| 0 | CFAP61 | 27.30050087 | 1.859304786 | 5.2709E−158 | 4.6695E−156 | 0.364441931 | 2.466628551 |
| 0 | PIGB | 27.26112556 | 1.896948695 | 1.6452E−157 | 1.4527E−155 | 0.345399698 | 2.3673563 |
| 0 | FILIP1 | 27.13337898 | 1.705604196 | 2.1529E−156 | 1.8884E−154 | 0.407616893 | 2.40392971 |
| 0 | SIL1 | 27.03792953 | 1.579869986 | 1.5489E−155 | 1.3541E−153 | 0.467571644 | 2.432379007 |
| 0 | MACROD2 | 26.98497772 | 1.690466642 | 7.7716E−155 | 6.772E−153 | 0.424019608 | 2.510978222 |
| 0 | PBX1 | 26.92970085 | 1.587498426 | 2.3751E−154 | 2.0561E−152 | 0.476621418 | 2.587225199 |
| 0 | SSH2 | 26.9202652 | 1.26530385 | 1.9701E−154 | 1.7111E−152 | 0.766968326 | 2.957236528 |
| 0 | PPARGC1A | 26.7924118 | 1.768297553 | 1.1162E−152 | 9.5077E−151 | 0.391214178 | 2.510117054 |
| 0 | LRRC2 | 26.79132462 | 1.791660786 | 1.4493E−152 | 1.2267E−150 | 0.367269985 | 2.366792202 |
| 0 | FILIP1L | 26.65704155 | 1.390505075 | 1.3934E−151 | 1.1683E−149 | 0.6510181 | 2.853925705 |
| 0 | THRB | 26.57149315 | 1.413954496 | 1.2499E−150 | 1.0221E−148 | 0.583898944 | 2.622326374 |
| 0 | ABCA5 | 26.50375748 | 1.638440967 | 1.3404E−149 | 1.0894E−147 | 0.417797888 | 2.428410053 |
| 0 | C10orf71 | 26.47002602 | 2.173267365 | 1.9604E−148 | 1.5836E−146 | 0.265082956 | 2.326788902 |
| 0 | YBX3 | 26.46485138 | 1.349503875 | 1.6938E−149 | 1.3724E−147 | 0.62254902 | 2.61923027 |
| 0 | TARID | 26.33161926 | 1.842792749 | 1.6245E−147 | 1.3044E−145 | 0.339555053 | 2.370361567 |
| 0 | ZFAND3 | 26.2837677 | 1.242448807 | 1.478E−147 | 1.1903E−145 | 0.718514329 | 2.777693033 |
| 0 | CTA-125H2.1 | 26.13569641 | 2.26801157 | 8.2514E−145 | 6.5073E−143 | 0.246229261 | 2.331311464 |
| 0 | PPP1R12B | 26.07549477 | 1.473625779 | 3.1182E−145 | 2.4665E−143 | 0.505279035 | 2.527329922 |
| 0 | CFLAR-AS1 | 26.02613449 | 1.721086025 | 2.1743E−144 | 1.7046E−142 | 0.370286576 | 2.404073238 |
| 0 | MAP4 | 25.88067055 | 1.345818996 | 3.1009E−143 | 2.4168E−141 | 0.595022624 | 2.619360685 |
| 0 | KLF13 | 25.70898056 | 1.974018693 | 1.2188E−140 | 9.3349E−139 | 0.302036199 | 2.659307241 |
| 0 | P4HA1 | 25.65909004 | 1.63716197 | 1.1794E−140 | 9.0594E−139 | 0.423642534 | 2.678839207 |
| 0 | SLC22A3 | 25.45442581 | 2.222711802 | 9.5535E−138 | 7.1927E−136 | 0.247737557 | 2.566180944 |
| 0 | VCL | 25.35151672 | 1.612629294 | 1.9544E−137 | 1.4673E−135 | 0.398944193 | 2.448857784 |
| 0 | MLK7-AS1 | 25.31246376 | 1.646990061 | 5.3774E−137 | 4.0258E−135 | 0.380090498 | 2.411206484 |
| 0 | NFATC3 | 25.25158501 | 1.543624282 | 1.8968E−136 | 1.4121E−134 | 0.421945701 | 2.439442635 |
| 0 | FSD2 | 25.16786766 | 2.018451691 | 4.7189E−135 | 3.455E−133 | 0.27168175 | 2.404105902 |
| 0 | HOMER2 | 25.16110229 | 1.957717061 | 5.0721E−135 | 3.7033E−133 | 0.279977376 | 2.362638712 |
| 0 | AC016723.4 | 25.07747269 | 2.032085896 | 3.7944E−134 | 2.7404E−132 | 0.28092006 | 2.648937225 |
| 0 | SAMD8 | 25.05935478 | 1.73467052 | 3.0965E−134 | 2.2424E−132 | 0.335030166 | 2.367583036 |
| 0 | STIM1 | 25.04545212 | 1.443477511 | 1.9941E−134 | 1.452E−132 | 0.480769231 | 2.517015219 |
| 0 | ACYP2 | 25.02656937 | 1.346690655 | 2.6224E−134 | 1.9043E−132 | 0.550904977 | 2.557458401 |
| 0 | SLC7A6OS | 24.99321938 | 1.506975293 | 8.0126E−134 | 5.771E−132 | 0.437028658 | 2.486439705 |
| 0 | PRKG1 | 24.94962502 | 1.373760581 | 1.4652E−133 | 1.0525E−131 | 0.62066365 | 2.920557737 |
| 0 | SLC8A3 | 24.87569618 | 2.099503517 | 5.7125E−132 | 4.0594E−130 | 0.248491704 | 2.341637135 |
| 0 | RIC1 | 24.71229172 | 1.462291002 | 5.6625E−131 | 3.9707E−129 | 0.447963801 | 2.47153163 |
| 0 | SORT1 | 24.5699749 | 1.895567894 | 4.5088E−129 | 3.1287E−127 | 0.279600302 | 2.343831062 |
| 0 | EIF4E2 | 24.50966835 | 1.684689879 | 1.0294E−128 | 7.088E−127 | 0.335218703 | 2.335145473 |
| 0 | RP11-118M9.3 | 24.46744156 | 1.881493449 | 4.2103E−128 | 2.8766E−126 | 0.282616893 | 2.346778393 |
| 0 | MSI2 | 24.32740784 | 1.444848776 | 4.1187E−127 | 2.7782E−125 | 0.437217195 | 2.398835659 |
| 0 | ITIH6 | 24.16289902 | 2.384972572 | 1.6835E−124 | 1.0913E−122 | 0.197586727 | 2.336009264 |
| 0 | COL14A1 | 24.06236458 | 1.665327191 | 2.6497E−124 | 1.7134E−122 | 0.342948718 | 2.495230913 |
| 0 | SLC24A1 | 24.04819489 | 1.765807748 | 4.6854E−124 | 3.0151E−122 | 0.300904977 | 2.32196641 |
| 0 | RP11-83A24.2 | 24.04401207 | 1.537723541 | 3.1121E−124 | 2.0075E−122 | 0.389517345 | 2.430303812 |
| 0 | C1orf21 | 23.99150658 | 1.24766314 | 6.8126E−124 | 4.3628E−122 | 0.593325792 | 2.632619619 |
| 0 | RP11-665C16.5 | 23.97736931 | 1.822702169 | 2.839E−123 | 1.8007E−121 | 0.283371041 | 2.341326952 |
| 0 | SPAG1 | 23.97341728 | 2.049767256 | 5.4544E−123 | 3.427E−121 | 0.238687783 | 2.369683743 |
| 0 | FAM168A | 23.9493618 | 1.642556071 | 3.7662E−123 | 2.3775E−121 | 0.346907994 | 2.538237095 |
| 0 | STAC3 | 23.90649223 | 1.735914826 | 1.0796E−122 | 6.7512E−121 | 0.304864253 | 2.296301126 |
| 0 | PLIN5 | 23.74397469 | 1.975425959 | 7.4693E−121 | 4.6058E−119 | 0.245098039 | 2.282773733 |
| 0 | RP11-111E14.1 | 23.70503807 | 2.14430809 | 2.8324E−120 | 1.7345E−118 | 0.218514329 | 2.361370564 |
| 0 | ZNF385B | 23.68051529 | 1.807623029 | 2.1347E−120 | 1.3103E−118 | 0.291855204 | 2.579540014 |
| 0 | C2orf88 | 23.68046188 | 1.869626045 | 2.8408E−120 | 1.7356E−118 | 0.269042232 | 2.427902699 |
| 0 | NRG2 | 23.63715172 | 1.960453868 | 8.8571E−120 | 5.399E−118 | 0.24754902 | 2.378124237 |
| 0 | FBXL13 | 23.57162476 | 1.778501272 | 2.3228E−119 | 1.4094E−117 | 0.285822021 | 2.3534832 |
| 0 | NEDD4L | 23.56943512 | 1.860380054 | 3.1162E−119 | 1.8822E−117 | 0.269230769 | 2.428873539 |
| 0 | FAM160A1 | 23.54128647 | 1.689959168 | 3.7258E−119 | 2.2403E−117 | 0.312971342 | 2.394453287 |
| 0 | AC013461.1 | 23.5241642 | 1.312118888 | 2.9157E−119 | 1.7651E−117 | 0.503770739 | 2.514899969 |
| 0 | RP11-701P16.2 | 23.46611786 | 2.113039255 | 6.2582E−118 | 3.7209E−116 | 0.23453997 | 2.696735859 |
| 0 | HECTD1 | 23.43330956 | 1.366761327 | 2.4669E−118 | 1.4767E−116 | 0.451357466 | 2.448099852 |
| 0 | RPS6KA3 | 23.43174744 | 1.497025967 | 3.1579E−118 | 1.886E−116 | 0.385746606 | 2.453210115 |
| 0 | PPM1B | 23.4263401 | 1.227343321 | 2.3416E−118 | 1.4048E−116 | 0.582579186 | 2.60257268 |
| 0 | RP5-1042K10.10 | 23.30681419 | 1.841368914 | 9.7466E−117 | 5.7692E−115 | 0.263197587 | 2.304958344 |
| 0 | PLCD4 | 23.29527473 | 2.043464184 | 2.081E−116 | 1.229E−114 | 0.2239819 | 2.282598019 |
| 0 | ANO5 | 23.26485252 | 1.900155544 | 2.9062E−116 | 1.7088E−114 | 0.248868778 | 2.303283453 |
| 0 | EEPD1 | 23.23394394 | 1.501790643 | 2.3955E−116 | 1.4117E−114 | 0.382352941 | 2.46750164 |
| 0 | HOMER1 | 23.20811272 | 2.109878063 | 1.6373E−115 | 9.5638E−114 | 0.215874811 | 2.414557457 |
| 0 | ATP1B1 | 23.12930107 | 1.522641301 | 2.42E−115 | 1.4074E−113 | 0.374057315 | 2.459162951 |
| 0 | ARID5B | 23.12306404 | 1.179017425 | 1.9217E−115 | 1.12E−113 | 0.626696833 | 2.711341381 |
| 0 | NRAP | 23.05957031 | 1.815979838 | 2.0141E−114 | 1.1662E−112 | 0.263951735 | 2.295211792 |
| 0 | CTA-125H2.2 | 23.01950645 | 2.382419825 | 1.5899E−113 | 9.1261E−112 | 0.180241327 | 2.284170151 |
| 0 | BEST3 | 23.00809097 | 1.7206707 | 5.447E−114 | 3.1402E−112 | 0.286199095 | 2.317137003 |
| 0 | CAPN9 | 22.96586037 | 2.117233038 | 2.8023E−113 | 1.6016E−111 | 0.210784314 | 2.354087353 |
| 0 | RP11-724O16.1 | 22.93104362 | 2.478504419 | 1.1635E−112 | 6.6075E−111 | 0.171945701 | 2.290398836 |
| 0 | ATP2A2 | 22.8723259 | 1.404018998 | 5.7453E−113 | 3.2697E−111 | 0.427978884 | 2.551897526 |
| 0 | EGF | 22.83487701 | 1.916780949 | 3.4213E−112 | 1.9305E−110 | 0.240573152 | 2.362320662 |
| 0 | ABHD15-AS1 | 22.82660294 | 1.250247717 | 1.3258E−112 | 7.5132E−111 | 0.539781297 | 2.590791225 |
| 0 | SORBS1 | 22.80602837 | 1.345227718 | 2.206E−112 | 1.2474E−110 | 0.484728507 | 2.650812864 |
| 0 | TPM3 | 22.74324989 | 1.279677153 | 7.8725E−112 | 4.4048E−110 | 0.559577677 | 2.788368225 |
| 0 | VWA8 | 22.71010208 | 1.444474697 | 2.2834E−111 | 1.2696E−109 | 0.375377074 | 2.371481419 |
| 0 | CSPP1 | 22.68803406 | 1.463824749 | 3.8312E−111 | 2.1213E−109 | 0.370098039 | 2.400758505 |
| 0 | PHKA1 | 22.63566399 | 1.735862374 | 1.9035E−110 | 1.0496E−108 | 0.274321267 | 2.388457775 |
| 0 | ATP2A1 | 22.54701614 | 2.077362537 | 2.353E−109 | 1.2894E−107 | 0.212104072 | 2.545579433 |
| 0 | ITGAD | 22.52986336 | 2.110688925 | 3.6565E−109 | 1.9954E−107 | 0.201546003 | 2.262410164 |
| 0 | MALAT1 | 22.49385262 | 0.655844808 | 2.8481E−109 | 1.5575E−107 | 0.998303167 | 6.769818306 |
| 0 | MAP4K3 | 22.48801231 | 1.314148188 | 2.3107E−109 | 1.2688E−107 | 0.44306184 | 2.420478344 |
| 0 | BIRC6-AS2 | 22.44724655 | 1.255597115 | 5.1367E−109 | 2.7918E−107 | 0.485294118 | 2.431963444 |
| 0 | NPAS2 | 22.36970139 | 1.482756138 | 3.4335E−108 | 1.8435E−106 | 0.360671192 | 2.421921492 |
| 0 | ANKRD23 | 22.2217083 | 1.79494381 | 1.4342E−106 | 7.6538E−105 | 0.247171946 | 2.310085535 |
| 0 | SPTB | 22.19586182 | 1.788213372 | 2.4569E−106 | 1.3008E−104 | 0.246606335 | 2.244307518 |
| 0 | ACACA | 22.18205452 | 1.432570457 | 1.9527E−106 | 1.038E−104 | 0.36821267 | 2.450649977 |
| 0 | TIAM2 | 22.17705727 | 1.34013164 | 1.8155E−106 | 9.6695E−105 | 0.413650075 | 2.387989521 |
| 0 | CKMT2-AS1 | 22.16284561 | 1.635911703 | 3.9385E−106 | 2.0769E−104 | 0.288273002 | 2.39309597 |
| 0 | PPM1L | 22.13741875 | 1.216492414 | 3.5686E−106 | 1.8856E−104 | 0.541666667 | 2.636632681 |
| 0 | ZFAND1 | 21.97161293 | 1.588538885 | 2.0053E−104 | 1.0491E−102 | 0.30260181 | 2.467734575 |
| 0 | MEF2C-AS1 | 21.95544624 | 1.696461916 | 3.306E−104 | 1.7228E−102 | 0.268853695 | 2.394052267 |
| 0 | AAK1 | 21.9505043 | 1.329108715 | 2.1854E−104 | 1.1411E−102 | 0.411953243 | 2.402109146 |
| 0 | FHL1 | 21.83530998 | 1.356438279 | 2.3345E−103 | 1.2118E−101 | 0.428355958 | 2.582528591 |
| 0 | SLC22A5 | 21.78883743 | 1.845539808 | 1.3861E−102 | 7.1253E−101 | 0.226621418 | 2.279653549 |
| 0 | RP11-227G15.12 | 21.77121544 | 1.794914007 | 1.8707E−102 | 9.5981E−101 | 0.236236802 | 2.266448259 |
| 0 | SH3RF2 | 21.74894905 | 1.943309546 | 3.9147E−102 | 2.0008E−100 | 0.211915535 | 2.401911259 |
| 0 | CFAP100 | 21.71637535 | 1.701435447 | 4.9761E−102 | 2.5384E−100 | 0.256598793 | 2.285129547 |
| 0 | NRDC | 21.68037796 | 1.165398121 | 5.3047E−102 | 2.7008E−100 | 0.52846908 | 2.475710154 |
| 0 | SMYD1 | 21.64245605 | 2.240609646 | 5.4609E−101 | 2.754E−99 | 0.17326546 | 2.328833103 |
| 0 | QKI | 21.51298714 | 1.144506216 | 1.6943E−100 | 8.512E−99 | 0.547322775 | 2.548404217 |
| 0 | PLEKHG6 | 21.51076508 | 2.016224623 | 5.4239E−100 | 2.70959E−98 | 0.196266968 | 2.287702799 |
| 0 | SLC2A4 | 21.45952415 | 1.902432203 | 1.3734E−99 | 6.80963E−98 | 0.21285822 | 2.40580368 |
| 0 | DHRSX | 21.44564438 | 1.353174806 | 8.3458E−100 | 4.16149E−98 | 0.380844646 | 2.421519756 |
| 0 | CECR2 | 21.43332481 | 1.471108198 | 1.2133E−99 | 6.03852E−98 | 0.322021116 | 2.318580866 |
| 0 | DCAF6 | 21.41809464 | 1.272251606 | 1.359E−99 | 6.75094E−98 | 0.422699849 | 2.398510218 |
| 0 | ABCC5 | 21.41381454 | 1.488714814 | 1.8322E−99 | 9.05102E−98 | 0.314856712 | 2.307276726 |
| 0 | LRRC39 | 21.39042473 | 1.826222658 | 4.9051E−99 | 2.40521E−97 | 0.222473605 | 2.266232967 |
| 0 | REV3L | 21.3791275 | 1.099208236 | 2.5767E−99 | 1.26817E−97 | 0.608974359 | 2.653390169 |
| 0 | PLAC8L1 | 21.3356266 | 2.060801029 | 2.13069E−98 | 1.03523E−96 | 0.187971342 | 2.328006744 |
| 0 | TMEM38A | 21.31019211 | 1.737818241 | 2.1699E−98 | 1.05236E−96 | 0.240007541 | 2.278663158 |
| 0 | DIAPH1 | 21.30989838 | 1.483226299 | 1.62298E−98 | 7.89998E−97 | 0.310143288 | 2.319092751 |
| 0 | NME9 | 21.24907494 | 1.691372395 | 7.17349E−98 | 3.4538E−96 | 0.249057315 | 2.317557335 |
| 0 | RC3H1 | 21.23828316 | 1.344949603 | 5.76955E−98 | 2.78289E−96 | 0.377262443 | 2.402335167 |
| 0 | CTD-2545M3.8 | 21.22595024 | 2.270614386 | 2.63536E−97 | 1.26426E−95 | 0.163650075 | 2.29227376 |
| 0 | MNAT1 | 21.14803505 | 1.297943115 | 3.51576E−97 | 1.68357E−95 | 0.395550528 | 2.40461731 |
| 0 | BIN1 | 21.03395081 | 1.46200633 | 3.94256E−96 | 1.8778E−94 | 0.320701357 | 2.356264591 |
| 0 | MSRB3 | 21.02565765 | 1.356385112 | 4.37868E−96 | 2.08179E−94 | 0.362368024 | 2.380004883 |
| 0 | FBXO34 | 21.01484299 | 1.475103498 | 6.38742E−96 | 3.02061E−94 | 0.30505279 | 2.323891878 |
| 0 | ENAH | 20.96415901 | 1.47441268 | 1.74127E−95 | 8.20525E−94 | 0.30938914 | 2.375952721 |
| 0 | COBL | 20.89888573 | 1.815428257 | 1.01323E−94 | 4.71604E−93 | 0.215874811 | 2.285074472 |
| 0 | P4HA3 | 20.86585045 | 1.480325818 | 1.26614E−94 | 5.88291E−93 | 0.301093514 | 2.330119133 |
| 0 | AMOTL1 | 20.80055237 | 1.473708272 | 4.70183E−94 | 2.18081E−92 | 0.300527903 | 2.339409351 |
| 0 | ARHGEF26 | 20.76397896 | 1.551800132 | 1.06128E−93 | 4.88832E−92 | 0.275452489 | 2.356694698 |
| 0 | PGM1 | 20.76395035 | 1.494803548 | 9.82095E−94 | 4.53145E−92 | 0.295060332 | 2.357512951 |
| 0 | DLG1 | 20.75449181 | 1.184107304 | 8.7229E−94 | 4.0318E−92 | 0.465686275 | 2.47281456 |
| 0 | IP6K3 | 20.75021172 | 1.559506178 | 1.407E−93 | 6.45837E−92 | 0.270173454 | 2.30109334 |
| 0 | EGLN1 | 20.65325546 | 1.32351315 | 7.30926E−93 | 3.33204E−91 | 0.367269985 | 2.368978977 |
| 0 | CYSTM1 | 20.63236046 | 1.21915853 | 1.04006E−92 | 4.73318E−91 | 0.437028658 | 2.496388912 |
| 0 | FBXL17 | 20.53580475 | 1.22678411 | 7.01772E−92 | 3.16655E−90 | 0.422134238 | 2.425061464 |
| 0 | SNAP23 | 20.50263786 | 1.26635766 | 1.41338E−91 | 6.35593E−90 | 0.388386124 | 2.364133358 |
| 0 | SLC7A6 | 20.41003609 | 1.177495837 | 8.11846E−91 | 3.6324E−89 | 0.459276018 | 2.479373932 |
| 0 | SCAPER | 20.38922119 | 1.230075121 | 1.28479E−90 | 5.73881E−89 | 0.407428356 | 2.405692339 |
| 0 | LARGE | 20.33263016 | 1.151516676 | 3.53101E−90 | 1.57192E−88 | 0.506033183 | 2.594677687 |
| 0 | UBE2G1 | 20.3101635 | 1.354402065 | 6.83589E−90 | 3.02292E−88 | 0.333144796 | 2.350923777 |
| 0 | GOLGA4 | 20.29752541 | 1.150561333 | 7.29826E−90 | 3.22202E−88 | 0.463800905 | 2.421747684 |
| 0 | PARK2 | 20.21364212 | 1.091457486 | 3.61323E−89 | 1.58464E−87 | 0.529788839 | 2.540163755 |
| 0 | NCOA1 | 20.21144485 | 1.004058957 | 3.67062E−89 | 1.60715E−87 | 0.646304676 | 2.740235329 |
| 0 | TK2 | 20.19812965 | 2.086274147 | 1.37992E−88 | 5.973E−87 | 0.165158371 | 2.235195398 |
| 0 | HADHA | 20.17575455 | 1.425387979 | 1.01189E−88 | 4.40148E−87 | 0.298076923 | 2.343864202 |
| 0 | PLIN2 | 20.17028427 | 1.364424586 | 1.08163E−88 | 4.69714E−87 | 0.356900452 | 2.779135704 |
| 0 | HOOK2 | 20.1023407 | 1.436550856 | 4.23748E−88 | 1.82824E−86 | 0.290346908 | 2.279594898 |
| 0 | AVIL | 20.08211136 | 1.695271611 | 8.376E−88 | 3.59047E−86 | 0.218325792 | 2.224918842 |
| 0 | OPTN | 20.07860374 | 1.336960316 | 6.08158E−88 | 2.61961E−86 | 0.334276018 | 2.35220027 |
| 0 | PPP3CC | 20.04811287 | 1.255401731 | 1.0099E−87 | 4.32208E−86 | 0.380656109 | 2.390338182 |
| 0 | AGBL1 | 20.01127052 | 1.353321671 | 2.06988E−87 | 8.83003E−86 | 0.369720965 | 2.697470427 |
| 0 | NOCT | 19.94989586 | 1.557746649 | 8.92473E−87 | 3.79508E−85 | 0.251319759 | 2.30614233 |
| 0 | MED13 | 19.88672829 | 1.190607429 | 2.20289E−86 | 9.30784E−85 | 0.408748115 | 2.389514446 |
| 0 | RNF157 | 19.85758972 | 1.901255012 | 8.10053E−86 | 3.39571E−84 | 0.181372549 | 2.324206114 |
| 0 | VPS13A | 19.84959984 | 1.333095074 | 4.92848E−86 | 2.07253E−84 | 0.328431373 | 2.323202372 |
| 0 | MITF | 19.82074738 | 1.322472215 | 8.81912E−86 | 3.67952E−84 | 0.334276018 | 2.416857243 |
| 0 | RP13-270P17.2 | 19.81352234 | 1.725600719 | 1.49554E−85 | 6.21048E−84 | 0.210972851 | 2.417802334 |
| 0 | SPATC1 | 19.79484367 | 2.191540956 | 3.52373E−85 | 1.45419E−83 | 0.150263952 | 2.338451385 |
| 0 | PLCB1 | 19.75309753 | 1.296819329 | 3.10404E−85 | 1.28299E−83 | 0.365384615 | 2.661675692 |
| 0 | RBM24 | 19.71492195 | 1.858980536 | 1.13851E−84 | 4.68391E−83 | 0.182315234 | 2.24503994 |
| 0 | EIF4ENIF1 | 19.66641617 | 1.660101414 | 2.28317E−84 | 9.34976E−83 | 0.218325792 | 2.315158129 |
| 0 | LMOD2 | 19.66296387 | 1.919983149 | 3.29757E−84 | 1.34623E−82 | 0.178544495 | 2.311804056 |
| 0 | PTPN3 | 19.65386391 | 1.574362278 | 2.63834E−84 | 1.07876E−82 | 0.237745098 | 2.295919418 |
| 0 | CACNG1 | 19.63109207 | 2.144591808 | 7.15998E−84 | 2.9052E−82 | 0.151206637 | 2.265474558 |
| 0 | GPI | 19.58740997 | 1.731316686 | 1.11312E−83 | 4.50965E−82 | 0.202865762 | 2.315764666 |
| 0 | NRG4 | 19.58057785 | 2.012823343 | 1.66508E−83 | 6.72534E−82 | 0.162518854 | 2.263978481 |
| 0 | OSBPL11 | 19.57642174 | 1.643151641 | 1.20952E−83 | 4.89277E−82 | 0.218891403 | 2.251123428 |
| 0 | INADL | 19.51492119 | 1.17450881 | 2.58675E−83 | 1.04164E−81 | 0.408936652 | 2.410250187 |
| 0 | ZBTB38 | 19.47099686 | 1.453395367 | 7.78988E−83 | 3.11795E−81 | 0.268853695 | 2.442140102 |
| 0 | MAX | 19.43815422 | 1.318956256 | 1.2569E−82 | 5.02324E−81 | 0.316930618 | 2.361189365 |
| 0 | SYNPO2 | 19.41036034 | 1.34395957 | 2.07622E−82 | 8.27283E−81 | 0.315422323 | 2.415464878 |
| 0 | SRSF12 | 19.38882828 | 1.877543449 | 5.30694E−82 | 2.10511E−80 | 0.176282051 | 2.274590015 |
| 0 | PFKM | 19.36846161 | 1.618903756 | 6.08527E−82 | 2.41026E−80 | 0.219457014 | 2.320452929 |
| 0 | DIO2-AS1 | 19.30417061 | 1.867074609 | 2.66701E−81 | 1.04544E−79 | 0.189291101 | 2.84785533 |
| 0 | RP11-131H24.4 | 19.28994942 | 2.097311735 | 4.21243E−81 | 1.64638E−79 | 0.148001508 | 2.169793129 |
| 0 | PKD2L2 | 19.2875576 | 1.490066409 | 2.37425E−81 | 9.32061E−80 | 0.251508296 | 2.296911001 |
| 0 | CEP85L | 19.27736282 | 1.239166141 | 2.36838E−81 | 9.31129E−80 | 0.354072398 | 2.381110907 |
| 0 | TNNT1 | 19.22320938 | 1.088797569 | 5.59532E−81 | 2.18045E−79 | 0.546945701 | 2.683222771 |
| 0 | LMCD1-AS1 | 19.17193413 | 1.619754553 | 2.38669E−80 | 9.26E−79 | 0.220022624 | 2.474068403 |
| 0 | RP1-117O3.2 | 19.16625404 | 1.682880878 | 2.79695E−80 | 1.08359E−78 | 0.200226244 | 2.230357885 |
| 0 | PPP3CB | 19.14553452 | 1.306399941 | 2.82759E−80 | 1.09387E−78 | 0.320889894 | 2.393736362 |
| 0 | ANO6 | 19.10656738 | 1.205892801 | 5.55508E−80 | 2.14278E−78 | 0.365573152 | 2.36345315 |
| 0 | ELMOD3 | 18.99400139 | 1.416475058 | 5.19709E−79 | 1.98453E−77 | 0.264328808 | 2.306157112 |
| 0 | ASB14 | 18.95897293 | 1.627929688 | 1.18492E−78 | 4.5117E−77 | 0.207956259 | 2.266341209 |
| 0 | ZNF827 | 18.81018829 | 1.753664374 | 1.98356E−77 | 7.48823E−76 | 0.181749623 | 2.274096966 |
| 0 | PHTF2 | 18.80431557 | 1.347280979 | 1.56059E−77 | 5.89983E−76 | 0.286764706 | 2.321904659 |
| 0 | SCN4A | 18.78740311 | 1.978144765 | 3.62211E−77 | 1.36353E−75 | 0.152526395 | 2.197559834 |
| 0 | SEC13 | 18.74956322 | 1.502748251 | 4.73396E−77 | 1.77704E−75 | 0.233785822 | 2.257550716 |
| 0 | SNX24 | 18.74044228 | 1.584287882 | 6.24302E−77 | 2.33691E−75 | 0.215686275 | 2.478454113 |
| 0 | DIAPH2-AS1 | 18.7371769 | 1.167911887 | 4.60388E−77 | 1.73066E−75 | 0.404600302 | 2.57460022 |
| 0 | ALPK3 | 18.73145676 | 1.616522908 | 7.59445E−77 | 2.83879E−75 | 0.203242836 | 2.239436626 |
| 0 | MYOM2 | 18.70695305 | 2.043946266 | 1.72152E−76 | 6.38114E−75 | 0.14668175 | 2.368929625 |
| 0 | ABCC9 | 18.70468712 | 1.47476387 | 1.05783E−76 | 3.93753E−75 | 0.239441931 | 2.283888102 |
| 0 | NOS1 | 18.67178726 | 1.628685236 | 2.27133E−76 | 8.4074E−75 | 0.203054299 | 2.361010075 |
| 0 | OIP5 | 18.64021683 | 1.723742008 | 4.19435E−76 | 1.54181E−74 | 0.184200603 | 2.263207197 |
| 0 | TRIM54 | 18.63317299 | 1.509993196 | 4.05801E−76 | 1.49376E−74 | 0.2239819 | 2.212064266 |
| 0 | CACNA2D3 | 18.63073349 | 1.400458694 | 3.91964E−76 | 1.44483E−74 | 0.259803922 | 2.32923913 |
| 0 | MAPT | 18.62649345 | 1.492426515 | 4.43984E−76 | 1.6298E−74 | 0.231334842 | 2.263072014 |
| 0 | AGL | 18.56879616 | 1.571789861 | 1.40418E−75 | 5.14035E−74 | 0.211726998 | 2.366036654 |
| 0 | UNQ6494 | 18.51959229 | 1.6502074 | 3.56247E−75 | 1.29698E−73 | 0.197021116 | 2.408855438 |
| 0 | THRAP3 | 18.50999832 | 1.125159621 | 2.8442E−75 | 1.03691E−73 | 0.389705882 | 2.366123438 |
| 0 | DTNA | 18.49381065 | 1.305574536 | 4.09824E−75 | 1.48796E−73 | 0.301659125 | 2.407066584 |
| 0 | UNC13C | 18.45017242 | 2.307907343 | 2.07796E−74 | 7.47308E−73 | 0.122360483 | 2.363838673 |
| 0 | TNS3 | 18.43144798 | 1.375910044 | 1.33592E−74 | 4.81093E−73 | 0.262820513 | 2.277597666 |
| 0 | NR6A1 | 18.39040184 | 1.528244376 | 3.15401E−74 | 1.1297E−72 | 0.219834087 | 2.325729847 |
| 0 | USP2 | 18.37050819 | 1.692984343 | 5.03067E−74 | 1.79463E−72 | 0.183634992 | 2.227475882 |
| 0 | LMO7 | 18.34247589 | 1.499092698 | 7.21197E−74 | 2.55905E−72 | 0.222096531 | 2.243437052 |
| 0 | ACSF2 | 18.34053612 | 1.881338954 | 1.0185E−73 | 3.58525E−72 | 0.15592006 | 2.268602848 |
| 0 | ESR2 | 18.31093597 | 1.1641891 | 9.8873E−74 | 3.48508E−72 | 0.369532428 | 2.377988815 |
| 0 | RP11-21A7A.2 | 18.27960777 | 1.550349355 | 2.30361E−73 | 8.06627E−72 | 0.209653092 | 2.281793833 |
| 0 | PIGF | 18.27829933 | 1.586144567 | 2.45191E−73 | 8.56301E−72 | 0.201546003 | 2.31261754 |
| 0 | PPP6R3 | 18.27804947 | 0.979604602 | 1.67515E−73 | 5.87341E−72 | 0.52224736 | 2.484341383 |
| 0 | RFX3-AS1 | 18.25957489 | 1.488577366 | 3.16197E−73 | 1.10283E−71 | 0.223793363 | 2.294851542 |
| 0 | CDKL4 | 18.24741745 | 1.6736058 | 4.52193E−73 | 1.57509E−71 | 0.184012066 | 2.224570036 |
| 0 | MLXIP | 18.20706749 | 1.252080321 | 6.79534E−73 | 2.36387E−71 | 0.302413273 | 2.299991608 |
| 0 | RPTOR | 18.16088486 | 1.331749201 | 1.63439E−72 | 5.67063E−71 | 0.266591252 | 2.306171179 |
| 0 | JPH1 | 18.12937164 | 1.695486188 | 3.67687E−72 | 1.26745E−70 | 0.178733032 | 2.319608688 |
| 0 | PHF20 | 18.11268044 | 1.093140841 | 3.28937E−72 | 1.13682E−70 | 0.402149321 | 2.422985315 |
| 0 | FEZ2 | 18.09367371 | 1.116663933 | 4.6169E−72 | 1.58942E−70 | 0.42081448 | 2.660176039 |
| 0 | GHR | 18.01223946 | 1.028234124 | 1.85787E−71 | 6.36295E−70 | 0.47341629 | 2.574320078 |
| 0 | PARD3 | 18.0058918 | 0.897201538 | 2.01811E−71 | 6.90288E−70 | 0.669117647 | 2.763483524 |
| 0 | AC011288.2 | 17.92776299 | 1.722795129 | 1.25833E−70 | 4.27108E−69 | 0.172511312 | 2.328503847 |
| 0 | FGF13 | 17.90705681 | 1.232451797 | 1.27211E−70 | 4.31235E−69 | 0.314856712 | 2.429938555 |
| 0 | PEBP4 | 17.8906517 | 1.410799861 | 1.91018E−70 | 6.45888E−69 | 0.236236802 | 2.279040098 |
| 0 | ASH1L | 17.88291168 | 0.95764482 | 1.75189E−70 | 5.93121E−69 | 0.52168175 | 2.504011631 |
| 0 | RP11-415J8.3 | 17.84030151 | 1.082346082 | 3.84327E−70 | 1.29458E−68 | 0.398567119 | 2.442633867 |
| 0 | DNAH11 | 17.82740784 | 1.180394411 | 5.01594E−70 | 1.68745E−68 | 0.330693816 | 2.357321501 |
| 0 | TDRD3 | 17.82636833 | 1.279032707 | 5.49465E−70 | 1.84616E−68 | 0.276395173 | 2.306956291 |
| 0 | SLC3A1 | 17.81783485 | 1.735740781 | 8.46477E−70 | 2.83693E−68 | 0.165723982 | 2.244956732 |
| 0 | ZRANB3 | 17.81241035 | 1.23585999 | 6.77625E−70 | 2.27389E−68 | 0.29581448 | 2.301890612 |
| 0 | IL6R | 17.79824829 | 1.412739038 | 9.79548E−70 | 3.27877E−68 | 0.229260935 | 2.275303364 |
| 0 | DCST2 | 17.77056122 | 2.16742754 | 2.60876E−69 | 8.64502E−68 | 0.121606335 | 2.220575571 |
| 0 | TXLNB | 17.76815796 | 1.459523559 | 1.6635E−69 | 5.55411E−68 | 0.21719457 | 2.233676434 |
| 0 | BRE | 17.76275444 | 1.050187945 | 1.45126E−69 | 4.8516E−68 | 0.424019608 | 2.472531796 |
| 0 | SLC25A33 | 17.75013733 | 1.392471194 | 2.20181E−69 | 7.32385E−68 | 0.235671192 | 2.295519114 |
| 0 | SYPL2 | 17.71124458 | 1.802222371 | 5.7599E−69 | 1.90399E−67 | 0.152903469 | 2.260393858 |
| 0 | TMEM135 | 17.63331413 | 1.095579982 | 1.39751E−68 | 4.60242E−67 | 0.379524887 | 2.494039059 |
| 0 | CBX1 | 17.6192646 | 1.613857269 | 2.3862E−68 | 7.8294E−67 | 0.182315234 | 2.25194025 |
| 0 | GMPR | 17.60829735 | 1.49193573 | 2.65232E−68 | 8.69183E−67 | 0.205316742 | 2.224158764 |
| 0 | G3BP2 | 17.50944138 | 1.419972777 | 1.37336E−67 | 4.48399E−66 | 0.22153092 | 2.266646624 |
| 0 | UBR3 | 17.49085999 | 1.016369581 | 1.54109E−67 | 5.0193E−66 | 0.428167421 | 2.394164801 |
| 0 | PPP1R3C | 17.48988533 | 1.937995434 | 2.83671E−67 | 9.19405E−66 | 0.143476621 | 2.429639816 |
| 0 | TNNT3 | 17.47590065 | 1.339488268 | 2.29456E−67 | 7.44598E−66 | 0.269419306 | 2.722733259 |
| 0 | EFTUD2 | 17.45556641 | 1.652798176 | 4.05755E−67 | 1.30871E−65 | 0.170625943 | 2.263745308 |
| 0 | USP13 | 17.45141411 | 1.361573696 | 3.53772E−67 | 1.14382E−65 | 0.237745098 | 2.250944614 |
| 0 | RP11-145A3.1 | 17.39904022 | 1.528439999 | 9.79561E−67 | 3.1366E−65 | 0.19494721 | 2.354938507 |
| 0 | FAM222B | 17.39069748 | 1.311974645 | 9.67138E−67 | 3.10056E−65 | 0.253205128 | 2.290510416 |
| 0 | USP34 | 17.37841606 | 0.910429895 | 1.01127E−66 | 3.23036E−65 | 0.546568627 | 2.519139051 |
| 0 | CHN2 | 17.35637093 | 1.415685773 | 1.86017E−66 | 5.92069E−65 | 0.231334842 | 2.657305956 |
| 0 | USO1 | 17.35306549 | 1.176837564 | 1.74672E−66 | 5.56626E−65 | 0.305241327 | 2.339381218 |
| 0 | ARG2 | 17.34252548 | 1.617995977 | 2.63373E−66 | 8.36279E−65 | 0.176847662 | 2.301152706 |
| 0 | ADCY2 | 17.30888557 | 1.207603931 | 3.66463E−66 | 1.16085E−64 | 0.293740573 | 2.346117735 |
| 0 | ANK1 | 17.22611237 | 1.451314569 | 1.68543E−65 | 5.30104E−64 | 0.205693816 | 2.269577265 |
| 0 | ZBTB16 | 17.21910858 | 0.670217931 | 1.59889E−65 | 5.03479E−64 | 0.885558069 | 3.14014864 |
| 0 | RBM20 | 17.21783638 | 1.543208718 | 2.06746E−65 | 6.47958E−64 | 0.186840121 | 2.321971416 |
| 0 | MAMSTR | 17.21395493 | 2.515387774 | 3.96494E−65 | 1.23826E−63 | 0.095776772 | 2.271016121 |
| 0 | ZNF438 | 17.12117004 | 1.106924176 | 8.27249E−65 | 2.57144E−63 | 0.338046757 | 2.36618638 |
| 0 | RERE | 17.01954842 | 0.882836521 | 4.171E−64 | 1.2845E−62 | 0.557315234 | 2.520747185 |
| 0 | TRIM72 | 16.90210724 | 1.573284388 | 3.90831E−63 | 1.19118E−61 | 0.174019608 | 2.231470108 |
| 0 | FAM179A | 16.89718056 | 1.67205286 | 4.53886E−63 | 1.3802E−61 | 0.160633484 | 2.404221535 |
| 0 | RUSC2 | 16.89629173 | 1.452486157 | 4.10074E−63 | 1.2484E−61 | 0.19739819 | 2.311003447 |
| 0 | SCML1 | 16.87116051 | 1.042011499 | 5.11049E−63 | 1.55047E−61 | 0.371606335 | 2.372078419 |
| 0 | HACD1 | 16.8257103 | 1.595159531 | 1.3798E−62 | 4.1577E−61 | 0.169306184 | 2.254306555 |
| 0 | RP11-82O19.1 | 16.8163929 | 1.430505633 | 1.4821E−62 | 4.4609E−61 | 0.201923077 | 2.262339115 |
| 0 | KMT2C | 16.81148148 | 0.915655255 | 1.30619E−62 | 3.94484E−61 | 0.497737557 | 2.442172766 |
| 0 | PTPN14 | 16.77837563 | 1.181063175 | 2.46384E−62 | 7.37404E−61 | 0.28714178 | 2.334687233 |
| 0 | RP11-16L14.2 | 16.77301407 | 1.738576651 | 3.57857E−62 | 1.06862E−60 | 0.145739065 | 2.189573526 |
| 0 | PPTC7 | 16.72522163 | 1.347937226 | 6.47271E−62 | 1.91992E−60 | 0.216251885 | 2.230639696 |
| 0 | RP11-296O14.3 | 16.72070694 | 1.640796304 | 8.0382E−62 | 2.37897E−60 | 0.159125189 | 2.261808395 |
| 0 | SH2D1B | 16.71588898 | 2.533542871 | 1.34364E−61 | 3.96335E−60 | 0.091251885 | 2.412657499 |
| 0 | JPX | 16.70307922 | 1.005693674 | 7.95441E−62 | 2.35679E−60 | 0.394984917 | 2.394914389 |
| 0 | PALLD | 16.69462013 | 0.987196505 | 9.09298E−62 | 2.68516E−60 | 0.41892911 | 2.466726542 |
| 0 | ZNF749 | 16.67695236 | 2.227047682 | 2.19025E−61 | 6.41083E−60 | 0.104449472 | 2.234355927 |
| 0 | FAM20A | 16.64090729 | 1.22423315 | 2.35155E−61 | 6.86781E−60 | 0.272435897 | 2.423431396 |
| 0 | ABCC1 | 16.640625 | 1.192810297 | 2.37789E−61 | 6.93712E−60 | 0.272624434 | 2.323126316 |
| 0 | SVIL-AS1 | 16.61931992 | 1.272624969 | 3.49139E−61 | 1.01522E−59 | 0.240007541 | 2.296357155 |
| 0 | CAMKMT | 16.61318207 | 1.030198216 | 3.45152E−61 | 1.00472E−59 | 0.37311463 | 2.393567801 |
| 0 | BNC2 | 16.6000843 | 0.938436508 | 4.16794E−61 | 1.21062E−59 | 0.48887632 | 2.67073822 |
| 0 | JARID2 | 16.54021835 | 1.23473382 | 1.22731E−60 | 3.53011E−59 | 0.253393665 | 2.334017754 |
| 0 | CDH20 | 16.50033379 | 1.757992625 | 3.12726E−60 | 8.88912E−59 | 0.145550528 | 2.98066473 |
| 0 | MIR181A2HG | 16.46705627 | 1.713909984 | 4.94044E−60 | 1.4028E−58 | 0.145739065 | 2.252746344 |
| 0 | RASA4B | 16.44724464 | 1.66064477 | 6.67478E−60 | 1.89121E−58 | 0.150641026 | 2.222771645 |
| 0 | SAR1B | 16.40873909 | 1.332426786 | 1.06855E−59 | 3.02116E−58 | 0.214177979 | 2.242059708 |
| 0 | RP11-166O4.6 | 16.34135818 | 1.788661718 | 3.86196E−59 | 1.08269E−57 | 0.1336727 | 2.188122988 |
| 0 | ERBB4 | 16.33798599 | 1.286193848 | 3.20581E−59 | 8.99693E−58 | 0.231523379 | 2.324207544 |
| 0 | DAG1 | 16.32616425 | 1.372845531 | 4.05991E−59 | 1.13579E−57 | 0.20173454 | 2.276916265 |
| 0 | DNHD1 | 16.32529831 | 1.434533596 | 4.2239E−59 | 1.18043E−57 | 0.187405732 | 2.269569874 |
| 0 | ADCK3 | 16.29577065 | 1.116419792 | 5.85794E−59 | 1.63023E−57 | 0.297699849 | 2.26932168 |
| 0 | RP11-293P20.2 | 16.27103615 | 1.959477067 | 1.2665E−58 | 3.50989E−57 | 0.116704374 | 2.176229954 |
| 0 | RETSAT | 16.23784828 | 1.701403141 | 1.92754E−58 | 5.32518E−57 | 0.14046003 | 2.225717545 |
| 0 | UBR5 | 16.22808075 | 0.964127302 | 1.66743E−58 | 4.61138E−57 | 0.39479638 | 2.350933075 |
| 0 | IN080 | 16.19464684 | 1.158841133 | 3.02253E−58 | 8.32434E−57 | 0.27168175 | 2.313791275 |
| 0 | AC010976.2 | 16.15413475 | 1.675938845 | 7.05058E−58 | 1.92781E−56 | 0.143476621 | 2.203706503 |
| 0 | RP11-545I5.3 | 16.14826965 | 1.428564668 | 6.98283E−58 | 1.91125E−56 | 0.184954751 | 2.225213289 |
| 0 | KIF9-AS1 | 16.12478065 | 1.378516436 | 9.94874E−58 | 2.71187E−56 | 0.195701357 | 2.259277821 |
| 0 | CLIP4 | 16.05187607 | 1.183624029 | 2.91117E−57 | 7.90298E−56 | 0.254147813 | 2.289397717 |
| 0 | ASPH | 16.04857063 | 1.015013933 | 2.89648E−57 | 7.87113E−56 | 0.3489819 | 2.372408152 |
| 0 | MPRIP | 16.02899742 | 1.125928879 | 4.05369E−57 | 1.0971E−55 | 0.281862745 | 2.309693336 |
| 0 | PWAR6 | 16.00473976 | 1.816303492 | 7.97734E−57 | 2.15242E−55 | 0.125188537 | 2.213516235 |
| 0 | DIO2 | 16.00029182 | 1.821836829 | 8.76002E−57 | 2.36121E−55 | 0.130090498 | 2.759577036 |
| 0 | OGDH | 15.99643326 | 1.215083241 | 6.98728E−57 | 1.8872E−55 | 0.240950226 | 2.262580395 |
| 0 | RP11-94C24.6 | 15.97014713 | 2.290425539 | 1.68331E−56 | 4.50981E−55 | 0.092383107 | 2.239403009 |
| 0 | AC026202.3 | 15.97001553 | 1.081814051 | 1.006E−56 | 2.70611E−55 | 0.305995475 | 2.336813688 |
| 0 | AC004076.9 | 15.96415424 | 1.866314769 | 1.5308E−56 | 4.10536E−55 | 0.119343891 | 2.195821285 |
| 0 | CTBP2 | 15.95919609 | 1.00650382 | 1.16195E−56 | 3.11931E−55 | 0.354826546 | 2.366051435 |
| 0 | CCPG1 | 15.93528175 | 1.13858211 | 1.78949E−56 | 4.78464E−55 | 0.266591252 | 2.28431654 |
| 0 | DOCK3 | 15.89577389 | 1.339196801 | 3.53664E−56 | 9.41816E−55 | 0.200603318 | 2.304512262 |
| 0 | ST3GAL3 | 15.87503338 | 1.148240924 | 4.58134E−56 | 1.21637E−54 | 0.262443439 | 2.299495459 |
| 0 | PDZD2 | 15.85541534 | 0.890840232 | 5.72523E−56 | 1.51704E−54 | 0.473227753 | 2.519250393 |
| 0 | SLC38A1 | 15.8033905 | 1.145225525 | 1.37929E−55 | 3.62585E−54 | 0.268288084 | 2.432721376 |
| 0 | CNNM2 | 15.79618645 | 1.164433837 | 1.54655E−55 | 4.05753E−54 | 0.258861237 | 2.335786343 |
| 0 | PFKFB2 | 15.77776051 | 1.564805388 | 2.42121E−55 | 6.3273E−54 | 0.150452489 | 2.185872555 |
| 0 | ANKUB1 | 15.77569866 | 1.006205797 | 2.01969E−55 | 5.29362E−54 | 0.349924585 | 2.398211718 |
| 0 | GGT7 | 15.71939087 | 1.348219275 | 5.59582E−55 | 1.45519E−53 | 0.198340875 | 2.585859537 |
| 0 | FGGY | 15.68927479 | 1.242921352 | 8.31485E−55 | 2.15594E−53 | 0.222285068 | 2.315636396 |
| 0 | SLC25A12 | 15.66139984 | 1.203821421 | 1.26794E−54 | 3.28441E−53 | 0.232466063 | 2.268038511 |
| 0 | CFLAR | 15.65371227 | 0.937332332 | 1.30525E−54 | 3.37447E−53 | 0.395173454 | 2.380142212 |
| 0 | BLCAP | 15.64676189 | 1.144592881 | 1.54995E−54 | 4.00321E−53 | 0.254713424 | 2.258275032 |
| 0 | TRIM41 | 15.64253426 | 1.818199396 | 2.12095E−54 | 5.45679E−53 | 0.120098039 | 2.247891188 |
| 0 | PFDN1 | 15.6197958 | 1.158570886 | 2.36562E−54 | 6.08037E−53 | 0.250754148 | 2.326673031 |
| 0 | HDAC8 | 15.55946064 | 1.023276329 | 5.67615E−54 | 1.45332E−52 | 0.323152338 | 2.368926764 |
| 0 | ITGA7 | 15.54717445 | 1.442262292 | 7.75764E−54 | 1.98245E−52 | 0.171757164 | 2.306205034 |
| 0 | RARRES1 | 15.52923679 | 1.220838785 | 9.49459E−54 | 2.41935E−52 | 0.226055807 | 2.27863431 |
| 0 | KPNA1 | 15.52049923 | 1.159834027 | 1.07613E−53 | 2.7395E−52 | 0.24321267 | 2.243037224 |
| 0 | SPEG | 15.51967144 | 1.544146419 | 1.25477E−53 | 3.19121E−52 | 0.148001508 | 2.235529661 |
| 0 | MBNL1 | 15.50355434 | 0.691259444 | 1.28425E−53 | 3.26305E−52 | 0.771493213 | 2.855930567 |
| 0 | FOXP2 | 15.50218201 | 1.024141669 | 1.34311E−53 | 3.40609E−52 | 0.32484917 | 2.35075736 |
| 0 | GATS | 15.47367954 | 1.223187685 | 2.23676E−53 | 5.6508E−52 | 0.220776772 | 2.265581846 |
| 0 | RP11-577H5.5 | 15.45517635 | 1.205093622 | 2.93119E−53 | 7.39111E−52 | 0.224358974 | 2.219513178 |
| 0 | UGP2 | 15.43696308 | 1.032930732 | 3.67407E−53 | 9.25552E−52 | 0.303544495 | 2.299382925 |
| 0 | C3orf35 | 15.43685818 | 1.611587167 | 4.43483E−53 | 1.11509E−51 | 0.138763198 | 2.178259373 |
| 0 | RYR3 | 15.43056202 | 1.21413672 | 4.266E−53 | 1.07365E−51 | 0.228695324 | 2.389222622 |
| 0 | TAC4 | 15.36862469 | 1.507579446 | 1.20613E−52 | 3.02125E−51 | 0.151960784 | 2.220090151 |
| 0 | NUP54 | 15.33423996 | 1.431720495 | 2.01342E−52 | 5.02447E−51 | 0.165158371 | 2.369675398 |
| 0 | FHIT | 15.32340717 | 0.991145015 | 1.99881E−52 | 4.99271E−51 | 0.342006033 | 2.416106462 |
| 0 | C9orf147 | 15.3225317 | 2.131672382 | 3.03416E−52 | 7.55044E−51 | 0.092948718 | 2.22565937 |
| 0 | RILPL1 | 15.29295063 | 1.261289001 | 3.43845E−52 | 8.54052E−51 | 0.203808446 | 2.218065262 |
| 0 | GBAS | 15.29242706 | 1.110419631 | 3.3246E−52 | 8.26548E−51 | 0.260180995 | 2.331459761 |
| 0 | KANSL1L | 15.26628399 | 0.920988321 | 4.68677E−52 | 1.15978E−50 | 0.391214178 | 2.434716702 |
| 0 | FOXN4 | 15.23561192 | 2.378076792 | 1.20747E−51 | 2.96316E−50 | 0.081259427 | 2.233010769 |
| 0 | HSDL2 | 15.20335674 | 1.327059984 | 1.34592E−51 | 3.29987E−50 | 0.184954751 | 2.273465872 |
| 0 | VPS13D | 15.19289207 | 0.879760206 | 1.39243E−51 | 3.41074E−50 | 0.422134238 | 2.395941019 |
| 0 | ME1 | 15.12923717 | 1.282018185 | 3.99475E−51 | 9.70463E−50 | 0.194381599 | 2.2682302 |
| 0 | R3HDM1 | 15.11114979 | 1.137142777 | 5.00905E−51 | 1.21576E−49 | 0.244155354 | 2.335669518 |
| 0 | C14orf159 | 15.06742477 | 1.193273067 | 9.77159E−51 | 2.35448E−49 | 0.218325792 | 2.26219511 |
| 0 | RP4-791M13.4 | 15.05053425 | 1.994030714 | 1.64561E−50 | 3.94724E−49 | 0.097473605 | 2.168473005 |
| 0 | CRLF3 | 15.04998779 | 1.077667832 | 1.21722E−50 | 2.92495E−49 | 0.268099548 | 2.333545685 |
| 0 | FAF1 | 15.01508904 | 0.860432208 | 1.94554E−50 | 4.66245E−49 | 0.431749623 | 2.395051479 |
| 0 | ZNF423 | 15.00989819 | 1.426249862 | 2.45304E−50 | 5.86809E−49 | 0.158559578 | 2.263974428 |
| 0 | ANK3 | 14.99538708 | 1.363517046 | 2.96365E−50 | 7.07682E−49 | 0.173076923 | 2.274002552 |
| 0 | TBC1D1 | 14.97391891 | 1.030160427 | 3.69774E−50 | 8.82181E−49 | 0.294683258 | 2.401354313 |

Cluster 2 Myocyte

| | | | | | | | |
|---|---|---|---|---|---|---|---|
| 2 | TTN | 18.91510391 | 1.567040563 | 8.79956E−75 | 2.59851E−71 | 0.921542553 | 3.60868001 |
| 2 | NEB | 18.90369987 | 1.710253716 | 4.87598E−75 | 1.61986E−71 | 0.880319149 | 3.65627265 |
| 2 | MLIP | 18.5883503 | 1.632962823 | 5.9037E−73 | 1.42639E−69 | 0.828457447 | 3.21810174 |
| 2 | SVIL | 18.37194443 | 1.549738407 | 4.12933E−71 | 7.83895E−68 | 0.894281915 | 3.355479956 |
| 2 | DMD | 18.27946281 | 1.538463712 | 2.34784E−70 | 4.1599E−67 | 0.926861702 | 3.868873596 |
| 2 | NEXN | 17.91363144 | 1.618423223 | 3.64652E−68 | 4.84568E−65 | 0.817154255 | 3.189834595 |
| 2 | RP11-362A9.3 | 17.87204361 | 1.703782916 | 6.39172E−68 | 8.08918E−65 | 0.792553191 | 3.258601427 |
| 2 | PDE4D | 17.65384865 | 1.654889226 | 4.79066E−66 | 5.30506E−63 | 0.912234043 | 4.051409721 |
| 2 | FBXO32 | 17.2364521 | 1.65746367 | 1.57106E−63 | 1.60592E−60 | 0.790558511 | 3.375625849 |
| 2 | TRDN | 17.10459709 | 1.649006844 | 1.17851E−62 | 1.11862E−59 | 0.750664894 | 3.251871586 |
| 2 | MYBPC1 | 16.97781944 | 1.91556406 | 8.32761E−62 | 7.63182E−59 | 0.728058511 | 3.969906807 |
| 2 | TRIM63 | 15.28352737 | 1.454261899 | 7.95172E−51 | 4.91472E−48 | 0.763297872 | 3.167193174 |
| 2 | TACC2 | 15.16292 | 1.530524135 | 4.54642E−50 | 2.68512E−47 | 0.594414894 | 2.78809762 |
| 2 | MYPN | 14.93404293 | 1.405670881 | 1.06197E−48 | 5.42769E−46 | 0.71143617 | 2.910595417 |
| 2 | NEAT1 | 14.53893661 | 1.009650588 | 3.47819E−46 | 1.59379E−43 | 0.964760638 | 4.773561954 |
| 2 | AGBL1 | 14.37777805 | 1.690928936 | 2.7647E−45 | 1.22462E−42 | 0.465425532 | 2.94357872 |
| 2 | PEX5L | 14.28782177 | 1.553905129 | 7.97892E−45 | 3.31337E−42 | 0.557845745 | 2.971868515 |
| 2 | ABLIM2 | 14.25771618 | 1.347255588 | 1.11243E−44 | 4.54848E−42 | 0.674867021 | 2.762402534 |
| 2 | PDLIM5 | 13.76612473 | 1.203850269 | 8.39566E−42 | 2.97509E−39 | 0.845744681 | 3.208065748 |
| 2 | FAM134B | 13.67683315 | 1.447466254 | 2.45652E−41 | 8.37012E−39 | 0.563164894 | 2.873705387 |
| 2 | PRKG1 | 13.48586178 | 1.345178962 | 2.77072E−40 | 8.98017E−38 | 0.664893617 | 3.147129774 |
| 2 | EYA4 | 13.48381615 | 1.251600027 | 2.90148E−40 | 9.29067E−38 | 0.755319149 | 3.019938946 |
| 2 | ASB5 | 13.3914957 | 1.348494411 | 9.30118E−40 | 2.90821E−37 | 0.582446809 | 2.679147005 |
| 2 | TBC1D8 | 13.3588953 | 1.22247386 | 1.40252E−39 | 4.33427E−37 | 0.749335106 | 3.038730145 |
| 2 | AKAP6 | 13.3441267 | 1.407507777 | 1.76062E−39 | 5.37838E−37 | 0.527260638 | 2.68428731 |
| 2 | SLC36A2 | 13.18357944 | 1.93830514 | 1.97459E−38 | 5.89648E−36 | 0.283244681 | 2.558409691 |
| 2 | GLRA1 | 13.10096645 | 1.837557793 | 5.20511E−38 | 1.52601E−35 | 0.296542553 | 2.434786558 |
| 2 | RIF1 | 12.93259335 | 1.285613656 | 2.883E−37 | 8.15122E−35 | 0.601728723 | 2.705685616 |
| 2 | RP11-532N4.2 | 12.90403938 | 1.433244944 | 4.1612E−37 | 1.152E−34 | 0.537234043 | 2.974453211 |
| 2 | PDE4DIP | 12.36886883 | 1.06093967 | 2.64742E−34 | 6.33879E−32 | 0.787234043 | 3.013641596 |
| 2 | ATP1B1 | 12.31983185 | 1.392726302 | 5.00718E−34 | 1.16733E−31 | 0.441489362 | 2.560085535 |
| 2 | CAP2 | 12.08350945 | 1.158466578 | 7.37974E−33 | 1.62092E−30 | 0.700797872 | 2.964101553 |
| 2 | RYR1 | 12.07474327 | 1.134073496 | 8.18633E−33 | 1.78334E−30 | 0.689494681 | 2.798129797 |
| 2 | TTN-AS1 | 12.0442915 | 1.07553494 | 1.17632E−32 | 2.44244E−30 | 0.753324468 | 2.909742594 |
| 2 | MPV17 | 12.02166748 | 1.214659452 | 1.52453E−32 | 3.11672E−30 | 0.550531915 | 2.591000557 |
| 2 | LARGE | 12.00811863 | 1.222070336 | 1.80824E−32 | 3.64073E−30 | 0.5625 | 2.782058001 |
| 2 | SPRTN | 11.99744606 | 1.368681312 | 2.13409E−32 | 4.23267E−30 | 0.43018617 | 2.567420006 |
| 2 | MALAT1 | 11.77150917 | 0.664788306 | 2.64105E−31 | 4.87439E−29 | 0.995345745 | 6.952069283 |
| 2 | PCNT | 11.67835712 | 1.178779364 | 7.62123E−31 | 1.36858E−28 | 0.549202128 | 2.568871498 |
| 2 | ADCY9 | 11.588871 | 1.369272113 | 2.24335E−30 | 3.94845E−28 | 0.390292553 | 2.543217421 |
| 2 | BACH1 | 11.42694855 | 1.083581328 | 1.24316E−29 | 2.06497E−27 | 0.67087766 | 2.799019337 |
| 2 | PPARGC1A | 11.42185783 | 1.307081342 | 1.39241E−29 | 2.29852E−27 | 0.426196809 | 2.634195328 |
| 2 | SALRNA2 | 11.41543865 | 1.74123013 | 1.78787E−29 | 2.93311E−27 | 0.248005319 | 2.40765357 |
| 2 | LRRC2 | 11.13176823 | 1.266040683 | 3.25984E−28 | 4.92255E−26 | 0.406914894 | 2.423300743 |
| 2 | KIF1B | 11.07899284 | 1.023881435 | 5.48844E−28 | 8.24104E−26 | 0.695478723 | 2.829248667 |
| 2 | PDLIM3 | 10.91890621 | 1.154265881 | 3.0312E−27 | 4.3546E−25 | 0.724734043 | 3.359336853 |
| 2 | FAM160A1 | 10.72207642 | 1.296125531 | 2.52163E−26 | 3.43679E−24 | 0.362367021 | 2.436731815 |
| 2 | SLC20A2 | 10.67056179 | 1.1010499 | 4.11515E−26 | 5.46842E−24 | 0.533244681 | 2.676830769 |
| 2 | THRB | 10.61974049 | 1.019972801 | 6.90988E−26 | 9.00215E−24 | 0.611037234 | 2.674141884 |
| 2 | TP63 | 10.53596687 | 1.10275054 | 1.63491E−25 | 2.09909E−23 | 0.575797872 | 2.83540082 |
| 2 | FAM184B | 10.533288 | 1.038636446 | 1.6785E−25 | 2.14469E−23 | 0.611702128 | 2.744561195 |
| 2 | OBSCN | 10.44314957 | 1.05041039 | 4.22248E−25 | 5.26859E−23 | 0.575797872 | 2.67788887 |
| 2 | IL12RB2 | 10.37972641 | 1.185966134 | 8.23875E−25 | 1.02318E−22 | 0.408909574 | 2.470058441 |
| 2 | SLC38A1 | 10.34130764 | 1.275879025 | 1.23863E−24 | 1.51005E−22 | 0.345079787 | 2.454803467 |
| 2 | FAM20A | 10.32001877 | 1.303017259 | 1.55527E−24 | 1.87883E−22 | 0.337765957 | 2.523349047 |
| 2 | ALK | 10.28872013 | 1.114429832 | 2.02317E−24 | 2.41121E−22 | 0.496010638 | 2.658817291 |
| 2 | NPAS2 | 10.27796555 | 1.189062834 | 2.31772E−24 | 2.73769E−22 | 0.39893617 | 2.535564899 |
| 2 | RP11-64D24.2 | 10.27102375 | 0.993639231 | 2.39256E−24 | 2.81359E−22 | 0.642952128 | 2.814280987 |
| 2 | RBFOX1 | 10.25261688 | 1.011546493 | 2.87634E−24 | 3.35283E−22 | 0.644946809 | 2.867855787 |
| 2 | ARHGAP11B | 10.24400139 | 1.42457211 | 3.43708E−24 | 3.95443E−22 | 0.269946809 | 2.362348318 |
| 2 | CLIP1 | 10.24391651 | 0.957416773 | 3.13793E−24 | 3.62594E−22 | 0.653590426 | 2.724636793 |
| 2 | NEDD4 | 10.12351418 | 1.054112315 | 1.04291E−23 | 1.15972E−21 | 0.553191489 | 2.823931456 |
| 2 | CABYR | 10.01727772 | 1.929701805 | 3.69428E−23 | 4.00746E−21 | 0.159574468 | 2.3196311 |
| 2 | LYVE1 | 9.936989784 | 1.435082316 | 6.96123E−23 | 7.34161E−21 | 0.263297872 | 2.558595657 |
| 2 | EFHC2 | 9.878255844 | 1.866165042 | 1.38055E−22 | 1.45023E−20 | 0.162234043 | 2.192156315 |
| 2 | OSBPL6 | 9.855507851 | 1.122792482 | 1.44816E−22 | 1.51527E−20 | 0.412234043 | 2.49508214 |
| 2 | GBE1 | 9.842813492 | 0.970255792 | 1.60649E−22 | 1.6678E−20 | 0.688164894 | 3.104842186 |
| 2 | MAFG | 9.823610306 | 1.43303597 | 2.10303E−22 | 2.1748E−20 | 0.253989362 | 2.525716066 |
| 2 | GPATCH8 | 9.772167206 | 0.865730166 | 3.17637E−22 | 3.19767E−20 | 0.74268617 | 2.926024437 |
| 2 | ACACB | 9.759922981 | 0.900359094 | 3.5882E−22 | 3.59863E−20 | 0.734707447 | 2.94533515 |
| 2 | PPM1B | 9.715722084 | 0.923463047 | 5.43049E−22 | 5.40547E−20 | 0.607712766 | 2.665096045 |
| 2 | EEPD1 | 9.696422577 | 1.093864083 | 6.629E−22 | 6.52515E−20 | 0.416223404 | 2.516578674 |
| 2 | TARID | 9.673109055 | 1.151974559 | 8.37063E−22 | 8.20908E−20 | 0.362367021 | 2.411636114 |
| 2 | OSBPL9 | 9.534450531 | 0.901503384 | 3.00012E−21 | 2.82745E−19 | 0.632978723 | 2.706339121 |
| 2 | LINC01584 | 9.483860016 | 1.870488167 | 5.66631E−21 | 5.30259E−19 | 0.150930851 | 2.291846037 |
| 2 | ASB2 | 9.44735527 | 1.022662044 | 6.82587E−21 | 6.34305E−19 | 0.436170213 | 2.405553579 |
| 2 | RP11-83A24.2 | 9.427179337 | 1.057042837 | 8.26976E−21 | 7.60503E−19 | 0.414893617 | 2.485334158 |
| 2 | DEPTOR | 9.424127579 | 0.989740551 | 8.41766E−21 | 7.71435E−19 | 0.505319149 | 2.646375179 |
| 2 | PHYHIPL | 9.384076118 | 1.4225595 | 1.30172E−20 | 1.17274E−18 | 0.231382979 | 2.428275824 |
| 2 | COL14A1 | 9.344724655 | 1.122022629 | 1.78481E−20 | 1.59178E−18 | 0.36768617 | 2.558378935 |
| 2 | SLC16A10 | 9.320101738 | 0.955679357 | 2.18484E−20 | 1.94203E−18 | 0.553191489 | 2.71603322 |
| 2 | BEST3 | 9.31470871 | 1.170551062 | 2.36769E−20 | 2.09754E−18 | 0.317819149 | 2.353159189 |
| 2 | DNAH11 | 9.299433708 | 1.076768398 | 2.69237E−20 | 2.36938E−18 | 0.378324468 | 2.433912516 |
| 2 | FAM155A | 9.262131691 | 1.371308208 | 3.94695E−20 | 3.4506E−18 | 0.23537234 | 2.352062225 |
| 2 | MAST2 | 9.225768089 | 0.934371293 | 5.16647E−20 | 4.48723E−18 | 0.527925532 | 2.54109621 |
| 2 | PEBP4 | 9.219557762 | 1.228525877 | 5.70626E−20 | 4.92388E−18 | 0.279920213 | 2.369147301 |
| 2 | CFAP100 | 9.217924118 | 1.199949741 | 5.74078E−20 | 4.93763E−18 | 0.293882979 | 2.300785542 |
| 2 | GRIK1 | 9.088432312 | 0.987134993 | 1.78551E−19 | 1.50112E−17 | 0.491356383 | 2.683677912 |
| 2 | SPPL2A | 9.088224411 | 0.950577438 | 1.79048E−19 | 1.50112E−17 | 0.477393617 | 2.476287365 |
| 2 | CMYA5 | 9.023292542 | 0.883891165 | 3.17903E−19 | 2.64028E−17 | 0.591090426 | 2.639010429 |
| 2 | KCNMA1 | 9.018539429 | 1.011719227 | 3.34156E−19 | 2.76662E−17 | 0.440824468 | 2.60061717 |
| 2 | KCNQ5 | 8.965885162 | 0.956833065 | 5.29094E−19 | 4.31341E−17 | 0.587765957 | 3.040906668 |
| 2 | UBAC1 | 8.906652451 | 1.001608729 | 8.99776E−19 | 7.2685E−17 | 0.399601064 | 2.376380682 |
| 2 | AC016831.7 | 8.857182503 | 0.77382791 | 1.377E−18 | 1.099E−16 | 0.774601064 | 3.111475706 |
| 2 | SLC24A1 | 8.845750809 | 1.101889372 | 1.55603E−18 | 1.23447E−16 | 0.319148936 | 2.379269838 |
| 2 | DENND2C | 8.752186775 | 1.03368485 | 3.46418E−18 | 2.69203E−16 | 0.378989362 | 2.531555414 |
| 2 | ASB8 | 8.726119041 | 0.953082442 | 4.313E−18 | 3.30336E−16 | 0.446808511 | 2.538386345 |
| 2 | UBE2E2 | 8.683895111 | 0.790107071 | 6.16727E−18 | 4.69649E−16 | 0.696143617 | 2.86138463 |
| 2 | PKD2L2 | 8.569160461 | 1.125851274 | 1.68633E−17 | 1.26247E−15 | 0.283909574 | 2.361500978 |
| 2 | RP11-380P13.1 | 8.551259041 | 1.430726767 | 2.04885E−17 | 1.52101E−15 | 0.186170213 | 2.574045897 |
| 2 | PDXDC1 | 8.524419785 | 0.702211797 | 2.39924E−17 | 1.77124E−15 | 0.777260638 | 2.910685301 |
| 2 | FILIP1L | 8.515379906 | 0.823797643 | 2.58471E−17 | 1.90287E−15 | 0.640957447 | 2.917728901 |
| 2 | ATP2A2 | 8.500027657 | 0.930512011 | 2.95614E−17 | 2.17031E−15 | 0.446143617 | 2.588398695 |
| 2 | GATS | 8.444508553 | 1.130021214 | 4.80014E−17 | 3.46666E−15 | 0.271276596 | 2.269706011 |
| 2 | TPM3 | 8.438203812 | 0.85878402 | 4.93857E−17 | 3.55697E−15 | 0.57712766 | 2.807992458 |
| 2 | EIF4G3 | 8.40528965 | 0.854926169 | 6.51862E−17 | 4.66969E−15 | 0.519281915 | 2.659113169 |
| 2 | ZFAND1 | 8.402084351 | 1.048342943 | 6.78622E−17 | 4.83532E−15 | 0.325132979 | 2.506274939 |
| 2 | LRRFIP2 | 8.34126091 | 0.823379874 | 1.10601E−16 | 7.7763E−15 | 0.549202128 | 2.569671154 |
| 2 | CTNNA3 | 8.311929703 | 0.866425931 | 1.41056E−16 | 9.8914E−15 | 0.503989362 | 2.598663568 |
| 2 | PTPN3 | 8.299781799 | 1.117576003 | 1.59387E−16 | 1.11182E−14 | 0.267287234 | 2.335477352 |
| 2 | EPS15L1 | 8.271939278 | 0.853805006 | 1.96166E−16 | 1.35768E−14 | 0.488696809 | 2.521912575 |
| 2 | MAPT | 8.258669853 | 1.113215327 | 2.23206E−16 | 1.54082E−14 | 0.26462766 | 2.295737505 |
| 2 | P4HA1 | 8.247336388 | 0.935984075 | 2.40419E−16 | 1.65534E−14 | 0.432845745 | 2.670775414 |
| 2 | SYT2 | 8.231595993 | 0.960722983 | 2.74384E−16 | 1.87946E−14 | 0.373005319 | 2.456026077 |
| 2 | ERAP1 | 8.219478607 | 0.891569376 | 3.01582E−16 | 2.04468E−14 | 0.458776596 | 2.609760046 |
| 2 | RP11-133L19.1 | 8.214758873 | 1.749738216 | 3.41567E−16 | 2.30988E−14 | 0.124335106 | 2.209592819 |
| 2 | DOCK10 | 8.143771172 | 1.49682188 | 5.92012E−16 | 3.95324E−14 | 0.15625 | 2.539573431 |
| 2 | IRAK2 | 8.133694649 | 1.430084586 | 6.36126E−16 | 4.23717E−14 | 0.162898936 | 2.216449261 |
| 2 | RP11-445F6.2 | 8.12286377 | 1.370827556 | 6.89805E−16 | 4.56043E−14 | 0.174202128 | 2.224144936 |
| 2 | NFE2L3 | 8.095326424 | 0.951837718 | 8.28042E−16 | 5.46076E−14 | 0.366356383 | 2.468808413 |
| 2 | ATP1A2 | 8.089222908 | 0.867038071 | 8.64772E−16 | 5.68888E−14 | 0.44481383 | 2.456968546 |
| 2 | ARSG | 7.999431133 | 1.072402239 | 1.79935E−15 | 1.1579E−13 | 0.265292553 | 2.239055395 |
| 2 | EIF4E2 | 7.970070839 | 0.947133601 | 2.252E−15 | 1.44569E−13 | 0.342420213 | 2.356128931 |
| 2 | MLK7-AS1 | 7.928092957 | 0.908273697 | 3.13058E−15 | 1.98572E−13 | 0.380984043 | 2.442573547 |
| 2 | RP11-428F8.2 | 7.919212818 | 1.002526164 | 3.37391E−15 | 2.13496E−13 | 0.306515957 | 2.375439405 |
| 2 | THRAP3 | 7.899129868 | 0.856650889 | 3.92422E−15 | 2.47142E−13 | 0.417553191 | 2.407128572 |
| 2 | DIAPH2-AS1 | 7.895850658 | 0.88786751 | 4.02971E−15 | 2.53186E−13 | 0.423537234 | 2.674157381 |
| 2 | RAB7A | 7.849746227 | 0.843481243 | 5.7778E−15 | 3.6131E−13 | 0.433510638 | 2.49677825 |
| 2 | LRRTM3 | 7.812693596 | 0.938993394 | 7.72365E−15 | 4.78488E−13 | 0.356382979 | 2.513574362 |
| 2 | ZFAND3 | 7.810518742 | 0.681160331 | 7.80935E−15 | 4.81173E−13 | 0.707446809 | 2.791204214 |
| 2 | TBC1D1 | 7.80654335 | 0.942913294 | 8.12265E−15 | 4.97409E−13 | 0.33643617 | 2.44467473 |
| 2 | SSH2 | 7.682690144 | 0.678723037 | 2.09375E−14 | 1.25046E−12 | 0.734042553 | 3.044606686 |
| 2 | USP13 | 7.640108109 | 1.018036366 | 2.94052E−14 | 1.73667E−12 | 0.265957447 | 2.29551959 |
| 2 | ARPP21 | 7.630685806 | 0.841538072 | 3.11973E−14 | 1.83436E−12 | 0.447473404 | 2.60148859 |
| 2 | CFAP61 | 7.621867657 | 0.903961539 | 3.35116E−14 | 1.95745E−12 | 0.357047872 | 2.470448256 |
| 2 | KBTBD12 | 7.585996151 | 1.319558024 | 4.52771E−14 | 2.60461E−12 | 0.164228723 | 2.318667173 |
| 2 | CPED1 | 7.579452038 | 0.829537511 | 4.59863E−14 | 2.6397E−12 | 0.492021277 | 2.779601812 |
| 2 | TRIM72 | 7.573701382 | 1.158331871 | 4.9084E−14 | 2.81143E−12 | 0.20412234 | 2.252102613 |
| 2 | COL4A3 | 7.567313671 | 1.109659672 | 5.12323E−14 | 2.92189E−12 | 0.223404255 | 2.323210001 |
| 2 | HIGD1C | 7.490761757 | 1.303749919 | 9.25077E−14 | 5.11139E−12 | 0.160239362 | 2.224802971 |
| 2 | PDE7A | 7.487314224 | 0.746995151 | 9.18754E−14 | 5.08702E−12 | 0.541888298 | 2.599315166 |
| 2 | ABHD15-AS1 | 7.473214626 | 0.752368629 | 1.02167E−13 | 5.62173E−12 | 0.533244681 | 2.659134388 |
| 2 | ZBTB43 | 7.469352722 | 0.852468967 | 1.05467E−13 | 5.79129E−12 | 0.371010638 | 2.345040321 |
| 2 | CHRNA1 | 7.462652683 | 1.217321992 | 1.13203E−13 | 6.20329E−12 | 0.182180851 | 2.284247875 |
| 2 | MSI2 | 7.445295811 | 0.791202128 | 1.25917E−13 | 6.87165E−12 | 0.4375 | 2.401218891 |
| 2 | NME7 | 7.430614471 | 1.04057765 | 1.42328E−13 | 7.75131E−12 | 0.248670213 | 2.420376539 |
| 2 | ASB14 | 7.410612106 | 1.054909945 | 1.64883E−13 | 8.90669E−12 | 0.232047872 | 2.258043289 |
| 2 | TIAM2 | 7.376161575 | 0.796732664 | 2.10204E−13 | 1.12633E−11 | 0.421542553 | 2.398531914 |
| 2 | MACROD2 | 7.370851994 | 0.825578034 | 2.18733E−13 | 1.16967E−11 | 0.408244681 | 2.539567947 |
| 2 | MYO18B | 7.350703239 | 0.752539873 | 2.52859E−13 | 1.34136E−11 | 0.583776596 | 2.822394609 |
| 2 | CSPP1 | 7.348880768 | 0.834422827 | 2.5712E−13 | 1.36125E−11 | 0.380984043 | 2.377294302 |
| 2 | ADCY2 | 7.321660995 | 0.892778933 | 3.14894E−13 | 1.65722E−11 | 0.322473404 | 2.356746912 |
| 2 | DLG1 | 7.31735611 | 0.75628072 | 3.23429E−13 | 1.69877E−11 | 0.474069149 | 2.507163763 |
| 2 | LINC00691 | 7.308915138 | 1.610185266 | 3.6071E−13 | 1.89085E−11 | 0.11037234 | 2.206365347 |
| 2 | GPAT3 | 7.292189598 | 1.305723786 | 4.01126E−13 | 2.08217E−11 | 0.153590426 | 2.250618219 |
| 2 | CLTCL1 | 7.243478775 | 0.799707294 | 5.54103E−13 | 2.85395E−11 | 0.428856383 | 2.532205343 |
| 2 | TMCC1 | 7.229106903 | 0.791059196 | 6.15695E−13 | 3.14679E−11 | 0.409574468 | 2.442850351 |
| 2 | SGCD | 7.191203594 | 0.806258261 | 8.0903E−13 | 4.10336E−11 | 0.408909574 | 2.51824975 |
| 2 | ALPK2 | 7.183315277 | 1.264003873 | 8.77787E−13 | 4.41836E−11 | 0.15625 | 2.265405655 |
| 2 | COL4A4 | 7.179478645 | 1.164447427 | 8.95119E−13 | 4.49708E−11 | 0.18018617 | 2.246918917 |
| 2 | SOX6 | 7.16583252 | 0.795013547 | 9.70982E−13 | 4.86902E−11 | 0.415558511 | 2.5423491 |
| 2 | TXLNB | 7.127572536 | 0.994462132 | 1.28957E−12 | 6.43021E−11 | 0.236702128 | 2.283907652 |
| 2 | CACNA2D1 | 7.103878498 | 0.670570374 | 1.50809E−12 | 7.44989E−11 | 0.627659574 | 2.766659737 |
| 2 | LINC-PINT | 7.060790539 | 0.626786828 | 2.04782E−12 | 1.00415E−10 | 0.76462766 | 3.220552683 |
| 2 | DTNA | 7.053276062 | 0.878751934 | 2.17401E−12 | 1.06211E−10 | 0.313164894 | 2.491229534 |
| 2 | RP11-266N13.2 | 7.053152084 | 0.859615445 | 2.17066E−12 | 1.06211E−10 | 0.328457447 | 2.394334316 |
| 2 | C1orf21 | 7.033145905 | 0.672531784 | 2.48985E−12 | 1.21418E−10 | 0.585771277 | 2.634563684 |
| 2 | FEZ2 | 7.001177311 | 0.784137785 | 3.1247E−12 | 1.51819E−10 | 0.436835106 | 2.704600573 |
| 2 | ZNF385B | 7.000661373 | 0.920648813 | 3.15161E−12 | 1.52569E−10 | 0.290558511 | 2.595630169 |
| 2 | DLGAP4 | 6.988506317 | 0.908205986 | 3.43426E−12 | 1.65348E−10 | 0.270611702 | 2.278238773 |
| 2 | LGR5 | 6.97383976 | 1.221495271 | 3.86352E−12 | 1.8501E−10 | 0.15625 | 2.319181204 |
| 2 | HDAC4 | 6.968994617 | 0.70619911 | 3.90869E−12 | 1.86837E−10 | 0.525930851 | 2.645893335 |
| 2 | RUVBL1 | 6.956312656 | 0.993451178 | 4.31675E−12 | 2.05087E−10 | 0.222739362 | 2.244528055 |
| 2 | PPP1R12B | 6.941595554 | 0.717509031 | 4.73536E−12 | 2.24334E−10 | 0.482712766 | 2.561249495 |
| 2 | SORBS1 | 6.93281126 | 0.73868221 | 5.03104E−12 | 2.37495E−10 | 0.484707447 | 2.639477253 |
| 2 | RP11-96C21.2 | 6.89608717 | 1.503910899 | 6.71719E−12 | 3.14855E−10 | 0.107047872 | 2.083877087 |
| 2 | SGCG | 6.890183449 | 1.089723945 | 6.85666E−12 | 3.20263E−10 | 0.186170213 | 2.260030508 |
| 2 | AC013461.1 | 6.889243126 | 0.701131463 | 6.80935E−12 | 3.18613E−10 | 0.496010638 | 2.52626133 |
| 2 | TMEM233 | 6.881015778 | 0.834152579 | 7.23403E−12 | 3.37296E−10 | 0.333111702 | 2.505241394 |
| 2 | GATSL2 | 6.858131409 | 1.320169568 | 8.6462E−12 | 4.02434E−10 | 0.130319149 | 2.146317244 |
| 2 | MAP4 | 6.836865902 | 0.654626906 | 9.76442E−12 | 4.52895E−10 | 0.577792553 | 2.609838724 |
| 2 | SLC25A33 | 6.834188461 | 0.920866072 | 1.00238E−11 | 4.64115E−10 | 0.253324468 | 2.32438612 |
| 2 | CDKL4 | 6.825937271 | 1.029004693 | 1.06446E−11 | 4.90296E−10 | 0.201462766 | 2.229254723 |
| 2 | ANO5 | 6.769399166 | 0.922652483 | 1.56019E−11 | 7.05189E−10 | 0.248005319 | 2.306553364 |
| 2 | CTBP2 | 6.733307838 | 0.759192407 | 1.9836E−11 | 8.89008E−10 | 0.378324468 | 2.395258188 |
| 2 | ABCC5 | 6.721916676 | 0.818307459 | 2.14662E−11 | 9.57225E−10 | 0.315159574 | 2.339352846 |
| 2 | AMOTL1 | 6.712009907 | 0.832210779 | 2.29666E−11 | 1.01901E−09 | 0.303191489 | 2.375254154 |
| 2 | FASTKD1 | 6.703478336 | 0.81862843 | 2.42977E−11 | 1.07626E−09 | 0.319148936 | 2.389727116 |
| 2 | PSME4 | 6.695572376 | 0.771811306 | 2.56162E−11 | 1.1309E−09 | 0.356382979 | 2.372953176 |
| 2 | TTC19 | 6.692688465 | 0.654589534 | 2.60384E−11 | 1.14763E−09 | 0.54787234 | 2.531679869 |
| 2 | ESR2 | 6.691162109 | 0.756260395 | 2.63503E−11 | 1.15946E−09 | 0.381648936 | 2.405297279 |
| 2 | BHLHE40 | 6.678644657 | 1.099456191 | 2.90425E−11 | 1.27581E−09 | 0.168882979 | 2.183031082 |
| 2 | BLCAP | 6.672067642 | 0.850983441 | 3.00658E−11 | 1.31858E−09 | 0.28125 | 2.261330366 |
| 2 | RP11-768F21.1 | 6.661456585 | 0.75990802 | 3.2195E−11 | 1.40963E−09 | 0.393617021 | 2.58310914 |
| 2 | LINC00958 | 6.637942791 | 1.567791581 | 3.89233E−11 | 1.68754E−09 | 0.093085106 | 2.155334234 |
| 2 | EMC10 | 6.634161949 | 0.729967237 | 3.85608E−11 | 1.6773E−09 | 0.60106383 | 3.165352583 |
| 2 | PRUNE2 | 6.63179636 | 1.108187437 | 3.98181E−11 | 1.72353E−09 | 0.176196809 | 2.86322546 |
| 2 | AP000318.2 | 6.62654829 | 1.657520413 | 4.22312E−11 | 1.81909E−09 | 0.085106383 | 2.133541346 |
| 2 | NOCT | 6.625837803 | 0.886966407 | 4.10187E−11 | 1.76973E−09 | 0.256648936 | 2.346415043 |
| 2 | PHKA1 | 6.609696865 | 0.86346215 | 4.55992E−11 | 1.94838E−09 | 0.276595745 | 2.355988741 |
| 2 | PPP2R3A | 6.606728077 | 0.729199946 | 4.63602E−11 | 1.97771E−09 | 0.400930851 | 2.421422482 |
| 2 | RP3-355L5.4 | 6.594410896 | 0.801982045 | 5.04221E−11 | 2.14754E−09 | 0.318484043 | 2.382540226 |
| 2 | VPS13A | 6.575816154 | 0.784998536 | 5.70615E−11 | 2.42644E−09 | 0.327792553 | 2.393537998 |
| 2 | SLCO5A1 | 6.554997921 | 1.385593295 | 6.67802E−11 | 2.80826E−09 | 0.111037234 | 2.1230762 |
| 2 | MYOT | 6.547663212 | 0.705812931 | 6.85211E−11 | 2.87691E−09 | 0.447473404 | 2.539206505 |
| 2 | RP11-17E2.2 | 6.502860069 | 1.31861937 | 9.39876E−11 | 3.89689E−09 | 0.11768617 | 2.196060419 |
| 2 | CNNM2 | 6.496202469 | 0.838362455 | 9.64911E−11 | 3.98206E−09 | 0.279920213 | 2.362019062 |
| 2 | RP5-1042K10.10 | 6.49488306 | 0.868191123 | 9.74152E−11 | 4.00774E−09 | 0.255984043 | 2.32771492 |
| 2 | CAMK2D | 6.493876457 | 0.71522969 | 9.77187E−11 | 4.01402E−09 | 0.400930851 | 2.471184254 |
| 2 | AC005237.4 | 6.490449905 | 0.778861642 | 1.00101E−10 | 4.09288E−09 | 0.359707447 | 2.74353385 |
| 2 | UBE2G1 | 6.490374565 | 0.766047239 | 1.00061E−10 | 4.09288E−09 | 0.337101064 | 2.360214233 |
| 2 | ACTN2 | 6.484542847 | 0.718066335 | 1.03785E−10 | 4.23631E−09 | 0.421542553 | 2.586266994 |
| 2 | TMEM219 | 6.454517841 | 1.030274987 | 1.27573E−10 | 5.15274E−09 | 0.177526596 | 2.287629843 |
| 2 | COL21A1 | 6.446806431 | 1.698665142 | 1.37601E−10 | 5.52419E−09 | 0.078457447 | 2.175926924 |
| 2 | AMPD3 | 6.428993225 | 1.043485403 | 1.50524E−10 | 6.03391E−09 | 0.170212766 | 2.195843935 |
| 2 | VAV1 | 6.421996117 | 1.270571589 | 1.58609E−10 | 6.31985E−09 | 0.120345745 | 2.094852686 |
| 2 | ANK3 | 6.416523933 | 0.983450234 | 1.62824E−10 | 6.47812E−09 | 0.195478723 | 2.291696787 |
| 2 | PIGB | 6.40383625 | 0.773530662 | 1.75676E−10 | 6.979E−09 | 0.322473404 | 2.356812 |
| 2 | MEF2C-AS1 | 6.379877567 | 0.846469343 | 2.05466E−10 | 8.0779E−09 | 0.265292553 | 2.412787676 |
| 2 | TRIM54 | 6.373447418 | 0.874460161 | 2.14255E−10 | 8.38624E−09 | 0.236037234 | 2.19879365 |
| 2 | FER1L5 | 6.365951061 | 1.664257288 | 2.31001E−10 | 9.01513E−09 | 0.078457447 | 2.045360327 |
| 2 | GPR75-ASB3 | 6.356722355 | 0.760176897 | 2.37948E−10 | 9.25906E−09 | 0.324468085 | 2.336653471 |
| 2 | RBM20 | 6.328431129 | 0.952661514 | 2.8672E−10 | 1.11081E−08 | 0.201462766 | 2.331152916 |
| 2 | CORO1C | 6.311127186 | 0.804936707 | 3.18976E−10 | 1.23218E−08 | 0.278590426 | 2.276013851 |
| 2 | ENAH | 6.306545258 | 0.777756095 | 3.28117E−10 | 1.26199E−08 | 0.308510638 | 2.384060144 |
| 2 | CTD-3252C9.4 | 6.304284573 | 0.982546866 | 3.35082E−10 | 1.28136E−08 | 0.183510638 | 2.259699345 |
| 2 | SLITRK4 | 6.271499157 | 1.00402844 | 4.12776E−10 | 1.56051E−08 | 0.178856383 | 2.339720249 |
| 2 | WIPI1 | 6.250328064 | 0.894716024 | 4.70211E−10 | 1.76257E−08 | 0.216755319 | 2.196087837 |
| 2 | RP11-82O19.1 | 6.198344231 | 0.896507144 | 6.52306E−10 | 2.41453E−08 | 0.214095745 | 2.295628071 |
| 2 | DIAPH2 | 6.198203564 | 0.593374729 | 6.4927E−10 | 2.40664E−08 | 0.628324468 | 2.927456856 |
| 2 | FAM214A | 6.195316315 | 0.731180668 | 6.62454E−10 | 2.44868E−08 | 0.333111702 | 2.372669458 |
| 2 | CUX1 | 6.172894955 | 0.58512336 | 7.60554E−10 | 2.79575E−08 | 0.589760638 | 2.639996767 |
| 2 | SPTB | 6.163532257 | 0.842561901 | 8.10368E−10 | 2.96655E−08 | 0.238031915 | 2.283783913 |
| 2 | CLCN4 | 6.117886066 | 1.201776743 | 1.08571E−09 | 3.9152E−08 | 0.119680851 | 2.141138315 |
| 2 | ZNF565 | 6.099840641 | 0.630702078 | 1.19802E−09 | 4.29621E−08 | 0.46875 | 2.511870861 |
| 2 | USP54 | 6.092183113 | 0.632852077 | 1.2562E−09 | 4.48951E−08 | 0.478058511 | 2.608493328 |
| 2 | LMOD3 | 6.081940651 | 1.026607752 | 1.34949E−09 | 4.80127E−08 | 0.156914894 | 2.213111877 |
| 2 | ALPK3 | 6.080654621 | 0.877632439 | 1.35421E−09 | 4.81161E−08 | 0.213430851 | 2.200994015 |
| 2 | AC016723.4 | 6.075102329 | 0.84170711 | 1.40046E−09 | 4.96929E−08 | 0.259973404 | 2.686074734 |
| 2 | HPN | 6.025946617 | 1.155745029 | 1.90982E−09 | 6.68738E−08 | 0.124335106 | 2.191125631 |
| 2 | TMEM65 | 6.023669243 | 0.997532547 | 1.92659E−09 | 6.73722E−08 | 0.16356383 | 2.209017277 |
| 2 | MYLK4 | 6.004740238 | 1.049763322 | 2.16494E−09 | 7.52125E−08 | 0.157579787 | 2.668658733 |
| 2 | OPTN | 6.004301071 | 0.709042132 | 2.15512E−09 | 7.49695E−08 | 0.333776596 | 2.346712351 |
| 2 | TTC39C | 6.004148483 | 1.119683146 | 2.18029E−09 | 7.5647E−08 | 0.13231383 | 2.413716793 |
| 2 | OVCH1-AS1 | 6.000938416 | 1.118994951 | 2.22102E−09 | 7.69591E−08 | 0.132978723 | 2.290840387 |
| 2 | MEGF11 | 5.976119995 | 1.030354619 | 2.57563E−09 | 8.86691E−08 | 0.151595745 | 2.285352945 |
| 2 | RP11-381K20.2 | 5.962713242 | 0.667358041 | 2.77139E−09 | 9.49166E−08 | 0.397606383 | 2.488774776 |
| 2 | EGLN1 | 5.960404396 | 0.682305694 | 2.81177E−09 | 9.61756E−08 | 0.359707447 | 2.385695457 |
| 2 | OTOA | 5.957276344 | 1.392138004 | 2.91492E−09 | 9.94479E−08 | 0.089095745 | 2.161202192 |
| 2 | VCL | 5.946313381 | 0.678719401 | 3.06172E−09 | 1.04189E−07 | 0.369680851 | 2.47167325 |
| 2 | REV3L | 5.944608212 | 0.564657569 | 3.08985E−09 | 1.04856E−07 | 0.59375 | 2.665827513 |
| 2 | STAG3 | 5.942227364 | 0.863872409 | 3.15011E−09 | 1.0665E−07 | 0.207446809 | 2.265917063 |
| 2 | SRL | 5.939573288 | 0.739136755 | 3.19183E−09 | 1.07925E−07 | 0.294547872 | 2.354399681 |
| 2 | TBC1D4 | 5.926537037 | 0.792892277 | 3.45782E−09 | 1.16327E−07 | 0.248670213 | 2.320417643 |
| 2 | AC097662.2 | 5.875979424 | 1.056208014 | 4.70075E−09 | 1.54619E−07 | 0.140957447 | 2.221962452 |
| 2 | FGF13 | 5.849714279 | 0.723235667 | 5.46742E−09 | 1.78292E−07 | 0.309840426 | 2.524003267 |
| 2 | PGM2 | 5.827294827 | 1.274441242 | 6.31175E−09 | 2.04321E−07 | 0.099734043 | 2.095081806 |
| 2 | DYSF | 5.80066061 | 0.964402974 | 7.33624E−09 | 2.35287E−07 | 0.161569149 | 2.199360847 |
| 2 | PPFIBP2 | 5.785217285 | 1.210274935 | 8.08065E−09 | 2.57815E−07 | 0.105718085 | 2.120364428 |
| 2 | TTTY14 | 5.7664361 | 0.809435129 | 8.95262E−09 | 2.8461E−07 | 0.231382979 | 2.485110044 |
| 2 | PARVB | 5.763261795 | 0.753966093 | 9.10975E−09 | 2.89259E−07 | 0.259308511 | 2.319234133 |
| 2 | ZNF350-AS1 | 5.757673264 | 1.114758849 | 9.47817E−09 | 2.99882E−07 | 0.121675532 | 2.193636894 |
| 2 | RP11-23N2.4 | 5.753952503 | 1.040232778 | 9.67165E−09 | 3.05277E−07 | 0.134973404 | 2.152262449 |
| 2 | MTFR1 | 5.748864174 | 0.655172706 | 9.89632E−09 | 3.11629E−07 | 0.35837766 | 2.380037069 |
| 2 | SLC8B1 | 5.739226818 | 0.856653571 | 1.05012E−08 | 3.29504E−07 | 0.199468085 | 2.270848036 |
| 2 | OIP5 | 5.73619175 | 0.879495442 | 1.06924E−08 | 3.34321E−07 | 0.186835106 | 2.26888442 |
| 2 | MAP3K19 | 5.719334602 | 0.948348999 | 1.1818E−08 | 3.67355E−07 | 0.160904255 | 2.288817167 |
| 2 | MAP4K1 | 5.686931133 | 0.710603178 | 1.42011E−08 | 4.35321E−07 | 0.29587766 | 2.410723686 |
| 2 | MYOM1 | 5.669675827 | 0.610397696 | 1.56639E−08 | 4.77407E−07 | 0.519946809 | 2.898183346 |
| 2 | LRRC39 | 5.654405594 | 0.811781704 | 1.71657E−08 | 5.20792E−07 | 0.212765957 | 2.287535429 |
| 2 | RP11-382A20.3 | 5.653769016 | 0.938583553 | 1.72848E−08 | 5.23208E−07 | 0.160904255 | 2.460542917 |
| 2 | ATG14 | 5.649010658 | 0.656497955 | 1.76644E−08 | 5.32276E−07 | 0.352393617 | 2.461256027 |
| 2 | PFKFB2 | 5.621373653 | 0.9212569 | 2.07826E−08 | 6.21303E−07 | 0.162234043 | 2.173455954 |
| 2 | ADGRA3 | 5.613108158 | 0.903705001 | 2.1787E−08 | 6.506E−07 | 0.167553191 | 2.158976316 |
| 2 | COBL | 5.601424694 | 0.819148958 | 2.32658E−08 | 6.92016E−07 | 0.204787234 | 2.338227034 |
| 2 | FSD2 | 5.594927788 | 0.762256205 | 2.41159E−08 | 7.13729E−07 | 0.244015957 | 2.441618681 |
| 2 | ACYP2 | 5.581379414 | 0.558083236 | 2.59908E−08 | 7.64959E−07 | 0.511968085 | 2.580866098 |
| 2 | EGF | 5.581150532 | 0.78766185 | 2.60889E−08 | 7.66799E−07 | 0.22606383 | 2.360974312 |
| 2 | MBP | 5.553784847 | 0.893547058 | 3.05331E−08 | 8.92715E−07 | 0.170212766 | 2.262450218 |
| 2 | RP11-145A3.1 | 5.524035931 | 0.824083567 | 3.60749E−08 | 1.04668E−06 | 0.199468085 | 2.354933023 |
| 2 | CDR2 | 5.514461994 | 0.727995872 | 3.80346E−08 | 1.09874E−06 | 0.252659574 | 2.346160412 |
| 2 | PHF20 | 5.492816448 | 0.602127314 | 4.28817E−08 | 1.22282E−06 | 0.399601064 | 2.430570126 |
| 2 | BCL6 | 5.490375519 | 0.492126763 | 4.34693E−08 | 1.23825E−06 | 0.751329787 | 3.059695959 |
| 2 | GTF2IRD1 | 5.487867832 | 0.596718013 | 4.40811E−08 | 1.25433E−06 | 0.422207447 | 2.535461187 |
| 2 | C10orf71 | 5.486516953 | 0.749490798 | 4.44894E−08 | 1.26459E−06 | 0.238031915 | 2.301535368 |
| 2 | TPD52L1 | 5.475030899 | 0.60684067 | 4.736E−08 | 1.34475E−06 | 0.414893617 | 2.559541941 |
| 2 | PPP3CB | 5.473104954 | 0.675850034 | 4.79563E−08 | 1.35733E−06 | 0.299202128 | 2.538953066 |
| 2 | COLQ | 5.468707561 | 1.216439962 | 4.95959E−08 | 1.4002E−06 | 0.092420213 | 2.270092487 |
| 2 | SPG11 | 5.466549873 | 0.617772222 | 4.96817E−08 | 1.4002E−06 | 0.366356383 | 2.411705494 |
| 2 | RP11-21A7A.2 | 5.447811604 | 0.787402928 | 5.52587E−08 | 1.54428E−06 | 0.209441489 | 2.287673235 |
| 2 | TMEM161B-AS1 | 5.43091774 | 0.646918237 | 6.05872E−08 | 1.67907E−06 | 0.32912234 | 2.439799786 |
| 2 | RP11-118M9.3 | 5.422039509 | 0.718441963 | 6.37151E−08 | 1.75659E−06 | 0.254654255 | 2.379956245 |
| 2 | MYH14 | 5.407691956 | 0.857244253 | 6.91151E−08 | 1.88978E−06 | 0.17087766 | 2.243025303 |
| 2 | AVIL | 5.393999577 | 0.770653486 | 7.4437E−08 | 2.03112E−06 | 0.210771277 | 2.233373404 |
| 2 | ERMP1 | 5.377088547 | 0.624706566 | 8.15449E−08 | 2.21145E−06 | 0.357712766 | 2.470643997 |
| 2 | MYF6 | 5.370432377 | 0.771897435 | 8.46606E−08 | 2.2936E−06 | 0.217420213 | 2.241729498 |
| 2 | EPB41L5 | 5.367351532 | 0.643374443 | 8.60772E−08 | 2.32724E−06 | 0.310505319 | 2.344174385 |
| 2 | DIAPH1 | 5.356560707 | 0.658435285 | 9.13237E−08 | 2.44915E−06 | 0.29587766 | 2.31312418 |
| 2 | RB1CC1 | 5.338965416 | 0.630823076 | 1.00528E−07 | 2.67978E−06 | 0.320478723 | 2.299019337 |
| 2 | CPEB3 | 5.335753918 | 0.712529361 | 1.02423E−07 | 2.72483E−06 | 0.244015957 | 2.32701683 |
| 2 | SLC16A1-AS1 | 5.320877552 | 0.739677608 | 1.11104E−07 | 2.94399E−06 | 0.224069149 | 2.32413435 |
| 2 | XIRP2 | 5.297699928 | 0.885619402 | 1.26183E−07 | 3.3138E−06 | 0.158909574 | 2.304145575 |
| 2 | CUTC | 5.296145916 | 0.683580697 | 1.26971E−07 | 3.33119E−06 | 0.261303191 | 2.24063468 |
| 2 | LINC00693 | 5.29613018 | 1.074190617 | 1.27683E−07 | 3.34658E−06 | 0.109042553 | 2.297201395 |
| 2 | DPF3 | 5.288731098 | 0.629940391 | 1.32119E−07 | 3.45604E−06 | 0.32712766 | 2.466894865 |
| 2 | SIL1 | 5.284527302 | 0.565946341 | 1.35089E−07 | 3.53024E−06 | 0.419547872 | 2.44504714 |
| 2 | RP11-434D9.1 | 5.27664423 | 0.579019785 | 1.40944E−07 | 3.67603E−06 | 0.621010638 | 3.249646664 |
| 2 | CTC-431G16.2 | 5.273184776 | 0.660621464 | 1.43705E−07 | 3.73338E−06 | 0.292553191 | 2.362579823 |
| 2 | KIAA1217 | 5.273075581 | 0.50251925 | 1.4366E−07 | 3.73338E−06 | 0.666888298 | 3.040780067 |
| 2 | FAM222B | 5.270100117 | 0.699471056 | 1.46239E−07 | 3.79549E−06 | 0.248005319 | 2.345353842 |
| 2 | WIPF3 | 5.248694897 | 0.922923386 | 1.64599E−07 | 4.25125E−06 | 0.138962766 | 2.200718641 |
| 2 | ADCK3 | 5.245719433 | 0.638436437 | 1.66614E−07 | 4.29078E−06 | 0.299202128 | 2.286367178 |
| 2 | PARK2 | 5.230483532 | 0.522558272 | 1.80688E−07 | 4.63976E−06 | 0.506648936 | 2.570507526 |
| 2 | CECR2 | 5.230251789 | 0.639021277 | 1.81084E−07 | 4.64544E−06 | 0.299202128 | 2.359642506 |
| 2 | RP4-651E10.4 | 5.221274376 | 1.035326481 | 1.90936E−07 | 4.88402E−06 | 0.111702128 | 2.157044649 |
| 2 | IP6K3 | 5.217689991 | 0.682236016 | 1.93725E−07 | 4.94584E−06 | 0.255984043 | 2.307218075 |
| 2 | MRVI1-AS1 | 5.215574265 | 1.191663504 | 1.97327E−07 | 5.03299E−06 | 0.087101064 | 2.151721001 |
| 2 | SESN1 | 5.206480503 | 0.505252779 | 2.05417E−07 | 5.22927E−06 | 0.595744681 | 2.732893705 |
| 2 | SLC22A4 | 5.200951576 | 0.65760237 | 2.11794E−07 | 5.38131E−06 | 0.283244681 | 2.388278961 |
| 2 | NT5C2 | 5.200555801 | 0.604123592 | 2.12137E−07 | 5.38487E−06 | 0.33643617 | 2.332500696 |
| 2 | FOXP2 | 5.199259758 | 0.61342144 | 2.1359E−07 | 5.41659E−06 | 0.329787234 | 2.358930111 |
| 2 | RC3H1 | 5.178891182 | 0.594044328 | 2.38085E−07 | 6.01483E−06 | 0.352393617 | 2.440408468 |
| 2 | ANK1 | 5.165530205 | 0.748299301 | 2.55942E−07 | 6.44144E−06 | 0.20412234 | 2.292758942 |
| 2 | RP11-298J20.3 | 5.164250851 | 0.824507475 | 2.57795E−07 | 6.47582E−06 | 0.168218085 | 2.201919556 |
| 2 | DBR1 | 5.15776062 | 2.104744196 | 2.71974E−07 | 6.78073E−06 | 0.037898936 | 1.943034887 |
| 2 | RP11-138A9.2 | 5.157030582 | 0.599418938 | 2.67394E−07 | 6.68534E−06 | 0.340425532 | 2.424469709 |
| 2 | PHTF2 | 5.148463726 | 0.654963195 | 2.79892E−07 | 6.96506E−06 | 0.274601064 | 2.364459515 |
| 2 | PTPRQ | 5.132305622 | 1.003187299 | 3.05877E−07 | 7.56143E−06 | 0.114361702 | 2.144971609 |
| 2 | CTD-3131K8.2 | 5.131914616 | 0.930362582 | 3.06317E−07 | 7.56143E−06 | 0.130319149 | 2.194966078 |
| 2 | TMEM38A | 5.131445408 | 0.719143689 | 3.06418E−07 | 7.56143E−06 | 0.21875 | 2.345672369 |
| 2 | UBE2H | 5.129792213 | 0.582150757 | 3.08697E−07 | 7.60355E−06 | 0.355718085 | 2.408732414 |
| 2 | SYNJ2 | 5.128334522 | 0.817882717 | 3.11759E−07 | 7.67188E−06 | 0.167553191 | 2.286309242 |
| 2 | LINC00582 | 5.116597652 | 1.164651275 | 3.32989E−07 | 8.15654E−06 | 0.087765957 | 2.158380508 |
| 2 | ENKUR | 5.108791828 | 2.01131916 | 3.52156E−07 | 8.57075E−06 | 0.039228723 | 2.125641108 |
| 2 | PRKAR1A | 5.099963665 | 0.616424978 | 3.611E−07 | 8.77235E−06 | 0.302526596 | 2.314544439 |
| 2 | CASZ1 | 5.079336643 | 0.870612562 | 4.0321E−07 | 9.72585E−06 | 0.144946809 | 2.187793016 |
| 2 | TMEM164 | 5.075168133 | 0.780917168 | 4.11748E−07 | 9.91216E−06 | 0.181515957 | 2.260685444 |
| 2 | RP11-350J20.5 | 5.071537018 | 1.056420088 | 4.2102E−07 | 1.01262E−05 | 0.101728723 | 2.205650568 |
| 2 | MTERF4 | 5.06229496 | 0.655807614 | 4.39743E−07 | 1.0567E−05 | 0.267952128 | 2.427189589 |
| 2 | SACS | 5.056888103 | 0.929823041 | 4.53472E−07 | 1.08772E−05 | 0.127659574 | 2.19052124 |
| 2 | DST | 5.050804138 | 0.549804151 | 4.66428E−07 | 1.11779E−05 | 0.390957447 | 2.42303586 |
| 2 | UACA | 5.046833992 | 0.577182829 | 4.76143E−07 | 1.13901E−05 | 0.35106383 | 2.37626195 |
| 2 | TSNAX-DISC1 | 5.042760849 | 0.957462728 | 4.88095E−07 | 1.16446E−05 | 0.119015957 | 2.143039227 |
| 2 | SH3GL2 | 5.01850605 | 1.105840445 | 5.54292E−07 | 1.3083E−05 | 0.092420213 | 2.117537022 |
| 2 | MITF | 5.013531208 | 0.597330034 | 5.65661E−07 | 1.3304E−05 | 0.323803191 | 2.381858587 |
| 2 | C14orf159 | 5.007967472 | 0.695729315 | 5.82731E−07 | 1.36813E−05 | 0.221409574 | 2.293696165 |
| 2 | ABCC9 | 4.973855972 | 0.68419224 | 6.94246E−07 | 1.62135E−05 | 0.227393617 | 2.326007605 |
| 2 | ARL5A | 4.956633091 | 0.633221328 | 7.57881E−07 | 1.75914E−05 | 0.26462766 | 2.336920261 |
| 2 | VPS8 | 4.937561512 | 0.622744918 | 8.34941E−07 | 1.93126E−05 | 0.273271277 | 2.291983604 |
| 2 | TMPRSS12 | 4.93625927 | 1.126060367 | 8.44626E−07 | 1.95197E−05 | 0.085771277 | 2.159466982 |
| 2 | BTBD16 | 4.930104256 | 1.456743836 | 8.75716E−07 | 2.01855E−05 | 0.056515957 | 2.031391144 |
| 2 | RILPL1 | 4.927861214 | 0.708530009 | 8.77843E−07 | 2.0217E−05 | 0.20412234 | 2.263209105 |
| 2 | RP4-533D7.4 | 4.907061577 | 1.00498426 | 9.78076E−07 | 2.24282E−05 | 0.103058511 | 2.196311235 |
| 2 | TMOD1 | 4.884963036 | 0.754479051 | 1.09071E−06 | 2.47548E−05 | 0.175531915 | 2.249754429 |
| 2 | ANKRD27 | 4.8840518 | 0.823582768 | 1.09595E−06 | 2.48525E−05 | 0.14893617 | 2.12394619 |
| 2 | MPRIP | 4.883097649 | 0.615235269 | 1.09992E−06 | 2.49E−05 | 0.275265957 | 2.368479013 |
| 2 | IQGAP1 | 4.880649567 | 0.55669266 | 1.11307E−06 | 2.51548E−05 | 0.345079787 | 2.374480963 |
| 2 | USP32 | 4.872591019 | 0.565538287 | 1.15904E−06 | 2.60386E−05 | 0.333776596 | 2.352502823 |
| 2 | RP11-260M19.2 | 4.867860794 | 1.58446908 | 1.19977E−06 | 2.68856E−05 | 0.049202128 | 2.034079552 |
| 2 | RP11-729I10.2 | 4.866627693 | 0.983352423 | 1.19918E−06 | 2.68856E−05 | 0.105053191 | 2.193486452 |
| 2 | ANO6 | 4.864615917 | 0.556917489 | 1.20638E−06 | 2.7011E−05 | 0.345744681 | 2.398641586 |
| 2 | CTD-2140G10.2 | 4.852966309 | 1.120700598 | 1.28599E−06 | 2.85768E−05 | 0.083111702 | 2.172316074 |
| 2 | RASGRP3 | 4.852455616 | 0.764729381 | 1.28417E−06 | 2.85602E−05 | 0.170212766 | 2.28524065 |
| 2 | SLC3A1 | 4.851642609 | 0.78728497 | 1.28971E−06 | 2.86355E−05 | 0.158909574 | 2.282723188 |
| 2 | USP2 | 4.849550724 | 0.753689349 | 1.30298E−06 | 2.89059E−05 | 0.173537234 | 2.274746418 |
| 2 | LDB3 | 4.84837532 | 0.587759495 | 1.30875E−06 | 2.89985E−05 | 0.32912234 | 2.63176775 |
| 2 | SLC36A1 | 4.834427834 | 1.128569365 | 1.40966E−06 | 3.10394E−05 | 0.082446809 | 2.047160864 |
| 2 | CTB-171A8.1 | 4.820930958 | 0.87725246 | 1.50457E−06 | 3.30198E−05 | 0.126994681 | 2.254664183 |
| 2 | SCN1B | 4.81251812 | 0.885222971 | 1.56864E−06 | 3.43408E−05 | 0.125664894 | 2.219386578 |
| 2 | KLHL30 | 4.798117638 | 0.880001962 | 1.68433E−06 | 3.65126E−05 | 0.125664894 | 2.154909849 |
| 2 | RPS6KA5 | 4.793485165 | 0.647472978 | 1.72035E−06 | 3.72326E−05 | 0.236702128 | 2.319955111 |
| 2 | SDC4 | 4.783734798 | 0.795189261 | 1.80732E−06 | 3.88932E−05 | 0.152260638 | 2.19537425 |
| 2 | ANKUB1 | 4.781887531 | 0.551164448 | 1.82068E−06 | 3.91174E−05 | 0.347739362 | 2.403421879 |
| 2 | OGDH | 4.781089783 | 0.641642928 | 1.8292E−06 | 3.92687E−05 | 0.236702128 | 2.29778862 |
| 2 | INADL | 4.777573109 | 0.524470687 | 1.85976E−06 | 3.98926E−05 | 0.390292553 | 2.407418489 |
| 2 | TP53TG5 | 4.774615288 | 1.11797905 | 1.89567E−06 | 4.05972E−05 | 0.081781915 | 2.133109331 |
| 2 | SNX1 | 4.770822525 | 0.492551476 | 1.92223E−06 | 4.1133E−05 | 0.494680851 | 2.518059015 |
| 2 | RP11-138H8.2 | 4.766160965 | 0.735396028 | 1.96981E−06 | 4.21171E−05 | 0.176196809 | 2.213383198 |
| 2 | FXR1 | 4.765375137 | 0.613034606 | 1.97598E−06 | 4.22151E−05 | 0.25731383 | 2.238066435 |
| 2 | MNAT1 | 4.75870657 | 0.52800554 | 2.04073E−06 | 4.34936E−05 | 0.375 | 2.366028309 |
| 2 | MYB | 4.749276161 | 1.257892728 | 2.15006E−06 | 4.57137E−05 | 0.066489362 | 2.097510099 |
| 2 | GPRC5B | 4.746612549 | 0.963026881 | 2.173E−06 | 4.61592E−05 | 0.104388298 | 2.160535097 |
| 2 | RTN3 | 4.746134281 | 0.751122415 | 2.17449E−06 | 4.61592E−05 | 0.166888298 | 2.227902889 |
| 2 | TM4SF1-AS1 | 4.744234562 | 0.730098248 | 2.19384E−06 | 4.65329E−05 | 0.178191489 | 2.253133059 |
| 2 | SPON1 | 4.743897915 | 0.85684371 | 2.20054E−06 | 4.65931E−05 | 0.127659574 | 2.236042023 |
| 2 | P4HA3 | 4.741056919 | 0.594101727 | 2.22619E−06 | 4.70688E−05 | 0.280585106 | 2.317356348 |
| 2 | ABCC4 | 4.738475323 | 0.728698015 | 2.25724E−06 | 4.76117E−05 | 0.179521277 | 2.336945295 |
| 2 | DYX1C1 | 4.73783493 | 0.946880579 | 2.26784E−06 | 4.77973E−05 | 0.107047872 | 2.136003017 |
| 2 | TNIK | 4.735300541 | 0.625830412 | 2.29048E−06 | 4.82361E−05 | 0.25 | 2.328287125 |
| 2 | DGKG | 4.724738121 | 0.562551737 | 2.41066E−06 | 5.06467E−05 | 0.313829787 | 2.294270277 |
| 2 | TMEM120B | 4.722914696 | 0.895998359 | 2.43902E−06 | 5.11616E−05 | 0.117021277 | 2.199280262 |
| 2 | PPP1R27 | 4.721467018 | 0.543841958 | 2.44904E−06 | 5.12909E−05 | 0.390292553 | 2.621815443 |
| 2 | CEP128 | 4.703604698 | 0.602775395 | 2.67222E−06 | 5.56354E−05 | 0.315824468 | 2.793091297 |
| 2 | RP11-354H21.1 | 4.693300247 | 1.732629061 | 2.84217E−06 | 5.86918E−05 | 0.040558511 | 2.084527254 |
| 2 | ATP8A1 | 4.687127113 | 0.793894708 | 2.89989E−06 | 5.97446E−05 | 0.144946809 | 2.245044708 |
| 2 | BIRC6-AS2 | 4.668933392 | 0.480584174 | 3.16062E−06 | 6.45656E−05 | 0.449468085 | 2.430125237 |
| 2 | CMSS1 | 4.663343906 | 0.530028164 | 3.24813E−06 | 6.63022E−05 | 0.357047872 | 2.448280096 |
| 2 | RRAD | 4.660116196 | 0.923890114 | 3.30937E−06 | 6.74488E−05 | 0.10837766 | 2.311000824 |
| 2 | C8orf22 | 4.656207561 | 0.649439514 | 3.36205E−06 | 6.84175E−05 | 0.230718085 | 2.309743881 |
| 2 | NAA15 | 4.651958466 | 0.588736653 | 3.43212E−06 | 6.97365E−05 | 0.267287234 | 2.266418934 |
| 2 | PPP1R36 | 4.635500431 | 1.348193884 | 3.73693E−06 | 7.51828E−05 | 0.057180851 | 2.069847822 |
| 2 | PFKP | 4.625936508 | 0.986344278 | 3.89935E−06 | 7.80957E−05 | 0.09375 | 2.086039543 |
| 2 | CTD-2184D3.5 | 4.619717121 | 1.075563669 | 4.01971E−06 | 8.01439E−05 | 0.081781915 | 2.120187283 |
| 2 | GMPR | 4.598613739 | 0.670039475 | 4.43339E−06 | 8.77336E−05 | 0.196808511 | 2.228472471 |
| 2 | IQCK | 4.585191727 | 0.722120583 | 4.72641E−06 | 9.30473E−05 | 0.168882979 | 2.213634729 |
| 2 | HOMER2 | 4.585120201 | 0.613738537 | 4.72582E−06 | 9.30473E−05 | 0.242021277 | 2.358766556 |
| 2 | PFKFB3 | 4.579854012 | 0.717941284 | 4.84716E−06 | 9.51425E−05 | 0.17087766 | 2.175011396 |
| 2 | SLC6A6 | 4.577888489 | 0.873275816 | 4.89896E−06 | 9.59467E−05 | 0.115026596 | 2.135892153 |
| 2 | STAC3 | 4.577314377 | 0.582029879 | 4.90334E−06 | 9.59618E−05 | 0.268617021 | 2.298371315 |
| 2 | RP11-665C16.5 | 4.572444439 | 0.60165751 | 5.01824E−06 | 9.7994E−05 | 0.250664894 | 2.321034431 |
| 2 | AC074363.1 | 4.571641445 | 1.036847115 | 5.05382E−06 | 9.85818E−05 | 0.085771277 | 2.321335316 |
| 2 | IDNK | 4.568157196 | 0.622147262 | 5.12234E−06 | 9.97336E−05 | 0.232712766 | 2.390820265 |
| 2 | ADORA2B | 4.567165375 | 1.560429215 | 5.18681E−06 | 0.000100841 | 0.043882979 | 2.070227385 |
| 2 | KIAA0895 | 4.560043335 | 1.182671905 | 5.3474E−06 | 0.00010366 | 0.066489362 | 2.1240201 |
| 2 | THUMPD3-AS1 | 4.559513092 | 0.491155356 | 5.33259E−06 | 0.000103448 | 0.406914894 | 2.423635483 |
| 2 | RNF34 | 4.537159443 | 0.770193219 | 5.93488E−06 | 0.000113968 | 0.142952128 | 2.201545 |
| 2 | RAB30 | 4.536147594 | 0.616078615 | 5.958E−06 | 0.000114329 | 0.229388298 | 2.303420067 |
| 2 | VPS13D | 4.527026176 | 0.481384724 | 6.21533E−06 | 0.000118753 | 0.414228723 | 2.423736334 |
| 2 | RP11-144G7.2 | 4.523743629 | 0.921170712 | 6.32872E−06 | 0.000120572 | 0.10106383 | 2.179962635 |
| 2 | CH17-189H20.1 | 4.496156216 | 0.452778995 | 7.18132E−06 | 0.000135456 | 0.493351064 | 2.526069164 |
| 2 | GOLGA4 | 4.489851475 | 0.469936162 | 7.39645E−06 | 0.000139218 | 0.431515957 | 2.444284678 |
| 2 | ORC3 | 4.482268333 | 0.787237406 | 7.67565E−06 | 0.000143963 | 0.134308511 | 2.282672405 |
| 2 | ERBB4 | 4.480938911 | 0.62313056 | 7.7155E−06 | 0.000144507 | 0.220079787 | 2.385863304 |
| 2 | MLIP-AS1 | 4.477098465 | 0.587527156 | 7.8522E−06 | 0.000146654 | 0.248670213 | 2.306270599 |
| 2 | MYOM3 | 4.474375725 | 0.523002207 | 7.95028E−06 | 0.000148381 | 0.35106383 | 2.632186174 |
| 2 | PRKCA | 4.474197865 | 0.631493866 | 7.961E−06 | 0.000148477 | 0.214760638 | 2.301572323 |
| 2 | HOOK2 | 4.46565485 | 0.565872669 | 8.28049E−06 | 0.000154086 | 0.26662234 | 2.291747332 |
| 2 | SYTL3 | 4.465564728 | 0.782032728 | 8.29808E−06 | 0.000154222 | 0.13962766 | 2.33409977 |
| 2 | NKAIN2 | 4.465538502 | 0.601636946 | 8.28494E−06 | 0.000154086 | 0.241356383 | 2.347784519 |
| 2 | HSPB8 | 4.446873665 | 0.686398268 | 9.03745E−06 | 0.000166798 | 0.174867021 | 2.229338169 |
| 2 | STRIP2 | 4.446280956 | 0.951028824 | 9.08061E−06 | 0.000167478 | 0.091755319 | 2.202143431 |
| 2 | RP5-864K19.4 | 4.440191746 | 0.73697716 | 9.3263E−06 | 0.000171533 | 0.149601064 | 2.252022505 |
| 2 | PGM1 | 4.43453455 | 0.566936493 | 9.56262E−06 | 0.000175515 | 0.267287234 | 2.377950907 |
| 2 | ITGAD | 4.431231976 | 0.681656599 | 9.71462E−06 | 0.000177936 | 0.175531915 | 2.267474174 |
| 2 | RP11-15B24.5 | 4.427709103 | 0.849969447 | 9.88271E−06 | 0.000180517 | 0.113696809 | 2.18799758 |
| 2 | OSBPL1A | 4.422278881 | 0.67339623 | 1.01228E−05 | 0.000184777 | 0.182845745 | 2.357862234 |
| 2 | VLDLR | 4.419256687 | 0.973080099 | 1.02812E−05 | 0.000187281 | 0.087765957 | 2.086609602 |
| 2 | NRDC | 4.399225712 | 0.439257383 | 1.1242E−05 | 0.000203251 | 0.491356383 | 2.472644806 |
| 2 | ZC2HC1C | 4.395110607 | 0.77747792 | 1.14729E−05 | 0.000207144 | 0.130984043 | 2.182518721 |
| 2 | FBXO34 | 4.383724689 | 0.549224675 | 1.20723E−05 | 0.000216787 | 0.27393617 | 2.355151415 |
| 2 | RP13-650J16.1 | 4.381530762 | 1.104339838 | 1.22342E−05 | 0.000219398 | 0.069148936 | 2.066474676 |
| 2 | ALDH1L1-AS2 | 4.379236221 | 1.472185373 | 1.24005E−05 | 0.000222231 | 0.043882979 | 2.003632307 |
| 2 | LINC01504 | 4.375055313 | 1.127648711 | 1.26056E−05 | 0.000225148 | 0.067154255 | 2.151011229 |
| 2 | UBAP1 | 4.373998165 | 0.562529564 | 1.2619E−05 | 0.000225235 | 0.256648936 | 2.258039713 |
| 2 | LINC00222 | 4.365097046 | 1.07862246 | 1.31889E−05 | 0.000234619 | 0.071143617 | 2.260021448 |
| 2 | RHBDL2 | 4.358455658 | 0.829985797 | 1.35629E−05 | 0.000240789 | 0.114361702 | 2.194095373 |
| 2 | PHKA1-AS1 | 4.348987103 | 1.088021278 | 1.4188E−05 | 0.0002508 | 0.070478723 | 2.233006477 |
| 2 | LUCAT1 | 4.336723328 | 0.982026279 | 1.49816E−05 | 0.000263512 | 0.083111702 | 2.233794689 |
| 2 | RCAN2 | 4.334794521 | 0.664322972 | 1.50786E−05 | 0.000264691 | 0.179521277 | 2.170863628 |
| 2 | RP11-131H24.4 | 4.331271172 | 0.749518156 | 1.53291E−05 | 0.000268735 | 0.136968085 | 2.128373146 |
| 2 | ZRANB3 | 4.326035023 | 0.537874401 | 1.56821E−05 | 0.00027438 | 0.278590426 | 2.316257954 |
| 2 | ERBB2IP | 4.323107243 | 0.56600076 | 1.58919E−05 | 0.000277685 | 0.244680851 | 2.247745275 |
| 2 | RP11-305L7.6 | 4.319890499 | 1.06070292 | 1.61665E−05 | 0.000282298 | 0.072473404 | 2.033295155 |
| 2 | ZMYND8 | 4.314872742 | 0.567513168 | 1.6495E−05 | 0.000287279 | 0.250664894 | 2.449961662 |
| 2 | GATAD2B | 4.30777216 | 0.522739708 | 1.70245E−05 | 0.000295918 | 0.296542553 | 2.3401618 |
| 2 | CAPN9 | 4.306293964 | 0.660175383 | 1.71493E−05 | 0.000297893 | 0.178191489 | 2.401913166 |
| 2 | AJ006998.2 | 4.305747032 | 1.052289486 | 1.72372E−05 | 0.000299225 | 0.073803191 | 2.346470833 |
| 2 | NPIPB4 | 4.302113533 | 0.749290645 | 1.7479E−05 | 0.000302828 | 0.133643617 | 2.146938324 |
| 2 | STARD7 | 4.291378975 | 0.797507167 | 1.83496E−05 | 0.000316468 | 0.118351064 | 2.167705536 |
| 2 | REC114 | 4.288559437 | 0.85603857 | 1.85887E−05 | 0.000320383 | 0.103058511 | 2.240886211 |
| 2 | C14orf39 | 4.287269592 | 1.052510977 | 1.8723E−05 | 0.000322489 | 0.071808511 | 2.180736303 |
| 2 | KIAA0368 | 4.281984329 | 0.480920136 | 1.91076E−05 | 0.000328324 | 0.354388298 | 2.378825188 |
| 2 | HK1 | 4.2762146 | 0.702165425 | 1.96233E−05 | 0.000336687 | 0.151595745 | 2.191217422 |
| 2 | CEP85L | 4.273221493 | 0.497058362 | 1.98692E−05 | 0.000340247 | 0.332446809 | 2.361467838 |
| 2 | YBX3 | 4.270694733 | 0.405695707 | 2.00905E−05 | 0.000343594 | 0.569148936 | 2.568391323 |
| 2 | CTA-125H2.1 | 4.266901493 | 0.614403784 | 2.04457E−05 | 0.00034922 | 0.202792553 | 2.338463068 |
| 2 | KLF6 | 4.256903648 | 0.560994327 | 2.13717E−05 | 0.000363867 | 0.253324468 | 2.430104733 |
| 2 | AC026202.3 | 4.256497383 | 0.519266248 | 2.14065E−05 | 0.000364225 | 0.29587766 | 2.342800617 |
| 2 | AC011997.1 | 4.253777504 | 0.808385074 | 2.1695E−05 | 0.000368427 | 0.114361702 | 2.162931204 |
| 2 | MLXIP | 4.248038769 | 0.524927795 | 2.22274E−05 | 0.000376506 | 0.281914894 | 2.317705631 |
| 2 | LSMEM1 | 4.218584538 | 1.854095578 | 2.55376E−05 | 0.000426595 | 0.029920213 | 2.001717091 |
| 2 | PDZD2 | 4.217823982 | 0.43574217 | 2.53966E−05 | 0.000424506 | 0.463430851 | 2.505816698 |
| 2 | RP11-227G15.12 | 4.214144707 | 0.59601438 | 2.58261E−05 | 0.000430333 | 0.207446809 | 2.269631863 |
| 2 | RP11-689B22.2 | 4.209193707 | 0.939230561 | 2.64431E−05 | 0.000439786 | 0.083776596 | 2.206763744 |
| 2 | SPAG16 | 4.200991154 | 0.6940431 | 2.73778E−05 | 0.000454194 | 0.148271277 | 2.218439817 |
| 2 | RP4-633O19_A.1 | 4.189761162 | 1.139780641 | 2.88247E−05 | 0.000474938 | 0.060505319 | 2.104829073 |
| 2 | NXN | 4.189540386 | 0.540700555 | 2.8766E−05 | 0.000474264 | 0.256648936 | 2.301388741 |
| 2 | RP11-545I5.3 | 4.186182976 | 0.637062132 | 2.92028E−05 | 0.000480571 | 0.176861702 | 2.229890585 |
| 2 | MEF2C | 4.186130524 | 0.495769769 | 2.91934E−05 | 0.000480571 | 0.330452128 | 2.458991766 |
| 2 | SLC16A4 | 4.184505463 | 0.670722425 | 2.94304E−05 | 0.000483717 | 0.15625 | 2.28637743 |
| 2 | ARPP19 | 4.176621437 | 0.523282349 | 3.04374E−05 | 0.000497194 | 0.269281915 | 2.274440527 |
| 2 | CTB-12O2.1 | 4.174676895 | 1.200746894 | 3.08217E−05 | 0.000502236 | 0.053856383 | 2.13265729 |
| 2 | SF3A3 | 4.172715664 | 0.508602679 | 3.0962E−05 | 0.000503905 | 0.310505319 | 2.599680901 |
| 2 | ATP6V0A1 | 4.165957928 | 0.642266214 | 3.19044E−05 | 0.000518291 | 0.17087766 | 2.237739325 |
| 2 | PVT1 | 4.158743382 | 0.724934101 | 3.29325E−05 | 0.000534339 | 0.133643617 | 2.214698553 |
| 2 | SLC13A4 | 4.153281689 | 0.86888504 | 3.37505E−05 | 0.000546276 | 0.09375 | 2.245031357 |
| 2 | EXOC6 | 4.148400784 | 0.568780601 | 3.44211E−05 | 0.000556454 | 0.224069149 | 2.274961233 |
| 2 | ACSL1 | 4.143949032 | 0.440184027 | 3.50807E−05 | 0.000566084 | 0.438829787 | 2.514695406 |
| 2 | STAB2 | 4.143496037 | 1.086315513 | 3.52425E−05 | 0.00056835 | 0.064494681 | 2.115110397 |
| 2 | METTL7A | 4.132599831 | 0.495501339 | 3.68605E−05 | 0.000591215 | 0.304521277 | 2.415786982 |
| 2 | RALGAPA2 | 4.123486996 | 0.460755497 | 3.8331E−05 | 0.000611845 | 0.379654255 | 2.445731401 |
| 2 | ATPIF1 | 4.119070053 | 0.739237964 | 3.9107E−05 | 0.000622363 | 0.126329787 | 2.202701569 |
| 2 | TTLL11 | 4.117594242 | 0.730986893 | 3.93514E−05 | 0.000625422 | 0.129654255 | 2.123131275 |
| 2 | CLIP1-AS1 | 4.11471653 | 1.227391601 | 3.9969E−05 | 0.000634562 | 0.051196809 | 2.187669516 |
| 2 | MURC | 4.105834484 | 0.83299017 | 4.143E−05 | 0.000654629 | 0.10106383 | 2.255572557 |
| 2 | RP6-1O2.1 | 4.104628086 | 1.470376134 | 4.18025E−05 | 0.000658947 | 0.039228723 | 2.071115017 |
| 2 | CCPG1 | 4.100729465 | 0.52327919 | 4.2289E−05 | 0.00066622 | 0.258643617 | 2.261872292 |
| 2 | FAT2 | 4.100393295 | 1.447552443 | 4.25727E−05 | 0.000670293 | 0.039893617 | 2.109092712 |
| 2 | AC107218.3 | 4.096785069 | 1.096533298 | 4.31385E−05 | 0.0006788 | 0.061170213 | 2.208104372 |
| 2 | ARG2 | 4.094244003 | 0.648625016 | 4.35015E−05 | 0.000683702 | 0.16356383 | 2.315607071 |

Cluster 6 Myocyte

| | | | | | | | |
|---|---|---|---|---|---|---|---|
| 6 | MB | 39.83720016 | 5.848474026 | 1.2397E−170 | 3.2947E−166 | 0.984076433 | 4.411431313 |
| 6 | DES | 31.15672684 | 5.003727436 | 2.1267E−129 | 2.8261E−125 | 0.955414013 | 3.765634775 |
| 6 | CKM | 29.53772354 | 4.672203541 | 2.1414E−119 | 1.8971E−115 | 0.974522293 | 4.3091712 |
| 6 | ACTA1 | 29.14618683 | 4.024085999 | 8.406E−113 | 4.4681E−109 | 1 | 5.088143826 |
| 6 | TNNC1 | 28.60730362 | 5.138083935 | 7.7053E−115 | 5.1196E−111 | 0.926751592 | 4.129738808 |
| 6 | CA3 | 27.4859581 | 5.02908802 | 1.9241E−108 | 8.523E−105 | 0.914012739 | 3.980746031 |
| 6 | MT-CO1 | 27.27422333 | 4.710987568 | 5.2039E−105 | 1.9758E−101 | 0.987261146 | 4.879753113 |
| 6 | TNNI1 | 26.72697639 | 5.429759979 | 3.511E−99 | 9.3312E−96 | 0.853503185 | 3.790502548 |
| 6 | TPM2 | 26.55555153 | 4.384262085 | 1.6788E−104 | 5.5772E−101 | 0.952229299 | 3.758645535 |
| 6 | TCAP | 26.47858047 | 5.084837437 | 4.2077E−101 | 1.24252E−97 | 0.894904459 | 3.756009579 |
| 6 | MYH7 | 25.85125732 | 7.007844448 | 1.67015E−84 | 2.77423E−81 | 0.77388535 | 3.769806862 |
| 6 | MT-CO2 | 25.1748867 | 4.556524277 | 6.5541E−96 | 1.58353E−92 | 0.958598726 | 4.256231785 |
| 6 | MT-CO3 | 24.95495605 | 4.409442425 | 3.64701E−92 | 6.92333E−89 | 0.977707006 | 4.588115215 |
| 6 | TNNT1 | 24.9418335 | 4.417129993 | 7.20971E−96 | 1.59677E−92 | 0.933121019 | 4.080062389 |
| 6 | GAPDH | 24.46217918 | 4.216169357 | 3.29014E−93 | 6.72632E−90 | 0.926751592 | 3.574132204 |
| 6 | MYL2 | 24.11854362 | 4.330255508 | 2.25125E−91 | 3.98877E−88 | 0.939490446 | 4.47373867 |
| 6 | COX6A2 | 22.86060524 | 4.700991154 | 2.82024E−80 | 3.74767E−77 | 0.799363057 | 3.077271223 |
| 6 | ALDOA | 22.70361328 | 4.363072395 | 5.41699E−82 | 7.99818E−79 | 0.856687898 | 3.373957396 |
| 6 | CRYAB | 22.68585205 | 4.492691517 | 3.99141E−81 | 5.58315E−78 | 0.834394904 | 3.226887941 |
| 6 | RPLP1 | 22.51683044 | 3.852472305 | 1.70187E−82 | 2.66062E−79 | 0.926751592 | 3.499361277 |
| 6 | MT-ATP6 | 21.86286354 | 3.989280224 | 1.74332E−78 | 2.20629E−75 | 0.933121019 | 3.65584898 |
| 6 | NDUFA4 | 21.60279083 | 5.162376404 | 1.30485E−70 | 1.50779E−67 | 0.72611465 | 2.915601969 |
| 6 | MT-ND1 | 20.70478249 | 3.792089939 | 2.98556E−72 | 3.60669E−69 | 0.936305732 | 3.603648901 |
| 6 | MT-ND4 | 20.39348793 | 3.850634813 | 2.84541E−70 | 3.15093E−67 | 0.945859873 | 3.806608915 |
| 6 | MYL1 | 20.31270027 | 4.083716869 | 2.10591E−69 | 2.23875E−66 | 0.828025478 | 3.279913425 |
| 6 | MT-CYB | 20.11243057 | 3.805832624 | 4.45589E−69 | 4.55478E−66 | 0.929936306 | 3.698783398 |
| 6 | MYL3 | 19.7162571 | 4.685050488 | 5.10826E−63 | 4.68145E−60 | 0.710191083 | 3.224459171 |
| 6 | TPT1 | 19.47873878 | 3.606040239 | 2.52927E−66 | 2.48964E−63 | 0.882165605 | 3.481626749 |
| 6 | FHL1 | 19.27162933 | 3.673398495 | 4.1344E−65 | 3.92429E−62 | 0.840764331 | 3.296726942 |
| 6 | HSPB1 | 19.11385345 | 4.146230221 | 5.12594E−62 | 4.54107E−59 | 0.751592357 | 3.076497316 |
| 6 | SLN | 18.02791214 | 4.734770298 | 3.51884E−54 | 2.92251E−51 | 0.633757962 | 2.711476326 |
| 6 | ENO3 | 17.28436279 | 3.932779074 | 6.90429E−53 | 5.56046E−50 | 0.703821656 | 2.854046345 |
| 6 | FABP3 | 16.8943119 | 4.367267609 | 1.4498E−49 | 1.04138E−46 | 0.624203822 | 2.826528549 |
| 6 | SLC25A4 | 16.89264107 | 4.088418007 | 2.49601E−50 | 1.89533E−47 | 0.646496815 | 2.824111462 |
| 6 | S100A1 | 16.88267136 | 4.756689548 | 1.08777E−48 | 7.41276E−46 | 0.595541401 | 2.731713057 |
| 6 | RP11-451G4.2 | 16.81672668 | 3.551796198 | 1.27232E−51 | 9.94543E−49 | 0.742038217 | 3.018927574 |
| 6 | PYGM | 16.47625923 | 4.334189892 | 1.03667E−47 | 6.7199E−45 | 0.608280255 | 2.596897602 |
| 6 | MYL12A | 16.43404007 | 3.706966639 | 4.40405E−49 | 3.08017E−46 | 0.681528662 | 2.786414385 |
| 6 | TPM3 | 16.21154213 | 3.16280055 | 1.36886E−49 | 1.01056E−46 | 0.856687898 | 3.567439079 |
| 6 | CHCHD10 | 15.97803783 | 3.826856852 | 1.91274E−46 | 1.21035E−43 | 0.636942675 | 2.712611675 |
| 6 | MT-ND3 | 15.97677422 | 3.226217747 | 2.38101E−48 | 1.58201E−45 | 0.885350318 | 3.763057232 |
| 6 | COX7C | 15.90766048 | 3.768694639 | 2.91797E−46 | 1.76252E−43 | 0.649681529 | 2.743373871 |
| 6 | COX7A1 | 15.87713814 | 3.994883537 | 1.48311E−45 | 8.56881E−43 | 0.621019108 | 2.616437197 |
| 6 | KLHL41 | 15.71121883 | 3.576519251 | 1.0789E−45 | 6.372E−43 | 0.665605096 | 2.648685455 |
| 6 | MT2A | 15.58382702 | 3.02646184 | 2.35162E−46 | 1.45347E−43 | 0.891719745 | 3.63922286 |
| 6 | COX5B | 15.44740486 | 4.11659956 | 2.89092E−43 | 1.63472E−40 | 0.579617834 | 2.63394928 |
| 6 | COX7B | 15.35348797 | 4.487487793 | 3.3094E−42 | 1.79498E−39 | 0.547770701 | 2.571705818 |
| 6 | TNNC2 | 15.0782671 | 3.78750515 | 1.64401E−42 | 9.10265E−40 | 0.700636943 | 3.533701897 |
| 6 | COX6C | 14.69732475 | 4.373735905 | 1.64266E−39 | 8.73139E−37 | 0.525477707 | 2.567189217 |
| 6 | MYLPF | 14.09986305 | 3.802051544 | 8.99079E−38 | 4.68526E−35 | 0.630573248 | 3.323938131 |
| 6 | COX4I1 | 13.76220512 | 3.625170708 | 2.48114E−36 | 1.22113E−33 | 0.550955414 | 2.655890465 |
| 6 | ATP5B | 13.66136837 | 4.523721218 | 6.59897E−35 | 3.1318E−32 | 0.477707006 | 2.420374393 |
| 6 | CHCHD3 | 13.55173779 | 2.656811237 | 8.72811E−37 | 4.4609E−34 | 0.757961783 | 2.837158442 |
| 6 | FTH1 | 13.47929573 | 2.740170479 | 1.55171E−36 | 7.78109E−34 | 0.799363057 | 3.025433302 |
| 6 | FLNC | 13.35715103 | 4.380737782 | 9.44634E−34 | 4.25518E−31 | 0.464968153 | 2.578216076 |
| 6 | UQCRB | 13.3328104 | 3.403590679 | 9.31666E−35 | 4.34401E−32 | 0.563694268 | 2.615803003 |
| 6 | UBC | 13.06747532 | 2.517292261 | 1.11668E−34 | 5.11691E−32 | 0.818471338 | 3.144286394 |
| 6 | UQCRQ | 13.04396248 | 4.469062805 | 2.06509E−32 | 8.31576E−30 | 0.449044586 | 2.593964338 |
| 6 | RPS2 | 12.87554455 | 2.781955004 | 9.68323E−34 | 4.28919E−31 | 0.719745223 | 2.864788532 |
| 6 | ATP5J | 12.83750629 | 4.018354416 | 6.44649E−32 | 2.51953E−29 | 0.468152866 | 2.387762785 |
| 6 | MYOZ1 | 12.81481266 | 3.199861288 | 1.07257E−32 | 4.45399E−30 | 0.566878981 | 2.671786785 |
| 6 | COX5A | 12.77282906 | 4.068025112 | 1.30206E−31 | 4.94357E−29 | 0.461783439 | 2.472778082 |
| 6 | EIF1 | 12.76993656 | 2.644769669 | 2.73315E−33 | 1.1908E−30 | 0.732484076 | 2.870877028 |
| 6 | CFL2 | 12.7433939 | 3.713093281 | 8.26692E−32 | 3.1842E−29 | 0.493630573 | 2.525295734 |
| 6 | RPL13 | 12.69760895 | 2.620246887 | 5.04241E−33 | 2.12718E−30 | 0.767515924 | 2.98561573 |
| 6 | RPL32 | 12.67377949 | 2.868290424 | 1.2162E−32 | 4.97278E−30 | 0.656050955 | 2.877779484 |
| 6 | MT-ND2 | 12.52504635 | 2.765968323 | 2.95939E−32 | 1.17391E−29 | 0.834394904 | 3.603889704 |
| 6 | ATP5D | 12.52048874 | 3.574436426 | 5.38341E−31 | 2.01514E−28 | 0.493630573 | 2.519610405 |
| 6 | TPI1 | 12.45579529 | 4.165575981 | 2.98583E−30 | 1.05806E−27 | 0.445859873 | 2.367846489 |
| 6 | UBB | 12.39153671 | 3.875851393 | 3.24145E−30 | 1.13353E−27 | 0.458598726 | 2.401021957 |
| 6 | NDUFB10 | 12.16600037 | 3.846777439 | 2.51255E−29 | 8.14341E−27 | 0.445859873 | 2.382272243 |
| 6 | PGAM2 | 12.15643215 | 4.237393856 | 4.73619E−29 | 1.5079E−26 | 0.423566879 | 2.435847282 |
| 6 | TTN | 12.12346077 | 2.234381199 | 2.47687E−30 | 8.89564E−28 | 0.929936306 | 4.171300411 |
| 6 | RPS23 | 12.07213497 | 2.718776464 | 3.95571E−30 | 1.36534E−27 | 0.662420382 | 2.822665453 |
| 6 | RPS15 | 12.07129097 | 2.751295567 | 4.21056E−30 | 1.43467E−27 | 0.640127389 | 2.770020485 |
| 6 | ECH1 | 12.02353668 | 4.55356884 | 2.30454E−28 | 6.80531E−26 | 0.407643312 | 2.243359327 |
| 6 | RPL8 | 12.00917149 | 2.853495598 | 1.10489E−29 | 3.67059E−27 | 0.598726115 | 2.687901735 |
| 6 | UQCR10 | 11.99741936 | 3.72506547 | 9.51083E−29 | 2.97376E−26 | 0.445859873 | 2.398475647 |
| 6 | ATP5G3 | 11.9689045 | 3.703487635 | 1.178E−28 | 3.5986E−26 | 0.452229299 | 2.328253984 |
| 6 | RPS25 | 11.94757271 | 2.598839998 | 1.24516E−29 | 4.0855E−27 | 0.671974522 | 2.802983046 |
| 6 | LINC01405 | 11.86253738 | 3.920574665 | 4.54316E−28 | 1.32685E−25 | 0.433121019 | 2.535659075 |
| 6 | CALM1 | 11.83694935 | 3.278623819 | 2.05141E−28 | 6.12589E−26 | 0.49044586 | 2.499519587 |
| 6 | RPL10 | 11.77864933 | 2.420463324 | 4.76591E−29 | 1.5079E−26 | 0.75477707 | 2.998620272 |
| 6 | RPL13A | 11.71607018 | 2.570213079 | 1.01717E−28 | 3.14341E−26 | 0.691082803 | 2.941674948 |
| 6 | RPL7 | 11.69745445 | 2.704030991 | 1.68955E−28 | 5.10262E−26 | 0.617834395 | 2.721329451 |
| 6 | COX6B1 | 11.43009663 | 3.465876102 | 1.18582E−26 | 3.24902E−24 | 0.449044586 | 2.403296709 |
| 6 | RPL19 | 11.42047691 | 2.616695404 | 2.29327E−27 | 6.62482E−25 | 0.621019108 | 2.7061131 |
| 6 | SEPW1 | 11.36589527 | 3.028012276 | 1.01945E−26 | 2.82228E−24 | 0.5 | 2.429388762 |
| 6 | BDNF-AS | 11.35344505 | 2.594975233 | 4.28293E−27 | 1.21093E−24 | 0.621019108 | 2.620084524 |
| 6 | RPL41 | 11.34627247 | 2.429976463 | 3.1706E−27 | 9.06077E−25 | 0.703821656 | 2.850463629 |
| 6 | TNNI2 | 11.30660629 | 3.701234341 | 5.51006E−26 | 1.4792E−23 | 0.442675159 | 3.125465393 |
| 6 | GLUL | 11.26456356 | 2.468554974 | 6.87666E−27 | 1.9238E−24 | 0.694267516 | 2.909363031 |
| 6 | SLC25A3 | 11.16996765 | 3.387065649 | 1.13137E−25 | 2.94788E−23 | 0.433121019 | 2.472656727 |
| 6 | CHCHD2 | 11.03751659 | 2.566341162 | 9.57294E−26 | 2.51901E−23 | 0.579617834 | 2.501738548 |
| 6 | BIN1 | 11.02961349 | 2.539194584 | 9.56219E−26 | 2.51901E−23 | 0.595541401 | 2.659377575 |
| 6 | VDAC1 | 10.99265671 | 3.475937605 | 6.28746E−25 | 1.54724E−22 | 0.420382166 | 2.321255922 |
| 6 | HSPA8 | 10.98980331 | 4.113099575 | 1.23826E−24 | 3.0192E−22 | 0.369426752 | 2.305474281 |
| 6 | RPL35 | 10.9851799 | 2.697296143 | 1.74275E−25 | 4.4968E−23 | 0.554140127 | 2.702702522 |
| 6 | FHL3 | 10.97987843 | 3.158374548 | 4.31476E−25 | 1.09213E−22 | 0.458598726 | 2.328205824 |
| 6 | RPL29 | 10.96351624 | 2.633833408 | 1.92466E−25 | 4.91843E−23 | 0.570063694 | 2.695324659 |
| 6 | NDUFB2 | 10.94529629 | 4.02059269 | 1.79152E−24 | 4.32848E−22 | 0.378980892 | 2.367230654 |
| 6 | RPL23A | 10.86342716 | 2.645931005 | 4.90521E−25 | 1.22987E−22 | 0.554140127 | 2.624548197 |
| 6 | RPS24 | 10.8446722 | 2.568300962 | 5.14609E−25 | 1.2782E−22 | 0.582802548 | 2.69365263 |
| 6 | HSP90AB1 | 10.80989742 | 3.509408474 | 3.18426E−24 | 7.55608E−22 | 0.398089172 | 2.341883659 |
| 6 | PRDX6 | 10.72074986 | 2.689711571 | 2.19028E−24 | 5.24423E−22 | 0.522292994 | 2.420814991 |
| 6 | CYCS | 10.66394329 | 3.531106472 | 1.18301E−23 | 2.68725E−21 | 0.391719745 | 2.335688353 |
| 6 | OOEP | 10.55574417 | 2.681520939 | 9.02624E−24 | 2.06802E−21 | 0.522292994 | 2.607708693 |
| 6 | TOMM7 | 10.55457783 | 2.842906237 | 1.21181E−23 | 2.72935E−21 | 0.477707006 | 2.418735027 |
| 6 | RPS16 | 10.51268864 | 2.438357592 | 8.66964E−24 | 2.00359E−21 | 0.595541401 | 2.611675024 |
| 6 | ATP5A1 | 10.50959682 | 3.840837955 | 5.81138E−23 | 1.26598E−20 | 0.359872611 | 2.209000349 |
| 6 | RPS13 | 10.45961189 | 2.557323694 | 1.72277E−23 | 3.84756E−21 | 0.554140127 | 2.640197039 |
| 6 | RPL27 | 10.42077255 | 2.697085857 | 3.10428E−23 | 6.8752E−21 | 0.503184713 | 2.522346258 |
| 6 | NDRG2 | 10.39639854 | 2.599650145 | 3.32001E−23 | 7.29221E−21 | 0.522292994 | 2.350038528 |
| 6 | MDH2 | 10.30113506 | 3.875109673 | 3.39518E−22 | 6.99487E−20 | 0.347133758 | 2.288105965 |
| 6 | RPL37A | 10.28469372 | 2.388727427 | 7.14178E−23 | 1.54315E−20 | 0.585987261 | 2.622385025 |
| 6 | TXNRD1 | 10.2578249 | 2.295766592 | 7.7531E−23 | 1.64843E−20 | 0.633757962 | 2.885567665 |
| 6 | CYC1 | 10.24633884 | 3.424834013 | 3.77182E−22 | 7.71106E−20 | 0.378980892 | 2.35806942 |
| 6 | MYL6B | 10.23207569 | 3.346798658 | 4.08301E−22 | 8.22076E−20 | 0.394904459 | 2.598213196 |
| 6 | SKP1 | 10.22602654 | 2.658417702 | 1.84131E−22 | 3.85327E−20 | 0.487261146 | 2.37500453 |
| 6 | ANKRD2 | 10.20828152 | 2.421060085 | 1.06332E−22 | 2.24285E−20 | 0.636942675 | 3.002196312 |
| 6 | YBX1 | 10.1799221 | 2.957562685 | 3.92725E−22 | 7.96753E−20 | 0.426751592 | 2.526364088 |
| 6 | PKM | 10.17981052 | 3.21432209 | 5.26783E−22 | 1.0448E−19 | 0.398089172 | 2.351135731 |
| 6 | RPL21 | 10.11885548 | 2.416298866 | 3.06635E−22 | 6.36675E−20 | 0.563694268 | 2.592464209 |
| 6 | HSPB6 | 10.07480907 | 4.218875885 | 2.91723E−21 | 5.61821E−19 | 0.334394904 | 2.28572464 |
| 6 | RPL15 | 10.06388664 | 2.316087008 | 4.24501E−22 | 8.48268E−20 | 0.605095541 | 2.631431103 |
| 6 | IDH2 | 10.04474068 | 2.701833725 | 9.14712E−22 | 1.80076E−19 | 0.468152866 | 2.465368986 |
| 6 | RPS27A | 9.975423813 | 2.364619732 | 9.45982E−22 | 1.84863E−19 | 0.579617834 | 2.727686644 |
| 6 | EEF2 | 9.911677361 | 2.52556324 | 2.1952E−21 | 4.25852E−19 | 0.512738854 | 2.48076582 |
| 6 | UQCRFS1 | 9.886471748 | 3.539829016 | 7.98387E−21 | 1.47352E−18 | 0.350318471 | 2.211768389 |
| 6 | TPM1 | 9.83283329 | 2.695770979 | 5.56897E−21 | 1.04969E−18 | 0.496815287 | 2.754993677 |
| 6 | BAIAP2L1 | 9.819220543 | 2.399255991 | 4.18477E−21 | 8.00133E−19 | 0.538216561 | 2.656004906 |
| 6 | ATP5G2 | 9.817615509 | 2.825157642 | 7.24879E−21 | 1.3567E−18 | 0.426751592 | 2.364445448 |
| 6 | SOD2 | 9.776478767 | 2.158680201 | 5.0665E−21 | 9.61803E−19 | 0.656050955 | 2.971051693 |
| 6 | MIR1-1HG | 9.727311134 | 3.431264162 | 2.58981E−20 | 4.68226E−18 | 0.356687898 | 2.180076838 |
| 6 | XPO5 | 9.726285934 | 2.281081438 | 7.93109E−21 | 1.47352E−18 | 0.579617834 | 2.628914595 |
| 6 | RPS12 | 9.718472481 | 2.459528208 | 1.06256E−20 | 1.94756E−18 | 0.50955414 | 2.589418173 |
| 6 | DHFR | 9.67085743 | 3.177795172 | 3.51775E−20 | 6.31697E−18 | 0.382165605 | 2.372789383 |
| 6 | RPL30 | 9.538219452 | 2.351884127 | 4.43056E−20 | 7.90275E−18 | 0.52866242 | 2.704521418 |
| 6 | LGALS1 | 9.537519455 | 3.09080863 | 9.66749E−20 | 1.65763E−17 | 0.378980892 | 2.443509102 |
| 6 | FAU | 9.515076637 | 2.323873997 | 5.05937E−20 | 8.9642E−18 | 0.52866242 | 2.455996752 |
| 6 | ADCK3 | 9.510059357 | 2.219378948 | 5.3894E−20 | 9.48571E−18 | 0.550955414 | 2.492744446 |
| 6 | LDHA | 9.499980927 | 2.929221869 | 1.17696E−19 | 1.99236E−17 | 0.398089172 | 2.43539238 |
| 6 | ZNF90 | 9.474774361 | 2.419223309 | 7.82325E−20 | 1.36789E−17 | 0.496815287 | 2.430942297 |
| 6 | EEF1B2 | 9.471288681 | 2.988833904 | 1.48714E−19 | 2.48577E−17 | 0.378980892 | 2.311306953 |
| 6 | RPLP0 | 9.458724976 | 2.293602705 | 8.07651E−20 | 1.40294E−17 | 0.544585987 | 2.53962183 |
| 6 | ATP5G1 | 9.448322296 | 3.633936644 | 2.89542E−19 | 4.80948E−17 | 0.321656051 | 2.26373148 |
| 6 | RPL27A | 9.448232651 | 2.233145475 | 8.2716E−20 | 1.4275E−17 | 0.566878981 | 2.717511654 |
| 6 | RPS15A | 9.438272476 | 2.432855368 | 1.09978E−19 | 1.87365E−17 | 0.49044586 | 2.56759429 |
| 6 | NDUFB7 | 9.404938698 | 3.510664701 | 3.83067E−19 | 6.28443E−17 | 0.321656051 | 2.274533749 |
| 6 | BTF3 | 9.344248772 | 2.742713213 | 3.31471E−19 | 5.47174E−17 | 0.407643312 | 2.363652706 |
| 6 | NDUFB11 | 9.323998451 | 3.363935709 | 6.38708E−19 | 1.03506E−16 | 0.340764331 | 2.157721043 |
| 6 | SOD1 | 9.288621902 | 3.347531319 | 8.54787E−19 | 1.37683E−16 | 0.334394904 | 2.203011036 |
| 6 | LINC00116 | 9.267432213 | 3.859466791 | 1.36329E−18 | 2.1313E−16 | 0.296178344 | 2.19382596 |
| 6 | MRPL41 | 9.25715065 | 3.537045956 | 1.25272E−18 | 1.98175E−16 | 0.318471338 | 2.223280191 |
| 6 | HSP90AA1 | 9.231017113 | 3.058588982 | 1.13407E−18 | 1.80481E−16 | 0.369426752 | 2.345919132 |
| 6 | MYH2 | 9.227009773 | 5.726491451 | 3.43614E−18 | 5.27874E−16 | 0.257961783 | 2.804262877 |
| 6 | RPS6 | 9.221483231 | 2.266475439 | 5.58095E−19 | 9.09969E−17 | 0.531847134 | 2.656741381 |
| 6 | RPL38 | 9.139832497 | 2.395781994 | 1.29502E−18 | 2.03655E−16 | 0.47133758 | 2.627782106 |
| 6 | NDUFB9 | 9.098916054 | 2.362968683 | 1.81381E−18 | 2.81904E−16 | 0.47133758 | 2.45674324 |
| 6 | POLR2L | 9.044656754 | 3.13692379 | 4.97847E−18 | 7.56074E−16 | 0.337579618 | 2.18762517 |
| 6 | C8orf22 | 9.026469231 | 2.566725492 | 4.04745E−18 | 6.18214E−16 | 0.423566879 | 3.039581299 |
| 6 | RPS4X | 9.009278297 | 2.249082804 | 3.05188E−18 | 4.71569E−16 | 0.525477707 | 2.623367786 |
| 6 | ATP5E | 8.987334251 | 2.892910957 | 6.67686E−18 | 9.91346E−16 | 0.359872611 | 2.324560404 |
| 6 | NDUFS5 | 8.928931236 | 2.940683126 | 1.10391E−17 | 1.62091E−15 | 0.343949045 | 2.431798458 |
| 6 | RPL14 | 8.928092957 | 2.240753889 | 6.1128E−18 | 9.17853E−16 | 0.506369427 | 2.577026367 |
| 6 | RPS20 | 8.927796364 | 2.275644302 | 6.46956E−18 | 9.65963E−16 | 0.49044586 | 2.648290873 |
| 6 | SMPX | 8.915446281 | 3.609665871 | 1.84237E−17 | 2.64675E−15 | 0.296178344 | 2.216426849 |
| 6 | YBX3 | 8.908596992 | 1.762720466 | 5.70631E−18 | 8.61685E−16 | 0.738853503 | 3.045453072 |
| 6 | NDUFS7 | 8.903167725 | 3.393578053 | 1.7885E−17 | 2.58332E−15 | 0.305732484 | 2.296674013 |
| 6 | MRPL33 | 8.890336037 | 2.694920063 | 1.2361E−17 | 1.80504E−15 | 0.385350318 | 2.320268393 |
| 6 | EIF4A2 | 8.873211861 | 2.338876724 | 1.03322E−17 | 1.52555E−15 | 0.458598726 | 2.306423903 |
| 6 | NDUFA1 | 8.853235245 | 3.218792915 | 2.33911E−17 | 3.30673E−15 | 0.315286624 | 2.291879892 |
| 6 | RPS5 | 8.817440033 | 2.396107197 | 1.66133E−17 | 2.41274E−15 | 0.445859873 | 2.473510027 |
| 6 | PLEKHA7 | 8.78331852 | 2.262879372 | 1.96833E−17 | 2.81249E−15 | 0.48089172 | 2.590897083 |
| 6 | RPS14 | 8.766196251 | 2.1856668 | 2.1176E−17 | 3.0096E−15 | 0.506369427 | 2.552545071 |
| 6 | RPL35A | 8.751791 | 2.23281312 | 2.38524E−17 | 3.3541E−15 | 0.493630573 | 2.564797878 |
| 6 | PRDX1 | 8.679678917 | 3.031894445 | 7.96873E−17 | 1.08608E−14 | 0.321656051 | 2.275750399 |
| 6 | RPL4 | 8.655236244 | 2.50569272 | 6.69334E−17 | 9.26505E−15 | 0.401273885 | 2.407427788 |
| 6 | AK1 | 8.641857147 | 3.228976727 | 1.20626E−16 | 1.62735E−14 | 0.315286624 | 2.298559189 |
| 6 | RPS3 | 8.607617378 | 2.206466198 | 7.53502E−17 | 1.03226E−14 | 0.477707006 | 2.626362324 |
| 6 | TNNT3 | 8.60564518 | 2.425774336 | 9.1013E−17 | 1.23411E−14 | 0.484076433 | 3.13482523 |
| 6 | GAMT | 8.546174049 | 3.54144001 | 2.70868E−16 | 3.54624E−14 | 0.277070064 | 2.17445612 |
| 6 | RPS8 | 8.528190613 | 2.111901522 | 1.30927E−16 | 1.75739E−14 | 0.50955414 | 2.572012186 |
| 6 | BNIP3 | 8.516699791 | 2.975203991 | 2.56294E−16 | 3.37204E−14 | 0.318471338 | 2.15829134 |
| 6 | RPL23 | 8.49579525 | 2.377194643 | 2.07621E−16 | 2.75898E−14 | 0.414012739 | 2.408300161 |
| 6 | RPS9 | 8.475420952 | 2.054802656 | 1.89964E−16 | 2.53702E−14 | 0.522292994 | 2.562847853 |
| 6 | RPS11 | 8.460236549 | 2.259877205 | 2.5308E−16 | 3.34632E−14 | 0.449044586 | 2.527521849 |
| 6 | XIRP2 | 8.42093277 | 2.581347704 | 4.20853E−16 | 5.48284E−14 | 0.375796178 | 2.376542807 |
| 6 | RP11-189B4.7 | 8.332479477 | 3.467294455 | 1.2823E−15 | 1.63061E−13 | 0.27388535 | 2.178607702 |
| 6 | HINT1 | 8.324763298 | 2.46413517 | 8.31655E−16 | 1.07819E−13 | 0.382165605 | 2.3344841 |
| 6 | C14orf2 | 8.291002274 | 2.68281126 | 1.18185E−15 | 1.51009E−13 | 0.347133758 | 2.215747356 |
| 6 | PSMB5 | 8.165976524 | 2.92429924 | 3.32254E−15 | 4.10713E−13 | 0.305732484 | 2.087266445 |
| 6 | NDUFB3 | 8.139015198 | 3.723731279 | 5.54331E−15 | 6.60649E−13 | 0.251592357 | 2.122669935 |
| 6 | RPL28 | 8.097658157 | 2.100669146 | 3.46451E−15 | 4.24314E−13 | 0.464968153 | 2.537796259 |
| 6 | SBDS | 8.096521378 | 2.887359858 | 5.41337E−15 | 6.48069E−13 | 0.312101911 | 2.159906149 |
| 6 | MT1E | 8.083218575 | 2.306572199 | 4.19425E−15 | 5.11333E−13 | 0.404458599 | 2.563595057 |
| 6 | RPS3A | 8.064387321 | 2.299508095 | 5.08677E−15 | 6.17311E−13 | 0.391719745 | 2.363358498 |
| 6 | PARK7 | 8.063223839 | 2.898157835 | 7.09E−15 | 8.26452E−13 | 0.299363057 | 2.250478745 |
| 6 | UQCRC1 | 8.053022385 | 3.664382696 | 1.00073E−14 | 1.1464E−12 | 0.248407643 | 2.11391592 |
| 6 | NDUFA6 | 8.043370247 | 3.470588446 | 1.00045E−14 | 1.1464E−12 | 0.261146497 | 1.995762825 |
| 6 | RPS19 | 8.021867752 | 2.049139023 | 5.9787E−15 | 7.0308E−13 | 0.474522293 | 2.636981487 |
| 6 | ATP2A2 | 7.995005608 | 1.824626684 | 6.71806E−15 | 7.86546E−13 | 0.582802548 | 2.848850727 |
| 6 | UBA52 | 7.963665485 | 2.189913988 | 9.95055E−15 | 1.1464E−12 | 0.410828025 | 2.434386015 |
| 6 | UQCRH | 7.961845398 | 2.645255566 | 1.25726E−14 | 1.42796E−12 | 0.318471338 | 2.123942614 |
| 6 | RPL11 | 7.945840359 | 1.888521791 | 9.52428E−15 | 1.10536E−12 | 0.557324841 | 2.627432823 |
| 6 | RPL34 | 7.904686451 | 2.022476196 | 1.36037E−14 | 1.53849E−12 | 0.484076433 | 2.647021294 |
| 6 | RPS29 | 7.897175789 | 2.271136284 | 1.68753E−14 | 1.89238E−12 | 0.388535032 | 2.220174313 |
| 6 | RPL7A | 7.890697956 | 2.117283106 | 1.63999E−14 | 1.84686E−12 | 0.433121019 | 2.514858484 |
| 6 | MAMDC2 | 7.845431805 | 1.958404422 | 2.07732E−14 | 2.30038E−12 | 0.493630573 | 2.578822613 |
| 6 | AC072062.1 | 7.843384743 | 2.110438347 | 2.31959E−14 | 2.558E−12 | 0.429936306 | 2.470484018 |
| 6 | APOO | 7.79784441 | 1.961782098 | 2.96171E−14 | 3.23923E−12 | 0.49044586 | 2.561257839 |
| 6 | MT-ND5 | 7.796954155 | 2.18674922 | 3.35789E−14 | 3.64256E−12 | 0.414012739 | 2.408314228 |
| 6 | RPS18 | 7.794633865 | 2.126239061 | 3.27039E−14 | 3.56218E−12 | 0.420382166 | 2.562694788 |
| 6 | RPLP2 | 7.792443752 | 1.739435911 | 2.86204E−14 | 3.14316E−12 | 0.598726115 | 2.693204165 |
| 6 | GHITM | 7.791748047 | 3.630871296 | 6.10083E−14 | 6.45983E−12 | 0.232484076 | 2.13280344 |
| 6 | RPL6 | 7.772835255 | 2.028194904 | 3.6592E−14 | 3.93727E−12 | 0.445859873 | 2.450348377 |
| 6 | RPL36 | 7.676739693 | 2.096570015 | 7.48828E−14 | 7.86625E−12 | 0.417197452 | 2.485702515 |
| 6 | KDM5B | 7.667917252 | 2.365488768 | 9.29398E−14 | 9.72465E−12 | 0.347133758 | 2.235472918 |
| 6 | FITM1 | 7.664886951 | 3.93430686 | 1.55177E−13 | 1.58621E−11 | 0.219745223 | 2.068377733 |
| 6 | EIF4G2 | 7.661688328 | 2.982192755 | 1.18998E−13 | 1.23059E−11 | 0.27388535 | 1.939021349 |
| 6 | SRP14 | 7.651302814 | 2.408586979 | 1.03432E−13 | 1.078E−11 | 0.337579618 | 2.162361383 |
| 6 | ATP5L | 7.62871027 | 2.231702805 | 1.15028E−13 | 1.19418E−11 | 0.369426752 | 2.245927095 |
| 6 | ADSSL1 | 7.622551441 | 3.264981985 | 1.7368E−13 | 1.76854E−11 | 0.248407643 | 2.115981102 |
| 6 | NDUFS6 | 7.565887928 | 3.214720249 | 2.53726E−13 | 2.54464E−11 | 0.251592357 | 2.055836678 |
| 6 | CST3 | 7.536521912 | 1.707644582 | 1.75142E−13 | 1.77662E−11 | 0.566878981 | 2.431108952 |
| 6 | MYH1 | 7.531177998 | 4.293118477 | 4.31625E−13 | 4.1866E−11 | 0.207006369 | 2.935866833 |
| 6 | GPX4 | 7.517097473 | 2.560539961 | 2.83229E−13 | 2.81924E−11 | 0.308917197 | 2.223956347 |
| 6 | EIF3K | 7.507629395 | 2.609425068 | 3.07297E−13 | 3.02483E−11 | 0.299363057 | 2.128605366 |
| 6 | NMRK2 | 7.493109703 | 2.5185287 | 3.25771E−13 | 3.19484E−11 | 0.308917197 | 2.172497988 |
| 6 | RPS17 | 7.477878094 | 2.032080889 | 2.92471E−13 | 2.90038E−11 | 0.417197452 | 2.303973913 |
| 6 | PPP1R1A | 7.443159103 | 4.23666811 | 7.2761E−13 | 6.88174E−11 | 0.197452229 | 2.039610147 |
| 6 | ATP5H | 7.439585686 | 2.769608498 | 5.13899E−13 | 4.94851E−11 | 0.270700637 | 2.168497324 |
| 6 | RPL10A | 7.430924416 | 1.828505039 | 3.80112E−13 | 3.71406E−11 | 0.487261146 | 2.498752594 |
| 6 | STAC3 | 7.387535572 | 1.840727329 | 5.33933E−13 | 5.12287E−11 | 0.461783439 | 2.425086498 |
| 6 | SH3BGR | 7.375995159 | 3.37429595 | 9.58753E−13 | 8.9721E−11 | 0.232484076 | 2.092885494 |
| 6 | RPS27 | 7.366811275 | 1.880596399 | 5.97037E−13 | 5.68726E−11 | 0.468152866 | 2.527093172 |
| 6 | ANAPC11 | 7.362109661 | 3.255420208 | 1.00667E−12 | 9.38742E−11 | 0.24522293 | 1.935515761 |
| 6 | RPL24 | 7.341626644 | 2.07626915 | 7.9721E−13 | 7.51328E−11 | 0.382165605 | 2.412303925 |
| 6 | PPDPF | 7.268880367 | 2.499433994 | 1.50477E−12 | 1.37905E−10 | 0.296178344 | 2.236425638 |
| 6 | PSMB6 | 7.263288498 | 3.45813942 | 2.02166E−12 | 1.82401E−10 | 0.21656051 | 2.063978195 |
| 6 | NDUFB4 | 7.262528896 | 2.700976849 | 1.68253E−12 | 1.53139E−10 | 0.267515924 | 2.194888115 |
| 6 | EEF1D | 7.262202263 | 2.127994776 | 1.37841E−12 | 1.26761E−10 | 0.366242038 | 2.285905361 |
| 6 | FEM1A | 7.234508991 | 2.951592445 | 2.16273E−12 | 1.94186E−10 | 0.251592357 | 2.017433405 |
| 6 | TCEB2 | 7.227610111 | 2.953775644 | 2.26807E−12 | 2.02958E−10 | 0.248407643 | 2.123178244 |
| 6 | BAG3 | 7.214540958 | 3.144127846 | 2.62987E−12 | 2.3376E−10 | 0.23566879 | 2.089898825 |
| 6 | HSPB8 | 7.213495255 | 2.106677294 | 1.92354E−12 | 1.74478E−10 | 0.363057325 | 2.276497364 |
| 6 | RAD23A | 7.206068039 | 2.120320082 | 2.02462E−12 | 1.82401E−10 | 0.359872611 | 2.192104816 |
| 6 | GSTP1 | 7.193813801 | 2.74570322 | 2.66635E−12 | 2.36212E−10 | 0.26433121 | 2.127870321 |
| 6 | AKR1C3 | 7.165279865 | 3.852488518 | 4.21511E−12 | 3.67295E−10 | 0.197452229 | 2.145964861 |
| 6 | PEBP1 | 7.148080349 | 2.265790462 | 3.16774E−12 | 2.78771E−10 | 0.321656051 | 2.2539289 |
| 6 | UQCRC2 | 7.121963978 | 2.52025795 | 4.00078E−12 | 3.49765E−10 | 0.28343949 | 2.104828835 |
| 6 | ISCU | 7.090894699 | 2.85912323 | 5.43666E−12 | 4.69124E−10 | 0.248407643 | 2.075759411 |
| 6 | JSRP1 | 7.084882259 | 3.015845537 | 5.94454E−12 | 5.11288E−10 | 0.23566879 | 2.135466814 |
| 6 | PDLIM1 | 7.077542305 | 2.01615119 | 4.59345E−12 | 3.97655E−10 | 0.369426752 | 2.243749857 |
| 6 | NDUFS8 | 7.062077522 | 2.558043718 | 6.03072E−12 | 5.17028E−10 | 0.270700637 | 2.142878294 |
| 6 | NDUFAB1 | 7.011957645 | 2.720952511 | 8.67542E−12 | 7.29641E−10 | 0.25477707 | 2.028431654 |
| 6 | MRLN | 6.998677731 | 2.306497097 | 8.45251E−12 | 7.13151E−10 | 0.302547771 | 2.129113197 |
| 6 | MYF6 | 6.996456146 | 2.041824818 | 8.10754E−12 | 6.86224E−10 | 0.363057325 | 2.577839613 |
| 6 | CASQ1 | 6.996096611 | 2.656916142 | 9.30871E−12 | 7.80434E−10 | 0.257961783 | 2.047023773 |
| 6 | TBC1D3P1-DHX40P1 | 6.98350668 | 2.278057098 | 9.35026E−12 | 7.81452E−10 | 0.305732484 | 2.347392082 |
| 6 | AURKAIP1 | 6.962955952 | 3.165995121 | 1.32586E−11 | 1.0809E−09 | 0.219745223 | 2.051373959 |
| 6 | PLEKHA6 | 6.951977253 | 2.002034187 | 1.04725E−11 | 8.67068E−10 | 0.366242038 | 2.35953331 |
| 6 | MKNK2 | 6.948469639 | 2.108167648 | 1.11089E−11 | 9.16899E−10 | 0.337579618 | 2.194122076 |
| 6 | GYG1 | 6.938387871 | 2.148659945 | 1.19485E−11 | 9.80106E−10 | 0.324840764 | 2.121073484 |
| 6 | PSMA7 | 6.926577091 | 2.343035221 | 1.37783E−11 | 1.11983E−09 | 0.296178344 | 2.154445171 |
| 6 | SERF2 | 6.906811714 | 1.737329841 | 1.28988E−11 | 1.0548E−09 | 0.461783439 | 2.273600101 |
| 6 | NPM1 | 6.905290127 | 2.769876957 | 1.73712E−11 | 1.39901E−09 | 0.242038217 | 2.096319437 |
| 6 | COX17 | 6.904477119 | 3.011754036 | 1.87516E−11 | 1.50562E−09 | 0.22611465 | 2.085191011 |
| 6 | MRPL14 | 6.868896484 | 3.118288755 | 2.39361E−11 | 1.90464E−09 | 0.21656051 | 2.053973436 |
| 6 | ACADVL | 6.866710186 | 1.792278647 | 1.71153E−11 | 1.38259E−09 | 0.423566879 | 2.272702932 |
| 6 | PPP1R27 | 6.83230114 | 1.561700702 | 2.00419E−11 | 1.60438E−09 | 0.585987261 | 2.631145239 |
| 6 | NEB | 6.826388836 | 1.413852572 | 2.10453E−11 | 1.67965E−09 | 0.843949045 | 3.738567114 |
| 6 | KIF5B | 6.816554546 | 2.773733616 | 3.10187E−11 | 2.43181E−09 | 0.238853503 | 2.132715225 |
| 6 | NFE2L1 | 6.811998367 | 2.299496412 | 2.8391E−11 | 2.23239E−09 | 0.292993631 | 2.073231697 |
| 6 | CSDE1 | 6.798049927 | 1.740706205 | 2.68685E−11 | 2.11894E−09 | 0.429936306 | 2.325774193 |
| 6 | USMG5 | 6.791281223 | 2.569707394 | 3.4804E−11 | 2.69675E−09 | 0.25477707 | 2.186696768 |
| 6 | PNRC1 | 6.775472641 | 1.830525279 | 3.12283E−11 | 2.44105E−09 | 0.394904459 | 2.265459776 |
| 6 | VDAC2 | 6.773963451 | 2.521859169 | 3.80321E−11 | 2.93831E−09 | 0.261146497 | 2.098874569 |
| 6 | NDUFA5 | 6.767721653 | 2.579209089 | 4.00897E−11 | 3.07937E−09 | 0.257961783 | 2.068995714 |
| 6 | RRAD | 6.743462563 | 2.379454851 | 4.42018E−11 | 3.36605E−09 | 0.27388535 | 2.136816025 |
| 6 | C19orf70 | 6.735521317 | 3.261544704 | 5.67193E−11 | 4.30694E−09 | 0.197452229 | 2.10765934 |
| 6 | RPS26 | 6.723680019 | 2.940528631 | 5.70365E−11 | 4.31869E−09 | 0.22611465 | 1.977766633 |
| 6 | CEBPB | 6.720240593 | 1.683670282 | 4.33099E−11 | 3.30761E−09 | 0.455414013 | 2.494323015 |
| 6 | RPL36AL | 6.696223259 | 2.332781792 | 5.89412E−11 | 4.45022E−09 | 0.27388535 | 2.129158735 |
| 6 | NDUFV2 | 6.680206299 | 2.67843318 | 7.07144E−11 | 5.29402E−09 | 0.242038217 | 2.018069029 |
| 6 | POMP | 6.663758755 | 2.445292711 | 7.44998E−11 | 5.54616E−09 | 0.261146497 | 2.105223179 |
| 6 | AC007325.4 | 6.654358864 | 3.730597973 | 1.00867E−10 | 7.44649E−09 | 0.181528662 | 1.99651742 |
| 6 | GNB2L1 | 6.649068356 | 1.761354089 | 6.87222E−11 | 5.15941E−09 | 0.414012739 | 2.432559967 |
| 6 | TMEM52 | 6.648231983 | 3.758371353 | 1.0219E−10 | 7.50252E−09 | 0.175159236 | 1.876889586 |
| 6 | BANF1 | 6.631938934 | 2.995990992 | 1.01818E−10 | 7.49592E−09 | 0.213375796 | 1.94349277 |
| 6 | NHSL2 | 6.607458115 | 2.416492462 | 1.0614E−10 | 7.74968E−09 | 0.261146497 | 2.178742886 |
| 6 | ATP5C1 | 6.577458382 | 2.724171162 | 1.32919E−10 | 9.6256E−09 | 0.22611465 | 1.964098811 |
| 6 | PHYH | 6.56237793 | 3.19760251 | 1.6252E−10 | 1.16423E−08 | 0.197452229 | 2.04442811 |
| 6 | NDUFA12 | 6.558354378 | 2.672056198 | 1.5123E−10 | 1.08628E−08 | 0.22611465 | 2.141530514 |
| 6 | SCN1B | 6.546739101 | 2.228321791 | 1.47699E−10 | 1.06379E−08 | 0.28343949 | 2.142511606 |
| 6 | SAP18 | 6.54515934 | 2.761873722 | 1.6486E−10 | 1.17782E−08 | 0.229299363 | 1.932505965 |
| 6 | EIF3G | 6.508601189 | 2.832638741 | 2.12389E−10 | 1.48936E−08 | 0.219745223 | 2.046257734 |
| 6 | PLEC | 6.486624718 | 3.050023079 | 2.49555E−10 | 1.7408E−08 | 0.200636943 | 1.954956174 |
| 6 | PRDX3 | 6.479523659 | 2.954427242 | 2.57085E−10 | 1.78862E−08 | 0.203821656 | 2.034767389 |
| 6 | UBL5 | 6.447841167 | 2.708240986 | 2.95489E−10 | 2.01882E−08 | 0.222929936 | 1.991869569 |
| 6 | LDB3 | 6.442974567 | 1.499522805 | 2.35632E−10 | 1.648E−08 | 0.531847134 | 2.436339378 |
| 6 | RPL5 | 6.437081814 | 1.790205479 | 2.63289E−10 | 1.81751E−08 | 0.372611465 | 2.34837079 |
| 6 | ST13 | 6.4275527 | 2.657666445 | 3.36578E−10 | 2.28195E−08 | 0.222929936 | 2.07605958 |
| 6 | MRFAP1 | 6.406045437 | 2.606434822 | 3.7564E−10 | 2.53385E−08 | 0.232484076 | 2.018181324 |
| 6 | AMBRA1 | 6.399333954 | 1.536644936 | 3.16845E−10 | 2.15365E−08 | 0.48089172 | 2.4746387 |
| 6 | RAN | 6.396848202 | 3.213811159 | 4.41389E−10 | 2.94744E−08 | 0.197452229 | 1.929839969 |
| 6 | TXNIP | 6.395721912 | 1.440415502 | 3.1319E−10 | 2.13427E−08 | 0.630573248 | 2.89118886 |
| 6 | RPS7 | 6.39133358 | 1.845736265 | 3.50741E−10 | 2.37192E−08 | 0.353503185 | 2.41274333 |
| 6 | TUBA4A | 6.387747288 | 2.665637732 | 4.20963E−10 | 2.82524E−08 | 0.21656051 | 2.032911301 |
| 6 | HFE2 | 6.352933407 | 4.083770752 | 6.39455E−10 | 4.17562E−08 | 0.162420382 | 1.999455214 |
| 6 | EID1 | 6.341440678 | 2.316843987 | 5.16678E−10 | 3.42438E−08 | 0.25477707 | 1.989193439 |
| 6 | EIF5A | 6.323873043 | 2.374746084 | 5.89145E−10 | 3.89495E−08 | 0.242038217 | 2.116633654 |
| 6 | ARPP19 | 6.322855473 | 1.577735186 | 5.11766E−10 | 3.4003E−08 | 0.439490446 | 2.324085236 |
| 6 | DAPK1 | 6.307159424 | 2.132127047 | 6.20663E−10 | 4.09314E−08 | 0.280254777 | 2.231853724 |
| 6 | RPSA | 6.294689178 | 1.794357419 | 6.23009E−10 | 4.09845E−08 | 0.356687898 | 2.253111362 |
| 6 | MGST3 | 6.273551941 | 2.118710756 | 7.57047E−10 | 4.89539E−08 | 0.27388535 | 2.175055265 |
| 6 | SLC26A3 | 6.230389118 | 1.695058465 | 9.10993E−10 | 5.87657E−08 | 0.382165605 | 2.23344183 |
| 6 | SYNM | 6.227944374 | 4.264685631 | 1.34755E−09 | 8.44663E−08 | 0.143312102 | 2.113853455 |
| 6 | SMDT1 | 6.226396084 | 2.941030264 | 1.15657E−09 | 7.31864E−08 | 0.194267516 | 1.970792651 |
| 6 | GABARAPL2 | 6.226214409 | 2.436339617 | 1.07326E−09 | 6.87327E−08 | 0.238853503 | 2.015880823 |
| 6 | FXYD1 | 6.223843575 | 2.499244928 | 1.09031E−09 | 6.9657E−08 | 0.229299363 | 1.96728313 |
| 6 | CNBP | 6.208116531 | 1.669372082 | 1.03412E−09 | 6.6386E−08 | 0.385350318 | 2.221218824 |
| 6 | HNRNPK | 6.198496342 | 1.964977145 | 1.15116E−09 | 7.30175E−08 | 0.299363057 | 2.124610662 |
| 6 | GOT2 | 6.195437908 | 3.324482918 | 1.46058E−09 | 9.13361E−08 | 0.171974522 | 1.99050951 |
| 6 | PSMD8 | 6.193841457 | 1.913351655 | 1.18579E−09 | 7.48569E−08 | 0.308917197 | 2.214182377 |
| 6 | HNRNPA2B1 | 6.175429344 | 1.55418539 | 1.22684E−09 | 7.70823E−08 | 0.429936306 | 2.205040693 |
| 6 | MDH1 | 6.153199673 | 2.001390934 | 1.50904E−09 | 9.3705E−08 | 0.289808917 | 2.100305796 |
| 6 | PTP4A3 | 6.147714615 | 3.015898705 | 1.84225E−09 | 1.13337E−07 | 0.184713376 | 1.955428839 |
| 6 | TUFM | 6.131894112 | 2.396129847 | 1.83672E−09 | 1.13258E−07 | 0.23566879 | 1.967435122 |
| 6 | NDUFB5 | 6.118462086 | 2.804485083 | 2.10736E−09 | 1.28457E−07 | 0.194267516 | 1.904133916 |
| 6 | ATPIF1 | 6.112909794 | 2.041523457 | 1.93976E−09 | 1.18786E−07 | 0.28343949 | 2.078413725 |
| 6 | DUSP26 | 6.071725845 | 3.420865297 | 3.0594E−09 | 1.83486E−07 | 0.162420382 | 2.132484913 |
| 6 | PSMA4 | 6.063336372 | 2.940116405 | 2.95101E−09 | 1.77441E−07 | 0.184713376 | 1.79628706 |
| 6 | PSMC3 | 6.057133198 | 3.269263983 | 3.21148E−09 | 1.90943E−07 | 0.168789809 | 1.871047258 |
| 6 | PCBP1 | 6.051906586 | 2.238918543 | 2.85733E−09 | 1.72589E−07 | 0.242038217 | 2.099956274 |
| 6 | PINK1 | 6.051905155 | 2.94956708 | 3.20444E−09 | 1.90943E−07 | 0.187898089 | 2.00139761 |
| 6 | PDHA1 | 6.032065868 | 3.084900856 | 3.60252E−09 | 2.11356E−07 | 0.175159236 | 1.880829811 |
| 6 | LINGO1 | 6.022808552 | 1.281723261 | 2.94636E−09 | 1.77441E−07 | 0.614649682 | 2.735240459 |
| 6 | PTP4A1 | 6.006503105 | 3.049422264 | 4.21438E−09 | 2.44774E−07 | 0.178343949 | 2.00026989 |
| 6 | SLC25A11 | 6.00530529 | 3.614897251 | 4.45981E−09 | 2.5767E−07 | 0.152866242 | 1.940012097 |
| 6 | RPS28 | 6.003494263 | 1.65488112 | 3.44604E−09 | 2.03976E−07 | 0.375796178 | 2.44879508 |
| 6 | GLRX | 5.999146938 | 1.660098314 | 3.54715E−09 | 2.08568E−07 | 0.369426752 | 2.273231506 |
| 6 | NDUFA2 | 5.981087685 | 2.809121847 | 4.66322E−09 | 2.68256E−07 | 0.191082803 | 1.95520401 |
| 6 | TIMM8B | 5.976626873 | 2.8593328 | 4.8476E−09 | 2.77661E−07 | 0.178343949 | 2.089822531 |
| 6 | CD59 | 5.966298103 | 1.534965277 | 4.21818E−09 | 2.44774E−07 | 0.410828025 | 2.311969757 |
| 6 | ADRM1 | 5.959323883 | 3.196215153 | 5.54809E−09 | 3.171E−07 | 0.168789809 | 1.911719084 |
| 6 | RPL18 | 5.956804276 | 1.716073751 | 4.53076E−09 | 2.61202E−07 | 0.347133758 | 2.265806198 |
| 6 | RGS5 | 5.955615997 | 3.563237429 | 5.96104E−09 | 3.36362E−07 | 0.146496815 | 2.761947393 |
| 6 | ZNF703 | 5.950178623 | 2.848650455 | 5.68377E−09 | 3.23464E−07 | 0.178343949 | 2.180998802 |
| 6 | MRPL51 | 5.938969612 | 2.98470211 | 6.09725E−09 | 3.43319E−07 | 0.175159236 | 1.989200115 |
| 6 | PRDX5 | 5.934810638 | 2.705857515 | 5.91432E−09 | 3.34436E−07 | 0.191082803 | 1.85509944 |
| 6 | HCFC1R1 | 5.917536736 | 2.997827768 | 6.81312E−09 | 3.81205E−07 | 0.168789809 | 2.045362949 |
| 6 | FABP4 | 5.91459322 | 2.993757725 | 6.91979E−09 | 3.8636E−07 | 0.165605096 | 2.73898983 |
| 6 | KLF10 | 5.907343864 | 3.091506481 | 7.37974E−09 | 4.08607E−07 | 0.168789809 | 1.981500506 |
| 6 | UTP11L | 5.898550987 | 2.526643991 | 7.21902E−09 | 4.01381E−07 | 0.200636943 | 2.073848963 |
| 6 | NDUFB1 | 5.888445377 | 2.221292734 | 7.28649E−09 | 4.04286E−07 | 0.232484076 | 2.074218035 |
| 6 | GTF3C6 | 5.879767895 | 3.294322252 | 8.77127E−09 | 4.79659E−07 | 0.159235669 | 2.012773752 |
| 6 | NDUFS3 | 5.870203495 | 3.088198423 | 9.02865E−09 | 4.9272E−07 | 0.168789809 | 1.864878178 |
| 6 | ATP5EP2 | 5.845889568 | 2.845281363 | 1.01023E−08 | 5.46843E−07 | 0.181528662 | 2.001421928 |
| 6 | RPL18A | 5.845119953 | 2.369808197 | 9.53726E−09 | 5.18347E−07 | 0.210191083 | 2.125791311 |
| 6 | RPL3L | 5.826365471 | 2.293392181 | 1.04575E−08 | 5.64896E−07 | 0.21656051 | 2.132558346 |
| 6 | DHRS7 | 5.78990221 | 1.899280667 | 1.22306E−08 | 6.56674E−07 | 0.277070064 | 2.207913399 |
| 6 | LDHB | 5.77646637 | 2.754955053 | 1.46482E−08 | 7.75844E−07 | 0.181528662 | 1.97334981 |
| 6 | YWHAG | 5.759435177 | 2.054791451 | 1.46022E−08 | 7.75844E−07 | 0.24522293 | 1.97055459 |
| 6 | PACSIN3 | 5.754849911 | 3.293042183 | 1.73808E−08 | 9.12905E−07 | 0.149681529 | 1.931787968 |
| 6 | B2M | 5.746181488 | 1.363481283 | 1.43416E−08 | 7.64722E−07 | 0.579617834 | 2.861454725 |
| 6 | ETFB | 5.726268291 | 3.079695702 | 2.0047E−08 | 1.04264E−06 | 0.162420382 | 1.977451682 |
| 6 | OST4 | 5.708218575 | 2.180375576 | 1.98284E−08 | 1.0333E−06 | 0.222929936 | 2.102248192 |
| 6 | GADD45GIP1 | 5.697387695 | 2.886531115 | 2.27733E−08 | 1.17523E−06 | 0.168789809 | 1.833654046 |
| 6 | NDUFA3 | 5.695615292 | 3.357994556 | 2.40836E−08 | 1.23565E−06 | 0.149681529 | 1.810893059 |
| 6 | FKBP3 | 5.692882538 | 2.268659353 | 2.17324E−08 | 1.12589E−06 | 0.21656051 | 1.93050468 |
| 6 | HNRNPA1 | 5.664699078 | 1.703234434 | 2.35028E−08 | 1.20819E−06 | 0.321656051 | 2.061017752 |
| 6 | ATP5F1 | 5.630986691 | 2.253083944 | 3.08685E−08 | 1.56863E−06 | 0.210191083 | 2.14928484 |
| 6 | ROMO1 | 5.617026806 | 3.063133955 | 3.60278E−08 | 1.79983E−06 | 0.149681529 | 1.989020705 |
| 6 | RASD1 | 5.616467953 | 2.06227994 | 3.18177E−08 | 1.60855E−06 | 0.24522293 | 2.341269016 |
| 6 | IGFBP5 | 5.607498169 | 1.47006762 | 3.13265E−08 | 1.58887E−06 | 0.420382166 | 2.335376263 |
| 6 | MYBPC1 | 5.604218483 | 1.243079901 | 3.25981E−08 | 1.64083E−06 | 0.805732484 | 3.240627527 |
| 6 | NDUFA8 | 5.601580143 | 3.076759577 | 3.88456E−08 | 1.93333E−06 | 0.149681529 | 1.813774586 |
| 6 | FAM177B | 5.594673634 | 3.812748432 | 4.34103E−08 | 2.14048E−06 | 0.133757962 | 2.043183327 |
| 6 | G0S2 | 5.578322411 | 2.557990074 | 4.14036E−08 | 2.04913E−06 | 0.181528662 | 1.94494617 |
| 6 | EIF3I | 5.571023464 | 2.823252916 | 4.46294E−08 | 2.19157E−06 | 0.162420382 | 1.903284192 |
| 6 | EDF1 | 5.566483498 | 1.874505639 | 4.13063E−08 | 2.04813E−06 | 0.261146497 | 2.050153255 |
| 6 | MAF1 | 5.562680721 | 2.294467449 | 4.4694E−08 | 2.19157E−06 | 0.203821656 | 2.090269327 |
| 6 | RASGEF1B | 5.523096561 | 1.407991052 | 4.99246E−08 | 2.42568E−06 | 0.414012739 | 2.293123484 |
| 6 | AKR1B1 | 5.498461246 | 2.369675636 | 6.31626E−08 | 3.03557E−06 | 0.187898089 | 2.025401831 |
| 6 | PABPC4 | 5.479220867 | 2.232622147 | 6.88979E−08 | 3.28743E−06 | 0.207006369 | 1.926041484 |
| 6 | SNX3 | 5.467146873 | 1.952953935 | 7.16466E−08 | 3.40635E−06 | 0.24522293 | 2.094460964 |
| 6 | H2AFZ | 5.452272892 | 2.176935196 | 7.93938E−08 | 3.76123E−06 | 0.207006369 | 2.023434639 |
| 6 | FIS1 | 5.451827526 | 2.30611062 | 8.09548E−08 | 3.82155E−06 | 0.194267516 | 2.07320857 |
| 6 | SYNPO | 5.440788746 | 2.475070477 | 8.68238E−08 | 4.07688E−06 | 0.178343949 | 1.91786921 |
| 6 | PDHB | 5.436583996 | 3.601681232 | 9.72408E−08 | 4.52604E−06 | 0.130573248 | 1.881632566 |
| 6 | PGK1 | 5.428820133 | 2.24627471 | 9.0937E−08 | 4.24751E−06 | 0.197452229 | 2.107058287 |
| 6 | MPC2 | 5.417705059 | 2.485899687 | 9.74191E−08 | 4.52641E−06 | 0.178343949 | 1.800565243 |
| 6 | PDLIM7 | 5.384880066 | 1.805688739 | 1.09222E−07 | 5.03958E−06 | 0.257961783 | 2.109219313 |
| 6 | LAMTOR5 | 5.381673336 | 1.958625078 | 1.11618E−07 | 5.13228E−06 | 0.22611465 | 1.909109592 |
| 6 | C20orf24 | 5.372620106 | 2.913415909 | 1.29732E−07 | 5.89381E−06 | 0.146496815 | 1.985575676 |
| 6 | FABP5 | 5.360343933 | 2.752927065 | 1.3579E−07 | 6.14803E−06 | 0.156050955 | 2.50101757 |
| 6 | ATF4 | 5.355871201 | 1.87923491 | 1.28399E−07 | 5.84795E−06 | 0.24522293 | 2.144037724 |
| 6 | HSF1 | 5.343218327 | 2.491482496 | 1.46358E−07 | 6.58166E−06 | 0.171974522 | 2.115264893 |
| 6 | ATP5I | 5.334709644 | 2.283126354 | 1.4979E−07 | 6.71328E−06 | 0.187898089 | 2.096036673 |
| 6 | ACADM | 5.329933167 | 2.257218599 | 1.51768E−07 | 6.79045E−06 | 0.187898089 | 2.017669916 |
| 6 | DDX3X | 5.314071655 | 1.923619151 | 1.59974E−07 | 7.14559E−06 | 0.23566879 | 2.032529831 |
| 6 | SRP9 | 5.305036068 | 2.050950289 | 1.69954E−07 | 7.52812E−06 | 0.213375796 | 2.003128529 |
| 6 | TMEM70 | 5.302285671 | 2.282974958 | 1.75126E−07 | 7.73144E−06 | 0.184713376 | 2.011030912 |
| 6 | KLHL40 | 5.300553799 | 2.881038666 | 1.87368E−07 | 8.23088E−06 | 0.143312102 | 1.928377151 |
| 6 | POLR2J | 5.29797554 | 2.90795207 | 1.90256E−07 | 8.34396E−06 | 0.143312102 | 1.96175766 |
| 6 | FTL | 5.295433998 | 1.313599586 | 1.65006E−07 | 7.35799E−06 | 0.512738854 | 2.712485075 |
| 6 | XIRP1 | 5.287570953 | 3.515011549 | 2.09258E−07 | 9.10222E−06 | 0.121019108 | 1.946791172 |
| 6 | PFN1 | 5.284497261 | 2.159352541 | 1.90991E−07 | 8.36239E−06 | 0.194267516 | 2.087047338 |
| 6 | ECHS1 | 5.280287266 | 3.114085674 | 2.10854E−07 | 9.15665E−06 | 0.136942675 | 1.850707054 |
| 6 | PSMB1 | 5.267874718 | 1.833682537 | 2.01226E−07 | 8.7816E−06 | 0.238853503 | 2.00788188 |
| 6 | VAMP5 | 5.263011932 | 2.421914577 | 2.18496E−07 | 9.4576E−06 | 0.171974522 | 2.03412962 |
| 6 | RBM8A | 5.261924267 | 1.880540013 | 2.06907E−07 | 9.0147E−06 | 0.23566879 | 1.842763662 |
| 6 | DLD | 5.258964539 | 2.444443703 | 2.23402E−07 | 9.65425E−06 | 0.171974522 | 1.948717594 |
| 6 | TUBB | 5.244320393 | 2.403403759 | 2.40014E−07 | 1.03385E−05 | 0.168789809 | 2.017887831 |
| 6 | ALKBH5 | 5.243580818 | 2.492257118 | 2.43157E−07 | 1.04569E−05 | 0.165605096 | 1.965886474 |
| 6 | GGACT | 5.229195118 | 2.238362312 | 2.5611E−07 | 1.09962E−05 | 0.184713376 | 2.052083969 |
| 6 | CCT7 | 5.22233057 | 3.239728689 | 2.87243E−07 | 1.22341E−05 | 0.127388535 | 1.982443571 |
| 6 | ANKRD9 | 5.20839262 | 2.45579958 | 2.90788E−07 | 1.23455E−05 | 0.168789809 | 1.989962935 |
| 6 | SNU13 | 5.206681728 | 2.186698198 | 2.85474E−07 | 1.21783E−05 | 0.191082803 | 2.029727221 |
| 6 | GMPR | 5.201354504 | 1.493198276 | 2.76112E−07 | 1.17978E−05 | 0.328025478 | 2.120718956 |
| 6 | SUCLA2 | 5.194231033 | 2.084656715 | 3.01971E−07 | 1.27998E−05 | 0.197452229 | 2.041510582 |
| 6 | PFKM | 5.190926075 | 1.47170198 | 2.90398E−07 | 1.23455E−05 | 0.337579618 | 2.200068235 |
| 6 | EEF1A1 | 5.162725449 | 1.382174611 | 3.31144E−07 | 1.39474E−05 | 0.423566879 | 2.693480968 |
| 6 | MYEOV2 | 5.156341076 | 2.467969418 | 3.80191E−07 | 1.58873E−05 | 0.162420382 | 2.052271366 |
| 6 | PDK4 | 5.147656441 | 1.125119567 | 3.62937E−07 | 1.51902E−05 | 0.847133758 | 3.527707577 |
| 6 | SELK | 5.137813568 | 2.480949879 | 4.14076E−07 | 1.71416E−05 | 0.162420382 | 1.896333933 |
| 6 | MYL12B | 5.132428169 | 2.27077651 | 4.18225E−07 | 1.72864E−05 | 0.175159236 | 1.982166886 |
| 6 | CKB | 5.128163815 | 3.109157324 | 4.53692E−07 | 1.8579E−05 | 0.130573248 | 1.891112208 |
| 6 | EMC6 | 5.114579201 | 2.541312456 | 4.6746E−07 | 1.9084E−05 | 0.156050955 | 1.866751671 |
| 6 | NAA38 | 5.111631393 | 2.163758516 | 4.60105E−07 | 1.88126E−05 | 0.184713376 | 1.91917944 |
| 6 | MBNL1-AS1 | 5.102065563 | 3.325333834 | 5.26853E−07 | 2.13653E−05 | 0.124203822 | 1.896459222 |
| 6 | RPL39 | 5.093800545 | 2.500615597 | 5.20467E−07 | 2.11505E−05 | 0.152866242 | 2.087671518 |
| 6 | SMTNL2 | 5.068628311 | 1.685076237 | 5.4868E−07 | 2.21615E−05 | 0.257961783 | 1.939144015 |
| 6 | RTN2 | 5.044530392 | 1.787690639 | 6.25509E−07 | 2.50742E−05 | 0.23566879 | 2.004944563 |
| 6 | FUNDC2 | 5.036552429 | 2.302867889 | 6.81781E−07 | 2.71253E−05 | 0.165605096 | 2.066447496 |
| 6 | MORF4L1 | 5.029250145 | 1.727575898 | 6.72044E−07 | 2.68182E−05 | 0.24522293 | 2.011368275 |
| 6 | ABRA | 5.023675919 | 2.094393253 | 7.11698E−07 | 2.8189E−05 | 0.187898089 | 1.979112387 |
| 6 | FAM96B | 5.020671844 | 1.850688457 | 7.10521E−07 | 2.81843E−05 | 0.21656051 | 2.055383444 |
| 6 | MRPL20 | 5.015284061 | 2.105089664 | 7.38687E−07 | 2.90415E−05 | 0.187898089 | 1.839118481 |
| 6 | EIF3E | 5.013790607 | 1.584902525 | 7.1589E−07 | 2.83128E−05 | 0.280254777 | 1.984084725 |
| 6 | BSG | 5.01049614 | 1.536850214 | 7.27372E−07 | 2.86391E−05 | 0.28343949 | 2.054036856 |
| 6 | MYOZ2 | 5.005451679 | 2.285573483 | 7.91806E−07 | 3.09924E−05 | 0.168789809 | 1.939579487 |
| 6 | ARMC9 | 5.00484848 | 1.470685244 | 7.45034E−07 | 2.92478E−05 | 0.315286624 | 2.240439653 |
| 6 | CSRP3 | 5.000202656 | 3.952128649 | 8.96384E−07 | 3.45765E−05 | 0.105095541 | 2.043411255 |
| 6 | SUCLG1 | 4.997919559 | 2.443722725 | 8.29229E−07 | 3.22199E−05 | 0.152866242 | 1.981320739 |
| 6 | NOP10 | 4.98609066 | 2.422877312 | 8.76839E−07 | 3.39211E−05 | 0.152866242 | 1.934735179 |
| 6 | PGM1 | 4.965318203 | 1.27323091 | 8.94792E−07 | 3.45652E−05 | 0.398089172 | 2.290755749 |
| 6 | MRPL57 | 4.957705498 | 2.986958027 | 1.04603E−06 | 3.99429E−05 | 0.127388535 | 1.876609802 |
| 6 | EEF1A2 | 4.951004028 | 2.332803249 | 1.04497E−06 | 3.99429E−05 | 0.156050955 | 2.14514184 |
| 6 | ALKBH7 | 4.948956966 | 2.746426105 | 1.07437E−06 | 4.09077E−05 | 0.133757962 | 1.898383498 |
| 6 | CHMP2A | 4.92509079 | 2.875642538 | 1.21174E−06 | 4.56154E−05 | 0.140127389 | 1.638657928 |
| 6 | BAG1 | 4.921467304 | 2.393688917 | 1.20352E−06 | 4.53703E−05 | 0.149681529 | 2.029991865 |
| 6 | COX7A2 | 4.918399334 | 1.947986722 | 1.18925E−06 | 4.49599E−05 | 0.197452229 | 2.094945669 |
| 6 | HSPA2 | 4.909451008 | 3.196259975 | 1.34718E−06 | 5.03572E−05 | 0.114649682 | 2.067631006 |
| 6 | PLN | 4.907315254 | 2.296881914 | 1.2762E−06 | 4.77713E−05 | 0.165605096 | 1.880653977 |
| 6 | C1QBP | 4.904576302 | 3.165123224 | 1.37345E−06 | 5.11952E−05 | 0.114649682 | 2.017274141 |
| 6 | SQSTM1 | 4.902759552 | 1.335787296 | 1.22642E−06 | 4.61027E−05 | 0.356687898 | 2.178190708 |
| 6 | RAC1 | 4.898254395 | 1.522731543 | 1.26861E−06 | 4.7554E−05 | 0.277070064 | 2.167897224 |
| 6 | MZT2B | 4.876043797 | 2.121439695 | 1.47109E−06 | 5.41513E−05 | 0.175159236 | 1.991259098 |
| 6 | UCP3 | 4.874461651 | 1.643891454 | 1.44152E−06 | 5.35074E−05 | 0.24522293 | 2.175022364 |
| 6 | RBX1 | 4.872908115 | 1.790524364 | 1.45314E−06 | 5.37136E−05 | 0.219745223 | 1.893694282 |
| 6 | C14orf166 | 4.869864941 | 2.45891571 | 1.53825E−06 | 5.64669E−05 | 0.149681529 | 1.75276196 |
| 6 | RPL37 | 4.869229794 | 1.52412653 | 1.45545E−06 | 5.37244E−05 | 0.277070064 | 2.087898016 |
| 6 | CYB5R1 | 4.859915257 | 1.430460215 | 1.51873E−06 | 5.58275E−05 | 0.308917197 | 2.051305056 |
| 6 | ACAA2 | 4.858776569 | 3.297741175 | 1.70667E−06 | 6.22197E−05 | 0.111464968 | 1.806630254 |
| 6 | IDI2 | 4.851500511 | 2.292547941 | 1.68196E−06 | 6.14032E−05 | 0.156050955 | 1.987762213 |
| 6 | RPL31 | 4.844508648 | 1.099471569 | 1.60814E−06 | 5.89512E−05 | 0.522292994 | 2.484936476 |
| 6 | RP11-608021.1 | 4.841675282 | 1.37138021 | 1.64767E−06 | 6.02341E−05 | 0.334394904 | 2.279502392 |
| 6 | ADI1 | 4.837037563 | 2.23508811 | 1.78749E−06 | 6.48992E−05 | 0.165605096 | 1.878730655 |
| 6 | RHOB | 4.82534647 | 1.338142633 | 1.78526E−06 | 6.48992E−05 | 0.340764331 | 2.50396204 |
| 6 | STRAP | 4.810926437 | 2.211161137 | 2.01012E−06 | 7.22909E−05 | 0.165605096 | 1.791734338 |
| 6 | PPP2R1A | 4.807990074 | 2.250423193 | 2.05947E−06 | 7.38658E−05 | 0.162420382 | 1.886559486 |
| 6 | GRB14 | 4.787487984 | 2.066797972 | 2.2373E−06 | 7.99203E−05 | 0.175159236 | 1.863456011 |
| 6 | MT-ND4L | 4.775947571 | 1.628862739 | 2.29774E−06 | 8.18514E−05 | 0.24522293 | 2.01039362 |
| 6 | AES | 4.774116993 | 2.071639061 | 2.3901E−06 | 8.45827E−05 | 0.171974522 | 1.927768946 |
| 6 | HSPB3 | 4.769365311 | 2.830386877 | 2.55507E−06 | 8.94679E−05 | 0.121019108 | 1.927147508 |
| 6 | SLC35F1 | 4.766919613 | 1.417949677 | 2.37566E−06 | 8.41839E−05 | 0.299363057 | 2.169786215 |
| 6 | GNG5 | 4.766741753 | 1.922469974 | 2.45191E−06 | 8.64248E−05 | 0.191082803 | 1.978766918 |
| 6 | POLR3GL | 4.765984058 | 3.022897005 | 2.63859E−06 | 9.19078E−05 | 0.117834395 | 1.949764371 |
| 6 | SIK3 | 4.758876801 | 1.050217152 | 2.42305E−06 | 8.56348E−05 | 0.576433121 | 2.708378792 |
| 6 | GLO1 | 4.758647919 | 2.325679064 | 2.62254E−06 | 9.14687E−05 | 0.149681529 | 1.998428822 |
| 6 | SGCA | 4.757004738 | 1.799354911 | 2.54938E−06 | 8.93863E−05 | 0.203821656 | 2.081376791 |
| 6 | NDUFS2 | 4.752978802 | 1.763674021 | 2.59849E−06 | 9.0749E−05 | 0.21656051 | 2.063047409 |
| 6 | NACA | 4.737595558 | 1.404080868 | 2.72397E−06 | 9.45105E−05 | 0.305732484 | 2.180892467 |
| 6 | KEAP1 | 4.736166954 | 2.694928885 | 2.9819E−06 | 0.000102789 | 0.127388535 | 2.022416353 |
| 6 | MRPL34 | 4.733576775 | 2.542482376 | 2.95748E−06 | 0.000102212 | 0.136942675 | 1.721592903 |
| 6 | MAP7D1 | 4.732653141 | 2.39517951 | 2.98086E−06 | 0.000102789 | 0.143312102 | 1.99684906 |
| 6 | MRPL36 | 4.712633133 | 3.039215326 | 3.36977E−06 | 0.000114233 | 0.114649682 | 1.907862306 |
| 6 | NRAP | 4.702322483 | 1.295476556 | 3.21028E−06 | 0.000109524 | 0.340764331 | 2.274826288 |
| 6 | ZMYM4 | 4.701823711 | 1.235048056 | 3.20477E−06 | 0.000109477 | 0.372611465 | 2.196824789 |
| 6 | RPL22 | 4.690321445 | 2.047569036 | 3.54979E−06 | 0.000119785 | 0.168789809 | 2.031428814 |
| 6 | TUBB4B | 4.678945065 | 2.759359598 | 3.90611E−06 | 0.000130582 | 0.117834395 | 1.991942763 |
| 6 | HLA-B | 4.674929619 | 1.638124228 | 3.70588E−06 | 0.000124201 | 0.229299363 | 2.497019053 |
| 6 | CMBL | 4.672047615 | 2.607050419 | 3.97721E−06 | 0.000132625 | 0.127388535 | 1.864283323 |
| 6 | FAM162A | 4.65880537 | 2.533495426 | 4.23531E−06 | 0.000140431 | 0.130573248 | 2.039367437 |
| 6 | TAF7 | 4.653473854 | 1.721054554 | 4.14015E−06 | 0.000137713 | 0.21656051 | 2.036043167 |
| 6 | SERBP1 | 4.652863979 | 1.57020402 | 4.0945E−06 | 0.000136365 | 0.242038217 | 1.909261942 |
| 6 | RBBP7 | 4.649040699 | 1.99748528 | 4.27099E−06 | 0.000141358 | 0.171974522 | 1.925380945 |
| 6 | UBE2D1 | 4.645304203 | 1.68236208 | 4.29888E−06 | 0.000142104 | 0.219745223 | 2.051260948 |

In some cases, the nuclei specific genes may be one or more from Table 1B. In some cases, the nucleic specific genes may be one or more from cluster 1 of Table 1B. In some cases, the nucleic specific genes may be one or more from cluster 3 of Table 1B. In some cases, the nucleic specific genes may be one or more from cluster 4 of Table 1B. In some cases, the nucleic specific genes may be one or more from cluster 5 of Table 1B. In some cases, the nucleic specific genes may be one or more from cluster 7 of Table 1B. In some cases, the nucleic specific genes may be one or more from cluster 8 of Table 1B. In some cases, the nucleic specific genes may be one or more from cluster 9 of Table 1B. In some cases, the nucleic specific genes may be one or more from cluster 10 of Table 1B.

TABLE 1B
Single Nuclei TST Preparation (Top ranked genes for each cluster)

| | | | Cluster 5 | Cluster 7 | | | |
|---|---|---|---|---|---|---|---|
| Cluster 1 | Cluster 3 | Cluster 4 | Satellite | Smooth | Cluster 8 | Cluster 9 | Cluster 10 |
| Fibroblast | Endothelial | Macrophage | cell | muscle | Lymphatic | Adipocyte | Unclassified |
| gene | gene | gene | gene | gene | gene | gene | gene |
| DCN | ST6GALNAC3 | RBPJ | MEG3 | ACTA2 | PKHD1L1 | CPM | MAFG |
| LAMA2 | LDB2 | FMN1 | CADM2 | CALD1 | MMRN1 | SLC19A3 | ANKRD2 |
| NEGR1 | ANO2 | MRC1 | LAMA2 | RBPMS | TSHZ2 | PPARG | AGBL1 |
| APOD | PTPRB | DPYD | MUSK | NR2F2-AS1 | ST6GALNAC3 | RP11-665G4.1 | PRKG1 |
| GSN | MECOM | RBM47 | TRHDE | PDGFRB | CD36 | ADH1B | LARGE |
| GPX3 | VWF | CD163 | PON2 | IGFBP7 | PPFIBP1 | RP11-563P16.1 | SLC38A1 |
| CFD | PECAM1 | CELF2 | PAX7 | DLC1 | GPM6A | SVEP1 | MALAT1 |
| COL6A3 | B2M | ZEB2 | ABCA8 | KCNAB1 | TIMP3 | GSN | MYLK4 |
| NOVA1 | DOCK9 | TBXAS1 | TLN2 | SPARCL1 | EFNA5 | PLIN1 | AFF1 |
| LAMC1 | TACC1 | MS4A6A | AC005237.4 | CACNA1C | TFPI | ADIPOQ | FAM134B |
| COL4A1 | SPARCL1 | F13A1 | APOE | MYH11 | RELN | FRMD4A | SALRNA2 |
| LINC01088 | PLEKHG1 | FRMD4B | MARCH1 | LPP | CNTNAP3B | PDE8B | AC097662.2 |
| EBF1 | EPAS1 | CPM | PLXDC2 | RP11-759A24.3 | TGFBR2 | PTGER3 | ASB5 |
| PID1 | NEDD9 | GNAQ | DYNC1I1 | CARMN | STOX2 | LAMB1 | AC016831.7 |
| ABCA6 | PTPRM | SYK | PLEKHH2 | CLMN | GLIS3 | EBF1 | GLRA1 |
| COL4A2 | TIMP3 | SRGAP2 | NLGN4X | TACC1 | SASH1 | MGST1 | RAB7A |
| NID1 | FLT1 | HDAC9 | CNKSR3 | JUNB | CLDN5 | PARD3B | RP11-96C21.2 |
| IGFBP7 | TMTC1 | SMAP2 | DANT2 | TAGLN | PGM5 | TMEM132C | DTNA |
| TGFBR3 | HLA-B | PIK3R5 | SPARCL1 | CPM | KALRN | PDE3B | TACC2 |
| GALNT15 | KLF2 | SLC1A3 | RBMS3 | RGS6 | VAV3 | LIPE-AS1 | ARHGAP11B |
| SCN7A | PLCB4 | ELMO1 | GPM6B | MYL9 | DOCK5 | ACSL1 | LYVE1 |
| ABCA8 | ELMO1 | FGD2 | PTPRG | TPM1 | MAGI1 | RP1-193H18.3 | DNAH11 |
| TSC22D3 | PITPNC1 | MERTK | CDH4 | PRKG1 | DOCK9 | PTEN | SLC36A2 |
| MAN1A1 | RP3-510L9.1 | MT-CO1 | ELL2 | EPS8 | CCL21 | LEP | PKD2L2 |
| TGFBR2 | SEC14L1 | DOCK8 | FBXL7 | SORBS2 | PLCB4 | SLC7A6 | DIAPH2-AS1 |
| VIT | EGFL7 | C10orf11 | TGFBR3 | ARHGAP10 | MAN1A1 | SLC39A11 | TMEM65 |
| COL6A2 | HLA-E | CSGALNACT1 | APOC1 | NOTCH3 | PLXDC2 | COL4A2 | TMOD1 |
| LAMB1 | SNTG2 | IQGAP2 | MEG8 | DSTN | APP | ITSN1 | FAM155A |
| MFAP5 | SRGN | FCHSD2 | PRKD1 | EBF1 | LRRC1 | MARC1 | NPAS2 |
| PTPRG | DOCK4 | ARHGAP15 | SNED1 | INPP4B | AC007319.1 | CIDEC | MLK7-AS1 |
| GREB1L | EMCN | PTPRC | CALCR | CPE | FRMD4B | FZD4 | GAK |
| FTL | ID1 | MT-CO2 | ADAMTS9-AS2 | CACNB2 | TLL1 | NHS | MPV17 |
| MAP1B | RASGRF2 | CHST11 | ECHDC2 | PDE3A | HIF3A | PTPRS | RORA |
| LHFP | CLDN5 | FAM49B | GPC6 | LMOD1 | TFF3 | RP11-427J23.1 | NEDD4 |
| ARHGAP24 | SASH1 | SRGAP2B | RP11-556I14.1 | RNF152 | ARL4A | ACACB | XIRP2 |
| RBMS3 | TCF4 | TRPS1 | NAV2 | FLNA | SLC22A23 | PNPLA2 | TRDN |
| EGFR | RIN2 | MT-ATP6 | CHN1 | RP11-394O4.5 | BACE2 | TENM3 | ANKRD33B |
| FSTL1 | MCTP1 | MT-CO3 | TPRG1 | ADAMTS9-AS2 | RP4-678D15.1 | GPAM | DAAM2 |
| MAML2 | MKL2 | SPTLC2 | NAV1 | SPARC | ZDHHC14 | AQP7 | MRPS6 |
| PLPP3 | TMSB10 | STARD13 | PNMT | TINAGL1 | CTD-3179P9.1 | GPX3 | CHRNA1 |
| ADH1B | MAGI1 | MT-ND2 | ARHGAP24 | RASAL2 | COLEC12 | FP325317.1 | ERAP1 |
| VIM | ASAP1 | SLC8A1 | LPP | MCAM | LDB2 | GPD1 | DLGAP4-AS1 |
| TXNIP | AKT3 | MSR1 | PTCHD1-AS | ID3 | PTPRE | SPON1 | TSPAN5 |
| CYP4B1 | APP | AGFG1 | AGFG1 | TIMP3 | SMAD1 | ANGPTL4 | KLF6 |
| CXCL14 | TANC1 | FOXP1 | SRPX2 | RERG | PRKCH | ATP10A | RP11-39M21.1 |
| CBLB | ERG | PIP4K2A | IPO9-AS1 | HEYL | GRAPL | RBPMS | TTN |
| CFH | EPB41L4A | VSIG4 | RPL3 | ACTB | IGFBP7 | PDZRN3 | ABCA5 |
| AOX1 | ADGRL4 | MT-ND4 | MT1E | HES4 | MPP7 | MYCBP2 | NEXN |
| GLUL | ARL15 | MT-CYB | IGFBP5 | ESYT2 | NRG3 | PLXNA4 | CTIF |
| S100A6 | C10orf11 | ANKRD44 | RNF150 | SLC38A11 | NR2F2-AS1 | ZBTB7C | RP11-133L19.1 |
| DCLK1 | ARHGAP31 | MS4A4E | BCL2 | UBA2 | C10orf11 | SOX5 | SH3GL2 |
| FTH1 | TJP1 | PARP8 | RORA | TPM4 | CALCRL | CALCRL | EIF4G3 |
| C1S | SYNE2 | RNASE1 | HMCN2 | NCALD | ZFPM2 | ITIH5 | LINC01584 |
| SH3PXD2B | HLA-C | STAB1 | DOCK9 | ADIRF | ASAP1 | AGPAT2 | DIAPH2 |
| TACC1 | TPO | LDLRAD4 | COL25A1 | RCAN2 | RP3-523E19.2 | MAGI2 | PVT1 |
| MT-ND2 | SYNE1 | DOCK2 | NCAM1 | PPP1R14A | RHOJ | RP11-507B12.2 | SSH2 |
| MT-ND3 | STOM | MT-ND3 | AASS | CRISPLD2 | ELMO1 | ADRBK2 | DSCAM |
| FBN1 | LIFR | SRGN | MSC-AS1 | ADAMTS1 | PLPP1 | TLN2 | GLG1 |
| SMOC2 | BACE2 | RGL1 | RPS2 | UTRN | SEMA3A | RP11-444D3.1 | JPH1 |
| AKT3 | WWTR1 | RNF149 | HS6ST2 | SLIT3 | NR2F1-AS1 | FAM213A | PHKA1-AS1 |
| LTBP4 | RASAL2 | SLC9A9 | WDFY2 | NTRK3 | ANKS1A | LPL | VAV1 |
| SVEP1 | ABLIM3 | MT-ND1 | PITPNC1 | A2M | SNTG2 | AOC3 | TRIM54 |
| TMSB4X | ID3 | SLCO2B1 | MT2A | FHL5 | RP11-554D15.1 | GHR | SLC25A33 |
| FBLN2 | TIE1 | RAB31 | EGFR | SGIP1 | PLSCR4 | PCOLCE2 | KHDRBS3 |
| MGST1 | CTTNBP2NL | CD74 | CROCC | HIP1 | PDE1A | SLC1A3 | AP3M1 |
| MT-CYB | CADPS2 | MAN1A1 | ECE1 | CEBPD | SEMA6A | SAT1 | NME7 |
| LRP1 | TMSB4X | APBB1IP | MYO1E | RGS5 | PARD6G | LIPE | SLC7A14 |
| SERPING1 | LDLRAD4 | VAV3 | LPL | ARHGEF7 | PLD1 | COL4A1 | LINC00691 |
| AC007319.1 | UTRN | FLI1 | IFNG-AS1 | FRY | EPB41L2 | TNFAIP8 | ANXA2 |
| ARHGAP10 | BTNL9 | C1QA | DLK1 | ACTN1 | UTRN | BNIP3L | ART3 |
| C3 | C10orf10 | TMSB4X | ZHX3 | RHOB | SDPR | FOXO1 | LINC01492 |
| MT-ND4 | MSN | MAFB | AKT3 | ADGRL3 | MAF | CIDEA | MYF6 |
| FBXL7 | MYH9 | MGAT5 | SAT1 | PLS3 | ITGA1 | BCL2 | MBP |
| EEF1A1 | ENG | CCND3 | SNHG23 | RP11-444D3.1 | TSPAN5 | MLXIPL | PEBP4 |
| COL6A1 | FLNB | RNF144B | DIO3OS | ZNF331 | STK32B | MAOA | TPD52 |
| MAMDC2 | PREX2 | PELI1 | SRSF7 | CAV2 | LYVE1 | PTPRG | RP11-83A24.2 |
| RPL11 | BMS1P14 | SRGAP1 | MTERF4 | PTPRG | TANC2 | EHBP1 | FAM20A |
| SASH1 | POSTN | TET3 | KANK1 | PPP1R12A | ZNF521 | PIK3R1 | GGT7 |
| C1R | SLC9A3R2 | MTSS1 | CEBPD | NBEAL1 | SYNE1 | PLIN4 | MTERF4 |
| EBF2 | CYYR1 | ADRBK2 | MEST | GRK5 | RASAL2 | MDFIC | DLGAP4 |
| IGF1 | DUSP1 | PRKCB | MYF5 | ITIH5 | AKAP12 | ST3GAL6 | ZBTB43 |
| DLC1 | RAPGEF5 | USP15 | KREMEN1 | EGFLAM | HLA-E | GPC6 | HIGD1C |
| AXL | HIF3A | HCLS1 | RYR3 | GJA4 | ITGA9 | PRKD1 | ATP1B3 |
| SAMHD1 | PRKCH | CMIP | RBMS3-AS3 | ATP1B3 | KDR | CTIF | SLC16A9 |
| LRRTM4 | ARHGAP29 | PICALM | PXDN | APBB2 | SNCAIP | GABRE | ADORA2A-AS1 |
| MMP2 | HLA-A | DISC1 | TFPI | TMSB4X | PIEZO2 | ADRA1A | UACA |
| COL12A1 | EPHA4 | RP11-624C23.1 | VAV3 | SOX5 | RAB11FIP1 | UVRAG | PCNXL4 |
| SRPX | NKTR | SRGAP2C | FOXP2 | CCDC107 | EGFL7 | PFKFB3 | MAFF |
| MT-ATP6 | CLIC4 | CTSB | EEF1A1 | DENND3 | NHSL1 | USP33 | ABCA9-AS1 |
| RPS2 | EVA1C | RIN3 | RPL13 | CSRP1 | IL6ST | FABP4 | RP11-830F9.5 |
| CTGF | AQP1 | EMB | RTN1 | MRVI1 | RAPGEF4 | ROCK2 | COL4A4 |
| DOCK5 | ANXA3 | IKZF1 | RPL41 | MYO1B | ADAMTS9-AS2 | EFNA5 | STK11 |
| ZFP36L2 | TEK | CAMKID | COL5A2 | CDH6 | RP11-115D19.1 | ELOVL5 | KLF7 |
| CRISPLD2 | CD74 | KYNU | PTPN12 | MYOCD | PPARG | TNS1 | ADCK3 |
| RPL13 | PELI1 | SAT1 | PTPRE | COL4A2 | TANC1 | CPAMD8 | BLCAP |
| CYBRD1 | ESYT2 | IRAK3 | LRRC8A | IGFBP5 | AFAP1L1 | PLA2G16 | TMEM219 |
| SEPP1 | A2M | STK4 | SYTL4 | MYLK | CSGALNACT1 | WWOX | RGMA |
| MEG3 | IFI27 | RCSD1 | NPAS3 | FABP4 | NKTR | LRP1B | VPS54 |
| SPARCL1 | ABLIM1 | LYVE1 | ZIC1 | TOB1 | DYNC1I1 | C19orf12 | SCN1B |
| TFPI | LRCH1 | ARHGAP26 | CLIC6 | PLPP3 | ST6GAL1 | STAT5A | CDKL4 |
| RPL3 | IFITM3 | MARCH1 | XPO5 | LINC01197 | SEMA6A-AS1 | LIMA1 | RORA-AS1 |
| OPHN1 | WNK1 | COLEC12 | RASD1 | AXL | RALGAPA2 | PARVA | RAB11FIP3 |
| MT-ND1 | MLLT4 | TBC1D14 | SNHG24 | PIP5K1B | PELI2 | GLUL | PRKRA |
| RPL41 | NFIB | LAPTM5 | RASSF4 | ETV6 | MGAT5 | RBMS3 | SLC6A13 |
| PRELP | CNTNAP3B | AOAH | CLCN5 | DGKH | ARHGAP31 | TPRG1 | SLC5A10 |
| MGP | TINAGL1 | INPP5D | TTLL7 | MYO1D | FOXP1 | TEAD1 | DYSF |
| EIF1 | DOCK1 | RP11-1143G9.4 | PDE7B | ARHGAP17 | EPS15 | ADAMTS9-AS2 | ZFAND1 |
| ITM2B | RP11-588H23.3 | MAML3 | RP11-39M21.1 | NR2F2 | SBF2 | PTPRF | KDM2B |
| SRPX2 | ADGRF5 | ITSN1 | MT-CO3 | MAML3 | RCSD1 | TCF7L2 | SSUH2 |
| PLXDC2 | F8 | CD14 | OLFML2B | LHFP | FLI1 | RCL1 | OR2L13 |
| TNXB | CLEC14A | PTK2B | RPLP0 | CNN1 | MYO1D | ZFP36L2 | DES |
| IGFBP6 | ADAMTS9 | MAML2 | MEGF10 | H3F3B | DENND2A | AKAP12 | ELOVL6 |
| MT-CO2 | AC129778.2 | GAB2 | ST3GAL5 | PCED1B | ARHGAP29 | ADCY6 | PDZD2 |
| PARD3B | CPNE8 | FYB | CHPT1 | C11orf96 | MRC1 | MT1M | COBL |
| FBLN1 | HIP1 | PDGFC | XKR6 | RBMS3 | JMJD1C | AC002066.1 | TENM4 |
| RPS15 | FLI1 | ATP8B4 | RALGPS2 | ATP10A | TMTC1 | PHLDB1 | AGAP1 |
| RORA | ST6GAL1 | GAS7 | MGC15885 | LINC01568 | IL7 | FGD4 | GATS |
| RPS27 | APBB2 | PTPRE | PLEKHA6 | ITGA8 | PTPN14 | DLC1 | RDH14 |
| FBLN5 | IGFBP4 | MAP3K8 | L3MBTL4 | MYL6 | SMTN | MAGI1 | MPHOSPH9 |
| PTPRS | CRIP2 | SIPA1L1 | RBMS3-AS2 | WTIP | MLLT4 | EYA2 | PCGF5 |
| COL5A2 | ADIPOR2 | PTPRJ | ENPP5 | PTEN | TSPAN14 | TACC1 | ADCY9 |
| SPRED2 | PLXDC2 | STX7 | PRKX | PLN | TBX1 | PRKAR2B | MLF1 |
| CEBPD | TPM4 | CCDC88A | RP13-143G15.4 | SMTN | SH3BGRL2 | ABCA1 | TSPAN15 |
| ARL15 | MEF2A | CXCR4 | VIT | KALRN | LHFP | CLMP | FMNL2 |
| IGFBP3 | SDPR | GNG2 | GNA14-AS1 | HES1 | ADD3 | MYO1C | TIAM2 |
| ABCA9 | ITGA6 | SAMSN1 | FAM49A | ADCY3 | RPGR | BCKDHB | ZNF350-AS1 |
| RP11-608O21.1 | RALGAPA2 | CD53 | CTNND2 | AC005358.3 | PECAM1 | TRPS1 | PRKCA-AS1 |
| S100A13 | SPC25 | ETV6 | RP11-123M6.2 | PPP1CB | RPS6KA2 | ARHGAP20 | FLNC |
| RPL13A | LNX1 | C2CD5 | HIF1A | PRMT9 | ARL15 | HSPB7 | KLHL29 |
| NCOA7 | LIMS2 | WDFY2 | MT1M | ITGA1 | EPB41L4A | AGBL4 | THRAP3 |
| FIBIN | MYOF | C1QB | TPBG | FIGN | PLEKHG1 | ELL2 | PPM1B |
| BMP5 | PLS3 | PSD3 | LINC01239 | LINC01088 | PROX1 | CDKN1C | RP11-115J16.2 |
| ANTXR2 | ARHGAP26 | CLEC7A | MDFIC | JAG1 | SRGAP3 | FIGN | C1orf216 |
| DUSP1 | RBMS2 | STK17B | SLC25A6 | NTRK2 | CD99 | SH3KBP1 | TBL1Y |
| RPL34 | SULF1 | RTN1 | PPP1R14C | EDNRA | L3MBTL4 | FASN | HMP19 |
| UST | FRYL | ARRB2 | ALDH7A1 | EBF2 | EMCN | MT-ND2 | TNFRSF12A |
| RNF144A | LYST | CYBA | RPS5 | PLCE1 | CNTNAP3 | AP000304.12 | SVIL |
| MT-CO3 | ITGA1 | CPVL | DYNC2H1 | CLIC4 | HIP1 | PPP1R12B | DEPTOR |
| TIMP2 | EFNB2 | ROCK1 | C10orf54 | RP5-857K21.4 | CDKL1 | NME3 | RP11-252A24.5 |
| LTBP2 | CAV1 | DOCK11 | CH507-513H4.1 | RASSF1 | DNAJC18 | FKBP5 | PKD1L3 |
| BMPER | PICALM | MS4A4A | ANKS1B | COL18A1 | TRIO | ARHGAP26 | LRRC2 |
| IFITM3 | RUNX1T1 | TYROBP | RAP1GAP2 | COL6A2 | CLU | EMP1 | LONP2 |
| LRRC16A | RPS6KA2 | B2M | BTG1 | ACTN4 | MDFIC | FERMT2 | RP11-40109.4 |
| LPAR1 | SNHG7 | SLA | ANTXR1 | RAD51B | PDE7B | AOX1 | NLRP1 |
| CIRBP | MGLL | LPAR6 | FBN1 | SLC39A11 | WFS1 | TRHDE | CTBP2 |
| COL1A2 | FRY | C1QC | APBB2 | FOS | EVA1C | APCDD1 | SNED1 |
| CCNG2 | FCHSD2 | TG | RP11-983G14.1 | TBC1D1 | PPP1R2 | AMOTL2 | OIT3 |
| TCF7L2 | CALCRL | SKAP2 | PHLPP1 | SMIM12 | LYN | MIRLET7BHG | FAM126A |
| AGTR1 | DACH1 | P2RY14 | SPARC | SLMAP | KIAA1671 | HIPK3 | NSUN5 |
| BICC1 | GFOD1 | WIPF1 | RP11-556I14.2 | MYH9 | C6orf141 | AFAP1L1 | TARID |
| PTGDS | CAV2 | HLA-DRB1 | TFDP2 | ELL2 | LAYN | AGFG1 | AMPD3 |
| PDGFRA | ICAM2 | NHSL1 | RP11-337C18.8 | RYR2 | PARD3B | LAMC1 | COG5 |
| RPL28 | HMCN1 | PHACTR2 | ABLIM1 | CPNE4 | AC139100.3 | MAST4 | MURC |
| TIMP3 | FBXL7 | FNBP1 | LONRF1 | DGKB | DGKH | MAML2 | PRKAR1A |
| XPO5 | CTA-276F8.2 | MGAT1 | RP11-262H14.4 | STEAP4 | TC2N | SPARC | PTPRO |
| SLC9A9 | NEBL | FOXN3 | AC004053.1 | CSDC2 | CNKSR3 | ZBED3-AS1 | TMC1 |
| ADH1C | PPP1R16B | ZSWIM6 | NTRK3 | SH3BGRL | SPTAN1 | TUSC5 | SLC10A1 |
| RPS4X | TSPAN9 | HCK | SYBU | FAM162B | CEP112 | CYB5A | ATP1B1 |
| ADGRD1 | ACTN4 | CCDC141 | HIF3A | SPECC1 | DSCAML1 | ACER3 | CD46 |
| RP11-444D3.1 | CMTM8 | SH3KBP1 | C14orf132 | PLCB4 | ERG | PSMA1 | GPAT3 |
| H3F3B | SPTBN1 | JAML | MT-CO2 | NFASC | FBXL7 | APOL6 | PCMT1 |
| RPL10 | S1PR1 | LYN | TPD52L1 | LGI4 | CHST15 | PQLC2L | EGF |
| CXCL12 | CD93 | ARHGAP18 | DDX17 | AC013463.2 | KLF6 | APBB2 | ZNF146 |
| RPS23 | ABCG1 | ACAP2 | SRSF5 | KCNJ8 | ARHGAP26 | OXR1 | CTD-3252C9.4 |
| RPL14 | EDN1 | RBMS1 | CDK8 | DOCK8 | PIEZO1 | GPT2 | RP11-666O2.2 |
| RPS27A | TM4SF1 | CSGALNACT2 | ZNF518A | LINC00702 | NR2F2 | MAN1A1 | PLA2G12B |
| RPL15 | SLC2A3 | FCGR2A | GRAMD3 | GUCY1A2 | DSP | FAM126B | TBL1X |
| SHISA6 | CXorf36 | TPK1 | ABCA6 | GUCY1A3 | NALCN | SNTB1 | LINC01572 |
| STXBP6 | TGM2 | TMSB10 | BCAT1 | LRRC16A | SNCA | FAM13A | FAM13C |
| PODN | FABP5 | MYO1F | CD82 | TNC | TIE1 | C2CD2 | SMTNL2 |
| RP1-15D23.2 | FABP4 | ATF6 | CLMP | DGKG | AOAH | TXNIP | CSNK1A1 |
| RPL27A | ESAM | FCHO2 | PARVA | SCN3A | ADAM9 | FBXL5 | RP11-580I16.2 |
| CYR61 | ST8SIA6 | CERS6 | MUM1 | CCDC3 | TMSB10 | RP11-160E2.6 | RP11-6N13.1 |
| RP11-296E23.1 | CCDC85A | BCAT1 | RPL18 | FABP5 | LOX | ABHD5 | HES1 |
| SOX5 | ADIRF | PDE4B | DIRC3 | CAV1 | RP11-759A24.3 | ADIRF | TEAD1 |
| RPL35A | CDH5 | MCTP1 | TUBB2B | SLC8A1 | GYPC | PRELID2 | OSBPL9 |
| GFRA1 | HYAL2 | FOXN2 | CTB-32H22.1 | PHLDB2 | SLC26A5 | RASSF4 | ESR2 |
| CELF2 | FGD5 | HLA-B | SCARNA2 | CDC42EP4 | EHBP1 | PXDN | CAST |
| RPS9 | CD34 | GRB2 | WWOX | SYN3 | HECW2 | ATP2B4 | RP11-118M9.3 |
| RPS16 | CSGALNACT1 | LCP1 | TPCN1 | CD9 | MAP3K3 | IDH1 | GRIP1 |
| S100A4 | HLA-DRB1 | LRRC16A | KEL | STK38L | ART4 | ARHGAP21 | CNNM2 |
| CILP | NOTCH4 | TNFRSF1B | PROX1 | EFHD1 | ERICH1-AS1 | STARD13 | EEPD1 |
| NUPR1 | ITGA9 | RUNX1 | FGFR4 | LBH | PCCA | TTC39C | ATPIF1 |
| SERF2 | CLU | CSF1R | FBXO42 | ADCY5 | MVB12B | DPT | FRMD3 |
| PROCR | ITPRIP | PPP1R21 | KCND2 | ISYNA1 | AC129778.2 | DDX17 | AC011997.1 |
| B2M | ITGA5 | SLC11A1 | TEAD1 | WFDC1 | ICA1 | TSKU | ARL5A |
| CTDSPL | KIAA0355 | ARHGAP24 | VIM | IGFBP2 | CDH5 | SEMA3A | H2AFZ |
| NAV2 | MMRN2 | IFI16 | SLC16A12-AS1 | RP11-499P20.2 | SHANK3 | PALM2-AKAP2 | C16orf45 |
| PKD2 | ID2 | ALCAM | PKNOX2 | PICALM | TSTA3 | PDGFD | MANSC1 |
| CALD1 | CDK17 | ANKS1A | MT-ND1 | CTD-2009A10.1 | ITGB4 | C1QTNF1 | ATF1 |
| KCNT2 | C8orf4 | FAR2 | CNR1 | APOLD1 | KLHL4 | CEBPA | LINC-PINT |
| LRP1B | PPP1R13B | CSF3R | SPTBN1 | ZBTB7C | FAF1 | MYEOV | PSMD6-AS2 |
| RPS17 | MYRIP | MDFIC | MEF2A | CREB3L2 | RPL36 | TGFBR3 | LMTK2 |
| FAU | TMTC2 | AKAP13 | PTCHD1 | GJC1 | EMP1 | ADCY5 | CTD-2537O9.1 |
| NCAM2 | CMIP | ELMO1-AS1 | CBLB | SSBP3 | EXOC6 | SRSF11 | OSER1 |
| PCDH9 | PIK3C2A | FCER1G | SLC25A27 | BTG1 | MAP3K1 | MED13L | CLIP1 |
| DDX5 | LIMCH1 | ADAM28 | TRA2A | PKD2 | SPDYE3 | GTDC1 | MORC2-AS1 |
| USP53 | STK38L | RNF130 | SLC16A12 | MARK1 | RUNX1T1 | MT-CO3 | FSD1L |
| FGFR1 | ENTPD1-AS1 | CYTIP | CDK6 | ARHGEF17 | NUMB | GYS2 | ARF6 |
| RPS13 | CCDC68 | SP100 | CIRBP | RP11-394O4.6 | CD47 | STK39 | LRRC8B |
| RPS28 | ELMO1-AS1 | PLXDC2 | TMX4 | FRMD3 | DAPK1 | DCXR | SDC4 |
| RPLP0 | ADCY4 | C1orf162 | TMEFF2 | CCNH | VIM | DIO3OS | TSPAN8 |
| DDIT4 | ELN | KIF13B | KIAA1456 | SNTB1 | CPNE8 | USP24 | ARG2 |
| PLAC9 | PALMD | DENND1A | PPP1R9A | NDUFA4L2 | CXorf36 | CLSTN2 | KANSL2 |
| LPP | GMDS | ALOX5 | RPL15 | ADGRF5 | PDE4B | CCDC69 | TM4SF1-AS1 |
| SLC44A1 | PPFIBP1 | H2AFY | LINC01138 | MTHFD2 | CNN3 | RP11-507B12.1 | BCL2L14 |
| RBMS3-AS3 | CD9 | MYO5A | CDK18 | TNS1 | PALD1 | COBLL1 | CAMSAP1 |
| PI16 | ENTPD1 | AGTPBP1 | SLC2A9 | TSC22D1 | GNAS | CISH | CNTNAP2 |
| MYOC | HEG1 | HLA-DPB1 | CSNK1E | FRMD4A | SGIP1 | VKORC1L1 | OSBPL8 |
| SLC25A6 | IFI44L | C20orf194 | PPP1R21 | STK39 | IRAK3 | SLIT3 | UBA6-AS1 |
| RPS8 | CAPZA2 | RP11-1080G15.1 | RPL10A | WDR45 | NRP2 | NRIP1 | MGLL |
| RPL10A | SWAP70 | NCKAP1L | TGFBI | ITPR1 | MAML2 | RGS3 | IGF1R |
| NOX4 | SORBS2 | HLA-DRA | MCC | ROCK1 | UVRAG | XIST | RBBP8 |
| DDR2 | CD59 | NAIP | NOSIP | STXBP4 | SLCO2B1 | ADIPOR2 | TSTD2 |
| GNB2L1 | CCNY | CCDC91 | EGLN3 | NR4A1 | GIMAP8 | RNF217 | GATSL2 |
| ZFAND5 | HIPK3 | PLXND1 | RP11-712B9.2 | SLC6A16 | CTB-107G13.1 | PARD3 | CTD-2066L21.3 |
| RPL7 | PLPP1 | SH3TC1 | LEPR | SOD3 | AC008937.2 | RCOR1 | APOBEC2 |
| SCARA5 | RP4-613B23.1 | VOPP1 | EIF3J-AS1 | PDE4C | PHLDB2 | RTN4 | UBE2D1 |
| DIO3OS | KLF6 | NRP1 | CCND3 | TBX2 | S100A10 | COL5A2 | FBXO32 |
| RPS14 | RFTN1 | BMP2K | CEP112 | PELI2 | RP13-143G15.4 | AZGP1 | MYC |
| EFHD1 | CD36 | ATM | ACSM5 | ACTG2 | KLF2 | IRS2 | PACSIN2 |
| MME | RPH3AL | TLR2 | GNB2L1 | VASP | SEMA3D | HERC4 | RIF1 |
| NLGN1 | GNAQ | CLEC2D | TCEB3-AS1 | ACTG1 | CCND3 | HEBP2 | ARHGAP23 |
| ZNF90 | RAPGEF1 | LYZ | TDRP | VASN | ABLIM3 | LAMA4 | SPPL2A |
| RPS25 | ADD1 | IL1R2 | MET | MAP3K5 | RP11-436K8.1 | FAM199X | LAT2 |
| CDO1 | RAMP3 | PLTP | NBEA | NTN4 | ZMAT4 | HIP1 | PDE4DIP |
| PRICKLE2 | HLA-DRB5 | SARAF | ASB13 | COBLL1 | ARHGEF10 | WDR41 | PEX5L |
| SESTD1 | RNF125 | CTSS | RPL11 | SNCG | REEP3 | G0S2 | ZC3H11A |
| LARP6 | SHROOM4 | MAP3K1 | TMED10 | ASAP1 | GLS | KCTD7 | DTNB |
| EEF2 | KLF4 | RP11-426C22.5 | VDAC3 | BCAM | GGT5 | RSRC1 | TNRC6A |
| PLA2G2A | MYO5C | CDC42SE2 | EMP2 | ADGRB3 | NR2F1 | FOXN2 | POT1-AS1 |
| PLAGL1 | CCND3 | CEBPD | ZC2HC1A | SRSF3 | THSD7A | FAM214A | RP11-123B3.2 |
| SDC2 | HSPG2 | FTL | SPRY1 | OPN4 | RBMS3 | ACO1 | SCN8A |
| CST3 | FGF2 | SEPPI | RBBP6 | TTLL7 | CADPS2 | ABCD2 | ST7 |
| PLEKHA7 | DGKH | ITGAM | CHST7 | ASAP2 | RAI14 | AZI2 | CENPP |
| RP11-160H12.2 | ICA1 | PAN3 | RPL6 | CSRP2 | MPDZ | SLC25A1 | RBFOX1 |
| MT-CO1 | SLCO4A1 | PARP4 | ADAMTS10 | NRP1 | PEAR1 | PDXK | RC3H2 |
| RPS15A | MGP | MIR181A1HG | RP11-160E2.6 | LAMA4 | LAMA4 | EIF4EBP2 | TTC39A |
| RPL7A | PLVAP | ITPR2 | ATF7IP2 | CNTN4 | RP11-776H12.1 | TJP2 | B3GNT5 |
| RPL30 | PTGIS | STK10 | NTN4 | RP11-315E17.1 | AUTS2 | TNRC6A | RP11-169E6.4 |
| RPS3 | SYN3 | RP11-277P12.20 | DENND6A | RASL12 | PALM | TAF15 | THBD |
| CYP4Z1 | TAGLN2 | DPYD-AS1 | ALDH1A1 | AC097724.3 | STX7 | PCCA | ABLIM2 |
| WASF3 | NRP1 | EPS15 | C9orf84 | AC100830.3 | PPP3CA | TSC22D2 | N4BP1 |
| RND3 | XAF1 | SUSD6 | CNTLN | ACKR3 | PXN | TUBB6 | ARHGAP6 |
| RPS18 | TSHZ2 | SNCA | AC022182.1 | SEPT7 | PPP1R16B | ASPH | TRANK1 |
| VCAN | DOCK6 | DLEU2 | SLC24A4 | HEY2 | DOCK4 | MIR193BHG | CTD-2184D3.5 |
| SIK3 | IRAK3 | CD68 | CACHD1 | CHD1 | TJP1 | STK24 | ABCC4 |
| EFEMP1 | RPGR | RP6-159A1.4 | RPS18 | REM1 | SKAP2 | ZEB2 | MEF2A |
| TXNRD1 | ABCB1 | FPR1 | TUBA1C | AC002066.1 | TUBB6 | RP11-407P15.2 | AC079790.2 |
| UBA52 | ABR | AGO4 | SUN2 | ANO1 | ATXN3 | HIVEP3 | DICER1-AS1 |
| OLFML3 | PTK2 | SIGLEC1 | BCKDHB | MAML2 | CRACR2B | ACADL | FAM160A1 |
| RPL32 | CD2AP | TRIM14 | SRSF6 | SDK1 | KBTBD11 | MT-ND1 | LINC01504 |
| SH3D19 | CDC42BPA | MT-ND5 | MAL | TMEM51 | SMAD9 | DDI2 | TTN-AS1 |
| SERPINF1 | RNASE1 | GSAP | PKD2 | C9orf3 | SH3D19 | RP11-736K20.5 | SLFNL1 |
| DYNC2H1 | PTMA | FAM65B | IFITM2 | SLC2A3 | TIMP2 | JAG1 | TRIM37 |
| RPLP1 | ATP8B1 | REL | TMUB2 | TGFB1I1 | MT-ND2 | ECHDC1 | CDH15 |
| SPATA6 | MYO1E | LRRFIP1 | CYBRD1 | LAMA5 | RP11-473M20.9 | ZNF521 | HSPA2 |
| RUNX1T1 | FOXP1 | RHBDF2 | CDC42EP4 | PAWR | SULT1C4 | DOCK11 | KLHL8 |
| WISP2 | CDH13 | DAPK1 | DNAJB14 | CASQ2 | FGL2 | PRICKLE2 | RP11-46107.1 |
| APOO | SHE | GPR34 | NOTCH3 | RBPMS2 | CATSPERB | LINC01301 | NUP88 |
| CCNI | BMPR2 | MAF | WEE1 | NHS | LAPTM5 | GYG2 | KIAA0232 |
| F3 | EMP1 | TNFAIP8 | ANKRD28 | FBXL3 | RNF38 | CSAD | SLC30A4 |
| FMO2 | NOSTRIN | CROCC | PDP1 | MAP4K4 | KAT6A | ENPP1 | EGLN3 |
| HIF3A | NUAK1 | CDK6 | SEMA3C | TMEM184B | CABLES1 | PPP3CA | CAMK2A |
| SLIT2 | RGCC | ST6GAL1 | GRIK4 | RGL3 | FCHSD2 | RP11-286B14.1 | LSM7 |
| RPS24 | FAM107A | ITGB2 | ZBTB20 | MYOF | ODF2L | TTC38 | CKAP5 |
| RPLP2 | RASSF1 | RPS19 | GNA14 | AOC3 | DOCK6 | RNF144A | FUNDC2 |
| BDNF-AS | MYCT1 | MVB12B | MT-ND2 | MFGE8 | ADAM10 | THSD7A | MYBPC1 |
| PIK3R1 | RAPGEF2 | RASA2 | CTD-2201G3.1 | LDB3 | KIAA1456 | ECHDC2 | PHTF1 |
| CYP4X1 | RP5-1101C3.1 | HLA-C | TENM4 | AC008440.5 | HIVEP3 | IL1RAP | BEAN1 |
| SDK1 | IFNGR1 | RP11-452H21.1 | ZNF536 | ARPC5 | CCDC102B | SLC16A7 | PRPF8 |
| RP11-210L7.3 | NAV1 | ST8SIA4 | GLI2 | ARHGDIB | FAT4 | AGMO | ENAH |
| RPS19 | GIMAP4 | CMTM7 | RHOB | CYCS | MAP4K2 | PQLC1 | SMG1 |
| FN1 | PODXL | PDE3B | HS3ST5 | FLT1 | USP31 | STAT5B | EXOSC5 |
| RPS6 | CPAMD8 | TGFBI | RPS27A | ZNF704 | PTPRK | FAM20C | PPP1R13B |
| APBB2 | GUK1 | IL13RA1 | EFHC1 | KCNMB1 | LINC01006 | SEMA3C | PPP2CB |
| MEDAG | SH3BGRL2 | SMCHD1 | RTKN | GPAT2 | TGFBR3 | ITGA7 | PFKFB3 |
| MMP14 | DENND3 | NPL | RPS19 | TEX41 | SYN3 | PHLPP1 | FAM73A |
| RPL8 | ACKR1 | GPR183 | SPICE1 | B2M | NOD1 | IRAK3 | SRP72 |
| RPL18 | PRKX | CD163L1 | EEF2 | SYTL2 | INPP4B | TBC1D5 | ARFGAP3 |
| LUM | SPRY1 | LCP2 | ZEB2 | PTMA | SH2D3C | ALDH2 | CTC-518B2.10 |
| UAP1 | PKP4 | TFEC | LPIN3 | SLC16A12 | TRIOBP | CDC37L1 | PLXDC2 |
| GRK5 | ADAM15 | IL18R1 | XYLT1 | RP11-665G4.1 | GNG11 | CCDC107 | CACNA1F |
| HS3ST1 | MYO1D | GTDC1 | RPS3 | LINC00989 | BMS1P14 | PTPRM | IL1RAPL1 |
| VGLL3 | EXT1 | ENTPD1-AS1 | APP | PAG1 | DPYSL3 | C10orf11 | MIR193BHG |
| PQLC2L | PLXNA2 | VCAN | STARD3 | NBL1 | PKN2 | MT-ND3 | PCDH11X |
| EYA2 | LRRFIP1 | CPNE8 | MTHFD2 | NMNAT2 | DKK3 | HSPG2 | ZNF124 |
| RAMP2-AS1 | ZNF385D | TYMP | GMCL1 | MYADM | GNAT3 | RUNX1T1 | MLIP |
| PDZRN4 | LMBR1 | NLK | MT-CYB | NUDT4 | TJP2 | AR | ANK3 |
| ARMC9 | RPS6 | HERPUD1 | HIP1 | GADD45B | FAM84A | RP11-1101K5.1 | DGKD |
| C10orf10 | LRRC1 | AP000476.1 | MT-CO1 | RAPGEF2 | PARP4 | MMD | LRP11 |
| RPS7 | SH2D3C | PTPN2 | UGCG | COL4A1 | ADGRG3 | AIFM2 | DIP2A |
| RPL19 | ACTN1 | GLIPR1 | MAGI2 | LTBP1 | SPATA6L | C10orf10 | UBE2G1 |
| SELM | WARS | FCGR2B | DIRC3-AS1 | GRAMD3 | ADIRF | BMP1 | RP11-532N4.2 |
| MTSS1 | RP11-638I2.8 | IFNGR1 | BBS7 | NPY1R | TM4SF1 | DHRS3 | MXD1 |
| RPL36 | ITPR2 | IL10RA | RPL13A | PRR16 | TACC1 | CAT | DDX18 |
| SPRY1 | SLCO2A1 | MANBA | TRIM56 | DPY19L2 | MBOAT1 | HRSP12 | RP11-350J20.5 |
| RP11-39M21.1 | TMEM204 | RB1 | MT-ND3 | P2RY14 | KLF3 | RP11-729L2.2 | PGM2 |
| APP | SERPINE1 | ENTPD1 | CLIC4 | RGCC | TLE1 | GTF2I | NFKBIA |
| CD99 | SHANK3 | IGSF21 | S100A6 | XKR6 | MT-ND4 | HSD17B4 | RP11-513G11.4 |
| RPL6 | LRRC32 | CEP170 | HDAC2 | FER | NR5A2 | SRGAP1 | FGF13 |
| CD81 | MMP28 | ASAP1 | ST5 | LAMA3 | SMOC1 | PHLDA3 | WWOX |
| DPT | STX12 | CLEC12A | PPP1R16A | SLCO3A1 | TCF7L1 | HILPDA | ANXA8L1 |
| RPL29 | UVRAG | DLEU1 | MKRN3 | COX4I2 | VPS13C | WDFY2 | RP4-680D5.2 |
| ZBTB20 | PLAT | FAM117B | TAB2 | LURAP1L | PDGFC | SRPX | RADIL |
| RPL23A | ARFGEF2 | PAG1 | MFSD6 | GPRIN3 | TM4SF18 | MEST | FILIP1L |
| RPL35 | BST2 | MKRN3 | SOX13 | LIN7A | GNG12 | RP11-39404.5 | TRIM44 |
| BMP4 | PKN3 | C3AR1 | NHLRC3 | MDM2 | F8 | PCK1 | CACNA1A |
| KCNH1 | LIMA1 | CHN2 | SRGAP2 | PTH1R | BBS9 | CEPT1 | CCDC178 |
| ITIH5 | TUSC3 | ARSB | CHIC2 | KLHL23 | PTPRB | F3 | PIK3C2G |
| UGGT2 | CXCL2 | PREX1 | SESN3 | ARHGAP6 | AGFG2 | APBB1IP | GIPC1 |
| RPS11 | BMP6 | TBC1D5 | WASF3 | CBR4 | KLF4 | CRLS1 | RP11-434D9.1 |
| TENM1 | GRASP | MIS18BP1 | CELF2 | HLA-C | SCNN1B | ECE1 | LINC00276 |
| PGF | MAP2 | CSF2RA | ZNF862 | RASGRP2 | NBEAL1 | RP5-857K21.4 | CLDN12 |
| ECE1 | PCAT19 | NPC2 | MT-ND4 | FCHSD2 | GNG12-AS1 | PIM3 | AC009264.1 |
| TC2N | RHOB | LST1 | SLC27A6 | ENPEP | BCL7C | WASF3 | RTN3 |
| PHLDB1 | IFI16 | CTSZ | CLK1 | PDK3 | SPTBN1 | INTS6 | ENO3 |
| MT2A | RP11-362K2.2 | NCF4 | H3F3B | TESC | TSHZ3 | PCED1B | RC3H1 |
| LAPTM4A | EEF1A1 | HLA-DRB5 | RBL2 | RP11-35G9.5 | INSIG2 | FGF2 | AC005281.1 |
| ADAMTS5 | APOLD1 | AHR | N4BP2L2 | NR3C2 | RP11-130F10.1 | AC129778.2 | SDK1 |
| S100A10 | MCF2L | MEF2C | CGGBP1 | RP11-156K13.1 | HLA-A | YWHAZ | CCDC181 |
| RPS20 | CRIM1 | ARAP2 | GARNL3 | NDE1 | RP11-290H9.2 | CDH23 | EGFL6 |
| COL15A1 | PPARG | ST6GALNAC3 | RP11-27M24.1 | RERGL | FRMD6 | CACHD1 | AC058791.1 |
| RP11-553L6.5 | HLA-DRA | STAT6 | SPIN1 | ESAM | PVRL3 | ADAMTS15 | ESPN |
| RPS5 | RP11-35G9.5 | EPS8 | MOCOS | TMEM184C | MAP4K5 | BTBD11 | AJ006998.2 |
| GAS1 | LY6E | NABP1 | SUPT16H | GAS6 | THSD4 | ACOX1 | PRNP |
| NFIA | GRK5 | AP2A2 | RPL28 | ITGB1 | DOCK1 | TTPA | STAG3 |
| RP11-289H16.1 | GNB1 | ARRB1 | FAS | CD151 | TMEM241 | MIR4435-2HG | FOXN4 |
| PLEKHH2 | STOX2 | DIP2B | ZKSCAN1 | MPPED2 | VPS50 | ORC2 | ARL17A |
| TMEM204 | RAPGEF3 | PRKCH | CDC16 | AC140912.1 | MIR4435-2HG | PEX11A | PLCD4 |
| TIPARP | RP4-678D15.1 | UTRN | RP11-793A3.2 | ELOVL5 | DANT2 | CDC14B | NKAIN2 |
| S100A11 | FAM196A | CD84 | MXRA8 | TJP1 | KCNN3 | BANK1 | INPP4A |
| RP1-193H18.3 | CHSY1 | TAOK3 | ZNF33B | PGM5 | RFTN2 | DDX5 | USP38 |
| EIF4A2 | HEY1 | ATF7IP | RPS8 | ITM2C | CDK6 | LVRN | LINC00534 |
| KAZN | GIMAP8 | FOS | GS1-433024.1 | EDIL3 | IGFBP4 | LGALS1 | TBC1D4 |
| WIPF1 | NDRG1 | RNASE6 | PAPSS1 | CD36 | RP11-289H16.1 | AASS | TRIM72 |
| MTCH1 | RPL3 | ATP2B1 | NLGN4Y | NEURL1B | CCDC53 | PRRC2B | PAWR |
| DSE | RP11-314N13.10 | ATG16L2 | MED22 | MAP2 | DUSP1 | LCORL | ARPC1A |
| THBS4 | ITM2B | MEF2A | PSMD5-AS1 | SPINT2 | STXBP6 | LDHB | RGS7BP |
| NFKBIA | CA8 | CYTH1 | RP3-508I15.14 | CACNA1A | PRKCZ | C1QTNF7 | MFAP3L |
| ADD3 | GALNT18 | MPPED2 | ANKRA2 | RANBP3L | SCN3B | PKD2 | PIK3R3.1 |
| SDCBP | NR4A1 | FGL2 | GAS2L1 | RNF180 | MED13L | OXCT1 | AVIL |
| MT-ND5 | RHOC | LRRK2 | TMX3 | SLC25A6 | OSBPL5 | MAPK14 | SMPX |
| FAM162B | PLSCR4 | RNF13 | ZBED3-AS1 | CLSTN2 | CTDSPL | RDH10 | C5AR2 |
| RPL21 | TMEM88 | EPB41L3 | PLEKHA5 | RP11-626H12.3 | MYH10 | MGST3 | TDRD5 |
| PTMA | RNF115 | HLA-DQB1 | RPS6 | PLCL1 | MYCT1 | MCAM | JADE3 |
| RBPMS | BTG1 | POU2F2 | ODF3B | PDE1A | GJC2 | KCNIP2-AS1 | GGA1 |
| C1RL | GNAI2 | ZCCHC2 | STARD13 | SEPT4 | SV2B | MIR22HG | NFE2L1 |
| SLC1A5 | DNAJC18 | HIF1A | TRIM55 | CNR1 | RAP1GDS1 | AFTPH | DHX29 |
| LIFR | SAMD12 | CD44 | PDCD6IP | POGZ | ZNF83 | DNAJC1 | EGLN1 |
| CALCRL | FGD4 | SHTN1 | N4BP2L1 | CACNA1H | ELK3 | APPBP2 | ZNF215 |
| FYN | MTHFD1L | RAP1A | VAMP5 | RAPGEF5 | CD9 | AHCTF1 | RP11-505P4.7 |
| AMPH | GNG11 | ADAP2 | POLA2 | GUCY1B3 | RAB6A | RP11-210L7.3 | SEPT9 |
| PSAP | RARB | ARL15 | CCZ1B | NBAT1 | SLF1 | RAB1A | TNPO1 |
| RPL38 | YES1 | PABPC1 | HTR4 | GPRC5C | TMEM87B | VEGFA | RICTOR |
| DENND2A | COL8A1 | AKR1A1 | RPL32 | AKT3 | ADAM19 | IL16 | TRIM63 |
| PDGFD | PHACTR2 | ZDHHC20 | CALD1 | PEAR1 | AGPAT4 | AC108142.1 | XPO1 |
| RPL23 | NOS1AP | TNFRSF11A | SCAMP3 | TRAF5 | ELF2 | CLMN | GPR176 |
| RPSA | IFITM2 | ADGRG6 | FNDC4 | PRDM16 | MSRB3 | BMS1P14 | RALYL |
| RPL12 | ITSN2 | SNX2 | TASP1 | RP11-1000B6.3 | FRYL | PITPNB | METTL7A |
| PLEKHA5 | MEOX2 | CAB39 | NRSN2-AS1 | SMIM3 | TMOD2 | DDR2 | SH3GL1 |
| WBP5 | GSN | CD4 | ZNF521 | ATP8B1 | MTHFD1L | DGAT2 | ZSWIM8 |
| NACA | PDCD4 | MED12L | RPL21 | ANKRD37 | IFT122 | CAV1 | MYO3B |
| ADH5 | PLCXD3 | DOCK10 | AC092839.3 | CSRNP3 | LDLRAD4 | AKR1C2 | FAM118A |
| TMA7 | RPL36 | RP11-16C1.3 | RP11-197K3.1 | GRID1 | HMCN1 | RBP4 | UNC45B |
| PCOLCE2 | MCC | WDFY4 | SMIM3 | CSPG4 | YES1 | GNG2 | JAKMIP2-AS1 |
| ERRFI1 | GIMAP7 | CR1 | AC098617.1 | HLA-B | KIAA1033 | GNG12 | RP11-184M15.2 |
| NBEAL1 | IPO9-AS1 | MAP2K1 | SECISBP2L | JUND | MT-CO3 | RAB2A | UBE2K |
| HSPG2 | ARAP3 | RNF175 | WDR11 | ITPRIP | PLEKHA7 | FAM49B | GRID2 |
| ARHGAP21 | TCF4-AS1 | SIRPB2 | RP11-517I3.1 | ECE1 | EEF1A1 | MCCC1 | COL0 |
| ANGPTL1 | KIAA1671 | CH507-513H4.1 | TUBA1A | BTG2 | PELI1 | MT-CO1 | TPST2 |
| PTEN | RUNDC3B | PPP3CA | TBRG1 | CDC42 | IQSEC1 | PFKFB1 | OSBPL6 |
| RPS12 | ATP10D | KIAA1551 | ASNS | L3MBTL4 | PARVA | LRP1 | NDRG2 |
| H2AFJ | CYYR1-AS1 | SPIDR | ABCA1 | CNN2 | PSMG4 | DIRC3 | XIAP |
| KIAA0040 | SPTAN1 | HAVCR2 | C9orf3 | PALLD | LMO2 | PDHX | MRVI1-AS1 |
| AZI2 | AKAP12 | TBC1D22A | SLC9A7 | MAP1B | CD59 | LINC01239 | FAM221B |
| EPS8 | SOX17 | ATP11A | CXCL14 | ILK | STARD8 | HACD1 | RP11-693J15.5 |
| OOEP | JUNB | CCR1 | TGFB2 | AVPR1A | SYT1 | LINC00869 | CTD-2515A14.1 |
| C6orf48 | KALRN | ZCCHC6 | GREM1 | KIF13A | MXD4 | EXOC1 | OMG |
| SMIM3 | CNOT6L | SPI1 | PTGFR | FBLIM1 | AKT3 | LINC00623 | THRA |
| ITGA5 | TMEM184B | NBPF19 | EXOC3L4 | MGP | RAD51B | NTRK2 | TPCN2 |
| TRPC1 | MGAT4A | KIAA1468 | WDR35 | RP11-203L2.4 | FAM43A | PVRL3 | LAGE3 |
| DOCK1 | RP11-767I20.1 | FCGR3A | UTRN | CTD-3239E11.2 | FLNB | GCLM | C7orf31 |
| ZEB1 | EML1 | NOTCH2 | FANCL | ENTPD1-AS1 | PML | URGCP | RP11-24I21.1 |
| ACVRL1 | BAIAP2L1 | FPR3 | WASF2 | CIRBP | RAB5A | FTL | PRKCA |
| PDGFRB | PARP14 | FYN | POGZ | C1QTNF1 | LINC01515 | OXSR1 | DDX11-AS1 |
| ACADL | TSPAN14 | ARID1A | CTD-2532K18.1 | EPN2 | TXNL4B | NOVA1 | EIF3B |
| MMP3 | FDPS | ID2 | AIG1 | SOX13 | POF1B | SCP2 | DENND2C |
| PDK3 | ZNF366 | FGR | ATXN3 | ATG16L2 | CSNK1E | PLEKHM3 | AC098617.1 |
| EMP2 | TANC2 | EPSTI1 | TMEM243 | FAM213A | CHIC2 | ZSWIM6 | MTO1 |
| NIPAL2 | IL4R | HLA-A | ARMC9 | HCFC1R1 | PDE2A | ZNF518A | RP11-77K12.1 |
| GPX4 | JAM2 | HLA-DPA1 | SPATA6 | ZFAND5 | SCN3A | RNF168 | CD81-AS1 |
| PDGFRL | ABCC4 | AP000304.12 | HS3ST3B1 | TSPYL1 | ENOSF1 | ESYT1 | ALX4 |
| MAGI2 | SMAD6 | PCED1B | ABHD17B | CASC15 | COBLL1 | HMGB1 | NXN |
| MAP1LC3A | PTRF | KLHL2 | AC037445.1 | MT1M | SNX16 | SEPT11 | VSIG10 |
| IMMP2L | RPS27A | PRMT2 | UPF3B | ATP6V1G1 | RECK | JAK2 | RP3-453C12.15 |
| DPYSL2 | FAM65B | CORO1A | INSIG2 | FSTL4 | YWHAQ | HIF1AN | TOX |
| STK24 | ASAP2 | RASSF3 | LINC01481 | ZNF385D | TRPC4AP | EPB41L1 | RP11-1060G2.1 |
| LMO3 | HSD17B12 | GPX1 | HEYL | PTMS | LINC01443 | ORAOV1 | SGF29 |
| WDFY3 | CNTNAP3 | GRN | HYKK | MICU3 | CRIM1 | CFLAR | MGAT4C |
| COL3A1 | MTUS1 | C22orf34 | RPS12 | DTX3 | CD151 | ZNF704 | LINC01479 |
| SLC39A14 | ADAMTS6 | NUMB | CCDC67 | ID4 | IQCK | MTMR3 | DMGDH |
| WWOX | WSB1 | CBL | NABP1 | LGALS1 | WSB1 | ACVR1C | TMC8 |
| STEAP4 | GPIHBP1 | DRAM2 | FAM196A | FADS3 | ROBO4 | APMAP | CPEB1 |
| HTRA3 | ARHGEF10 | CD86 | DENND6A-AS1 | CAMK2G | SOCS2 | STAT2 | RP11-156P1.2 |
| MED13L | CEP112 | C10orf54 | RP3-399L15.3 | DOCK1 | GRIA1 | TOP1 | ULK4 |
| BAIAP2L1 | RBMS3 | KCTD12 | TSC22D3 | RP11-157I4.4 | STON2 | GDAP2 | FSD2 |
| MT1G | ZFP36 | PSAP | HSD17B11 | CBX7 | TMTC2 | CABLES1 | AF127936.9 |
| HMCN2 | LAMA5 | BIN2 | EBF3 | ENTPD3 | ANKIB1 | TNIP1 | CTC-308K20.1 |
| RPL37A | PTMS | MAP3K5 | RP11-692D12.1 | HAPLN2 | KLHL2 | NNT | AFAP1L1 |
| NLGN4Y | RP5-965F6.2 | PPP1R12A | RP11-379B18.6 | ELN | MAML3 | MIDI | SLC20A2 |
| ABCA9-AS1 | SNCG | CNTRL | ATOH8 | RP11-152K4.2 | SRGAP2C | HEXDC | CTD-2552K11.2 |
| ZFP36 | CDC27 | SLC2A13 | LURAP1L-AS1 | PRKCDBP | NFIB | ITGB1 | AC009299.2 |
| GLI3 | ZBTB46 | SRSF7 | CCDC122 | RBMS3-AS3 | EPHA2 | CYP4B1 | WIPI1 |
| LAMC2 | PELI2 | SPRED1 | CASD1 | SH3RF1 | GPR146 | RP11-83M16.5 | HSF1 |
| MKL1 | S100A10 | CHKA | MAGED1 | STAMBPL1 | ANKDD1A | SMIM3 | ETS2 |
| RP11-138I17.1 | RB1 | BLVRB | SNHG7 | HS6ST3 | PKP4 | TNK2 | YTHDF2 |
| MFAP4 | IFI6 | ARPC1B | RP11-544A12.4 | PACSIN2 | RP1-90G24.10 | SCRN2 | STARD7 |
| SAT1 | CHN1 | SLC8A1-AS1 | ZNF701 | ITM2B | APBA2 | ITPKB | MPP7 |
| RTN4 | FNBP1L | MEGF9 | DOCK8 | ST6GALNAC6 | EDNRB | DDA1 | SNX10 |
| PLXDC1 | CTNNB1 | KCNAB2 | TSHZ3 | OR51E1 | ZNF33B | BMPER | COL13A1 |
| BTF3 | ARHGEF3 | FAM49A | KCNQ1OT1 | ATF4 | PEAK1 | PECR | ABHD15-AS1 |
| DDAH2 | C22orf34 | TTYH3 | PCDH7 | ZEB2 | DTX4 | PYGB | ATF6 |
| FAM198B | FOXN3 | DIRC3 | FAM81A | TPM2 | ITGA6 | CAV2 | DPP6 |
| PLEKHA6 | KAT6A | SERINC5 | TBCEL | HIGD1B | NTAN1 | SP3 | CCDC86 |
| MXRA8 | GJA5 | ADCY7 | HNMT | TASP1 | ANXA4 | RAP1GDS1 | DMD |
| GPNMB | PLEKHA7 | DPEP2 | FAM129B | VIPR1 | CCNY | WDR20 | EXD3 |
| OSR2 | CD151 | TMEM2 | SLC47A1 | FHL2 | ZBTB46 | PER3 | TMEM105 |
| RP11-563P16.1 | JAG2 | FMNL1 | FMO5 | ARHGAP44 | FLT4 | CCDC91 | BACH1 |
| SYTL4 | CLEC1A | TANC2 | TBC1D16 | MYO10 | PPM1F | ACER2 | RP11-452K12.7 |
| RP4-678D15.1 | VEGFC | CTD-2282P23.2 | LINC01482 | FAM46B | LRRFIP1 | USPL1 | TRPV2 |
| JUND | BCR | SLCO3A1 | OTX2-AS1 | PDE1C | SPPL3 | ZNF148 | ARMC2 |
| ELMO1 | FRMD4A | NUDT16 | MIRLET7BHG | TWSG1 | HID1 | SIK2 | TTL |
| RPL27 | S100A16 | SLC2A9 | ZNF330 | MOB2 | FBXO42 | ASS1 | HNRNPD |
| AFF3 | PLCG2 | LRRK1 | EMID1 | PDZRN3 | HECTD2 | SUCLG2-AS1 | PRDM2 |
| ECHDC2 | NR5A2 | EMP1 | SRP54 | PTPRD | RNF144A | MEIS1 | TBC1D19 |
| SMIM14 | LHFP | HMHA1 | REPS1 | PLEKHG3 | RAPGEF5 | RBBP6 | ACTG1 |
| ADAM33 | NFKBIA | SH3BGRL | EPC1 | AIG1 | FAM73A | CKB | FAM171A1 |
| ALDH1A2 | MT-ND4 | RFWD2 | GGNBP2 | GNAO1 | DGKE | PICALM | LAPTM4B |
| PDE1A | PCSK5 | ZNF846 | TSPYL2 | RSU1 | TRA2A | UBE2E2 | ANTXR1 |
| AC018890.6 | CX3CL1 | IL17RA | KCTD3 | EHD2 | KLHL3 | FER | RP11-573D15.2 |
| AP3S1 | LRRC8A | FOLR2 | ENOSF1 | SH3KBP1 | C1QTNF3-AMACR | SRPX2 | TLCD2 |
| CYP1B1-AS1 | ATP1A1 | COLGALT1 | RHOBTB3 | EIF4A2 | PAPLN | MT-CO2 | NR2C2 |
| SPSB1 | MEF2C | THEMIS2 | CCL2 | VIM | ANKRD6 | SLC25A43 | SRPRB |
| MAMDC2-AS1 | BMX | WASF2 | RSRP1 | COL27A1 | TSPAN9 | AACS | PIEZO2 |
| ATP1A1 | DGKE | ETNK1 | VRK3 | UNC5C | BMP2K | PIEZO1 | STAB2 |
| EEF1D | RPS18 | SLC4A7 | ESR1 | SELM | FABP4 | HACD2 | RP11-545G3.1 |
| EID1 | SLC35G2 | ADGRE5 | PLOD1 | WSB1 | EPN2 | CSPG4 | RAF1 |
| RPL37 | UBC | AFF3 | RP5-903G2.2 | ARHGAP42 | GLT8D2 | SLTM | KLHL15 |
| CSTB | KCTD12 | PLEKHA2 | CACUL1 | NMD3 | FAM102A | PRPF3 | RP11-518L10.5 |
| AKAP12 | NLRC5 | DRAM1 | SARAF | IQCJ-SCHIP1 | LYST | ITGB5 | PARK2 |
| TASP1 | CYTH1 | ATG7 | THRA | ZFP36 | CREB5 | ADRA2A | CNTN4 |
| LSP1 | ZNF264 | ZC3HAV1 | EXT1 | BGN | SMURF2 | ELMOD3 | MMP20 |
| RASSF4 | RNF144B | FCGRT | ANO10 | ADAMTS15 | FAM189A2 | PPP2R2A | ERBB2IP |
| EMP1 | HERPUD1 | ZNF562 | PCM1 | ANXA6 | NPEPPS | ACSS3 | HELB |
| MT1E | HECW2 | CYTH4 | COMMD2 | EMILIN1 | BAZ2B | UGGT2 | FAM104B |
| MPZL1 | PARP9 | NCOA2 | MLLT3 | SERTAD3 | TNKS | LINC00909 | NEB |
| NGFRAP1 | MIER2 | CTTNBP2NL | ITGA9 | OSBPL5 | AC108938.5 | FGF1 | ANKRD52 |
| CYTH3 | GATA2 | RASSF2 | PIK3R1 | OAZ2 | EFCC1 | CD151 | POLR1C |
| PAK3 | HLA-DQB1 | WDFY3 | FGF1 | A2ML1-AS1 | GRIN2B | COL15A1 | NUP54 |
| FER | PPM1D | FAM196B | TAF15 | RBBP6 | FANCL | ARHGAP29 | RBM20 |
| KLF9 | PIK3C2B | ARHGAP31 | ASAP2 | RP11-403A3.3 | TXNIP | NET1 | RP11-871F6.3 |
| ADAMTSL3 | ENPP2 | ACER3 | A1BG | FOXC2 | SMARCA2 | HEPACAM | RP11-648L3.2 |
| PRKAA1 | PLSCR1 | LYST | GBP2 | TANC1 | HDAC7 | RBMS1 | MTHFD2 |
| SLC2A13 | FXYD5 | RP11-289H16.1 | NEK7 | LINC00670 | RP11-184E9.1 | MON2 | HLX-AS1 |
| TOMM7 | REEP3 | CTSC | ST8SIA1 | CLIC1 | PTBP2 | SLC7A6OS | RP11-20E24.1 |
| PCOLCE | STK38 | CMKLR1 | ANKRD13D | RP11-466A19.3 | RERE | BCAP31 | REEP5 |
| SLC12A1 | MAP3K1 | TLR4 | DEC1 | PALM2-AKAP2 | FAM213A | PIKFYVE | RP11-775H9.2 |
| MEG8 | NES | MPEG1 | RPL7A | LAPTM4A | SLC12A6 | FAM35A | CUTC |
| PTPN9 | NOD1 | NHLRC3 | TMEM45A | TBL1X | AGMO | AIMP1 | MAP7D1 |
| ISLR | CHSY3 | TRERF1 | RHD | PTP4A3 | RP11-96H19.1 | ATF4 | MIOS |
| STK17B | HPCAL1 | ADPGK | ZNF37A | AKAP12 | PDE9A | DSEL | RP11-689B22.2 |
| PTPN13 | SULF2 | CD247 | RPL7 | ZFP36L1 | ITSN1 | SLC3A2 | NOM1 |
| ODF2L | COBLL1 | PSMA1 | CHRDL2 | SYNPO2 | NPAS3 | ANTXR2 | CTC-338M12.7 |
| CYB5R3 | OSMR | ARHGAP25 | UST | SIPA1L1 | CHST12 | NAT8L | ETHE1 |
| RHOBTB3 | SMAD1 | INPP4A | RNF20 | TMTC1 | HNRNPA1 | EPB41L2 | ZFAND3 |
| ADAMTS9-AS2 | TPST1 | TLR1 | RPS17 | PHACTR1 | LINC01197 | MT-CYB | HK2 |
| CPQ | RAPGEF4 | WDR36 | FAM13C | NME3 | FGD5 | SLC2A4RG | PHKA1 |
| BZW1 | MT-ATP6 | LRRC25 | HSCB | SRSF5 | PAWR | MCCC2 | RP11-666A8.8 |
| COX7C | KLHL5 | PLD3 | LRP4-AS1 | IGFBP4 | NFATC1 | SELENBP1 | POLG |
| EMX2OS | AL035610.2 | CLEC4E | ZFAND5 | ITGA7 | DCDC2C | FUBP3 | B3GLCT |
| TMSB10 | ANKS1A | PLEK | TUBA1B | ADRA2B | PREX2 | AC004160.4 | AC009120.4 |
| MT1M | MAST4 | ATRN | CTA-243E7.1 | SH3RF3 | KIAA1109 | AC002117.1 | EAF1 |
| SRSF7 | WNT2B | SMARCA2 | IFT122 | CENPO | RINT1 | DUSP4 | GRIN2A |
| RBMS1 | BCAM | PIK3AP1 | PPP1CB | EIF4A3 | PIK3C2A | MAP3K8 | DHRS7 |
| WSB1 | IGF2 | EVI2B | PDE1A | CBL | TCF4 | ADCK3 | RP11-348J24.2 |
| PCSK5 | RPL8 | RGS1 | EIF1 | ITGA3 | NXN | MPP6 | HPGDS |
| ZFP36L1 | MGST2 | EDA | MED6 | TUBB6 | FDPS | UBAC2 | RP11-472M19.2 |
| SOCS1 | TM6SF1 | ARHGAP30 | ZNF512 | FOSL2 | TTF1 | EIF4EBP1 | ASB15 |
| CTTNBP2 | KCNMB4 | HMGB2 | XXbac-BPG154L12.5 | SLC16A12-AS1 | RP11-274B18.4 | WBP1L | RP11-649A16.1 |
| PMP22 | MYL12B | LEPROTL1 | NAV3 | RP11-223C24.1 | RPL27A | TWIST2 | SAE1 |
| SRSF5 | KIAA1462 | HCST | CTDSPL | ZNF141 | STARD3 | KLHDC10 | HOOK2 |
| SOD3 | THSD7A | FGD4 | RP11-39E3.3 | NCKAP5 | FAM179B | HSDL2 | ITPKC |
| TBRG1 | CDC42EP3 | ITK | HMGN3 | EMD | RP11-522B15.3 | DDHD2 | EIF2S2 |
| TSKU | PRDM16 | EAF2 | IL1RAP | BRD3 | TMSB4X | MGAT3 | RP11-356C4.3 |
| CH507-513H4.1 | OSBPL1A | SKAP1 | USP12 | LINC00924 | CCDC39 | UPP2 | LINC01090 |
| TSHZ2 | PGM5 | EMILIN2 | KRIT1 | SLC6A1-AS1 | PRELID2 | PAXBP1 | TAF13 |
| LONRF1 | PDE10A | ALOX5AP | TRPS1 | DLGAP3 | CEP162 | FAM89A | C16orf70 |
| TEX26-AS1 | FZD6 | LGMN | COPS4 | PARM1 | FAM188A | AKR1A1 | RP11-382A20.3 |
| GRINA | MT-CO1 | CAP1 | PPP1R14B | SIK3 | GMDS-AS1 | ASCC2 | PIM1 |

In some cases, the nucleic specific genes may be one or more from Table 2A. In some cases, the nucleic specific genes may be one or more from cluster 0 of Table 2A. In some cases, the nucleic specific genes may be one or more from cluster 2 of Table 2A. In some cases, the nucleic specific genes may be one or more from cluster 6 of Table 2A.

TABLE 2A
Single Nuclei CST Preparation (Myocyte clusters)

| | | | | | | | |
|---|---|---|---|---|---|---|---|
| cluster | gene | tstat | log2FC | pval | pval_adj | percent_exp | mean_exp |

Cluster 0 Myocyte

| | | | | | | | |
|---|---|---|---|---|---|---|---|
| 0 | PDE4D | 68.61958313 | 3.992664337 | 0 | 0 | 0.967267869 | 4.170296669 |
| 0 | NEB | 60.71369934 | 3.662475586 | 0 | 0 | 0.930193721 | 3.643766165 |
| 0 | TRIM63 | 59.33517075 | 3.742561579 | 0 | 0 | 0.846025384 | 3.243874311 |
| 0 | MLIP | 58.93635559 | 3.47367835 | 0 | 0 | 0.889111556 | 3.172783613 |
| 0 | EYA4 | 55.55070877 | 3.397393942 | 0 | 0 | 0.849031396 | 3.009150505 |
| 0 | FBXO32 | 54.86566925 | 3.569988489 | 0 | 0 | 0.837675351 | 3.382109642 |
| 0 | TBC1D8 | 52.62391281 | 3.153393269 | 0 | 0 | 0.850701403 | 3.057489872 |
| 0 | TTN | 51.73801422 | 3.044782639 | 0 | 0 | 0.952905812 | 3.547366619 |
| 0 | GBE1 | 49.94226837 | 3.167587519 | 0 | 0 | 0.815297261 | 3.117704391 |
| 0 | DMD | 49.35060501 | 2.847150564 | 0 | 0 | 0.96492986 | 3.827222109 |
| 0 | SVIL | 47.18991089 | 2.904101849 | 0 | 0 | 0.898129593 | 3.341062546 |
| 0 | RP11-362A9.3 | 47.18364716 | 3.238517046 | 0 | 0 | 0.811289245 | 3.294064522 |
| 0 | TPM3 | 46.39105988 | 3.049887419 | 0 | 0 | 0.771543086 | 2.781247139 |
| 0 | CAP2 | 45.9341507 | 3.008091688 | 0 | 0 | 0.774549098 | 2.927838326 |
| 0 | ATP2A2 | 44.59591675 | 3.308048248 | 0 | 0 | 0.63760855 | 2.562462568 |
| 0 | ACACB | 44.018116 | 2.811622381 | 0 | 0 | 0.822979292 | 2.974886894 |
| 0 | ALK | 43.55496216 | 3.329214334 | 0 | 0 | 0.63259853 | 2.734760284 |
| 0 | RYR1 | 42.47189713 | 2.804944515 | 0 | 0 | 0.753841015 | 2.777573586 |
| 0 | TP63 | 42.26844788 | 3.15656805 | 0 | 0 | 0.650300601 | 2.813651323 |
| 0 | ABLIM2 | 41.95310974 | 2.872680187 | 0 | 0 | 0.704074816 | 2.660443783 |
| 0 | MYPN | 41.42292023 | 2.770480633 | 0 | 0 | 0.749832999 | 2.786068439 |
| 0 | RP11-64D24.2 | 40.76169586 | 2.612731934 | 0 | 0 | 0.775551102 | 2.945263147 |
| 0 | MPV17 | 40.67562866 | 2.876346111 | 0 | 0 | 0.664662659 | 2.56467247 |
| 0 | ARPP21 | 39.77843475 | 3.256039619 | 2.0651E−300 | 2.1115E−297 | 0.558784235 | 2.59334445 |
| 0 | PDLIM5 | 39.75080872 | 2.490196943 | 8.8954E−305 | 9.459E−302 | 0.864395458 | 3.186439991 |
| 0 | AKAP6 | 39.35155869 | 2.991283417 | 1.9249E−297 | 1.8276E−294 | 0.603874415 | 2.548902988 |
| 0 | MYBPC1 | 39.20515823 | 3.137567759 | 8.9187E−299 | 8.7813E−296 | 0.722110888 | 3.852462769 |
| 0 | RBFOX1 | 38.94123459 | 2.669104815 | 1.2489E−295 | 1.1449E−292 | 0.72745491 | 2.866345406 |
| 0 | CEP128 | 38.81991577 | 3.645617485 | 7.3978E−283 | 6.1457E−280 | 0.500334001 | 2.921466827 |
| 0 | KCNQ5 | 38.47585297 | 2.843467474 | 2.0171E−288 | 1.7298E−285 | 0.677020708 | 2.960836172 |
| 0 | PEX5L | 37.77213669 | 2.878014803 | 1.056E−277 | 8.5068E−275 | 0.631596526 | 2.915778637 |
| 0 | OBSCN | 37.49141312 | 2.753453493 | 2.0501E−274 | 1.5571E−271 | 0.625918504 | 2.571044445 |
| 0 | PDLIM3 | 37.36595154 | 2.817101479 | 1.0131E−274 | 7.9212E−272 | 0.740480962 | 3.453696012 |
| 0 | TACC2 | 37.3216362 | 2.705667734 | 1.7152E−272 | 1.2666E−269 | 0.638276553 | 2.676099777 |
| 0 | PPARGC1A | 37.22993851 | 3.233568192 | 6.8656E−266 | 4.5629E−263 | 0.511022044 | 2.525043964 |
| 0 | GPATCH8 | 37.20300293 | 2.288942337 | 1.9841E−272 | 1.4256E−269 | 0.813627255 | 2.974091291 |
| 0 | TRDN | 36.98090363 | 2.610836983 | 1.2075E−269 | 8.4472E−267 | 0.710420842 | 3.004619598 |
| 0 | CPED1 | 36.93974304 | 2.763715506 | 3.8208E−267 | 2.6044E−264 | 0.626252505 | 2.790329218 |
| 0 | MAST2 | 36.46698761 | 2.58582592 | 1.0657E−261 | 6.7451E−259 | 0.63493654 | 2.527561665 |
| 0 | TPD52L1 | 36.42673111 | 2.998893261 | 4.7525E−258 | 2.8713E−255 | 0.539412158 | 2.474223375 |
| 0 | SPDYE2 | 36.29946136 | 2.371098042 | 7.1034E−261 | 4.3915E−258 | 0.743486974 | 2.923092842 |
| 0 | RIF1 | 36.12831116 | 2.629807472 | 1.9652E−257 | 1.1609E−254 | 0.634602538 | 2.737119198 |
| 0 | CTNNA3 | 35.98176193 | 2.551259518 | 8.8935E−256 | 5.1397E−253 | 0.640280561 | 2.566310883 |
| 0 | HDAC4 | 35.82993698 | 2.479841471 | 3.258E−254 | 1.8428E−251 | 0.665664663 | 2.670019627 |
| 0 | USP54 | 35.71902466 | 2.561039448 | 3.0246E−252 | 1.6409E−249 | 0.626920508 | 2.570672035 |
| 0 | UBE2E2 | 35.47087479 | 2.224067926 | 2.8144E−250 | 1.4964E−247 | 0.781229125 | 2.843953609 |
| 0 | EMC10 | 35.41286087 | 2.820920467 | 1.4267E−249 | 7.4369E−247 | 0.731128925 | 3.464151621 |
| 0 | PDE4DIP | 35.1595192 | 2.108855247 | 1.4979E−245 | 7.5133E−243 | 0.841015364 | 3.000817537 |
| 0 | GTF2IRD1 | 34.65812302 | 2.573727369 | 1.5297E−238 | 7.5308E−236 | 0.594522378 | 2.535318613 |
| 0 | KCNMA1 | 34.53581238 | 2.809384108 | 2.2654E−235 | 1.0754E−232 | 0.541082164 | 2.53961134 |
| 0 | PDE7A | 34.36991501 | 2.392346621 | 5.5005E−236 | 2.6586E−233 | 0.655978624 | 2.580513477 |
| 0 | NEXN | 34.2109642 | 2.270653009 | 3.336E−234 | 1.5558E−231 | 0.778223113 | 2.974342346 |
| 0 | SLC16A10 | 33.82657623 | 2.553561211 | 1.1572E−228 | 5.2143E−226 | 0.598530394 | 2.597869158 |
| 0 | CLIP1 | 33.81100464 | 2.233375072 | 1.7305E−229 | 7.9317E−227 | 0.721776887 | 2.71036768 |
| 0 | MYOT | 33.60438156 | 2.625977039 | 4.7099E−225 | 2.0526E−222 | 0.552104208 | 2.497097969 |
| 0 | NEAT1 | 33.42187119 | 1.504169345 | 1.0118E−218 | 4.0146E−216 | 0.988643955 | 4.88466692 |
| 0 | ASB5 | 33.38949966 | 2.594193697 | 9.1184E−223 | 3.7876E−220 | 0.558784235 | 2.477323294 |
| 0 | TTN-AS1 | 33.27072144 | 2.211858988 | 7.5482E−223 | 3.1851E−220 | 0.743486974 | 2.872821093 |
| 0 | SYT2 | 33.25154495 | 2.76418066 | 1.4231E−219 | 5.8204E−217 | 0.501002004 | 2.445770264 |
| 0 | PCNT | 32.84406281 | 2.497022629 | 3.1879E−216 | 1.2282E−213 | 0.566466266 | 2.459780693 |
| 0 | MYO18B | 32.80678558 | 2.405478477 | 3.4364E−217 | 1.3434E−214 | 0.675350701 | 2.854115248 |
| 0 | MYOM3 | 32.71341705 | 2.8204844 | 5.4722E−213 | 2.0782E−210 | 0.479291917 | 2.433751583 |
| 0 | RP11-381K20.2 | 31.75157547 | 2.582780123 | 9.6849E−203 | 3.4792E−200 | 0.514362057 | 2.489465237 |
| 0 | PDK4 | 31.64107132 | 2.523288488 | 2.8548E−203 | 1.0396E−200 | 0.782231129 | 3.977900982 |
| 0 | ZNF385B | 31.3874588 | 3.081628323 | 1.6033E−195 | 5.395E−193 | 0.405811623 | 2.499852657 |
| 0 | LRRTM3 | 31.21370506 | 2.765201807 | 2.1853E−195 | 7.2618E−193 | 0.458583834 | 2.449576378 |
| 0 | RP11-266N13.2 | 31.18343925 | 2.799528122 | 1.0204E−194 | 3.3079E−192 | 0.444555778 | 2.345142365 |
| 0 | RP11-532N4.2 | 31.12461281 | 2.584900379 | 6.7906E−196 | 2.3444E−193 | 0.542752171 | 3.095878839 |
| 0 | KIF1B | 30.90872383 | 2.047968626 | 9.38E−195 | 3.0785E−192 | 0.723780895 | 2.82414341 |
| 0 | P4HA1 | 30.55081558 | 2.51658082 | 7.7321E−189 | 2.3901E−186 | 0.508016032 | 2.62462163 |
| 0 | DEPTOR | 30.52898979 | 2.255293131 | 5.6513E−190 | 1.7675E−187 | 0.603540414 | 2.594959974 |
| 0 | HADHB | 30.35699272 | 2.422714949 | 6.4226E−187 | 1.9184E−184 | 0.51503006 | 2.419969559 |
| 0 | CFAP61 | 30.34096146 | 2.783755541 | 5.2913E−185 | 1.5458E−182 | 0.428857715 | 2.358040333 |
| 0 | BACH1 | 30.15842438 | 2.069145203 | 3.9202E−186 | 1.1579E−183 | 0.685370741 | 2.644852161 |
| 0 | ERMP1 | 30.04843521 | 2.533556938 | 3.9066E−183 | 1.1048E−180 | 0.480293921 | 2.465787172 |
| 0 | CUX1 | 30.03153038 | 1.906105399 | 1.0816E−184 | 3.1254E−182 | 0.736472946 | 2.672874689 |
| 0 | SPRTN | 29.98522949 | 2.606346607 | 4.6573E−182 | 1.2897E−179 | 0.461589846 | 2.478764296 |
| 0 | KIAA1217 | 29.97461891 | 1.919430852 | 7.1792E−184 | 2.0522E−181 | 0.798597194 | 3.011316299 |
| 0 | IL12RB2 | 29.62168884 | 2.475914955 | 1.4402E−178 | 3.8674E−176 | 0.482297929 | 2.385469437 |
| 0 | TNNT1 | 29.45252609 | 2.069316387 | 3.8732E−178 | 1.0296E−175 | 0.699732799 | 2.646347046 |
| 0 | CACNA2D1 | 29.15551376 | 1.898798943 | 7.3256E−175 | 1.9282E−172 | 0.717768871 | 2.706177235 |
| 0 | FILIP1L | 28.84715271 | 1.948173165 | 1.9216E−171 | 4.9597E−169 | 0.719772879 | 2.93017602 |
| 0 | OSBPL6 | 28.84336281 | 2.544007778 | 1.7439E−169 | 4.4577E−167 | 0.440547762 | 2.384403229 |
| 0 | DPF3 | 28.8122673 | 2.461374044 | 2.0014E−169 | 5.067E−167 | 0.455577822 | 2.353761196 |
| 0 | ASB2 | 28.70199013 | 2.344035864 | 9.7239E−169 | 2.3935E−166 | 0.483967936 | 2.286568642 |
| 0 | CFLAR-AS1 | 28.69623375 | 2.392811298 | 2.2407E−168 | 5.4649E−166 | 0.468269873 | 2.329404831 |
| 0 | MSI2 | 28.66431618 | 2.175275564 | 8.5702E−169 | 2.1293E−166 | 0.53507014 | 2.360114574 |
| 0 | EPS15L1 | 28.37550926 | 2.116185904 | 8.9353E−166 | 2.1209E−163 | 0.555444222 | 2.449676514 |
| 0 | CMYA5 | 28.32237244 | 2.002514362 | 1.0819E−165 | 2.5452E−163 | 0.64261857 | 2.544760704 |
| 0 | FAM184B | 28.06536102 | 2.001256466 | 6.349E−163 | 1.455E−160 | 0.655310621 | 2.687170267 |
| 0 | SLC22A4 | 28.06235123 | 2.753233433 | 2.7413E−160 | 6.0729E−158 | 0.37742151 | 2.250035524 |
| 0 | RP3-355L5.4 | 28.02748108 | 2.647592306 | 2.5698E−160 | 5.7784E−158 | 0.395791583 | 2.281990767 |
| 0 | RP11-309L24.2 | 27.96420479 | 2.876106977 | 9.1842E−159 | 1.969E−156 | 0.358717435 | 2.385373831 |
| 0 | ZFAND3 | 27.89073181 | 1.682770729 | 6.024E−161 | 1.3687E−158 | 0.794589178 | 2.84032464 |
| 0 | ATP1A2 | 27.88235474 | 2.195839643 | 2.5866E−160 | 5.7784E−158 | 0.506346025 | 2.350500345 |
| 0 | ACSL1 | 27.85741806 | 2.110976696 | 2.8655E−160 | 6.2955E−158 | 0.548430194 | 2.463222027 |
| 0 | PPP1R12B | 27.51546669 | 1.982313156 | 8.3005E−157 | 1.7653E−154 | 0.586172345 | 2.502932787 |
| 0 | MYOM1 | 27.49575043 | 2.055701494 | 9.0639E−157 | 1.9123E−154 | 0.624248497 | 2.84263587 |
| 0 | AGBL1 | 26.97872162 | 2.341879606 | 2.8633E−150 | 5.6805E−148 | 0.475951904 | 2.736681938 |
| 0 | SIL1 | 26.97843742 | 1.9487468 | 3.7986E−151 | 7.6501E−149 | 0.579492318 | 2.468524694 |
| 0 | BEST3 | 26.76652336 | 2.75079155 | 1.008E−146 | 1.9559E−144 | 0.347695391 | 2.230817556 |
| 0 | SF3A3 | 26.67542267 | 2.274380207 | 2.7864E−147 | 5.4467E−145 | 0.449231797 | 2.467421532 |
| 0 | PHYHIPL | 26.66475487 | 3.275778294 | 1.7365E−144 | 3.274E−142 | 0.295925184 | 2.372275829 |
| 0 | CH17-189H20.1 | 26.51670456 | 1.793819189 | 1.4376E−146 | 2.7693E−144 | 0.658984636 | 2.61508894 |
| 0 | MTFR1 | 26.50806999 | 2.122805119 | 7.9124E−146 | 1.5133E−143 | 0.485303941 | 2.34847641 |
| 0 | RP11-768F21.1 | 26.44731331 | 2.162206888 | 2.6395E−145 | 5.0121E−143 | 0.489311957 | 2.469034195 |
| 0 | AMOTL1 | 26.43068886 | 2.441403389 | 3.8932E−144 | 7.2885E−142 | 0.394121576 | 2.320879698 |
| 0 | CAPN9 | 26.37325668 | 3.244664192 | 1.4314E−141 | 2.5886E−139 | 0.288243153 | 2.280155182 |
| 0 | SNX1 | 26.20908546 | 1.954353213 | 2.7123E−143 | 4.9726E−141 | 0.569806279 | 2.489049911 |
| 0 | THRB | 26.12785912 | 1.799514413 | 1.4501E−142 | 2.6404E−140 | 0.648296593 | 2.584758759 |
| 0 | SLC24A1 | 25.89633751 | 2.418996096 | 7.1477E−139 | 1.2584E−136 | 0.387107548 | 2.259059906 |
| 0 | LPIN1 | 25.8287487 | 1.890947342 | 1.8342E−139 | 3.2508E−137 | 0.598196393 | 2.611103535 |
| 0 | LRRC2 | 25.81399155 | 2.325558424 | 2.6006E−138 | 4.5482E−136 | 0.406479626 | 2.246033192 |
| 0 | SOX6 | 25.72975159 | 2.070894003 | 5.2391E−138 | 9.1029E−136 | 0.482297929 | 2.416319847 |
| 0 | MAP4K1 | 25.55512428 | 2.350612879 | 1.1183E−135 | 1.9305E−133 | 0.39512358 | 2.357576609 |
| 0 | DENND2C | 25.39530373 | 2.248008966 | 2.7489E−134 | 4.6545E−132 | 0.419171677 | 2.472405195 |
| 0 | SLC20A2 | 25.35896492 | 1.912011862 | 1.1967E−134 | 2.0393E−132 | 0.549432198 | 2.546832561 |
| 0 | UBAC1 | 25.34472656 | 2.169549942 | 5.5038E−134 | 9.2602E−132 | 0.431863727 | 2.292119503 |
| 0 | CKMT2 | 25.18449402 | 2.456076622 | 1.2641E−131 | 2.0743E−129 | 0.363059452 | 2.349419117 |
| 0 | ANKRD2 | 25.1487484 | 2.572437048 | 3.6164E−131 | 5.8981E−129 | 0.364395458 | 2.895688534 |
| 0 | ATG14 | 25.0057373 | 2.115100145 | 1.0353E−130 | 1.668E−128 | 0.440547762 | 2.336565971 |
| 0 | DIO2-AS1 | 24.9681015 | 3.012221098 | 4.4595E−128 | 7.0566E−126 | 0.293921176 | 2.827899218 |
| 0 | ACTN2 | 24.86448479 | 2.00816226 | 1.1941E−129 | 1.9123E−127 | 0.490313961 | 2.445637941 |
| 0 | LARGE | 24.7818737 | 1.797219872 | 4.7148E−129 | 7.5053E−127 | 0.60253841 | 2.624568701 |
| 0 | SSH2 | 24.49364281 | 1.503690124 | 3.2151E−126 | 5.0276E−124 | 0.809619238 | 2.964446068 |
| 0 | SGCD | 24.48029709 | 1.965498328 | 7.5256E−126 | 1.1699E−123 | 0.476953908 | 2.383984804 |
| 0 | ABCC5 | 24.43843079 | 2.181693077 | 5.4139E−125 | 8.3193E−123 | 0.393787575 | 2.221780539 |
| 0 | LRRFIP2 | 24.36563301 | 1.76605463 | 5.0983E−125 | 7.8798E−123 | 0.570140281 | 2.502384901 |
| 0 | FKBP5 | 24.2828598 | 1.205073833 | 2.2608E−123 | 3.3956E−121 | 0.963593854 | 3.888625622 |
| 0 | C10orf71 | 24.28159332 | 2.528484583 | 7.7852E−123 | 1.1434E−120 | 0.322645291 | 2.183171272 |
| 0 | VPS13A | 24.25829887 | 2.119131327 | 2.396E−123 | 3.5785E−121 | 0.404141617 | 2.251445293 |
| 0 | WDPCP | 24.20631218 | 1.779358149 | 1.4393E−123 | 2.1864E−121 | 0.679024716 | 2.902488708 |
| 0 | PRKG1 | 24.18781471 | 1.661526799 | 2.1524E−123 | 3.2511E−121 | 0.709084836 | 2.892318249 |
| 0 | RERE | 24.16213226 | 1.581314921 | 3.7435E−123 | 5.5597E−121 | 0.683700735 | 2.584929466 |
| 0 | MEF2C-AS1 | 24.06758308 | 2.424969196 | 5.6206E−121 | 8.0332E−119 | 0.335337341 | 2.248305082 |
| 0 | RP11-145A3.1 | 24.02444077 | 2.832775831 | 7.7728E−120 | 1.0933E−117 | 0.280227121 | 2.244181156 |
| 0 | RP11-83A24.2 | 24.00654602 | 1.941798925 | 2.5076E−121 | 3.6033E−119 | 0.46760187 | 2.382537365 |
| 0 | RP11-144F15.1 | 23.95237732 | 2.011869907 | 7.6061E−121 | 1.0813E−118 | 0.457247829 | 2.456757784 |
| 0 | PPP1R27 | 23.83508682 | 1.999776125 | 1.2104E−119 | 1.6758E−117 | 0.460253841 | 2.563255548 |
| 0 | THUMPD3-AS1 | 23.8260994 | 1.857597947 | 1.1475E−119 | 1.6056E−117 | 0.487975952 | 2.360493183 |
| 0 | FAM134B | 23.80976295 | 1.863819718 | 1.171E−119 | 1.6298E−117 | 0.533734135 | 2.569919586 |
| 0 | ZNF565 | 23.79756927 | 1.745869756 | 1.4038E−119 | 1.9336E−117 | 0.555778223 | 2.427254677 |
| 0 | COL14A1 | 23.78093147 | 2.090671062 | 6.6471E−119 | 9.1086E−117 | 0.412157649 | 2.440153599 |
| 0 | SRL | 23.58191299 | 2.249143124 | 8.5748E−117 | 1.169E−114 | 0.358383434 | 2.215624332 |
| 0 | REV3L | 23.32861137 | 1.538290262 | 2.7723E−115 | 3.741E−113 | 0.68503674 | 2.636009693 |
| 0 | SLC22A5 | 23.18772316 | 2.635643005 | 2.0154E−112 | 2.6524E−110 | 0.282231129 | 2.145102978 |
| 0 | RP11-434D9.1 | 23.17916679 | 1.848979354 | 7.0133E−114 | 9.4163E−112 | 0.662324649 | 3.187238932 |
| 0 | GRIK1 | 23.01358032 | 1.791806459 | 2.8202E−112 | 3.6932E−110 | 0.533734135 | 2.57220006 |
| 0 | SPPL2A | 22.94172478 | 1.795203328 | 1.6445E−111 | 2.143E−109 | 0.486973948 | 2.34261322 |
| 0 | ZBTB38 | 22.92514801 | 2.308531046 | 1.5418E−110 | 1.9801E−108 | 0.32999332 | 2.329319239 |
| 0 | MACROD2 | 22.88597488 | 1.784253955 | 5.3711E−111 | 6.9313E−109 | 0.492317969 | 2.420938253 |
| 0 | ABCC9 | 22.85648727 | 2.350472927 | 6.4762E−110 | 8.2771E−108 | 0.315965264 | 2.177182674 |
| 0 | RP11-701P16.2 | 22.83846092 | 2.448347092 | 1.9534E−109 | 2.4495E−107 | 0.315965264 | 2.515683174 |
| 0 | CLTCL1 | 22.75870514 | 1.832921386 | 8.0152E−110 | 1.0195E−107 | 0.474281897 | 2.490790606 |
| 0 | TARID | 22.56588936 | 2.042217016 | 8.6198E−108 | 1.0511E−105 | 0.383099532 | 2.260473728 |
| 0 | NFE2L3 | 22.52220535 | 2.152030706 | 3.2921E−107 | 3.9781E−105 | 0.3500334 | 2.242167711 |
| 0 | VWA8 | 22.52144241 | 1.831857324 | 1.2854E−107 | 1.5603E−105 | 0.44756179 | 2.280318737 |
| 0 | PIGB | 22.48106384 | 1.961720467 | 4.3377E−107 | 5.2178E−105 | 0.39746159 | 2.259169102 |
| 0 | CFAP100 | 22.2830162 | 2.153555155 | 4.4697E−105 | 5.281E−103 | 0.342351369 | 2.184520721 |
| 0 | TMEM161B-AS1 | 22.27008438 | 2.029506207 | 4.2E−105 | 4.9845E−103 | 0.374415498 | 2.268304348 |
| 0 | PLEKHG6 | 22.19320107 | 2.523347378 | 9.6268E−104 | 1.1274E−101 | 0.276887108 | 2.175747633 |
| 0 | ASB8 | 22.10513687 | 1.83498311 | 6.0885E−104 | 7.1618E−102 | 0.445891784 | 2.426136017 |
| 0 | AC016723.4 | 22.10408592 | 2.492001534 | 5.0185E−103 | 5.8005E−101 | 0.292919172 | 2.560842276 |
| 0 | RP5-1042K10.10 | 22.06189346 | 2.119253159 | 3.8735E−103 | 4.4967E−101 | 0.343687375 | 2.208790064 |
| 0 | OSBPL9 | 22.04740906 | 1.531987071 | 1.2577E−103 | 1.4664E−101 | 0.628924516 | 2.685776472 |
| 0 | PPP2R3A | 21.99223137 | 1.758161306 | 5.3721E−103 | 6.1824E−101 | 0.470607882 | 2.372997046 |
| 0 | MLIP-AS1 | 21.98407364 | 2.116721392 | 1.8636E−102 | 2.1354E−100 | 0.338677355 | 2.200004578 |
| 0 | TMEM135 | 21.7348671 | 1.74168396 | 1.1466E−100 | 1.28611E−98 | 0.469271877 | 2.436779976 |
| 0 | MAPT | 21.71599388 | 2.230941296 | 6.3608E−100 | 7.04562E−98 | 0.308617234 | 2.186402082 |
| 0 | FAM179A | 21.67893982 | 2.930348158 | 9.1845E−99 | 9.96576E−97 | 0.226786907 | 2.275353193 |
| 0 | FAM214A | 21.67677307 | 1.849279046 | 4.838E−100 | 5.40397E−98 | 0.406813627 | 2.292209387 |
| 0 | FILIP1 | 21.57664871 | 1.745139718 | 2.5534E−99 | 2.79335E−97 | 0.445891784 | 2.25848937 |
| 0 | FOXO1 | 21.57473564 | 1.406141996 | 1.8845E−99 | 2.07019E−97 | 0.753173013 | 2.877528906 |
| 0 | AC016831.7 | 21.54086113 | 1.320760012 | 4.9136E−99 | 5.35345E−97 | 0.839011356 | 3.125647783 |
| 0 | PFKFB2 | 21.51947784 | 2.925614357 | 1.98214E−97 | 2.12473E−95 | 0.220440882 | 2.067521572 |
| 0 | LGR5 | 21.45572853 | 3.013949633 | 8.11628E−97 | 8.4613E−95 | 0.216098864 | 2.196482182 |
| 0 | KLF13 | 21.40502548 | 2.179929972 | 2.80319E−97 | 2.96893E−95 | 0.326319305 | 2.451854467 |
| 0 | DLG1 | 21.39621925 | 1.584941745 | 7.2622E−98 | 7.81612E−96 | 0.523714095 | 2.391743898 |
| 0 | PTPN3 | 21.37410736 | 2.19948864 | 5.18656E−97 | 5.44979E−95 | 0.306613226 | 2.208249807 |
| 0 | FASTKD1 | 21.36909866 | 1.891086936 | 2.18901E−97 | 2.32771E−95 | 0.391449566 | 2.338891268 |
| 0 | DIO2 | 21.34057045 | 3.255300045 | 1.67833E−95 | 1.69003E−93 | 0.203740815 | 2.551481009 |
| 0 | PPP3CB | 21.31006622 | 1.85434413 | 7.8249E−97 | 8.18965E−95 | 0.391115564 | 2.334931612 |
| 0 | TTC19 | 21.25661087 | 1.511730433 | 1.02675E−96 | 1.06622E−94 | 0.582832331 | 2.491256952 |
| 0 | PDXDC1 | 21.24469185 | 1.217458248 | 1.72968E−96 | 1.78224E−94 | 0.841349365 | 2.981678486 |
| 0 | EEPD1 | 21.22801018 | 1.799583197 | 3.06077E−96 | 3.12952E−94 | 0.425183701 | 2.407431841 |
| 0 | SLC8B1 | 21.22086716 | 2.265208244 | 1.33953E−95 | 1.35399E−93 | 0.291917168 | 2.274900198 |
| 0 | TMEM233 | 21.16337967 | 1.989211321 | 1.71125E−95 | 1.71667E−93 | 0.3500334 | 2.256234169 |
| 0 | RP11-665C16.5 | 21.13415718 | 1.962411165 | 2.85962E−95 | 2.83657E−93 | 0.35738143 | 2.234058619 |
| 0 | ACYP2 | 21.0949192 | 1.465622306 | 2.48746E−95 | 2.47665E−93 | 0.601870407 | 2.49151206 |
| 0 | ZBTB43 | 20.98623276 | 1.866601586 | 4.32776E−94 | 4.24536E−92 | 0.376753507 | 2.220710516 |
| 0 | ZFAND1 | 20.87759209 | 1.992804766 | 4.60422E−93 | 4.43473E−91 | 0.349031396 | 2.448693514 |
| 0 | FAM160A1 | 20.87600136 | 1.987884164 | 4.88861E−93 | 4.69165E−91 | 0.345691383 | 2.300773382 |
| 0 | MNAT1 | 20.87011337 | 1.657263875 | 2.73979E−93 | 2.64853E−91 | 0.453573814 | 2.286383867 |
| 0 | RALGAPA2 | 20.81952477 | 1.693157077 | 6.85694E−93 | 6.55701E−91 | 0.463259853 | 2.383963108 |
| 0 | PEBP4 | 20.76915741 | 2.103837967 | 5.20594E−92 | 4.92508E−90 | 0.307949232 | 2.168758631 |
| 0 | NEDD4 | 20.7681427 | 1.59745276 | 1.59766E−92 | 1.5223E−90 | 0.540748163 | 2.634882927 |
| 0 | PLIN2 | 20.75019646 | 1.928549767 | 5.34155E−92 | 5.03546E−90 | 0.377087508 | 2.661484241 |
| 0 | THRAP3 | 20.72493172 | 1.662337184 | 4.70834E−92 | 4.47023E−90 | 0.447227789 | 2.279787302 |
| 0 | PBX1 | 20.70412445 | 1.596565723 | 6.28303E−92 | 5.90206E−90 | 0.496659987 | 2.531932116 |
| 0 | HECTD1 | 20.69924927 | 1.590952754 | 6.72745E−92 | 6.29727E−90 | 0.484301937 | 2.312118053 |
| 0 | USP2 | 20.67139435 | 2.552933931 | 1.01237E−90 | 9.34475E−89 | 0.23747495 | 2.099382162 |
| 0 | INPP5B | 20.66734695 | 2.070136786 | 4.3053E−91 | 4.00182E−89 | 0.317301269 | 2.572843552 |
| 0 | HADHA | 20.65263557 | 1.98977375 | 3.84893E−91 | 3.59017E−89 | 0.333333333 | 2.216634274 |
| 0 | SAMD8 | 20.52372932 | 1.653929114 | 2.01103E−90 | 1.84349E−88 | 0.458917836 | 2.377847195 |
| 0 | RP11-380P13.1 | 20.39535904 | 2.907059431 | 3.8227E−88 | 3.46835E−86 | 0.208750835 | 2.301008463 |
| 0 | PLIN5 | 20.30496025 | 2.156401157 | 4.02071E−88 | 3.6356E−86 | 0.284569138 | 2.1334126 |
| 0 | SNX24 | 20.2521553 | 2.186967134 | 1.33145E−87 | 1.19579E−85 | 0.281563126 | 2.450469017 |
| 0 | BIRC6-AS2 | 20.1740551 | 1.468981266 | 1.36628E−87 | 1.22294E−85 | 0.531396126 | 2.339387178 |
| 0 | CBX1 | 20.17192841 | 2.09747839 | 4.58681E−87 | 4.03761E−85 | 0.292251169 | 2.206412077 |
| 0 | EGLN1 | 20.16244698 | 1.659001827 | 2.11386E−87 | 1.87943E−85 | 0.43253173 | 2.281057596 |
| 0 | HOMER2 | 20.12990379 | 2.021402597 | 8.64352E−87 | 7.55853E−85 | 0.310621242 | 2.24898386 |
| 0 | NCOA1 | 20.08740997 | 1.289980531 | 6.7187E−87 | 5.89472E−85 | 0.709418838 | 2.689352512 |
| 0 | EPB41L4B | 20.07483292 | 2.985620499 | 1.65147E−85 | 1.4208E−83 | 0.19238477 | 2.103430986 |
| 0 | TIAM2 | 19.99300766 | 1.519391418 | 4.51267E−86 | 3.92042E−84 | 0.491649967 | 2.330135584 |
| 0 | CFLAR | 19.9420948 | 1.568974495 | 1.24321E−85 | 1.07303E−83 | 0.460921844 | 2.299286604 |
| 0 | ADCY9 | 19.92547226 | 1.737368941 | 2.19007E−85 | 1.87809E−83 | 0.399131597 | 2.374012232 |
| 0 | RBM20 | 19.89913177 | 2.365393877 | 1.29801E−84 | 1.09892E−82 | 0.244154977 | 2.188441992 |
| 0 | TBC1D1 | 19.79904938 | 1.781040549 | 2.57238E−84 | 2.15723E−82 | 0.373079492 | 2.311218977 |
| 0 | FBXL13 | 19.76528549 | 1.808576822 | 4.82501E−84 | 4.03359E−82 | 0.357047428 | 2.239766121 |
| 0 | STIM1 | 19.68281937 | 1.469692349 | 1.38072E−83 | 1.15063E−81 | 0.515698063 | 2.421742678 |
| 0 | TNS3 | 19.66385078 | 1.793755651 | 3.08111E−83 | 2.55166E−81 | 0.35504342 | 2.182443142 |
| 0 | VCL | 19.65529442 | 1.612930059 | 2.73579E−83 | 2.27276E−81 | 0.436205745 | 2.349202394 |
| 0 | ALPK2 | 19.62467003 | 2.828544378 | 4.77957E−82 | 3.86201E−80 | 0.194722779 | 2.132289648 |
| 0 | CECR2 | 19.60434151 | 1.7224437 | 8.55112E−83 | 7.03786E−81 | 0.375751503 | 2.233904123 |
| 0 | DAPK2 | 19.54920387 | 1.797298193 | 2.44531E−82 | 1.98795E−80 | 0.35504342 | 2.262529135 |
| 0 | IDNK | 19.53313446 | 1.862297058 | 3.76344E−82 | 3.05022E−80 | 0.332331329 | 2.24181366 |
| 0 | PPM1L | 19.53234863 | 1.406003356 | 2.10286E−82 | 1.7148E−80 | 0.573480294 | 2.558473587 |
| 0 | ABHD15-AS1 | 19.48394585 | 1.351298213 | 5.01688E−82 | 4.04147E−80 | 0.624582498 | 2.614513397 |
| 0 | MAP4K3 | 19.47755051 | 1.481763005 | 6.23111E−82 | 5.00447E−80 | 0.491983968 | 2.33822751 |
| 0 | ESRRG | 19.4695034 | 2.612329245 | 5.17003E−81 | 4.06628E−79 | 0.211088844 | 2.201326609 |
| 0 | ENAH | 19.46032333 | 1.736765742 | 1.24177E−81 | 9.91331E−80 | 0.37241149 | 2.277317286 |
| 0 | SLC25A33 | 19.44282722 | 2.024656296 | 2.96665E−81 | 2.34718E−79 | 0.286573146 | 2.176585436 |
| 0 | SMG1 | 19.44171906 | 1.519714713 | 1.24896E−81 | 9.94081E−80 | 0.463593854 | 2.302022457 |
| 0 | ALPK3 | 19.31243896 | 2.117278099 | 3.66063E−80 | 2.84544E−78 | 0.266533066 | 2.118359327 |
| 0 | C1orf21 | 19.21152115 | 1.302275062 | 7.22262E−80 | 5.59784E−78 | 0.637942552 | 2.577660322 |
| 0 | RP13-650J16.1 | 19.20614624 | 3.952174425 | 3.03185E−78 | 2.29626E−76 | 0.143954576 | 1.95617795 |
| 0 | BRE | 19.12962532 | 1.468732238 | 3.6004E−79 | 2.76627E−77 | 0.484301937 | 2.385412693 |
| 0 | MAP4 | 19.07341385 | 1.32431674 | 8.82524E−79 | 6.7611E−77 | 0.603874415 | 2.562568903 |
| 0 | SCML1 | 18.94378853 | 1.534621239 | 1.11017E−77 | 8.3843E−76 | 0.432197729 | 2.292755842 |
| 0 | CKMT2-AS1 | 18.92766762 | 1.750220895 | 1.90122E−77 | 1.42774E−75 | 0.344021376 | 2.257285595 |
| 0 | AC011288.2 | 18.80574036 | 2.372079611 | 4.99494E−76 | 3.68848E−74 | 0.218770875 | 2.202263594 |
| 0 | SLC36A2 | 18.78504753 | 2.512962103 | 8.84466E−76 | 6.47731E−74 | 0.208416834 | 2.356535196 |
| 0 | GPHN | 18.75004578 | 1.258585215 | 2.9432E−76 | 2.19165E−74 | 0.676018704 | 2.605829954 |
| 0 | NFATC3 | 18.74440384 | 1.482643723 | 3.64773E−76 | 2.70115E−74 | 0.456245825 | 2.334542513 |
| 0 | EIF4E2 | 18.7302742 | 1.738047004 | 6.5111E−76 | 4.79476E−74 | 0.33500334 | 2.169491053 |
| 0 | FAM20A | 18.71826363 | 1.774633765 | 8.1444E−76 | 5.98096E−74 | 0.338677355 | 2.330924034 |
| 0 | SLC3A1 | 18.65872002 | 2.282169342 | 5.35777E−75 | 3.87041E−73 | 0.225450902 | 2.127299547 |
| 0 | LRRC39 | 18.65620613 | 2.212306976 | 4.99359E−75 | 3.62704E−73 | 0.23246493 | 2.073775291 |
| 0 | ZNF827 | 18.63449669 | 2.233467102 | 7.32998E−75 | 5.25229E−73 | 0.230794923 | 2.100619555 |
| 0 | RP1-117O3.2 | 18.6285305 | 1.997213721 | 5.80859E−75 | 4.17339E−73 | 0.267869071 | 2.109057665 |
| 0 | SLC38A1 | 18.62761688 | 1.897983909 | 5.21356E−75 | 3.77649E−73 | 0.294589178 | 2.390807867 |
| 0 | RP11-701H24.9 | 18.61888885 | 1.651981354 | 3.86414E−75 | 2.81437E−73 | 0.369071476 | 2.239754915 |
| 0 | PARVB | 18.56809998 | 1.775204778 | 1.20702E−74 | 8.60253E−73 | 0.321643287 | 2.207850218 |
| 0 | SESN1 | 18.54857063 | 1.27614677 | 1.02567E−74 | 7.32967E−73 | 0.654642619 | 2.630752087 |
| 0 | ACACA | 18.5346508 | 1.559861898 | 1.69719E−74 | 1.20636E−72 | 0.408149633 | 2.353070498 |
| 0 | DNAH11 | 18.50172997 | 1.497407675 | 2.73191E−74 | 1.93667E−72 | 0.43253173 | 2.286004782 |
| 0 | VPS13D | 18.49386787 | 1.369110823 | 2.78116E−74 | 1.96634E−72 | 0.509018036 | 2.337418556 |
| 0 | CLIP4 | 18.4895134 | 1.815882921 | 4.96127E−74 | 3.47995E−72 | 0.306947228 | 2.137875557 |
| 0 | GOLGA4 | 18.47997665 | 1.387383699 | 3.56822E−74 | 2.51611E−72 | 0.494655979 | 2.304025173 |
| 0 | PKD2L2 | 18.46858215 | 1.828761101 | 7.32021E−74 | 5.12106E−72 | 0.303941216 | 2.163956165 |
| 0 | BIN1 | 18.43944168 | 1.66579628 | 9.49761E−74 | 6.62689E−72 | 0.359719439 | 2.253041983 |
| 0 | DIAPH1 | 18.37310982 | 1.666906834 | 3.08276E−73 | 2.14534E−71 | 0.348363393 | 2.171512604 |
| 0 | USP13 | 18.3168354 | 1.804784536 | 1.00721E−72 | 6.97284E−71 | 0.303273213 | 2.156624079 |
| 0 | FAM166B | 18.19791412 | 2.194077253 | 1.48813E−71 | 1.01437E−69 | 0.229124916 | 2.435400248 |
| 0 | PHKG1 | 18.18750954 | 1.74599421 | 8.62306E−72 | 5.90813E−70 | 0.318303273 | 2.210735798 |
| 0 | TXLNB | 18.17458153 | 2.020838499 | 1.56219E−71 | 1.06213E−69 | 0.251837007 | 2.094725847 |
| 0 | LMO7 | 18.17265511 | 1.90785408 | 1.45163E−71 | 9.92031E−70 | 0.27254509 | 2.15757823 |
| 0 | ADCY2 | 18.11171913 | 1.608430862 | 2.68149E−71 | 1.81386E−69 | 0.363393454 | 2.23874712 |
| 0 | MED13 | 18.10502625 | 1.411686778 | 2.50537E−71 | 1.69905E−69 | 0.457915832 | 2.284040451 |
| 0 | EXOC6 | 18.10081482 | 1.811980486 | 4.19928E−71 | 2.83334E−69 | 0.298263193 | 2.216151237 |
| 0 | SLC7A6OS | 18.07090569 | 1.396759033 | 4.33263E−71 | 2.91591E−69 | 0.482631931 | 2.397374153 |
| 0 | CMSS1 | 18.06182098 | 1.451063514 | 5.47726E−71 | 3.67695E−69 | 0.435871743 | 2.304710865 |
| 0 | IP6K3 | 18.05794716 | 1.850676894 | 9.2899E−71 | 6.22072E−69 | 0.283567134 | 2.151963472 |
| 0 | FAM222B | 18.04060364 | 1.684691429 | 9.93789E−71 | 6.63791E−69 | 0.331329325 | 2.197519541 |
| 0 | RP11-445F6.2 | 18.02353859 | 2.475305319 | 3.95794E−70 | 2.59797E−68 | 0.191048764 | 2.054253578 |
| 0 | FBXO34 | 18.01599312 | 1.646805048 | 1.462E−70 | 9.74077E−69 | 0.340681363 | 2.21872282 |
| 0 | RYR3 | 17.98215866 | 1.851519108 | 3.5198E−70 | 2.32762E−68 | 0.285237141 | 2.231397152 |
| 0 | EPB41L5 | 17.98149681 | 1.54133141 | 2.39805E−70 | 1.58977E−68 | 0.38009352 | 2.201472044 |
| 0 | FOXP2 | 17.95003319 | 1.520770431 | 3.91099E−70 | 2.57351E−68 | 0.393453574 | 2.269137621 |
| 0 | DIAPH2-AS1 | 17.94959831 | 1.420237422 | 3.64806E−70 | 2.40645E−68 | 0.474281897 | 2.577909231 |
| 0 | DLGAP4 | 17.913414 | 1.634487033 | 8.58227E−70 | 5.60568E−68 | 0.34001336 | 2.19217968 |
| 0 | MEF2C | 17.91206741 | 1.528007388 | 7.1452E−70 | 4.67852E−68 | 0.401135605 | 2.287614346 |
| 0 | BCL6 | 17.86764526 | 1.142379642 | 1.5829E−69 | 1.02136E−67 | 0.799599198 | 3.004930973 |
| 0 | NRAP | 17.84988976 | 1.752396822 | 2.7698E−69 | 1.77856E−67 | 0.304275217 | 2.17458415 |
| 0 | PARD3 | 17.83728981 | 1.132203817 | 2.29523E−69 | 1.4774E−67 | 0.749832999 | 2.774553776 |
| 0 | MIR133A1HG | 17.78588486 | 1.86967802 | 9.23011E−69 | 5.89839E−67 | 0.277221109 | 2.297811985 |
| 0 | RP11-729I10.2 | 17.77885437 | 2.68171072 | 3.10362E−68 | 1.95978E−66 | 0.170674683 | 2.018299103 |
| 0 | CABYR | 17.77394295 | 2.868606091 | 4.15887E−68 | 2.61989E−66 | 0.161656647 | 2.214394808 |
| 0 | CAMKMT | 17.74222183 | 1.427802444 | 1.23922E−68 | 7.90009E−67 | 0.432197729 | 2.30706048 |
| 0 | CAPN7 | 17.71537399 | 1.396913052 | 1.85208E−68 | 1.17789E−66 | 0.459585838 | 2.374102116 |
| 0 | KBTBD12 | 17.71243095 | 2.306232691 | 5.90859E−68 | 3.70457E−66 | 0.204742819 | 2.143142462 |
| 0 | INADL | 17.70799065 | 1.324082971 | 2.06442E−68 | 1.3098E−66 | 0.495991984 | 2.342360258 |
| 0 | AAK1 | 17.69239044 | 1.389843822 | 2.75744E−68 | 1.74533E−66 | 0.454575818 | 2.289227247 |
| 0 | CTC-431G16.2 | 17.64535713 | 1.535451174 | 6.47254E−68 | 4.02965E−66 | 0.386773547 | 2.313188076 |
| 0 | PRUNE2 | 17.62888908 | 2.077908278 | 2.03222E−67 | 1.25932E−65 | 0.235136941 | 2.594388962 |
| 0 | NRDC | 17.61159325 | 1.264985681 | 1.02737E−67 | 6.38123E−66 | 0.539746159 | 2.413637161 |
| 0 | RP11-118M9.3 | 17.59334564 | 1.72823298 | 2.07306E−67 | 1.28163E−65 | 0.303273213 | 2.212949276 |
| 0 | RP11-428F8.2 | 17.58959007 | 1.802187085 | 2.32099E−67 | 1.43158E−65 | 0.283233133 | 2.173067808 |
| 0 | TRIM54 | 17.57185555 | 1.942181706 | 3.92841E−67 | 2.41743E−65 | 0.249164997 | 2.094444752 |
| 0 | SPATC1 | 17.54839706 | 2.21936202 | 7.69166E−67 | 4.71141E−65 | 0.209752839 | 2.179903269 |
| 0 | RIC1 | 17.48196983 | 1.41043663 | 9.7737E−67 | 5.97297E−65 | 0.429191717 | 2.322148085 |
| 0 | BNC2 | 17.47071648 | 1.248147964 | 1.0665E−66 | 6.50271E−65 | 0.58750835 | 2.638213396 |
| 0 | RP11-131H24.4 | 17.44713211 | 2.248373508 | 4.1677E−66 | 2.52379E−64 | 0.202070808 | 1.995686531 |
| 0 | OVCH2 | 17.37011719 | 3.415088892 | 5.00763E−65 | 2.97814E−63 | 0.13259853 | 1.974610925 |
| 0 | LMOD2 | 17.35362434 | 2.163807392 | 2.00884E−65 | 1.20548E−63 | 0.214762859 | 2.179991484 |
| 0 | DCAF6 | 17.33315086 | 1.383896232 | 1.13929E−65 | 6.85221E−64 | 0.435203741 | 2.280396938 |
| 0 | FGF13 | 17.27845383 | 1.576977134 | 3.3711E−65 | 2.01387E−63 | 0.346025384 | 2.322670221 |
| 0 | PSME4 | 17.2500515 | 1.458651423 | 4.79111E−65 | 2.85576E−63 | 0.389111556 | 2.238654137 |
| 0 | MYH14 | 17.24996758 | 2.112912416 | 9.65124E−65 | 5.70152E−63 | 0.213426854 | 2.076520681 |
| 0 | NPAS2 | 17.23174477 | 1.461622357 | 6.28549E−65 | 3.72976E−63 | 0.401135605 | 2.320052147 |
| 0 | RP11-227G15.12 | 17.23032951 | 1.756733537 | 8.8655E−65 | 5.24901E−63 | 0.279225117 | 2.137662649 |
| 0 | TMEM38A | 17.22249985 | 1.822098494 | 1.08298E−64 | 6.38359E−63 | 0.264195057 | 2.143599033 |
| 0 | TMCC1 | 17.19190598 | 1.368082166 | 1.15873E−64 | 6.79991E−63 | 0.443887776 | 2.323856831 |
| 0 | FAM168A | 17.17523384 | 1.512274981 | 1.7223E−64 | 1.0085E−62 | 0.377755511 | 2.41447258 |
| 0 | CTA-125H2.1 | 17.12713814 | 1.775598764 | 4.6952E−64 | 2.73722E−62 | 0.2748831 | 2.149944067 |
| 0 | ANKRD23 | 17.11882973 | 1.867393613 | 6.12249E−64 | 3.55372E−62 | 0.251169005 | 2.107456207 |
| 0 | ARHGEF26 | 17.09096718 | 1.56953764 | 7.04449E−64 | 4.07997E−62 | 0.337007348 | 2.204336405 |
| 0 | USP34 | 17.06003761 | 1.159852386 | 9.27987E−64 | 5.36296E−62 | 0.602872411 | 2.455590487 |
| 0 | RARRES1 | 17.05660439 | 1.608361959 | 1.30875E−63 | 7.53067E−62 | 0.321309285 | 2.189212561 |
| 0 | ANO5 | 17.03972435 | 1.863619924 | 2.18715E−63 | 1.25309E−61 | 0.250835003 | 2.09836936 |
| 0 | HOOK2 | 17.03933907 | 1.694941401 | 1.90759E−63 | 1.09528E−61 | 0.287909152 | 2.137226343 |
| 0 | IL6R | 17.03328133 | 1.736762881 | 2.20109E−63 | 1.25836E−61 | 0.27755511 | 2.116989851 |
| 0 | PIGF | 17.01307106 | 1.757862806 | 3.06754E−63 | 1.7462E−61 | 0.274549098 | 2.147962809 |
| 0 | KMT2C | 17.00210762 | 1.189289808 | 2.3968E−63 | 1.36731E−61 | 0.567802271 | 2.392055035 |
| 0 | RP11-21A7A.2 | 16.98153496 | 1.61068356 | 4.40258E−63 | 2.50081E−61 | 0.318637275 | 2.215461493 |
| 0 | PTPN14 | 16.96060562 | 1.532901525 | 5.78201E−63 | 3.27738E−61 | 0.347361389 | 2.226799965 |
| 0 | ELMOD3 | 16.94576645 | 1.631712198 | 8.29404E−63 | 4.67137E−61 | 0.303607214 | 2.176994562 |
| 0 | SLC8A3 | 16.93204308 | 1.765680075 | 1.1064E−62 | 6.21829E−61 | 0.271877088 | 2.151591301 |
| 0 | PADI2 | 16.90969658 | 2.418468237 | 3.36323E−62 | 1.86656E−60 | 0.173012692 | 2.080703735 |
| 0 | CTD-3252C9.4 | 16.90602493 | 2.086694956 | 2.40014E−62 | 1.34045E−60 | 0.209752839 | 2.043565989 |
| 0 | UBR5 | 16.89287186 | 1.305007577 | 1.49068E−62 | 8.34277E−61 | 0.458583834 | 2.26210928 |
| 0 | ATP1B1 | 16.87716484 | 1.645839095 | 2.4364E−62 | 1.35785E−60 | 0.304609218 | 2.195813894 |
| 0 | ITGAD | 16.8649559 | 1.847545147 | 3.53701E−62 | 1.95892E−60 | 0.249832999 | 2.095555782 |
| 0 | CYB5R1 | 16.86373329 | 2.031980753 | 4.46534E−62 | 2.46791E−60 | 0.216098864 | 2.043249369 |
| 0 | MLXIP | 16.86363029 | 1.506059527 | 2.76514E−62 | 1.53784E−60 | 0.347695391 | 2.183516502 |
| 0 | ACSM6 | 16.8403759 | 2.526212931 | 1.10937E−61 | 6.05574E−60 | 0.164662659 | 2.154239893 |
| 0 | STAC3 | 16.8305912 | 1.644484162 | 5.15763E−62 | 2.84462E−60 | 0.296259185 | 2.136692762 |
| 0 | FGGY | 16.82395554 | 1.782392383 | 6.8289E−62 | 3.75082E−60 | 0.262191049 | 2.177463293 |
| 0 | KIAA0368 | 16.78542137 | 1.372264504 | 8.83222E−62 | 4.84115E−60 | 0.411489646 | 2.253783464 |
| 0 | GLRA1 | 16.74773407 | 1.863850236 | 2.38087E−61 | 1.28907E−59 | 0.248496994 | 2.182640314 |
| 0 | PPP3CC | 16.71278191 | 1.328620672 | 2.70343E−61 | 1.46073E−59 | 0.43754175 | 2.289690971 |
| 0 | RP11-577H5.5 | 16.7032032 | 1.624282956 | 4.06863E−61 | 2.18948E−59 | 0.29759519 | 2.154344082 |
| 0 | CNST | 16.68095016 | 1.58660388 | 5.40952E−61 | 2.89349E−59 | 0.312625251 | 2.167744398 |
| 0 | SCAPER | 16.67474556 | 1.322697163 | 5.015E−61 | 2.69331E−59 | 0.436205745 | 2.305060148 |
| 0 | C14orf159 | 16.67274666 | 1.684675932 | 6.72218E−61 | 3.5884E−59 | 0.281229125 | 2.12819171 |
| 0 | CADM1 | 16.67023277 | 2.226395845 | 1.30638E−60 | 6.94577E−59 | 0.19004676 | 2.376095295 |
| 0 | RP11-82O19.1 | 16.63946533 | 1.792278051 | 1.31152E−60 | 6.95914E−59 | 0.255177021 | 2.18050909 |
| 0 | FAF1 | 16.58125114 | 1.240660787 | 2.14496E−60 | 1.12691E−58 | 0.491315965 | 2.351870775 |
| 0 | EFHC2 | 16.5811615 | 2.542708397 | 6.43316E−60 | 3.33371E−58 | 0.160320641 | 2.049661398 |
| 0 | ARHGAP11B | 16.56342506 | 1.842865586 | 4.55203E−60 | 2.36812E−58 | 0.240814963 | 2.178416967 |
| 0 | QKI | 16.54225731 | 1.163039207 | 3.87993E−60 | 2.03039E−58 | 0.573814295 | 2.462432384 |
| 0 | RP5-1096J16.1 | 16.5232048 | 2.908212185 | 2.40136E−59 | 1.22765E−57 | 0.136606546 | 2.144954681 |
| 0 | AVIL | 16.47790527 | 1.767310381 | 1.62066E−59 | 8.3334E−58 | 0.252839011 | 2.066231489 |
| 0 | PHF20 | 16.47452927 | 1.273760557 | 1.17137E−59 | 6.03482E−58 | 0.465263861 | 2.325999975 |
| 0 | MIR181A2HG | 16.46302414 | 2.12493968 | 2.94581E−59 | 1.50022E−57 | 0.195724783 | 2.123491526 |
| 0 | SNAP23 | 16.42890358 | 1.362793684 | 2.59605E−59 | 1.32463E−57 | 0.390781563 | 2.254184008 |
| 0 | GMPR | 16.36610794 | 1.781185865 | 9.70359E−59 | 4.89488E−57 | 0.245490982 | 2.096915483 |
| 0 | RP11-111E14.1 | 16.35162354 | 1.765524864 | 1.1306E−58 | 5.69239E−57 | 0.253841015 | 2.164790154 |
| 0 | FSD2 | 16.34236908 | 1.634960175 | 1.19309E−58 | 5.99567E−57 | 0.285237141 | 2.189876318 |
| 0 | PGM1 | 16.3094635 | 1.581648588 | 1.96668E−58 | 9.80904E−57 | 0.300601202 | 2.216086864 |
| 0 | HS6ST3 | 16.30773163 | 2.275685787 | 3.72322E−58 | 1.85006E−56 | 0.180694723 | 2.256123066 |
| 0 | AP000462.2 | 16.28178406 | 2.981984854 | 1.07717E−57 | 5.27359E−56 | 0.131596526 | 2.405679703 |
| 0 | R3HDM2 | 16.23307228 | 1.4524858 | 5.87952E−58 | 2.91064E−56 | 0.346025384 | 2.213901281 |
| 0 | LDB3 | 16.21657181 | 1.41775763 | 7.24361E−58 | 3.57926E−56 | 0.374749499 | 2.355337381 |
| 0 | AMPD3 | 16.20637703 | 2.154657602 | 1.61752E−57 | 7.88993E−56 | 0.183032732 | 2.046602964 |
| 0 | ZRANB3 | 16.19891357 | 1.41544044 | 9.82459E−58 | 4.82767E−56 | 0.354709419 | 2.185663223 |
| 0 | MLK7-AS1 | 16.19711113 | 1.339882255 | 9.36242E−58 | 4.60909E−56 | 0.408483634 | 2.291814327 |
| 0 | RP11-166O4.6 | 16.17972755 | 1.977491498 | 2.00358E−57 | 9.73731E−56 | 0.208416834 | 2.013403893 |
| 0 | PPP6R3 | 16.17147064 | 1.12992394 | 1.31624E−57 | 6.43214E−56 | 0.569806279 | 2.420997858 |
| 0 | PRKAG2 | 16.16262054 | 1.466998219 | 1.91686E−57 | 9.33292E−56 | 0.362725451 | 2.510795832 |
| 0 | MLXIPL | 16.11414719 | 1.971257806 | 5.39318E−57 | 2.60677E−55 | 0.21008684 | 2.162671804 |
| 0 | TMEM140 | 16.10300827 | 1.848827958 | 6.16403E−57 | 2.97395E−55 | 0.224448898 | 2.091304779 |
| 0 | SPG11 | 16.07538986 | 1.291307569 | 6.20814E−57 | 2.9898E−55 | 0.425851703 | 2.304808617 |
| 0 | MYO9A | 16.04323196 | 1.201533437 | 9.79885E−57 | 4.71054E−55 | 0.491649967 | 2.335290432 |
| 0 | NT5C2 | 16.03634453 | 1.324223876 | 1.15051E−56 | 5.52076E−55 | 0.399131597 | 2.239352465 |
| 0 | SEC13 | 16.01502037 | 1.500368237 | 1.75676E−56 | 8.41471E−55 | 0.315631263 | 2.131021023 |
| 0 | UNC13B | 16.0100441 | 1.739563227 | 2.23828E−56 | 1.06826E−54 | 0.247160989 | 2.060148954 |
| 0 | RP11-451G4.2 | 16.00411987 | 1.406407237 | 1.87105E−56 | 8.94606E−55 | 0.378089512 | 2.276890516 |
| 0 | G3BP2 | 15.97274208 | 1.647759914 | 3.8036E−56 | 1.80241E−54 | 0.265865063 | 2.127159357 |
| 0 | ABCC1 | 15.97153759 | 1.467082381 | 3.42493E−56 | 1.62586E−54 | 0.328323313 | 2.204559088 |
| 0 | ADCK3 | 15.94039917 | 1.430644751 | 5.29657E−56 | 2.50096E−54 | 0.341349365 | 2.1717031 |
| 0 | PARK2 | 15.93598843 | 1.116294146 | 5.00471E−56 | 2.36735E−54 | 0.572812291 | 2.490611553 |
| 0 | USP32 | 15.92546844 | 1.338526607 | 6.42353E−56 | 3.01702E−54 | 0.382765531 | 2.229199648 |
| 0 | ANK1 | 15.8854084 | 1.859022737 | 1.7195E−55 | 7.99149E−54 | 0.219104876 | 2.10004878 |
| 0 | ASH1L | 15.87381268 | 1.118839622 | 1.29937E−55 | 6.09214E−54 | 0.554442218 | 2.403971434 |
| 0 | HSPB7 | 15.86577225 | 1.390439034 | 1.60725E−55 | 7.48287E−54 | 0.365731463 | 2.281533957 |
| 0 | ANKRD17 | 15.86484909 | 1.225801706 | 1.54166E−55 | 7.19009E−54 | 0.456579826 | 2.32550931 |
| 0 | DCST2 | 15.83076286 | 2.384362221 | 5.92959E−55 | 2.7272E−53 | 0.156646627 | 2.019798756 |
| 0 | CSPP1 | 15.82659054 | 1.327772617 | 2.84643E−55 | 1.31599E−53 | 0.392785571 | 2.288116217 |
| 0 | CNNM2 | 15.81238651 | 1.45409596 | 3.91235E−55 | 1.80566E−53 | 0.326319305 | 2.246425629 |
| 0 | TTC7B | 15.80394363 | 1.597295403 | 4.86111E−55 | 2.23965E−53 | 0.273547094 | 2.135457516 |
| 0 | ACSF2 | 15.79014301 | 2.071959734 | 8.93298E−55 | 4.10146E−53 | 0.185704743 | 2.146255732 |
| 0 | NR6A1 | 15.76781845 | 1.686571479 | 8.98561E−55 | 4.11851E−53 | 0.254509018 | 2.199061394 |
| 0 | LINC00693 | 15.75965977 | 2.700784683 | 2.26417E−54 | 1.02714E−52 | 0.13493654 | 2.162194967 |
| 0 | UBE2G1 | 15.74550629 | 1.334120035 | 9.87534E−55 | 4.51852E−53 | 0.378089512 | 2.199925184 |
| 0 | PHTF2 | 15.71616459 | 1.494258761 | 1.70058E−54 | 7.76774E−53 | 0.305945224 | 2.184441566 |
| 0 | OGDH | 15.70246124 | 1.535066843 | 2.15303E−54 | 9.8007E−53 | 0.290581162 | 2.136951923 |
| 0 | CEP85L | 15.69300175 | 1.327121139 | 2.21151E−54 | 1.00497E−52 | 0.377755511 | 2.246088505 |
| 0 | RAB7A | 15.69249153 | 1.228598237 | 2.14857E−54 | 9.79717E−53 | 0.445223781 | 2.324440956 |
| 0 | OIP5 | 15.67027187 | 1.84188962 | 4.35672E−54 | 1.97307E−52 | 0.216432866 | 2.083689928 |
| 0 | MCU | 15.6508646 | 1.596221447 | 4.95254E−54 | 2.23529E−52 | 0.270541082 | 2.168714285 |
| 0 | NKAIN2 | 15.64875507 | 1.479581475 | 4.65055E−54 | 2.10256E−52 | 0.313293253 | 2.226980448 |
| 0 | SPAG1 | 15.64183235 | 1.566264749 | 5.60421E−54 | 2.52085E−52 | 0.279559118 | 2.226573467 |
| 0 | RGS22 | 15.64061165 | 1.766048312 | 6.43492E−54 | 2.8799E−52 | 0.229124916 | 2.071196318 |
| 0 | NOCT | 15.62962818 | 1.501644611 | 6.23214E−54 | 2.79857E−52 | 0.299265197 | 2.15253377 |
| 0 | DHRSX | 15.62945366 | 1.25400722 | 5.54694E−54 | 2.49932E−52 | 0.424515698 | 2.30031991 |
| 0 | MITF | 15.62094021 | 1.34111166 | 6.662E−54 | 2.97652E−52 | 0.371075484 | 2.278107643 |
| 0 | CES4A | 15.59087944 | 2.654187918 | 2.66079E−53 | 1.17891E−51 | 0.136272545 | 1.950292587 |
| 0 | ADGRA3 | 15.55497646 | 1.90304172 | 2.61979E−53 | 1.16268E−51 | 0.202070808 | 2.037593603 |
| 0 | ASB14 | 15.51764297 | 1.683738589 | 3.85201E−53 | 1.69539E−51 | 0.242818971 | 2.106280327 |
| 0 | NME9 | 15.51403332 | 1.587452531 | 3.77137E−53 | 1.66542E−51 | 0.269539078 | 2.154132128 |
| 0 | COBL | 15.49211979 | 1.721969604 | 5.59635E−53 | 2.45501E−51 | 0.236806947 | 2.110159397 |
| 0 | UBR3 | 15.44822598 | 1.161454558 | 8.19749E−53 | 3.57836E−51 | 0.47762191 | 2.336247921 |
| 0 | TNRC6B | 15.40819645 | 0.990381718 | 1.46588E−52 | 6.36746E−51 | 0.669004676 | 2.55092907 |
| 0 | CDKL4 | 15.39843559 | 1.868908286 | 2.56222E−52 | 1.10575E−50 | 0.204408818 | 2.064908504 |
| 0 | ZCCHC11 | 15.38135147 | 1.168267012 | 2.19353E−52 | 9.48178E−51 | 0.485303941 | 2.373306513 |
| 0 | CORO1C | 15.37974644 | 1.430346489 | 2.56955E−52 | 1.10712E−50 | 0.312291249 | 2.162448883 |
| 0 | CHRNA1 | 15.3513546 | 2.209694862 | 6.45163E−52 | 2.7574E−50 | 0.161990648 | 2.142338037 |
| 0 | RP11-724O16.1 | 15.34296036 | 1.803437114 | 5.53722E−52 | 2.37039E−50 | 0.214762859 | 2.102857113 |
| 0 | TSC22D1 | 15.32854366 | 1.238604426 | 4.92669E−52 | 2.11585E−50 | 0.422845691 | 2.312669992 |
| 0 | OPTN | 15.326437 | 1.360368967 | 5.44848E−52 | 2.33617E−50 | 0.341683367 | 2.205104351 |
| 0 | FXR1 | 15.27266312 | 1.450301051 | 1.26108E−51 | 5.37254E−50 | 0.299265197 | 2.073183537 |
| 0 | SLC16A1-AS1 | 15.24307632 | 1.482134342 | 1.99289E−51 | 8.46308E−50 | 0.288243153 | 2.151204109 |
| 0 | OSBPL11 | 15.21963692 | 1.701134801 | 3.2339E−51 | 1.3646E−49 | 0.230460922 | 2.11904335 |
| 0 | PTPRQ | 15.13894558 | 2.290489912 | 1.54596E−50 | 6.45177E−49 | 0.151636607 | 2.039720535 |
| 0 | CADM2 | 15.13889885 | 1.711046696 | 9.93205E−51 | 4.16457E−49 | 0.236138945 | 2.186518431 |
| 0 | SMAD3 | 15.09103107 | 1.351332545 | 1.71336E−50 | 7.13918E−49 | 0.334335337 | 2.152162552 |
| 0 | C20orfl96 | 15.06806946 | 1.860280156 | 3.22277E−50 | 1.33657E−48 | 0.19739479 | 2.063243151 |
| 0 | GALNT2 | 15.0591259 | 1.518690109 | 2.98178E−50 | 1.23856E−48 | 0.270875084 | 2.057989836 |
| 0 | HPN | 14.9997921 | 2.397597313 | 1.23741E−49 | 5.06861E−48 | 0.139612558 | 2.048086166 |
| 0 | SLC52A1 | 14.99556255 | 2.136133432 | 1.10244E−49 | 4.52274E−48 | 0.160320641 | 2.043700218 |
| 0 | EIF4ENIF1 | 14.98901653 | 1.551285505 | 8.42765E−50 | 3.46276E−48 | 0.260521042 | 2.166300774 |
| 0 | VGLL4 | 14.93440819 | 1.226816416 | 1.57558E−49 | 6.44388E−48 | 0.404475618 | 2.288664579 |
| 0 | ESR2 | 14.92832184 | 1.23162055 | 1.72143E−49 | 7.02956E−48 | 0.401469606 | 2.289363384 |
| 0 | AC013461.1 | 14.91084576 | 1.099633455 | 2.14888E−49 | 8.76163E−48 | 0.508350033 | 2.399133205 |
| 0 | DNHD1 | 14.89383793 | 1.665021896 | 3.60309E−49 | 1.46684E−47 | 0.228122912 | 2.130773067 |
| 0 | KIF9-AS1 | 14.82173538 | 1.570397854 | 9.48303E−49 | 3.83126E−47 | 0.24749499 | 2.129175186 |
| 0 | BCL2L13 | 14.82042885 | 1.418829203 | 9.02954E−49 | 3.6536E−47 | 0.294589178 | 2.173407316 |
| 0 | TFEB | 14.8154068 | 1.922819376 | 1.30401E−48 | 5.24445E−47 | 0.181362725 | 2.057920933 |
| 0 | ABCC6 | 14.73395634 | 2.130108118 | 4.65364E−48 | 1.85476E−46 | 0.156980628 | 2.0291605 |
| 0 | RBM24 | 14.73011684 | 1.749259353 | 3.8294E−48 | 1.53084E−46 | 0.206746827 | 2.032394886 |
| 0 | MKNK1 | 14.72910881 | 1.516642451 | 3.44338E−48 | 1.3786E−46 | 0.260521042 | 2.109861135 |
| 0 | ADAMTS17 | 14.71248341 | 1.323694706 | 4.01877E−48 | 1.60413E−46 | 0.339345357 | 2.30975461 |
| 0 | RC3H1 | 14.66734886 | 1.217507601 | 7.27693E−48 | 2.89163E−46 | 0.388443554 | 2.234576225 |
| 0 | TDRD3 | 14.65273952 | 1.39492178 | 9.73533E−48 | 3.85699E−46 | 0.298597194 | 2.1575315 |
| 0 | ZC2HC1C | 14.65106487 | 1.948270321 | 1.32676E−47 | 5.24082E−46 | 0.173680695 | 1.997741818 |
| 0 | SRSF12 | 14.63607597 | 1.594225168 | 1.34838E−47 | 5.31832E−46 | 0.237140949 | 2.122017384 |
| 0 | PPTC7 | 14.62787342 | 1.560946345 | 1.51614E−47 | 5.9711E−46 | 0.241482966 | 2.094564199 |
| 0 | DOCK3 | 14.61984825 | 1.446493506 | 1.58067E−47 | 6.21607E−46 | 0.278557114 | 2.167338133 |
| 0 | SLC15A4 | 14.61054516 | 1.918712378 | 2.35425E−47 | 9.20372E−46 | 0.177020708 | 2.029417038 |
| 0 | DTNA | 14.59143734 | 1.333076239 | 2.22971E−47 | 8.74258E−46 | 0.332999332 | 2.298611879 |
| 0 | TMEM38B | 14.57117271 | 1.810342193 | 3.83774E−47 | 1.49156E−45 | 0.191382766 | 2.035772562 |
| 0 | RPTOR | 14.54787445 | 1.349876404 | 4.1315E−47 | 1.60338E−45 | 0.312291249 | 2.117896557 |
| 0 | DIAPH2 | 14.52535534 | 0.945949912 | 5.24738E−47 | 2.03347E−45 | 0.692050768 | 2.78290534 |
| 0 | ME1 | 14.50990295 | 1.577570677 | 7.99499E−47 | 3.08923E−45 | 0.238476954 | 2.180466413 |
| 0 | ANKUB1 | 14.47717381 | 1.151030898 | 1.05369E−46 | 4.06552E−45 | 0.428189713 | 2.312220335 |
| 0 | ST3GAL3 | 14.47644997 | 1.339417338 | 1.15138E−46 | 4.43597E−45 | 0.310287241 | 2.162707567 |
| 0 | RUVBL1 | 14.42088413 | 1.600281477 | 2.84577E−46 | 1.08695E−44 | 0.225450902 | 2.073932648 |
| 0 | YBX3 | 14.40989017 | 0.988221109 | 2.67078E−46 | 1.02306E−44 | 0.614228457 | 2.492963791 |
| 0 | CAMK2D | 14.40826416 | 1.154650927 | 2.77656E−46 | 1.06204E−44 | 0.421175685 | 2.276432276 |
| 0 | ARPP19 | 14.39317131 | 1.358853459 | 3.74742E−46 | 1.42724E−44 | 0.29759519 | 2.134979486 |
| 0 | EFTUD2 | 14.38139629 | 1.40644896 | 4.37418E−46 | 1.66119E−44 | 0.284235137 | 2.130709887 |
| 0 | ZNF397 | 14.37499714 | 1.505043149 | 5.0856E−46 | 1.92861E−44 | 0.248496994 | 2.069564342 |
| 0 | RP11-545I5.3 | 14.36187077 | 1.578297377 | 6.3134E−46 | 2.38064E−44 | 0.228790915 | 2.082016468 |
| 0 | JARID2 | 14.35937786 | 1.382490635 | 6.02284E−46 | 2.27754E−44 | 0.29258517 | 2.22538805 |
| 0 | SDC4 | 14.33653927 | 2.123505116 | 1.20929E−45 | 4.54705E−44 | 0.149298597 | 2.046596766 |
| 0 | BHLHE40 | 14.33464813 | 2.023164749 | 1.18433E−45 | 4.45951E−44 | 0.159318637 | 2.036904097 |
| 0 | TEX2 | 14.31691647 | 1.734387279 | 1.31015E−45 | 4.91243E−44 | 0.196058784 | 2.15071702 |
| 0 | SLC28A1 | 14.30354404 | 2.483810425 | 2.23994E−45 | 8.31657E−44 | 0.123580494 | 2.008016825 |
| 0 | FEZ2 | 14.28799725 | 1.158370256 | 1.49928E−45 | 5.60575E−44 | 0.443219773 | 2.586641073 |
| 0 | GPR75-ASB3 | 14.23690224 | 1.253795505 | 3.11282E−45 | 1.15413E−43 | 0.34502338 | 2.216575623 |
| 0 | HIPK4 | 14.22350025 | 2.236025095 | 6.02196E−45 | 2.22036E−43 | 0.138610554 | 2.00927186 |
| 0 | RETSAT | 14.20501804 | 2.187057257 | 7.78891E−45 | 2.85995E−43 | 0.139278557 | 2.076481342 |
| 0 | MAMSTR | 14.18348694 | 2.654453039 | 1.26194E−44 | 4.60816E−43 | 0.113560454 | 2.098654509 |
| 0 | UBE2R2 | 14.17832947 | 1.268070936 | 7.12357E−45 | 2.62289E−43 | 0.328657315 | 2.187458515 |
| 0 | C4orf51 | 14.17056656 | 2.488343239 | 1.41372E−44 | 5.15534E−43 | 0.120908484 | 2.020274878 |
| 0 | NNMT | 14.16877174 | 1.014879584 | 7.54186E−45 | 2.77307E−43 | 0.550768203 | 2.424884796 |
| 0 | RP11-23N2.4 | 14.16810703 | 1.905688524 | 1.09988E−44 | 4.03299E−43 | 0.167000668 | 2.02091074 |
| 0 | ZNF749 | 14.14581776 | 2.321432352 | 1.7887E−44 | 6.5049E−43 | 0.131262525 | 2.025863647 |
| 0 | PPM1B | 14.13933563 | 1.001115561 | 1.1309E−44 | 4.14101E−43 | 0.560454242 | 2.460917234 |
| 0 | RP11-99J16_A.2 | 14.12081242 | 2.281021118 | 2.49824E−44 | 9.06046E−43 | 0.133266533 | 1.955157757 |
| 0 | TK2 | 14.06367302 | 1.416759849 | 3.52339E−44 | 1.27263E−42 | 0.26753507 | 2.14439559 |
| 0 | MPRIP | 14.04258537 | 1.221596241 | 4.49969E−44 | 1.62306E−42 | 0.346693387 | 2.201637745 |
| 0 | LINC-PINT | 14.00065994 | 0.861318767 | 7.83305E−44 | 2.81017E−42 | 0.822311289 | 3.075013638 |
| 0 | GBF1 | 13.99345303 | 1.264253974 | 8.85481E−44 | 3.17246E−42 | 0.322979292 | 2.156799793 |
| 0 | YY1AP1 | 13.98432064 | 1.468883157 | 1.09665E−43 | 3.91846E−42 | 0.246492986 | 2.151560783 |
| 0 | PDCD1LG2 | 13.98289967 | 1.702980518 | 1.23365E−43 | 4.39614E−42 | 0.19505678 | 2.130951405 |
| 0 | CRADD | 13.97923183 | 1.289109945 | 1.09216E−43 | 3.90766E−42 | 0.315297261 | 2.266654015 |
| 0 | ACSS3 | 13.96877766 | 1.431563616 | 1.32147E−43 | 4.69653E−42 | 0.258183033 | 2.163902998 |
| 0 | CRYM | 13.95514202 | 1.552756667 | 1.69377E−43 | 6.00363E−42 | 0.220774883 | 2.082353115 |
| 0 | ANK3 | 13.95062065 | 1.65453589 | 1.84491E−43 | 6.53064E−42 | 0.203740815 | 2.092957973 |
| 0 | USO1 | 13.93419838 | 1.211125612 | 1.95442E−43 | 6.90909E−42 | 0.346693387 | 2.160791874 |
| 0 | DGCR5 | 13.905303 | 2.345234871 | 4.70931E−43 | 1.65161E−41 | 0.125250501 | 2.102458477 |
| 0 | PLCD4 | 13.88477993 | 1.549333572 | 4.27034E−43 | 1.49964E−41 | 0.221776887 | 2.07218194 |
| 0 | TYW1 | 13.8692112 | 1.250993729 | 4.75508E−43 | 1.66547E−41 | 0.322979292 | 2.193983078 |
| 0 | NRG4 | 13.86677742 | 1.704890013 | 5.86005E−43 | 2.04978E−41 | 0.191716767 | 2.091192722 |
| 0 | MRPL19 | 13.84613705 | 1.506358743 | 7.20149E−43 | 2.50254E−41 | 0.228790915 | 2.067745447 |
| 0 | SETD5 | 13.84224606 | 1.053582788 | 6.51929E−43 | 2.27439E−41 | 0.458583834 | 2.306885719 |
| 0 | SYPL2 | 13.82175732 | 1.944892883 | 1.20326E−42 | 4.17045E−41 | 0.157648631 | 2.036775827 |
| 0 | P4HA3 | 13.81500721 | 1.217283964 | 9.71929E−43 | 3.37308E−41 | 0.342017368 | 2.197556734 |

Cluster 2 Myocyte

| | | | | | | | |
|---|---|---|---|---|---|---|---|
| 2 | RP11-434D9.1 | 34.18685913 | 3.524354696 | 6.7564E−215 | 1.7961E−210 | 0.811977716 | 3.928262711 |
| 2 | ART3 | 21.7507 | 2.696170092 | 1.7715E−95 | 7.84895E−92 | 0.472841226 | 2.889046669 |
| 2 | EMC10 | 21.7207737 | 2.569389343 | 7.95691E−97 | 4.23053E−93 | 0.69637883 | 3.991534233 |
| 2 | SVIL | 21.31579971 | 2.060155392 | 1.10254E−93 | 4.18712E−90 | 0.809192201 | 3.628729582 |
| 2 | NEXN | 21.22955704 | 2.068335056 | 5.19007E−93 | 1.72466E−89 | 0.745821727 | 3.354166746 |
| 2 | BCL6 | 20.78582001 | 1.906235695 | 1.70056E−89 | 3.76731E−86 | 0.806406685 | 3.649134159 |
| 2 | DMD | 20.77521324 | 1.946898222 | 2.69809E−89 | 5.51738E−86 | 0.871169916 | 4.09276104 |
| 2 | MYO18B | 20.5253582 | 2.198705435 | 2.87951E−87 | 5.46777E−84 | 0.648328691 | 3.293266296 |
| 2 | SESN1 | 19.87823105 | 1.92171669 | 2.08221E−82 | 3.25609E−79 | 0.701253482 | 3.100252628 |
| 2 | TTN | 19.83843231 | 1.896570325 | 4.73068E−82 | 6.98669E−79 | 0.83913649 | 3.764218092 |
| 2 | FAM184B | 19.83706284 | 2.039902449 | 5.23278E−82 | 7.32148E−79 | 0.648328691 | 3.116155624 |
| 2 | PDLIM5 | 19.75538635 | 1.89356339 | 1.50553E−81 | 1.90586E−78 | 0.789693593 | 3.499189854 |
| 2 | TNNT3 | 19.58838081 | 2.667191505 | 3.80464E−79 | 4.59738E−76 | 0.414345404 | 2.887404203 |
| 2 | WDPCP | 18.83184624 | 2.063080788 | 1.82092E−74 | 1.9363E−71 | 0.651114206 | 3.570447445 |
| 2 | RP11-362A9.3 | 18.64327812 | 1.986178637 | 2.59719E−73 | 2.38082E−70 | 0.71448468 | 3.4657166 |
| 2 | MYPN | 18.36316681 | 1.861833811 | 2.97022E−71 | 2.39273E−68 | 0.662952646 | 3.048729658 |
| 2 | PDLIM3 | 18.34846306 | 2.008563995 | 3.42434E−71 | 2.67743E−68 | 0.716573816 | 3.514009476 |
| 2 | GGT7 | 18.28709602 | 2.846465826 | 2.81957E−69 | 2.0821E−66 | 0.325208914 | 2.773211002 |
| 2 | TTN-AS1 | 17.81352043 | 1.748502135 | 2.14045E−67 | 1.45902E−64 | 0.698467967 | 3.13037467 |
| 2 | CTC-431G16.2 | 17.70883942 | 2.129842758 | 3.6818E−66 | 2.44692E−63 | 0.449860724 | 2.748227596 |
| 2 | NEDD4 | 17.47480965 | 1.879635334 | 6.27205E−65 | 3.96991E−62 | 0.580083565 | 2.969739676 |
| 2 | FAM134B | 17.45943451 | 1.92587018 | 9.21678E−65 | 5.69811E−62 | 0.54735376 | 2.934064865 |
| 2 | SLC7A2 | 17.28511047 | 2.197463751 | 3.90106E−63 | 2.35695E−60 | 0.400417827 | 2.683796883 |
| 2 | GRIK1 | 16.93069649 | 1.869630218 | 3.41929E−61 | 1.85507E−58 | 0.543175487 | 2.953582764 |
| 2 | BACH1 | 16.83221054 | 1.706934094 | 1.21251E−60 | 6.44666E−58 | 0.633008357 | 2.995364428 |
| 2 | NEAT1 | 16.54011536 | 1.171345472 | 9.76857E−59 | 4.99399E−56 | 0.959610028 | 5.033568382 |
| 2 | ATP2A1 | 16.27890396 | 2.425128937 | 2.52279E−56 | 1.15631E−53 | 0.313370474 | 2.678897142 |
| 2 | SORBS1 | 16.17632484 | 1.719982743 | 2.51391E−56 | 1.15631E−53 | 0.591225627 | 3.012720823 |
| 2 | CMYA5 | 16.04206276 | 1.676920295 | 1.92691E−55 | 8.39754E−53 | 0.585654596 | 2.949384212 |
| 2 | CD70 | 15.60215473 | 2.145250082 | 2.69725E−52 | 1.10313E−49 | 0.344707521 | 2.608177423 |
| 2 | ERAP1 | 15.57607079 | 1.755190849 | 2.06977E−52 | 8.59729E−50 | 0.505571031 | 2.944894791 |
| 2 | MYOM1 | 15.45697594 | 1.706034899 | 9.32127E−52 | 3.75449E−49 | 0.580083565 | 3.210166693 |
| 2 | TRDN | 15.34251499 | 1.602119565 | 4.05692E−51 | 1.60969E−48 | 0.646935933 | 3.072387218 |
| 2 | C2orf88 | 15.15385342 | 2.089624405 | 1.59235E−49 | 5.79878E−47 | 0.337047354 | 2.723729134 |
| 2 | SH2D1B | 14.98241997 | 3.444938421 | 8.76786E−48 | 3.0669E−45 | 0.185933148 | 2.473004818 |
| 2 | AHCYL1 | 14.83102036 | 1.929876089 | 1.08486E−47 | 3.74545E−45 | 0.36281337 | 2.668040276 |
| 2 | MYLK4 | 14.78714371 | 2.798489094 | 5.80037E−47 | 1.95186E−44 | 0.224930362 | 2.829749346 |
| 2 | KLHL29 | 14.66580105 | 2.006804466 | 1.09835E−46 | 3.60475E−44 | 0.340529248 | 2.723721981 |
| 2 | MLIP | 14.35825157 | 1.443481445 | 3.36263E−45 | 1.03944E−42 | 0.707520891 | 3.197644949 |
| 2 | PDK4 | 14.33672428 | 1.676450014 | 4.49152E−45 | 1.37244E−42 | 0.746518106 | 3.994188786 |
| 2 | NRG2 | 14.25489712 | 2.006336689 | 2.91946E−44 | 8.52867E−42 | 0.313370474 | 2.633573055 |
| 2 | NUP54 | 14.25245667 | 2.272078753 | 3.99224E−44 | 1.14118E−41 | 0.262534819 | 2.5811131 |
| 2 | ASB8 | 14.2252903 | 1.667847276 | 2.64908E−44 | 7.91273E−42 | 0.442200557 | 2.731928825 |
| 2 | PLCB1 | 14.21657848 | 1.66299212 | 2.81321E−44 | 8.3096E−42 | 0.48816156 | 3.106252432 |
| 2 | MYOM2 | 14.19118023 | 2.473006964 | 1.18702E−43 | 3.21997E−41 | 0.231197772 | 2.523180246 |
| 2 | MYBPC1 | 14.15761375 | 1.678514838 | 4.91749E−44 | 1.37607E−41 | 0.667827298 | 3.671769857 |
| 2 | RYR1 | 14.1014328 | 1.455786467 | 1.06989E−43 | 2.93217E−41 | 0.614902507 | 2.969972134 |
| 2 | NEB | 13.91592312 | 1.438664556 | 1.26458E−42 | 3.39572E−40 | 0.766713092 | 3.520876884 |
| 2 | TNNC2 | 13.70210648 | 1.815247178 | 2.43346E−41 | 6.34228E−39 | 0.371866295 | 2.646758795 |
| 2 | RP11-144F15.1 | 13.67904186 | 1.650965571 | 3.4688E−41 | 8.95287E−39 | 0.42270195 | 2.847386837 |
| 2 | RP11-532N4.2 | 13.2772007 | 1.556342602 | 4.54738E−39 | 1.10906E−36 | 0.516016713 | 3.020515203 |
| 2 | PDE4DIP | 13.24447823 | 1.222791553 | 6.58579E−39 | 1.57727E−36 | 0.746518106 | 3.125756264 |
| 2 | ACACB | 13.17049885 | 1.35765481 | 1.67609E−38 | 3.97832E−36 | 0.657381616 | 3.185276508 |
| 2 | KIF1B | 13.09702587 | 1.302709103 | 4.19572E−38 | 9.78413E−36 | 0.646935933 | 2.99387455 |
| 2 | SAMD8 | 12.70708179 | 1.47650826 | 5.87546E−36 | 1.23963E−33 | 0.433147632 | 2.750473738 |
| 2 | EGF | 12.4339304 | 1.862210274 | 2.04294E−34 | 3.9642E−32 | 0.263927577 | 2.538339376 |
| 2 | SNX1 | 12.38086987 | 1.375243545 | 2.73139E−34 | 5.26168E−32 | 0.485376045 | 2.884791374 |
| 2 | SLC22A3 | 12.26079941 | 1.845027804 | 1.52951E−33 | 2.80418E−31 | 0.268802228 | 2.672720194 |
| 2 | EYA4 | 12.20566559 | 1.242423773 | 1.92729E−33 | 3.48537E−31 | 0.655988858 | 2.986511946 |
| 2 | SORT1 | 12.06342697 | 1.631850481 | 1.31897E−32 | 2.33757E−30 | 0.308495822 | 2.548705578 |
| 2 | CLIP1 | 11.9009676 | 1.204910278 | 6.60857E−32 | 1.13108E−29 | 0.604456825 | 2.897204876 |
| 2 | PPM1B | 11.88626289 | 1.217567921 | 8.01903E−32 | 1.35782E−29 | 0.557103064 | 2.828015089 |
| 2 | CLTCL1 | 11.86348152 | 1.373698831 | 1.09429E−31 | 1.81816E−29 | 0.436629526 | 2.74962759 |
| 2 | ARID5B | 11.86039162 | 1.19337368 | 1.07109E−31 | 1.79081E−29 | 0.602367688 | 2.99244523 |
| 2 | MLK7-AS1 | 11.74491501 | 1.395440459 | 4.38208E−31 | 6.85254E−29 | 0.401114206 | 2.681766748 |
| 2 | ITIH6 | 11.65899849 | 1.83409512 | 1.49457E−30 | 2.19513E−28 | 0.236072423 | 2.588360548 |
| 2 | GPI | 11.65170574 | 1.72360146 | 1.47755E−30 | 2.18217E−28 | 0.259749304 | 2.475650787 |
| 2 | SH3RF2 | 11.50844669 | 1.823499084 | 7.70279E−30 | 1.11897E−27 | 0.233286908 | 2.553410292 |
| 2 | HOMER1 | 11.47681713 | 1.763560653 | 1.0682E−29 | 1.51047E−27 | 0.245125348 | 2.594516516 |
| 2 | PDE4D | 11.47119999 | 1.21724844 | 8.50171E−30 | 1.22167E−27 | 0.800139276 | 3.805612326 |
| 2 | RBFOX1 | 11.46440411 | 1.222707868 | 8.90645E−30 | 1.27295E−27 | 0.580779944 | 3.01186204 |
| 2 | AC013461.1 | 11.4593792 | 1.222441435 | 9.78016E−30 | 1.39035E−27 | 0.502089136 | 2.74403429 |
| 2 | TK2 | 11.4064312 | 1.607917428 | 2.1196E−29 | 2.95013E−27 | 0.280640669 | 2.5575881 |
| 2 | SLC16A10 | 11.32876968 | 1.27707684 | 4.0747E−29 | 5.5836E−27 | 0.475626741 | 2.816271544 |
| 2 | PHKA1 | 11.24694729 | 1.560922027 | 1.17442E−28 | 1.60107E−26 | 0.289693593 | 2.604842901 |
| 2 | PLAC8L1 | 11.24654484 | 1.808944583 | 1.32692E−28 | 1.79974E−26 | 0.227019499 | 2.563903809 |
| 2 | SULF2 | 11.24201488 | 1.743439555 | 1.3343E−28 | 1.80056E−26 | 0.239554318 | 2.495307207 |
| 2 | MAFG | 11.17643929 | 1.858551502 | 2.74859E−28 | 3.65342E−26 | 0.22005571 | 2.574011564 |
| 2 | RNF157 | 11.17363644 | 1.751851559 | 2.84435E−28 | 3.7619E−26 | 0.23189415 | 2.548920631 |
| 2 | SIMC1 | 11.09373093 | 1.83473897 | 6.83975E−28 | 8.8266E−26 | 0.215877437 | 2.593078613 |
| 2 | P4HA3 | 11.03998852 | 1.377724409 | 9.93481E−28 | 1.26367E−25 | 0.347493036 | 2.577624321 |
| 2 | LPIN1 | 11.0246973 | 1.200565934 | 1.07326E−27 | 1.35864E−25 | 0.511142061 | 2.898351669 |
| 2 | SLC20A2 | 10.94102001 | 1.209489107 | 2.62001E−27 | 3.2854E−25 | 0.477715877 | 2.776416063 |
| 2 | TTC19 | 10.79719353 | 1.136878729 | 1.18394E−26 | 1.43063E−24 | 0.518802228 | 2.808404207 |
| 2 | ZCCHC11 | 10.73632526 | 1.192679048 | 2.29341E−26 | 2.72178E−24 | 0.459610028 | 2.773108244 |
| 2 | PFKM | 10.70154762 | 1.589536548 | 3.80194E−26 | 4.45246E−24 | 0.247910864 | 2.480819941 |
| 2 | SALRNA2 | 10.67270088 | 1.664768457 | 5.26961E−26 | 6.03824E−24 | 0.230501393 | 2.525740385 |
| 2 | RP11-428F8.2 | 10.57173252 | 1.499569535 | 1.38459E−25 | 1.54656E−23 | 0.270194986 | 2.501222849 |
| 2 | NPAS2 | 10.56752014 | 1.294280052 | 1.3532E−25 | 1.51786E−23 | 0.371169916 | 2.712798357 |
| 2 | MYBPC2 | 10.43894672 | 2.368605614 | 7.01028E−25 | 7.66919E−23 | 0.135793872 | 2.488363266 |
| 2 | UNQ6494 | 10.37547302 | 1.605281472 | 1.06635E−24 | 1.14769E−22 | 0.23189415 | 2.650125027 |
| 2 | ATRNL1 | 10.24662209 | 2.008924246 | 4.27E−24 | 4.43413E−22 | 0.161559889 | 2.439844608 |
| 2 | CTA-125H2.1 | 10.24436092 | 1.476158977 | 3.7588E−24 | 3.93401E−22 | 0.257660167 | 2.516159058 |
| 2 | DAPK2 | 10.18653679 | 1.335023046 | 6.38842E−24 | 6.53192E−22 | 0.312674095 | 2.633814096 |
| 2 | IDNK | 10.18593693 | 1.367854118 | 6.4691E−24 | 6.58906E−22 | 0.29735376 | 2.570000172 |
| 2 | NME7 | 10.18251801 | 1.43119204 | 6.81123E−24 | 6.91106E−22 | 0.277158774 | 2.625412464 |
| 2 | GPATCH8 | 10.1769886 | 0.985487103 | 6.36933E−24 | 6.53192E−22 | 0.677576602 | 3.032249928 |
| 2 | MAX | 10.13594151 | 1.297172666 | 1.04123E−23 | 1.04849E−21 | 0.323119777 | 2.558755159 |
| 2 | CAPN7 | 10.11698532 | 1.170868993 | 1.21698E−23 | 1.22084E−21 | 0.411559889 | 2.776703835 |
| 2 | ABLIM2 | 10.10114098 | 1.082295299 | 1.36311E−23 | 1.36229E−21 | 0.519498607 | 2.800251007 |
| 2 | HSPB7 | 10.09638214 | 1.265838385 | 1.51756E−23 | 1.51097E−21 | 0.344011142 | 2.65472126 |
| 2 | DEPTOR | 10.06082535 | 1.117331147 | 2.04914E−23 | 2.02507E−21 | 0.479108635 | 2.828598976 |
| 2 | PPP2R3A | 10.01136208 | 1.170788765 | 3.41052E−23 | 3.26134E−21 | 0.402506964 | 2.687427521 |
| 2 | SPTB | 9.955547333 | 1.45314312 | 6.40171E−23 | 6.01353E−21 | 0.247214485 | 2.498726368 |
| 2 | FASTKD1 | 9.881091118 | 1.248188138 | 1.22613E−22 | 1.12787E−20 | 0.33913649 | 2.621939182 |
| 2 | RPS6KA3 | 9.876933098 | 1.204159498 | 1.27453E−22 | 1.16034E−20 | 0.36281337 | 2.690270185 |
| 2 | ABCA5 | 9.837368965 | 1.198592186 | 1.86671E−22 | 1.68791E−20 | 0.355849582 | 2.608632803 |
| 2 | AGL | 9.799097061 | 1.50841558 | 2.93247E−22 | 2.60725E−20 | 0.224233983 | 2.478471041 |
| 2 | RC3H1 | 9.767158508 | 1.169407487 | 3.62731E−22 | 3.17199E−20 | 0.366991643 | 2.604079962 |
| 2 | VPS54 | 9.617312431 | 1.232736111 | 1.52492E−21 | 1.30349E−19 | 0.325208914 | 2.658819437 |
| 2 | ITGA7 | 9.60340786 | 1.502045393 | 1.84219E−21 | 1.55469E−19 | 0.217966574 | 2.493636608 |
| 2 | ACTN2 | 9.597738266 | 1.144365668 | 1.78173E−21 | 1.50846E−19 | 0.393454039 | 2.780075312 |
| 2 | FSD2 | 9.545956612 | 1.3407197 | 3.0355E−21 | 2.5456E−19 | 0.265320334 | 2.530300856 |
| 2 | VGLL4 | 9.514321327 | 1.137398839 | 3.92051E−21 | 3.26718E−19 | 0.372562674 | 2.692630768 |
| 2 | UBE2G1 | 9.485586166 | 1.158781409 | 5.14028E−21 | 4.27028E−19 | 0.346796657 | 2.563081741 |
| 2 | GATS | 9.485530853 | 1.342911243 | 5.32946E−21 | 4.41366E−19 | 0.259052925 | 2.484338522 |
| 2 | MYLPF | 9.467499733 | 1.37778151 | 6.07695E−21 | 5.00154E−19 | 0.270194986 | 2.612981319 |
| 2 | OSBPL9 | 9.453330994 | 0.973991752 | 6.63935E−21 | 5.44755E−19 | 0.559192201 | 2.879956961 |
| 2 | HIBCH | 9.404313087 | 1.492532969 | 1.16997E−20 | 9.45367E−19 | 0.211002786 | 2.481214762 |
| 2 | CTA-125H2.2 | 9.385679245 | 1.639904261 | 1.43997E−20 | 1.1565E−18 | 0.178272981 | 2.471489191 |
| 2 | CH17-189H20.1 | 9.357336044 | 0.948179066 | 1.60482E−20 | 1.28502E−18 | 0.563370474 | 2.754024982 |
| 2 | JPH1 | 9.355898857 | 1.536561728 | 1.82541E−20 | 1.4529E−18 | 0.199164345 | 2.420407772 |
| 2 | TM4SF1-AS1 | 9.308712006 | 1.42626965 | 2.76487E−20 | 2.1746E−18 | 0.222841226 | 2.461272001 |
| 2 | CAP2 | 9.28083992 | 0.979027629 | 3.22003E−20 | 2.51769E−18 | 0.579387187 | 2.936589241 |
| 2 | FOXO1 | 9.254095078 | 0.904131472 | 4.10698E−20 | 3.20176E−18 | 0.670612813 | 3.107162237 |
| 2 | LRRFIP2 | 9.150596619 | 0.984850407 | 1.05893E−19 | 8.13602E−18 | 0.481197772 | 2.701161385 |
| 2 | LMCD1-AS1 | 9.14753437 | 1.286452651 | 1.15772E−19 | 8.81861E−18 | 0.26810585 | 2.739923239 |
| 2 | SMYD1 | 9.135910988 | 1.599646807 | 1.37596E−19 | 1.0451E−17 | 0.174791086 | 2.545118809 |
| 2 | NOCT | 9.122423172 | 1.239103675 | 1.42977E−19 | 1.07973E−17 | 0.277158774 | 2.478945494 |
| 2 | FHL1 | 9.094285965 | 1.089272141 | 1.79001E−19 | 1.33667E−17 | 0.394846797 | 2.86081028 |
| 2 | PDXDC1 | 9.035777092 | 0.782205462 | 2.91959E−19 | 2.14999E−17 | 0.766713092 | 3.167004585 |
| 2 | MURC | 9.010475159 | 2.05006218 | 4.47722E−19 | 3.24312E−17 | 0.121866295 | 2.348124981 |
| 2 | ACTN3 | 8.991279602 | 2.541647911 | 5.75328E−19 | 4.11143E−17 | 0.093314763 | 2.478112459 |
| 2 | UBAC1 | 8.976611137 | 1.11109364 | 5.11218E−19 | 3.67304E−17 | 0.337047354 | 2.567389488 |
| 2 | NEDD4L | 8.963418961 | 1.237206459 | 5.91048E−19 | 4.21244E−17 | 0.272980501 | 2.67182374 |
| 2 | RP11-701H24.9 | 8.956338882 | 1.154746413 | 6.20549E−19 | 4.41087E−17 | 0.309888579 | 2.64526391 |
| 2 | EFTUD2 | 8.913360596 | 1.242357731 | 9.17917E−19 | 6.47265E−17 | 0.260445682 | 2.546560526 |
| 2 | MPV17 | 8.89673996 | 0.982405186 | 1.00765E−18 | 7.0679E−17 | 0.465181058 | 2.744452 |
| 2 | CSPP1 | 8.841982841 | 1.077187181 | 1.66151E−18 | 1.15025E−16 | 0.350278552 | 2.662079334 |
| 2 | PPP1R27 | 8.832196236 | 1.071526051 | 1.78514E−18 | 1.23263E−16 | 0.383008357 | 2.747325659 |
| 2 | ANO6 | 8.75551796 | 1.038853407 | 3.499E−18 | 2.36085E−16 | 0.367688022 | 2.597005367 |
| 2 | SLC8A3 | 8.711203575 | 1.274811864 | 5.31079E−18 | 3.52955E−16 | 0.236768802 | 2.519943714 |
| 2 | ADAMTS17 | 8.699940681 | 1.122858167 | 5.70393E−18 | 3.78138E−16 | 0.313370474 | 2.650980234 |
| 2 | MIR133A1HG | 8.676164627 | 1.272400141 | 7.12595E−18 | 4.71234E−16 | 0.242339833 | 2.611818075 |
| 2 | ATP1A2 | 8.669501305 | 1.013550282 | 7.28978E−18 | 4.79682E−16 | 0.381615599 | 2.625000477 |
| 2 | AC016831.7 | 8.635335922 | 0.80081749 | 9.54735E−18 | 6.22076E−16 | 0.752785515 | 3.333477736 |
| 2 | YBX3 | 8.587471008 | 0.869916379 | 1.4415E−17 | 9.16766E−16 | 0.559888579 | 2.828118801 |
| 2 | NDRG2 | 8.517500877 | 1.265861273 | 2.73277E−17 | 1.70936E−15 | 0.231197772 | 2.502215385 |
| 2 | ASB5 | 8.461756706 | 0.988357008 | 4.20185E−17 | 2.60378E−15 | 0.398328691 | 2.707114458 |
| 2 | TARID | 8.426503181 | 1.096014261 | 5.74034E−17 | 3.53243E−15 | 0.300139276 | 2.574516535 |
| 2 | OSBP | 8.293802261 | 1.522951841 | 1.8211E−16 | 1.08547E−14 | 0.155988858 | 2.47021246 |
| 2 | RP11-381K20.2 | 8.291800499 | 0.991250634 | 1.71667E−16 | 1.02553E−14 | 0.376740947 | 2.64876008 |
| 2 | LDB3 | 8.240433693 | 1.042160749 | 2.63961E−16 | 1.54904E−14 | 0.328690808 | 2.69546175 |
| 2 | RP11-111E14.1 | 8.154454231 | 1.239096403 | 5.4578E−16 | 3.08703E−14 | 0.216573816 | 2.556805134 |
| 2 | STAG3 | 8.103200912 | 1.195616007 | 8.18879E−16 | 4.5542E−14 | 0.224233983 | 2.446111679 |
| 2 | PFDN1 | 8.083060265 | 1.097155929 | 9.52363E−16 | 5.25262E−14 | 0.270194986 | 2.544675827 |
| 2 | SMG1 | 8.0507164 | 0.927414656 | 1.21134E−15 | 6.59084E−14 | 0.385793872 | 2.600693464 |
| 2 | RP11-325B23.2 | 8.046936035 | 2.2081635 | 1.45199E−15 | 7.78219E−14 | 0.088440111 | 2.521135807 |
| 2 | PLCD4 | 8.044467926 | 1.257099986 | 1.32032E−15 | 7.16315E−14 | 0.201253482 | 2.449908018 |
| 2 | MEGF11 | 7.993710041 | 1.345814466 | 1.98729E−15 | 1.0503E−13 | 0.178272981 | 2.443405867 |
| 2 | FAM160A1 | 7.943561077 | 1.070365667 | 2.86396E−15 | 1.48702E−13 | 0.277855153 | 2.559786081 |
| 2 | RABEP2 | 7.939690113 | 1.660895348 | 3.16903E−15 | 1.63902E−13 | 0.125348189 | 2.57096076 |
| 2 | CCBL1 | 7.888772011 | 1.283893228 | 4.54523E−15 | 2.3192E−13 | 0.185236769 | 2.477937937 |
| 2 | ITGAD | 7.881355762 | 1.209865689 | 4.76724E−15 | 2.42318E−13 | 0.207520891 | 2.470207214 |
| 2 | MAPRE2 | 7.864051342 | 1.339866281 | 5.5226E−15 | 2.79643E−13 | 0.172005571 | 2.379411936 |
| 2 | SLC7A6OS | 7.784037113 | 0.886673033 | 9.82506E−15 | 4.90036E−13 | 0.408774373 | 2.698702335 |
| 2 | PCNT | 7.755803585 | 0.88328594 | 1.21878E−14 | 5.98891E−13 | 0.408774373 | 2.608603716 |
| 2 | BCAT2 | 7.693685532 | 1.498863459 | 2.08928E−14 | 1.01724E−12 | 0.135793872 | 2.458563805 |
| 2 | SAR1B | 7.604748726 | 1.161635399 | 3.98633E−14 | 1.90256E−12 | 0.204735376 | 2.451047182 |
| 2 | CYSTM1 | 7.597158432 | 0.855047941 | 4.11289E−14 | 1.95945E−12 | 0.415738162 | 2.715715647 |
| 2 | MAP4 | 7.584487438 | 0.783651412 | 4.49487E−14 | 2.1376E−12 | 0.52367688 | 2.778790474 |
| 2 | CDH20 | 7.581692696 | 1.439700842 | 4.8603E−14 | 2.29904E−12 | 0.149721448 | 3.055915356 |
| 2 | SLC7A6 | 7.552805424 | 0.836919785 | 5.72863E−14 | 2.68589E−12 | 0.435236769 | 2.702646971 |
| 2 | TBC1D8 | 7.492002964 | 0.760450542 | 8.9801E−14 | 4.14457E−12 | 0.622562674 | 2.948975563 |
| 2 | UCP3 | 7.491968155 | 1.373534441 | 9.42994E−14 | 4.34464E−12 | 0.149721448 | 2.380850792 |
| 2 | QKI | 7.482615948 | 0.782707155 | 9.67772E−14 | 4.45108E−12 | 0.499303621 | 2.750769138 |
| 2 | RP11-768F21.1 | 7.455995083 | 0.907964587 | 1.18811E−13 | 5.38991E−12 | 0.35724234 | 2.763791084 |
| 2 | PPP2CB | 7.451770306 | 1.228711009 | 1.25493E−13 | 5.67363E−12 | 0.179665738 | 2.392320871 |
| 2 | TNNI2 | 7.440026283 | 1.43921411 | 1.36576E−13 | 6.13298E−12 | 0.146935933 | 2.446006775 |
| 2 | PLCL1 | 7.424995899 | 1.099077344 | 1.51871E−13 | 6.78547E−12 | 0.222144847 | 2.530218601 |
| 2 | EEPD1 | 7.419616699 | 0.909545183 | 1.55257E−13 | 6.92511E−12 | 0.3454039 | 2.601792574 |
| 2 | YPEL1 | 7.359941959 | 1.545721054 | 2.54814E−13 | 1.12152E−11 | 0.118384401 | 2.390249729 |
| 2 | C1orf21 | 7.234695911 | 0.735199749 | 5.97287E−13 | 2.53647E−11 | 0.545961003 | 2.809204578 |
| 2 | RIF1 | 7.184455872 | 0.799603105 | 8.57836E−13 | 3.56324E−11 | 0.46448468 | 2.761728287 |
| 2 | RP11-724O16.1 | 7.179073334 | 1.174935699 | 9.18811E−13 | 3.78693E−11 | 0.178272981 | 2.477341413 |
| 2 | KCNQ5 | 7.177054882 | 0.832256138 | 9.05656E−13 | 3.7385E−11 | 0.473537604 | 3.087995052 |
| 2 | GREM2 | 7.158013344 | 2.362577677 | 1.16258E−12 | 4.75476E−11 | 0.064763231 | 2.61250639 |
| 2 | SF3A3 | 7.156067848 | 0.893221378 | 1.05966E−12 | 4.34724E−11 | 0.328690808 | 2.675466299 |
| 2 | AFF1 | 7.124284744 | 0.751128793 | 1.32348E−12 | 5.38798E−11 | 0.509749304 | 2.916652441 |
| 2 | PHKA1-AS1 | 7.10361433 | 1.795637369 | 1.63683E−12 | 6.59296E−11 | 0.089832869 | 2.407684326 |
| 2 | RP11-118M9.3 | 7.06819725 | 0.985042572 | 1.99263E−12 | 7.97774E−11 | 0.247214485 | 2.488824368 |
| 2 | ANO5 | 7.065400124 | 1.081579208 | 2.04882E−12 | 8.19035E−11 | 0.201253482 | 2.422908545 |
| 2 | KCNN3 | 7.031439781 | 1.725272894 | 2.71554E−12 | 1.08231E−10 | 0.092618384 | 2.417104006 |
| 2 | SGCD | 7.011361599 | 0.83352083 | 2.95147E−12 | 1.17458E−10 | 0.357938719 | 2.62675643 |
| 2 | ARG2 | 7.004193306 | 1.130879283 | 3.16516E−12 | 1.25399E−10 | 0.183147632 | 2.482298851 |
| 2 | UHMK1 | 7.004192829 | 1.633211374 | 3.25003E−12 | 1.2857E−10 | 0.100278552 | 2.348406315 |
| 2 | EIF4E2 | 6.989606857 | 0.929745317 | 3.45793E−12 | 1.35985E−10 | 0.267409471 | 2.432109833 |
| 2 | TRIM72 | 6.978973389 | 1.258201599 | 3.80899E−12 | 1.49348E−10 | 0.147632312 | 2.395759106 |
| 2 | FBXO32 | 6.926582813 | 0.757482648 | 5.30214E−12 | 2.04278E−10 | 0.610027855 | 3.126409054 |
| 2 | SRSF12 | 6.919035912 | 1.065464854 | 5.70706E−12 | 2.18612E−10 | 0.199164345 | 2.477087259 |
| 2 | GIPC2 | 6.886085033 | 1.436264634 | 7.31253E−12 | 2.77313E−10 | 0.116295265 | 2.335165977 |
| 2 | PRPF38A | 6.859508991 | 1.436969161 | 8.81813E−12 | 3.32041E−10 | 0.11281337 | 2.422100782 |
| 2 | MAP4K1 | 6.846466541 | 0.904442847 | 9.3134E−12 | 3.49207E−10 | 0.282033426 | 2.582720518 |
| 2 | EIF4EBP2 | 6.836361408 | 1.36964643 | 1.02844E−11 | 3.83449E−10 | 0.122562674 | 2.457288027 |
| 2 | CEP350 | 6.78497839 | 0.830313146 | 1.4169E−11 | 5.20982E−10 | 0.324512535 | 2.575741768 |
| 2 | GPC6 | 6.75184536 | 0.874689579 | 1.77285E−11 | 6.46496E−10 | 0.293871866 | 2.53576684 |
| 2 | SGPL1 | 6.7495327 | 1.012101173 | 1.81858E−11 | 6.62263E−10 | 0.204038997 | 2.385946035 |
| 2 | TP63 | 6.746804237 | 0.779379368 | 1.82358E−11 | 6.63173E−10 | 0.436629526 | 2.831641674 |
| 2 | LINC00691 | 6.738100052 | 1.577709198 | 2.02089E−11 | 7.30929E−10 | 0.098189415 | 2.432112217 |
| 2 | ROBO1 | 6.703614235 | 0.933861554 | 2.47323E−11 | 8.89694E−10 | 0.248607242 | 2.619490623 |
| 2 | LMCD1 | 6.701635361 | 1.128221393 | 2.52935E−11 | 9.06201E−10 | 0.165041783 | 2.468908548 |
| 2 | ATXN1 | 6.70138216 | 0.814601421 | 2.49728E−11 | 8.97131E−10 | 0.33356546 | 2.641089916 |
| 2 | DENND2C | 6.700279236 | 0.856641948 | 2.50997E−11 | 9.00474E−10 | 0.309888579 | 2.626244307 |
| 2 | C3orf35 | 6.690110207 | 1.183240056 | 2.73921E−11 | 9.73516E−10 | 0.150417827 | 2.381352425 |
| 2 | XIRP2 | 6.674599171 | 1.274981976 | 3.04419E−11 | 1.0763E−09 | 0.135793872 | 2.462682247 |
| 2 | FKBP5 | 6.648350239 | 0.514728904 | 3.53549E−11 | 1.24322E−09 | 0.901810585 | 3.89447403 |
| 2 | CACNA2D3 | 6.634839058 | 0.895377755 | 3.91389E−11 | 1.36724E−09 | 0.258356546 | 2.544224024 |
| 2 | RPTOR | 6.555390835 | 0.887068093 | 6.63254E−11 | 2.28393E−09 | 0.252785515 | 2.528850079 |
| 2 | HFE | 6.538581371 | 1.359131813 | 7.57718E−11 | 2.59243E−09 | 0.114206128 | 2.526535988 |
| 2 | TAC4 | 6.533810616 | 1.059311152 | 7.70214E−11 | 2.63179E−09 | 0.174094708 | 2.44306922 |
| 2 | RP11-83A24.2 | 6.520637989 | 0.781659007 | 8.2831E−11 | 2.81944E−09 | 0.348189415 | 2.608185291 |
| 2 | GLRA1 | 6.508709431 | 1.019299984 | 9.05534E−11 | 3.07442E−09 | 0.192896936 | 2.538327932 |
| 2 | PRKCA | 6.505006313 | 0.895421505 | 9.23527E−11 | 3.12752E−09 | 0.246518106 | 2.513574362 |
| 2 | COBL | 6.504925728 | 1.025705338 | 9.29714E−11 | 3.14447E−09 | 0.187325905 | 2.495893717 |
| 2 | RP11-371F15.3 | 6.500091076 | 1.140411139 | 9.63618E−11 | 3.255E−09 | 0.153899721 | 2.434439659 |
| 2 | LRRC38 | 6.491117477 | 1.679222345 | 1.0485E−10 | 3.53276E−09 | 0.080779944 | 2.344185352 |
| 2 | SPAG1 | 6.483561993 | 0.927696943 | 1.0631E−10 | 3.57741E−09 | 0.229108635 | 2.530188084 |
| 2 | SMTNL2 | 6.480492592 | 1.226150632 | 1.09848E−10 | 3.68712E−09 | 0.132311978 | 2.354197025 |
| 2 | JPX | 6.475807667 | 0.760470688 | 1.11204E−10 | 3.71856E−09 | 0.360027855 | 2.643571138 |
| 2 | MLIP-AS1 | 6.46105051 | 0.884789109 | 1.22794E−10 | 4.08368E−09 | 0.247214485 | 2.441102743 |
| 2 | VCL | 6.460318089 | 0.779491663 | 1.22891E−10 | 4.8368E−09 | 0.341922006 | 2.617013454 |
| 2 | AC016722.4 | 6.422340393 | 1.800293326 | 1.64957E−10 | 5.38724E−09 | 0.071727019 | 2.327499628 |
| 2 | NPR3 | 6.420158386 | 2.21854949 | 1.69559E−10 | 5.52396E−09 | 0.056406685 | 2.289549828 |
| 2 | ANKDD1B | 6.37232399 | 1.225426793 | 2.21771E−10 | 7.13748E−09 | 0.126044568 | 2.38174057 |
| 2 | UACA | 6.368700027 | 0.767962694 | 2.22489E−10 | 7.15192E−09 | 0.33356546 | 2.606976509 |
| 2 | AC097662.2 | 6.355463505 | 1.311779976 | 2.46931E−10 | 7.89942E−09 | 0.116991643 | 2.438013315 |
| 2 | MSS51 | 6.329762936 | 1.573665023 | 2.95558E−10 | 9.38724E−09 | 0.08356546 | 2.329020262 |
| 2 | ARHGEF26 | 6.313342571 | 0.842444062 | 3.1785E−10 | 1.00592E−08 | 0.263927577 | 2.531813622 |
| 2 | NODAL | 6.294103146 | 1.306126118 | 3.67354E−10 | 1.15434E−08 | 0.110724234 | 2.436511517 |
| 2 | HIGD1C | 6.235577106 | 1.114391446 | 5.25696E−10 | 1.62312E−08 | 0.144846797 | 2.409444332 |
| 2 | USP39 | 6.212731838 | 0.701708555 | 5.98486E−10 | 1.83932E−08 | 0.401810585 | 2.730509996 |
| 2 | PIGB | 6.207038403 | 0.792293847 | 6.21479E−10 | 1.90558E−08 | 0.288300836 | 2.521140575 |
| 2 | SPRTN | 6.165034294 | 0.77936244 | 8.06355E−10 | 2.4304E−08 | 0.312674095 | 2.62133193 |
| 2 | CTD-2193G5.1 | 6.154533386 | 3.582132339 | 9.42298E−10 | 2.82095E−08 | 0.034122563 | 2.28572607 |
| 2 | PHKG1 | 6.149110317 | 0.851213336 | 8.94283E−10 | 2.69183E−08 | 0.241643454 | 2.521023035 |
| 2 | DDIT4L | 6.133386612 | 1.341973901 | 1.00345E−09 | 2.99728E−08 | 0.102367688 | 2.355370045 |
| 2 | TACC2 | 6.109323025 | 0.680676758 | 1.13653E−09 | 3.37957E−08 | 0.443593315 | 2.696925402 |
| 2 | UNC13C | 6.107163429 | 1.186853886 | 1.1735E−09 | 3.48564E−08 | 0.124651811 | 2.474754333 |
| 2 | ZBTB43 | 6.103628635 | 0.788885057 | 1.18274E−09 | 3.50915E−08 | 0.277855153 | 2.480987549 |
| 2 | ADPRHL1 | 6.093958855 | 1.129997492 | 1.27184E−09 | 3.76091E−08 | 0.132311978 | 2.360782146 |
| 2 | STRIP2 | 6.019375801 | 1.43728292 | 2.02454E−09 | 5.85003E−08 | 0.088440111 | 2.355104446 |
| 2 | RIC1 | 5.99045372 | 0.714269519 | 2.36163E−09 | 6.77987E−08 | 0.341922006 | 2.60409236 |
| 2 | SLC7A8 | 5.983870029 | 1.03828299 | 2.48204E−09 | 7.11786E−08 | 0.151114206 | 2.443205595 |
| 2 | OPTN | 5.980839729 | 0.770238936 | 2.50625E−09 | 7.16411E−08 | 0.281337047 | 2.465133905 |
| 2 | GPD2 | 5.979398251 | 1.100984693 | 2.55513E−09 | 7.28815E−08 | 0.135793872 | 2.376873016 |
| 2 | HSF2 | 5.975235939 | 1.195581675 | 2.62834E−09 | 7.47037E−08 | 0.115598886 | 2.32460022 |
| 2 | IL12RB2 | 5.963813305 | 0.742729962 | 2.77671E−09 | 7.86114E−08 | 0.314763231 | 2.645468473 |
| 2 | RFX3-AS1 | 5.957685947 | 0.886065722 | 2.89678E−09 | 8.18364E−08 | 0.20264624 | 2.485669851 |
| 2 | ATG14 | 5.953915119 | 0.744897127 | 2.94847E−09 | 8.312E−08 | 0.307103064 | 2.591814518 |
| 2 | CTD-2545M3.8 | 5.951681137 | 1.111817956 | 3.02855E−09 | 8.51967E−08 | 0.129526462 | 2.489305258 |
| 2 | CDH4 | 5.873009205 | 1.095511317 | 4.8393E−09 | 1.32938E−07 | 0.132311978 | 2.458757639 |
| 2 | FTX | 5.849728584 | 0.516242743 | 5.48183E−09 | 1.49619E−07 | 0.722841226 | 3.080647707 |
| 2 | KLHL30 | 5.849437714 | 1.227560163 | 5.58979E−09 | 1.52097E−07 | 0.106545961 | 2.399178982 |
| 2 | ARL5A | 5.844782829 | 0.826559246 | 5.68048E−09 | 1.54407E−07 | 0.223537604 | 2.44156599 |
| 2 | DAAM2 | 5.839938641 | 0.853044868 | 5.8481E−09 | 1.57994E−07 | 0.215877437 | 2.524725437 |
| 2 | CLCA2 | 5.836750984 | 1.558387637 | 6.09763E−09 | 1.64235E−07 | 0.072423398 | 2.342546225 |
| 2 | HDAC4 | 5.832043171 | 0.62859869 | 6.08989E−09 | 1.64192E−07 | 0.470752089 | 2.720723867 |
| 2 | PRKCA-AS1 | 5.831533432 | 1.210987568 | 6.21719E−09 | 1.66947E−07 | 0.110724234 | 2.504149675 |
| 2 | NOS1 | 5.829775333 | 0.891424894 | 6.22507E−09 | 1.6699E−07 | 0.193593315 | 2.566907883 |
| 2 | ADCY9 | 5.826086521 | 0.744399965 | 6.32864E−09 | 1.69597E−07 | 0.298746518 | 2.652387381 |
| 2 | C9orf147 | 5.821271896 | 1.215771198 | 6.61341E−09 | 1.7705E−07 | 0.106545961 | 2.41872263 |
| 2 | COL4A4 | 5.79391098 | 1.049885988 | 7.7102E−09 | 2.04968E−07 | 0.139972145 | 2.427021503 |
| 2 | TPM1 | 5.787310123 | 0.813521087 | 7.95182E−09 | 2.10549E−07 | 0.248607242 | 2.619199753 |
| 2 | ASPH | 5.778774261 | 0.721340954 | 8.36533E−09 | 2.20838E−07 | 0.299442897 | 2.506700993 |
| 2 | NBPF19 | 5.752398491 | 0.919067919 | 9.80432E−09 | 2.55528E−07 | 0.178272981 | 2.473294497 |
| 2 | RHOBTB3 | 5.746473789 | 0.668528318 | 1.00904E−08 | 2.61701E−07 | 0.366991643 | 2.606042624 |
| 2 | MYOT | 5.734789848 | 0.677414119 | 1.07986E−08 | 2.7871E−07 | 0.364206128 | 2.633442402 |
| 2 | FBXO34 | 5.723662853 | 0.762316346 | 1.15569E−08 | 2.97127E−07 | 0.258356546 | 2.503517866 |
| 2 | TNIK | 5.722539425 | 0.816108584 | 1.16508E−08 | 2.9925E−07 | 0.222841226 | 2.520632505 |
| 2 | PRKCQ | 5.703382015 | 1.279626489 | 1.31775E−08 | 3.37487E−07 | 0.0954039 | 2.417065144 |
| 2 | ERMP1 | 5.69082737 | 0.709023356 | 1.39528E−08 | 3.56654E−07 | 0.318941504 | 2.630086184 |
| 2 | NR3C2 | 5.6850214 | 0.721586823 | 1.44468E−08 | 3.68928E−07 | 0.305013928 | 2.715060949 |
| 2 | PDZD2 | 5.678699493 | 0.603422284 | 1.4954E−08 | 3.81148E−07 | 0.47005571 | 2.768898249 |
| 2 | RP11-897M7.4 | 5.67139864 | 2.886715174 | 1.65381E−08 | 4.16334E−07 | 0.033426184 | 2.496524096 |
| 2 | RP11-21A7A.2 | 5.668232918 | 0.778700352 | 1.59367E−08 | 4.02719E−07 | 0.243036212 | 2.521104336 |
| 2 | ORC1 | 5.658322811 | 1.3919245 | 1.71732E−08 | 4.30691E−07 | 0.080083565 | 2.365468025 |
| 2 | RP11-96C21.2 | 5.601378441 | 1.360013962 | 2.37232E−08 | 5.82325E−07 | 0.082869081 | 2.386307001 |
| 2 | GTF2IRD1 | 5.598028183 | 0.639932036 | 2.37598E−08 | 5.82686E−07 | 0.393454039 | 2.676052094 |
| 2 | NRAP | 5.590621471 | 0.790347159 | 2.4873E−08 | 6.06072E−07 | 0.223537604 | 2.494631529 |
| 2 | RP11-96H19.1 | 5.589625359 | 0.934019744 | 2.51174E−08 | 6.10349E−07 | 0.163649025 | 2.62950182 |
| 2 | SRL | 5.589553356 | 0.769065976 | 2.50193E−08 | 6.08521E−07 | 0.239554318 | 2.497394323 |
| 2 | LINC00222 | 5.577963352 | 1.59605062 | 2.72833E−08 | 6.59963E−07 | 0.064066852 | 2.372701168 |
| 2 | TBC1D4 | 5.571581364 | 0.810743451 | 2.77413E−08 | 6.7043E−07 | 0.211002786 | 2.488338947 |
| 2 | RP4-791M13.4 | 5.568320751 | 1.210481882 | 2.85772E−08 | 6.86266E−07 | 0.096796657 | 2.385561228 |
| 2 | ATP1B1 | 5.551194191 | 0.783350825 | 3.11123E−08 | 7.45127E−07 | 0.228412256 | 2.523638248 |
| 2 | TRIM63 | 5.544465065 | 0.60120213 | 3.2175E−08 | 7.69883E−07 | 0.578690808 | 2.96773386 |
| 2 | PKIA | 5.541107655 | 1.316698074 | 3.33744E−08 | 7.95005E−07 | 0.084261838 | 2.35764575 |
| 2 | RP11-138H8.2 | 5.538577557 | 0.858448386 | 3.34641E−08 | 7.95716E−07 | 0.183147632 | 2.429870129 |
| 2 | PWAR6 | 5.535499096 | 1.076404333 | 3.42622E−08 | 8.12511E−07 | 0.116991643 | 2.430520058 |
| 2 | OBSCN | 5.535283566 | 0.627835393 | 3.39038E−08 | 8.05449E−07 | 0.412256267 | 2.646905184 |
| 2 | HADH | 5.527839184 | 1.297817588 | 3.59645E−08 | 8.4759E−07 | 0.085654596 | 2.391655922 |
| 2 | MDH1 | 5.518513203 | 1.031569123 | 3.75997E−08 | 8.82215E−07 | 0.128133705 | 2.347667217 |
| 2 | DGKG | 5.516396999 | 0.66420424 | 3.77652E−08 | 8.85317E−07 | 0.324512535 | 2.514449596 |
| 2 | CASQ1 | 5.511046886 | 1.345184684 | 3.95611E−08 | 9.24157E−07 | 0.080083565 | 2.375735283 |
| 2 | FBXL17 | 5.492256165 | 0.628690541 | 4.3246E−08 | 1.00231E−06 | 0.373259053 | 2.631012678 |
| 2 | ABRA | 5.489327908 | 1.4626894 | 4.47243E−08 | 1.03297E−06 | 0.071030641 | 2.332040071 |
| 2 | STIM1 | 5.439383984 | 0.609540999 | 5.80471E−08 | 1.3223E−06 | 0.401810585 | 2.659013033 |
| 2 | RP13-270P17.2 | 5.437913418 | 0.795121312 | 5.87348E−08 | 1.33682E−06 | 0.211002786 | 2.584181309 |
| 2 | PPP1R42 | 5.437757015 | 1.579106688 | 5.99426E−08 | 1.36314E−06 | 0.062674095 | 2.55819273 |
| 2 | PRSS23 | 5.399886608 | 0.712593496 | 7.24289E−08 | 1.63174E−06 | 0.26810585 | 2.725646257 |
| 2 | ATP2A3 | 5.373728275 | 1.126223564 | 8.43395E−08 | 1.88252E−06 | 0.102367688 | 2.393721104 |
| 2 | RP11-230G5.2 | 5.363270283 | 1.397930861 | 8.98182E−08 | 1.99643E−06 | 0.071727019 | 2.31652379 |
| 2 | LMTK2 | 5.358157635 | 1.16266048 | 9.18126E−08 | 2.03735E−06 | 0.097493036 | 2.359257698 |
| 2 | SPPL2A | 5.346503258 | 0.6294204 | 9.68974E−08 | 2.14303E−06 | 0.346796657 | 2.621865273 |
| 2 | PDE7A | 5.336833477 | 0.584426701 | 1.02042E−07 | 2.2512E−06 | 0.443593315 | 2.740092754 |
| 2 | ATP2A1-AS1 | 5.328604698 | 1.705129623 | 1.0935E−07 | 2.39848E−06 | 0.05362117 | 2.451464415 |
| 2 | RP11-665C16.5 | 5.325303078 | 0.719527483 | 1.08926E−07 | 2.39116E−06 | 0.247910864 | 2.524702072 |
| 2 | PDE5A | 5.320473671 | 0.599351346 | 1.11632E−07 | 2.44451E−06 | 0.412952646 | 2.799374819 |
| 2 | AC010976.2 | 5.320057392 | 0.9146052 | 1.12584E−07 | 2.46333E−06 | 0.145543175 | 2.438490391 |
| 2 | MLF1 | 5.312267303 | 1.561250448 | 1.18923E−07 | 2.59562E−06 | 0.060584958 | 2.304358721 |
| 2 | SEC13 | 5.301802158 | 0.726881862 | 1.23838E−07 | 2.69183E−06 | 0.235376045 | 2.493355513 |
| 2 | EIF4G3 | 5.28313303 | 0.567033887 | 1.36593E−07 | 2.95459E−06 | 0.456128134 | 2.692622423 |
| 2 | RP6-74O6.2 | 5.266332626 | 1.063654423 | 1.50992E−07 | 3.25018E−06 | 0.107938719 | 2.441189528 |
| 2 | RUSC2 | 5.261108875 | 0.81572777 | 1.54472E−07 | 3.31704E−06 | 0.181754875 | 2.457683086 |
| 2 | COL4A3 | 5.254999638 | 0.88800019 | 1.59722E−07 | 3.42423E−06 | 0.157381616 | 2.464712858 |
| 2 | ANKS1B | 5.251470566 | 0.68929261 | 1.62485E−07 | 3.47786E−06 | 0.275069638 | 2.725922108 |
| 2 | RP5-1042K10.10 | 5.237930775 | 0.724867165 | 1.74684E−07 | 3.71801E−06 | 0.233983287 | 2.461925507 |
| 2 | GHR | 5.237748623 | 0.583493829 | 1.74495E−07 | 3.71721E−06 | 0.424791086 | 2.761683702 |
| 2 | HSPB8 | 5.237487793 | 0.888152897 | 1.75564E−07 | 3.73375E−06 | 0.151810585 | 2.410430908 |
| 2 | MAP3K19 | 5.23617363 | 0.948098719 | 1.77005E−07 | 3.75839E−06 | 0.135793872 | 2.452893972 |
| 2 | FAM20A | 5.235136032 | 0.725234747 | 1.77362E−07 | 3.75996E−06 | 0.242339833 | 2.668307781 |
| 2 | RP11-350J20.5 | 5.212409973 | 1.161624908 | 2.01765E−07 | 4.2334E−06 | 0.092618384 | 2.349050283 |
| 2 | SPIDR | 5.207962036 | 0.593797565 | 2.04766E−07 | 4.29298E−06 | 0.396935933 | 2.799469471 |
| 2 | AJ006998.2 | 5.197962284 | 1.404061794 | 2.18621E−07 | 4.56546E−06 | 0.069637883 | 2.355150223 |
| 2 | PPIL2 | 5.192333698 | 1.150898933 | 2.24735E−07 | 4.67111E−06 | 0.092618384 | 2.452603102 |
| 2 | FEZ2 | 5.191616058 | 0.619407117 | 2.23327E−07 | 4.6564E−06 | 0.375348189 | 2.811244488 |
| 2 | PLCL2 | 5.183706284 | 0.863212109 | 2.33792E−07 | 4.83667E−06 | 0.158077994 | 2.43826437 |
| 2 | ENOX1 | 5.174427509 | 0.891754627 | 2.45982E−07 | 5.07306E−06 | 0.146935933 | 2.504847527 |
| 2 | EDARADD | 5.164002895 | 0.964813292 | 2.60461E−07 | 5.33443E−06 | 0.123259053 | 2.447891235 |
| 2 | RP11-840I19.3 | 5.161579132 | 0.881000698 | 2.6309E−07 | 5.37999E−06 | 0.151114206 | 2.494693279 |
| 2 | TRIM13 | 5.161230564 | 0.642785728 | 2.62523E−07 | 5.37254E−06 | 0.302228412 | 2.570054293 |
| 2 | NXN | 5.137310028 | 0.650929749 | 2.9794E−07 | 6.06E−06 | 0.28551532 | 2.50474143 |
| 2 | TMEM233 | 5.115921974 | 0.695633292 | 3.33556E−07 | 6.73292E−06 | 0.244428969 | 2.493121624 |
| 2 | CAST | 5.094389439 | 0.635387361 | 3.73176E−07 | 7.47027E−06 | 0.296657382 | 2.514803171 |
| 2 | ANKUB1 | 5.079537868 | 0.596240044 | 4.03054E−07 | 8.02606E−06 | 0.350278552 | 2.576567173 |
| 2 | RP11-191G24.1 | 5.076295853 | 1.132889152 | 4.13611E−07 | 8.21781E−06 | 0.091225627 | 2.439796686 |
| 2 | ASB2 | 5.064565182 | 0.621213555 | 4.35967E−07 | 8.62332E−06 | 0.309192201 | 2.531451464 |
| 2 | RP11-845C23.3 | 5.055278778 | 1.291176796 | 4.63044E−07 | 9.12495E−06 | 0.071030641 | 2.375842333 |
| 2 | AVIL | 5.042407036 | 0.77113831 | 4.90125E−07 | 9.60877E−06 | 0.182451253 | 2.379517317 |
| 2 | RP11-417L19.4 | 5.035621643 | 1.494982123 | 5.13626E−07 | 1.00104E−05 | 0.057103064 | 2.347417831 |
| 2 | RBM24 | 5.012312889 | 0.845932543 | 5.73523E−07 | 1.10723E−05 | 0.14902507 | 2.42898345 |
| 2 | C2orf68 | 4.986400127 | 1.203736186 | 6.58321E−07 | 1.25996E−05 | 0.078690808 | 2.346474171 |
| 2 | NEXN-AS1 | 4.979665756 | 0.94586575 | 6.78896E−07 | 1.29747E−05 | 0.11908078 | 2.327686548 |
| 2 | ITIH2 | 4.977892399 | 1.093369961 | 6.86648E−07 | 1.31134E−05 | 0.091922006 | 2.326326132 |
| 2 | ENO3 | 4.975479126 | 0.73831892 | 6.9045E−07 | 1.31671E−05 | 0.206128134 | 2.463131666 |
| 2 | RP11-141J13.5 | 4.960946083 | 1.284856796 | 7.51298E−07 | 1.42254E−05 | 0.069637883 | 2.256334782 |
| 2 | RMDN1 | 4.949790001 | 0.914476573 | 7.90288E−07 | 1.49212E−05 | 0.125348189 | 2.382666111 |
| 2 | GANC | 4.941973686 | 0.89636749 | 8.2188E−07 | 1.54956E−05 | 0.13091922 | 2.365914583 |
| 2 | CAPN3 | 4.94039011 | 1.093841314 | 8.31138E−07 | 1.5648E−05 | 0.090529248 | 2.358281612 |
| 2 | CDKL4 | 4.938215256 | 0.842688084 | 8.37549E−07 | 1.57575E−05 | 0.146239554 | 2.414891481 |
| 2 | CACNG1 | 4.937208652 | 1.082373261 | 8.44884E−07 | 1.58731E−05 | 0.091225627 | 2.39936924 |
| 2 | OIP5 | 4.923621178 | 0.814549446 | 9.01286E−07 | 1.67551E−05 | 0.155988858 | 2.404318333 |
| 2 | MTHFD1 | 4.905974865 | 0.997048259 | 9.88254E−07 | 1.81937E−05 | 0.105153203 | 2.406636 |
| 2 | MYO9B | 4.901232719 | 0.688358784 | 1.00806E−06 | 1.8507E−05 | 0.220752089 | 2.514403343 |
| 2 | MS4A5 | 4.888898849 | 1.180695176 | 1.07874E−06 | 1.97366E−05 | 0.079387187 | 2.291327477 |
| 2 | GDNF | 4.871778488 | 1.49559474 | 1.18207E−06 | 2.14787E−05 | 0.05362117 | 2.317410469 |
| 2 | NFE2L3 | 4.841413021 | 0.670194924 | 1.36034E−06 | 2.45508E−05 | 0.23189415 | 2.496360779 |
| 2 | RFXAP | 4.822390556 | 1.262985468 | 1.50927E−06 | 2.70367E−05 | 0.066852368 | 2.44219327 |
| 2 | SLC10A1 | 4.776978493 | 1.198511958 | 1.88456E−06 | 3.30469E−05 | 0.073816156 | 2.377542496 |
| 2 | NT5C3A | 4.758224964 | 0.754036367 | 2.05646E−06 | 3.5708E−05 | 0.169220056 | 2.452103615 |
| 2 | TSPAN5 | 4.755232334 | 1.136141896 | 2.09431E−06 | 3.62468E−05 | 0.082869081 | 2.557271719 |
| 2 | RP11-227G15.12 | 4.754445553 | 0.696479797 | 2.09279E−06 | 3.62441E−05 | 0.199164345 | 2.421836138 |
| 2 | FAM78B | 4.74349165 | 1.144863367 | 2.22089E−06 | 3.8288E−05 | 0.07729805 | 2.370228529 |
| 2 | AC004485.3 | 4.732640743 | 1.023942471 | 2.33835E−06 | 4.00791E−05 | 0.092618384 | 2.373409033 |
| 2 | PPP3CC | 4.695434093 | 0.559160054 | 2.78856E−06 | 4.70077E−05 | 0.330083565 | 2.612251043 |
| 2 | FAM168A | 4.689260483 | 0.60872823 | 2.87394E−06 | 4.84163E−05 | 0.282729805 | 2.69628191 |
| 2 | TMEM38A | 4.683812141 | 0.710946739 | 2.95437E−06 | 4.95828E−05 | 0.18454039 | 2.455602407 |
| 2 | LINC00866 | 4.683024406 | 1.197327256 | 2.9838E−06 | 5.0045E−05 | 0.070334262 | 2.426511765 |
| 2 | ASB15 | 4.67249918 | 1.099468827 | 3.13534E−06 | 5.22242E−05 | 0.080779944 | 2.33115387 |
| 2 | RNLS | 4.665720463 | 0.731162846 | 3.22546E−06 | 5.3591E−05 | 0.176880223 | 2.616832972 |
| 2 | STAC3 | 4.657020569 | 0.658814192 | 3.35983E−06 | 5.56843E−05 | 0.215181058 | 2.405588627 |
| 2 | FBXO40 | 4.646110058 | 1.162362933 | 3.56439E−06 | 5.87815E−05 | 0.071030641 | 2.408435583 |
| 2 | SLC9C2 | 4.637392521 | 1.175510526 | 3.71315E−06 | 6.10076E−05 | 0.071030641 | 2.415346861 |
| 2 | NBPF14 | 4.635867119 | 1.058641553 | 3.73287E−06 | 6.1256E−05 | 0.086350975 | 2.305544853 |
| 2 | TLE4 | 4.630598068 | 0.690673888 | 3.81696E−06 | 6.252E−05 | 0.192896936 | 2.480966091 |
| 2 | LRRC2 | 4.615401268 | 0.610852897 | 4.10145E−06 | 6.68094E−05 | 0.253481894 | 2.519150496 |
| 2 | C9orf24 | 4.611897945 | 1.07370317 | 4.18919E−06 | 6.80717E−05 | 0.082172702 | 2.28044939 |
| 2 | LINC00499 | 4.609989166 | 2.183970928 | 4.28851E−06 | 6.95786E−05 | 0.029247911 | 2.339988708 |
| 2 | TMOD1 | 4.606504917 | 0.755767822 | 4.28332E−06 | 6.95589E−05 | 0.159470752 | 2.379768133 |
| 2 | TNKS2 | 4.606076717 | 0.70644623 | 4.29239E−06 | 6.95786E−05 | 0.178969359 | 2.428109884 |
| 2 | C16orf45 | 4.583384514 | 0.638339698 | 4.77793E−06 | 7.67936E−05 | 0.227019499 | 2.546530962 |
| 2 | RP11-473C19.1 | 4.575073242 | 1.065599918 | 4.99545E−06 | 7.99512E−05 | 0.080779944 | 2.498821974 |
| 2 | TUBA4A | 4.563204765 | 1.233139515 | 5.28815E−06 | 8.41797E−05 | 0.063370474 | 2.377240658 |
| 2 | UBXN8 | 4.553378582 | 0.94791162 | 5.52383E−06 | 8.77379E−05 | 0.098885794 | 2.322859526 |
| 2 | AC073254.1 | 4.547802925 | 1.671471834 | 5.71952E−06 | 9.04507E−05 | 0.039693593 | 2.373834848 |
| 2 | GLRX | 4.539978981 | 0.649094343 | 5.86691E−06 | 9.26164E−05 | 0.211699164 | 2.463886261 |
| 2 | DYSF | 4.539159298 | 0.835072935 | 5.90207E−06 | 9.30609E−05 | 0.124651811 | 2.460660458 |
| 2 | NFIC | 4.533635616 | 0.521701992 | 6.04041E−06 | 9.5073E−05 | 0.355153203 | 2.612131596 |
| 2 | FILIP1 | 4.525385857 | 0.550269783 | 6.28005E−06 | 9.84847E−05 | 0.307103064 | 2.54828763 |
| 2 | MACROD2 | 4.525078773 | 0.530305207 | 6.28681E−06 | 9.84847E−05 | 0.352367688 | 2.613668442 |
| 2 | RHOBTB1 | 4.516298294 | 0.96162647 | 6.57823E−06 | 0.000102506 | 0.094707521 | 2.455159664 |
| 2 | ELOVL6 | 4.516298294 | 1.217215061 | 6.59464E−06 | 0.000102702 | 0.064066852 | 2.353335857 |
| 2 | SETBP1 | 4.503486156 | 0.563889563 | 6.95908E−06 | 0.000107746 | 0.290389972 | 2.59438777 |
| 2 | PCMT1 | 4.493499279 | 0.708875835 | 7.29844E−06 | 0.000112738 | 0.170612813 | 2.455319881 |
| 2 | RAB2A | 4.460028172 | 0.586815536 | 8.51945E−06 | 0.000129789 | 0.254874652 | 2.491251707 |
| 2 | MED14 | 4.45724678 | 1.019259334 | 8.66626E−06 | 0.000131723 | 0.082869081 | 2.441529274 |
| 2 | CTD-2012K14.6 | 4.455593109 | 1.334915996 | 8.76551E−06 | 0.000133156 | 0.052228412 | 2.358851671 |
| 2 | LYSMD4 | 4.451208115 | 1.074837685 | 8.91055E−06 | 0.000134933 | 0.075905292 | 2.306442022 |
| 2 | FOXN4 | 4.451116562 | 1.006162047 | 8.91297E−06 | 0.000134933 | 0.08356546 | 2.33046174 |
| 2 | PFKFB1 | 4.446200848 | 1.331564903 | 9.15354E−06 | 0.000138182 | 0.052228412 | 2.455097914 |
| 2 | AP3M1 | 4.432250023 | 1.016447186 | 9.71636E−06 | 0.000145932 | 0.08356546 | 2.294987202 |
| 2 | ANKRD1 | 4.429760933 | 1.666306138 | 9.88021E−06 | 0.000148142 | 0.039693593 | 2.345541954 |
| 2 | MTFR1 | 4.425400257 | 0.531777561 | 9.99149E−06 | 0.000149513 | 0.321727019 | 2.537325382 |
| 2 | CTD-2184D3.5 | 4.409750938 | 1.133828282 | 1.07882E−05 | 0.00016013 | 0.068941504 | 2.283704042 |
| 2 | CPLX2 | 4.406963825 | 1.556626558 | 1.09778E−05 | 0.000162762 | 0.041086351 | 2.326317549 |
| 2 | SLC24A1 | 4.39994812 | 0.598594308 | 1.12361E−05 | 0.000166315 | 0.236768802 | 2.519087076 |
| 2 | PRDM2 | 4.396284103 | 0.674488187 | 1.14326E−05 | 0.000168754 | 0.178969359 | 2.43966651 |
| 2 | IP6K3 | 4.390651703 | 0.650248408 | 1.17299E−05 | 0.000172853 | 0.191504178 | 2.470902205 |
| 2 | RP11-293P20.2 | 4.383620739 | 0.928428948 | 1.21526E−05 | 0.000178785 | 0.091922006 | 2.469679117 |
| 2 | MBNL1 | 4.372125149 | 0.384372205 | 1.27428E−05 | 0.000186231 | 0.720752089 | 3.058981895 |
| 2 | JMY | 4.367392063 | 0.777659357 | 1.30537E−05 | 0.000190461 | 0.13091922 | 2.439778328 |
| 2 | SLC38A4 | 4.351665497 | 1.040799022 | 1.40464E−05 | 0.000203493 | 0.076601671 | 2.374114513 |
| 2 | NUDCD2 | 4.337947845 | 1.113659024 | 1.49594E−05 | 0.000215428 | 0.067548747 | 2.338478088 |
| 2 | EIF4ENIF1 | 4.325204372 | 0.650254726 | 1.57853E−05 | 0.000226218 | 0.186629526 | 2.515657425 |
| 2 | GPAT3 | 4.323894024 | 0.855479479 | 1.5899E−05 | 0.000227725 | 0.107938719 | 2.368318796 |
| 2 | COL6A6 | 4.319997311 | 0.805256009 | 1.61737E−05 | 0.000231162 | 0.120473538 | 2.294594765 |
| 2 | CNOT7 | 4.292122841 | 0.774126947 | 1.83304E−05 | 0.000260169 | 0.126044568 | 2.37527132 |
| 2 | ZNF565 | 4.276602745 | 0.48028174 | 1.96058E−05 | 0.000275185 | 0.390668524 | 2.669239759 |
| 2 | AMPD1 | 4.267043591 | 0.851922631 | 2.05186E−05 | 0.000286785 | 0.103064067 | 2.369297266 |
| 2 | PBX1 | 4.241826534 | 0.487908036 | 2.28793E−05 | 0.000316618 | 0.376740947 | 2.660062313 |
| 2 | ACYP2 | 4.238454819 | 0.448799998 | 2.32226E−05 | 0.000320868 | 0.456128134 | 2.699268818 |
| 2 | PEX5L | 4.233957291 | 0.498390526 | 2.36853E−05 | 0.000326582 | 0.411559889 | 2.892577171 |
| 2 | TEAD1 | 4.233444214 | 0.573230445 | 2.37588E−05 | 0.000327283 | 0.238857939 | 2.482517004 |
| 2 | ZFAND1 | 4.232156754 | 0.593358397 | 2.39001E−05 | 0.000329032 | 0.230501393 | 2.750156403 |
| 2 | UBASH3B | 4.226789951 | 1.036763191 | 2.45327E−05 | 0.000336868 | 0.075208914 | 2.448854685 |
| 2 | CRLF3 | 4.221986294 | 0.543428361 | 2.49905E−05 | 0.000342623 | 0.266713092 | 2.55253315 |
| 2 | MIR4300HG | 4.216204643 | 0.771972835 | 2.56633E−05 | 0.000349504 | 0.123955432 | 2.335755348 |
| 2 | BLCAP | 4.211777687 | 0.625182033 | 2.61476E−05 | 0.00035519 | 0.189415042 | 2.371333838 |
| 2 | SMAD9 | 4.20653677 | 0.813837707 | 2.6803E−05 | 0.000363351 | 0.110027855 | 2.427425861 |
| 2 | DPF3 | 4.20170927 | 0.53338021 | 2.73164E−05 | 0.000369557 | 0.282033426 | 2.535052299 |
| 2 | ASB14 | 4.198912621 | 0.655549347 | 2.76786E−05 | 0.000373696 | 0.169916435 | 2.425019979 |
| 2 | RP11-415J8.3 | 4.19524622 | 0.471310765 | 2.81001E−05 | 0.000379003 | 0.38718663 | 2.720638752 |
| 2 | PPARGC1B | 4.189446926 | 0.865730047 | 2.88857E−05 | 0.000389005 | 0.097493036 | 2.334485292 |
| 2 | SOX6 | 4.160306454 | 0.502826691 | 3.27299E−05 | 0.000436355 | 0.323119777 | 2.587557077 |
| 2 | RP11-61I13.3 | 4.152643681 | 1.269664407 | 3.40072E−05 | 0.000451572 | 0.050139276 | 2.307988644 |
| 2 | AC008592.4 | 4.151975632 | 1.22481966 | 3.40946E−05 | 0.000452506 | 0.05362117 | 2.281617641 |
| 2 | VMP1 | 4.148012638 | 0.5133093 | 3.4534E−05 | 0.000457424 | 0.298746518 | 2.563537121 |
| 2 | LRTM1 | 4.1448946 | 0.86163789 | 3.5095E−05 | 0.00046347 | 0.096100279 | 2.550363779 |
| 2 | ZNF552 | 4.143170357 | 0.876558423 | 3.53479E−05 | 0.000465654 | 0.092618384 | 2.357055664 |
| 2 | NME9 | 4.120464325 | 0.614403903 | 3.89304E−05 | 0.000507068 | 0.188022284 | 2.507870674 |
| 2 | RP11-701H24.3 | 4.115634441 | 1.891359925 | 4.01399E−05 | 0.000521544 | 0.027855153 | 2.272927046 |
| 2 | CHN2 | 4.112382889 | 0.615279734 | 4.03094E−05 | 0.000523235 | 0.20264624 | 2.900796175 |
| 2 | RP11-1035H13.2 | 4.111886501 | 1.593580127 | 4.06923E−05 | 0.00052769 | 0.034818942 | 2.291850567 |
| 2 | MCFD2 | 4.110617638 | 0.797943354 | 4.06604E−05 | 0.000527533 | 0.109331476 | 2.414054871 |
| 2 | CNTFR | 4.107055664 | 1.07681191 | 4.13697E−05 | 0.000534697 | 0.063370474 | 2.341659784 |
| 2 | ARNTL | 4.106516838 | 0.689685106 | 4.13548E−05 | 0.000534697 | 0.143454039 | 2.373048782 |
| 2 | HOMER2 | 4.10157156 | 0.594580948 | 4.22192E−05 | 0.000544568 | 0.201949861 | 2.530786514 |
| 2 | CECR2 | 4.098083973 | 0.532681108 | 4.28433E−05 | 0.000551548 | 0.258356546 | 2.497214317 |
| 2 | NPAS3 | 4.08330822 | 0.649238706 | 4.56608E−05 | 0.000583581 | 0.171309192 | 2.520658255 |
| 2 | MALAT1 | 4.079178333 | 0.215242431 | 4.64276E−05 | 0.000592241 | 1 | 6.245911121 |
| 2 | GPA33 | 4.068855286 | 0.989072084 | 4.87057E−05 | 0.000617744 | 0.071030641 | 2.392682314 |
| 2 | PDZK1 | 4.051550865 | 1.068580389 | 5.24338E−05 | 0.000660815 | 0.063370474 | 2.321617842 |
| 2 | RP11-752G15.8 | 4.048782825 | 1.110365987 | 5.30743E−05 | 0.000667737 | 0.058495822 | 2.335519314 |
| 2 | SYNPO2 | 4.046543121 | 0.523881972 | 5.33878E−05 | 0.000670729 | 0.263927577 | 2.553916454 |
| 2 | RP11-973F15.2 | 4.033205509 | 2.257255077 | 5.7173E−05 | 0.000711558 | 0.021587744 | 2.274414063 |
| 2 | MFSD11 | 4.029252529 | 0.605549634 | 5.74665E−05 | 0.000713873 | 0.183844011 | 2.406802893 |
| 2 | TAS1R1 | 4.025084019 | 0.975622296 | 5.86229E−05 | 0.00072688 | 0.072423398 | 2.353468657 |
| 2 | GRIP2 | 4.010518074 | 0.98899579 | 6.236E−05 | 0.000769628 | 0.068941504 | 2.334583998 |
| 2 | UGP2 | 4.002491474 | 0.53459239 | 6.43116E−05 | 0.000791875 | 0.240947075 | 2.477511883 |
| 2 | PRKAA2 | 3.999798536 | 0.875177681 | 6.51687E−05 | 0.000801315 | 0.086350975 | 2.348588705 |
| 2 | HSDL2 | 3.999579668 | 0.647527456 | 6.51423E−05 | 0.000801315 | 0.155988858 | 2.478396416 |
| 2 | RAPSN | 3.99588418 | 1.119654059 | 6.63892E−05 | 0.000814064 | 0.055013928 | 2.384639978 |
| 2 | C10orf71 | 3.994290829 | 0.589156926 | 6.65796E−05 | 0.000815646 | 0.190807799 | 2.436016321 |
| 2 | UBFD1 | 3.989792347 | 0.798439205 | 6.79526E−05 | 0.000829408 | 0.100278552 | 2.405948162 |
| 2 | CFAP100 | 3.98927784 | 0.563872755 | 6.79969E−05 | 0.000829569 | 0.210306407 | 2.502967596 |
| 2 | RP11-69E11.4 | 3.982481718 | 0.825202763 | 7.0075E−05 | 0.000851016 | 0.093314763 | 2.406761885 |
| 2 | CTD-2555O16.2 | 3.971779585 | 0.902197897 | 7.32852E−05 | 0.000885551 | 0.081476323 | 2.411922932 |
| 2 | CHST15 | 3.968417406 | 1.036563516 | 7.43293E−05 | 0.000897075 | 0.06545961 | 2.31648159 |
| 2 | DCLRE1C | 3.964056253 | 0.720474303 | 7.56097E−05 | 0.000909506 | 0.123955432 | 2.448776245 |
| 2 | CD247 | 3.96282506 | 1.457734346 | 7.65162E−05 | 0.000919525 | 0.036211699 | 2.931155443 |
| 2 | SGCG | 3.958671808 | 0.698097527 | 7.73128E−05 | 0.00092497 | 0.13091922 | 2.393152952 |
| 2 | UNC45B | 3.954483747 | 1.121533871 | 7.88886E−05 | 0.000941405 | 0.055013928 | 2.281417608 |
| 2 | EGLN1 | 3.952862501 | 0.485445887 | 7.9116E−05 | 0.000943572 | 0.301532033 | 2.526679039 |
| 2 | COPS5 | 3.947946072 | 1.215881586 | 8.11264E−05 | 0.00096495 | 0.04735376 | 2.360778332 |
| 2 | GRPEL2-AS1 | 3.945527792 | 2.353130817 | 8.25075E−05 | 0.000979058 | 0.020194986 | 2.308031559 |
| 2 | MYH1 | 3.926732302 | 1.205734372 | 8.83289E−05 | 0.001039458 | 0.05362117 | 2.358515501 |
| 2 | SSUH2 | 3.909875154 | 0.995864451 | 9.47561E−05 | 0.001108225 | 0.066852368 | 2.291912556 |
| 2 | GPR157 | 3.909098387 | 1.100530148 | 9.51285E−05 | 0.001111114 | 0.055013928 | 2.341314077 |
| 2 | UTY | 3.900465727 | 0.550868332 | 9.82774E−05 | 0.001144375 | 0.209610028 | 2.41520381 |
| 2 | PPP6R3 | 3.889937878 | 0.410811394 | 0.000102569 | 0.001186547 | 0.449164345 | 2.671763182 |
| 2 | OARD1 | 3.869753361 | 0.824840486 | 0.00011161 | 0.001279449 | 0.089832869 | 2.395848513 |
| 2 | IGFN1 | 3.86814332 | 1.408191919 | 0.000112759 | 0.001290525 | 0.036211699 | 2.359891415 |
| 2 | CASZ1 | 3.867748737 | 0.691710353 | 0.000112441 | 0.001288368 | 0.124651811 | 2.333895445 |
| 2 | DNAH11 | 3.864104748 | 0.467287391 | 0.000114009 | 0.001302458 | 0.310584958 | 2.537545204 |
| 2 | CS | 3.860281944 | 0.875954509 | 0.000115999 | 0.001322353 | 0.081476323 | 2.305225849 |
| 2 | NPIPB4 | 3.858492136 | 0.65940392 | 0.000116735 | 0.001329029 | 0.13718663 | 2.369610786 |
| 2 | AC005355.1 | 3.854791164 | 0.840429604 | 0.00011865 | 0.001346216 | 0.086350975 | 2.478839636 |
| 2 | MPRIP | 3.831214905 | 0.492717326 | 0.00013031 | 0.00146291 | 0.262534819 | 2.48287487 |
| 2 | ARHGAP11B | 3.818361759 | 0.60826689 | 0.000137313 | 0.001535687 | 0.161559889 | 2.467779875 |
| 2 | LINC01344 | 3.817354202 | 1.145336747 | 0.00013831 | 0.001542297 | 0.050139276 | 2.757253885 |

Cluster 6 Myocyte

| | | | | | | | |
|---|---|---|---|---|---|---|---|
| 6 | MB | 43.02420044 | 6.143981934 | 1.1772E−179 | 3.1295E−175 | 0.996655518 | 4.343101501 |
| 6 | DES | 32.37994766 | 5.416199207 | 2.8265E−133 | 3.757E−129 | 0.956521739 | 3.885333776 |
| 6 | ACTA1 | 27.63533592 | 4.060711861 | 9.1202E−102 | 6.06126E−98 | 1 | 5.163728237 |
| 6 | CKM | 26.99481201 | 4.327866077 | 1.8088E−102 | 1.60283E−98 | 0.983277592 | 4.285290718 |
| 6 | MT-CO1 | 25.74646187 | 4.005593777 | 5.55384E−95 | 2.46072E−91 | 0.986622074 | 4.316649437 |
| 6 | TPM2 | 25.66440964 | 4.226867199 | 1.69313E−97 | 9.00203E−94 | 0.959866221 | 3.729408979 |
| 6 | TNNC1 | 24.38770103 | 4.713791847 | 1.11602E−90 | 4.23833E−87 | 0.899665552 | 3.975837946 |
| 6 | CA3 | 24.05115318 | 4.829266548 | 1.31526E−87 | 3.88499E−84 | 0.876254181 | 3.773276329 |
| 6 | ALDOA | 23.98087311 | 4.505093575 | 2.7341E−88 | 9.08543E−85 | 0.882943144 | 3.37108016 |
| 6 | MYH7 | 23.88047028 | 6.534370899 | 6.47993E−76 | 1.3251E−72 | 0.74916388 | 3.606462479 |
| 6 | MT-CO3 | 23.67384911 | 3.743752718 | 8.05654E−85 | 1.94705E−81 | 0.986622074 | 3.94788003 |
| 6 | TNNT1 | 23.16947556 | 4.4898386 | 7.32256E−85 | 1.94663E−81 | 0.903010033 | 4.22561121 |
| 6 | TCAP | 21.75367737 | 4.740222454 | 2.46088E−74 | 4.67286E−71 | 0.822742475 | 3.747442722 |
| 6 | GAPDH | 21.58976364 | 4.005534649 | 1.05976E−76 | 2.34772E−73 | 0.906354515 | 3.563717604 |
| 6 | TNNI1 | 21.47883606 | 4.925431728 | 1.56552E−71 | 2.4481E−68 | 0.79264214 | 3.604704142 |
| 6 | RPLP1 | 21.09957123 | 4.024948597 | 3.78187E−74 | 6.70248E−71 | 0.892976589 | 3.56896162 |
| 6 | MT-CO2 | 20.8260231 | 3.804091692 | 8.7656E−73 | 1.4564E−69 | 0.909698997 | 3.629992962 |
| 6 | CRYAB | 20.23134041 | 4.312905788 | 3.94859E−67 | 5.24847E−64 | 0.789297659 | 3.116901875 |
| 6 | MYL2 | 20.14282799 | 3.767322302 | 3.33314E−69 | 4.92268E−66 | 0.933110368 | 4.204955101 |
| 6 | COX6A2 | 20.02323914 | 4.165132046 | 5.69924E−67 | 7.21469E−64 | 0.802675585 | 3.14183712 |
| 6 | TPT1 | 19.78376389 | 3.629775763 | 2.43434E−67 | 3.40602E−64 | 0.89632107 | 3.324299335 |
| 6 | NDUFA4 | 19.63005829 | 4.866504192 | 3.03708E−61 | 3.66989E−58 | 0.698996656 | 2.838260174 |
| 6 | MT-CYB | 18.56700134 | 3.772286415 | 2.01924E−60 | 2.33389E−57 | 0.819397993 | 3.204665899 |
| 6 | FHL1 | 18.00640297 | 3.587930918 | 7.10621E−58 | 7.87131E−55 | 0.816053512 | 3.148322821 |
| 6 | ENO3 | 17.98171425 | 4.319149971 | 9.71856E−55 | 9.93686E−52 | 0.695652174 | 2.989589691 |
| 6 | SLC25A4 | 17.53217888 | 4.345602512 | 1.71586E−52 | 1.57291E−49 | 0.668896321 | 2.827461481 |
| 6 | MT-ND1 | 17.48502922 | 3.407728672 | 2.27765E−55 | 2.42196E−52 | 0.842809365 | 3.032212734 |
| 6 | HSPB1 | 17.30301285 | 4.0241189 | 1.55439E−52 | 1.47578E−49 | 0.712374582 | 3.04609704 |
| 6 | MYL1 | 17.05532837 | 3.573392153 | 9.25479E−53 | 9.11219E−50 | 0.799331104 | 3.247766495 |
| 6 | COX7A1 | 16.98825645 | 4.131437778 | 1.85492E−50 | 1.6437E−47 | 0.665551839 | 2.717325687 |
| 6 | COX7C | 16.68403053 | 3.916766882 | 1.25227E−49 | 1.0088E−46 | 0.678929766 | 2.737014771 |
| 6 | PYGM | 16.64027405 | 4.543382645 | 1.40538E−47 | 1.09884E−44 | 0.608695652 | 2.624840498 |
| 6 | TPM3 | 16.41174126 | 3.288799047 | 3.56829E−50 | 3.05998E−47 | 0.852842809 | 3.561155796 |
| 6 | MT-ATP6 | 16.37840652 | 3.388705492 | 1.23533E−49 | 1.0088E−46 | 0.785953177 | 3.024434328 |
| 6 | MYL3 | 16.37046432 | 4.489688873 | 1.17428E−46 | 8.67144E−44 | 0.62541806 | 3.198951244 |
| 6 | RP11-451G4.2 | 15.95181751 | 3.591951847 | 8.25596E−47 | 6.27075E−44 | 0.719063545 | 3.06833005 |
| 6 | MT-ND4 | 15.5667429 | 3.214053869 | 7.22516E−46 | 5.19118E−43 | 0.806020067 | 3.215845585 |
| 6 | TNNC2 | 15.54933453 | 4.401704311 | 3.02322E−43 | 2.11498E−40 | 0.655518395 | 3.784383535 |
| 6 | KLHL41 | 15.29343414 | 3.840161562 | 1.03261E−42 | 7.03871E−40 | 0.622073579 | 2.718484402 |
| 6 | SLN | 14.99790573 | 4.610948086 | 3.59912E−40 | 2.2251E−37 | 0.538461538 | 2.66330719 |
| 6 | FABP3 | 14.90071583 | 4.162660122 | 2.28079E−40 | 1.47884E−37 | 0.571906355 | 2.850380659 |
| 6 | MYLPF | 14.86269951 | 4.295003891 | 3.50172E−40 | 2.21642E−37 | 0.615384615 | 3.517362118 |
| 6 | S100A1 | 14.85147953 | 4.476154327 | 9.48395E−40 | 5.60269E−37 | 0.545150502 | 2.577243567 |
| 6 | EIF1 | 14.7449913 | 3.045196772 | 4.27722E−42 | 2.84264E−39 | 0.779264214 | 2.977620363 |
| 6 | TTN | 14.42502499 | 2.672262192 | 9.18618E−40 | 5.55012E−37 | 0.956521739 | 4.013521671 |
| 6 | CHCHD10 | 14.20298672 | 3.66378355 | 4.59116E−38 | 2.65329E−35 | 0.585284281 | 2.639450073 |
| 6 | COX6C | 14.18956184 | 4.335117817 | 4.01513E−37 | 2.17833E−34 | 0.52173913 | 2.610847235 |
| 6 | MT2A | 13.85520935 | 2.876731873 | 5.09472E−38 | 2.88166E−35 | 0.862876254 | 3.568318129 |
| 6 | CHCHD3 | 13.84421921 | 2.818068504 | 7.80309E−38 | 4.32161E−35 | 0.759197324 | 2.912197828 |
| 6 | MYOZ1 | 13.83152294 | 3.471854925 | 1.00504E−36 | 5.3436E−34 | 0.595317726 | 2.623748779 |
| 6 | COX7B | 13.79362965 | 4.323702812 | 2.02619E−35 | 9.79349E−33 | 0.501672241 | 2.610562801 |
| 6 | COX5B | 13.77100277 | 3.899089336 | 7.44234E−36 | 3.73296E−33 | 0.538461538 | 2.705583096 |
| 6 | COX5A | 13.5819006 | 4.139304161 | 9.11201E−35 | 4.3256E−32 | 0.508361204 | 2.489631653 |
| 6 | UQCRQ | 13.54492664 | 4.321259022 | 2.00366E−34 | 9.34477E−32 | 0.498327759 | 2.57087779 |
| 6 | MT-ND3 | 13.4827013 | 2.444972515 | 2.60461E−36 | 1.35767E−33 | 0.903010033 | 3.471642733 |
| 6 | YBX3 | 13.37496567 | 2.578788996 | 7.4124E−36 | 3.73296E−33 | 0.826086957 | 3.20269084 |
| 6 | CALM1 | 13.17319489 | 3.72787714 | 1.86154E−33 | 8.53227E−31 | 0.518394649 | 2.602033138 |
| 6 | SEPW1 | 12.94436169 | 3.410784245 | 7.41761E−33 | 3.23262E−30 | 0.555183946 | 2.57120657 |
| 6 | RPL19 | 12.82923222 | 2.995602369 | 5.17013E−33 | 2.29071E−30 | 0.635451505 | 2.73812294 |
| 6 | RPS2 | 12.78335285 | 2.888932228 | 4.83667E−33 | 2.17929E−30 | 0.685618729 | 2.864095449 |
| 6 | UQCRB | 12.72142982 | 3.331959009 | 4.75268E−32 | 2.00548E−29 | 0.555183946 | 2.544864416 |
| 6 | FLNC | 12.69698906 | 4.30724144 | 6.66016E−31 | 2.64259E−28 | 0.454849498 | 2.487817287 |
| 6 | RPL13 | 12.65026855 | 2.698569536 | 1.31957E−32 | 5.65796E−30 | 0.735785953 | 2.9123981 |
| 6 | MYL12A | 12.4918766 | 3.072890759 | 2.24041E−31 | 9.3061E−29 | 0.595317726 | 2.62784934 |
| 6 | MT-ND2 | 12.32853127 | 2.768182516 | 5.14231E−31 | 2.07126E−28 | 0.682274247 | 2.889026642 |
| 6 | UBC | 12.30245972 | 2.501234293 | 3.73982E−31 | 1.52953E−28 | 0.79264214 | 3.028244972 |
| 6 | RPS23 | 12.26102638 | 2.908042192 | 1.2974E−30 | 5.07205E−28 | 0.628762542 | 2.821702957 |
| 6 | COX4I1 | 12.24426365 | 3.507344007 | 8.28253E−30 | 3.14547E−27 | 0.494983278 | 2.605221748 |
| 6 | ATP5B | 12.22485161 | 4.168096542 | 3.55513E−29 | 1.29465E−26 | 0.43812709 | 2.482934475 |
| 6 | RPL10 | 12.08850574 | 2.54285264 | 3.35818E−30 | 1.29382E−27 | 0.74916388 | 3.010930538 |
| 6 | RPL8 | 12.04381561 | 2.986783743 | 1.39999E−29 | 5.24189E−27 | 0.578595318 | 2.686583519 |
| 6 | PRDX6 | 11.95144081 | 3.183866262 | 6.7295E−29 | 2.41753E−26 | 0.525083612 | 2.514933825 |
| 6 | RPS15 | 11.91438389 | 2.755200386 | 2.64042E−29 | 9.74903E−27 | 0.638795987 | 2.713558674 |
| 6 | UQCR10 | 11.81538773 | 3.887508392 | 8.87366E−28 | 3.02433E−25 | 0.441471572 | 2.406711102 |
| 6 | BIN1 | 11.77005196 | 2.744000912 | 1.39647E−28 | 4.94983E−26 | 0.612040134 | 2.604118586 |
| 6 | COX6B1 | 11.74778175 | 3.68074441 | 1.18484E−27 | 3.93721E−25 | 0.451505017 | 2.535528898 |
| 6 | ATP5D | 11.71023178 | 3.431571484 | 1.07318E−27 | 3.61132E−25 | 0.47826087 | 2.461026192 |
| 6 | FTH1 | 11.67383289 | 2.459845543 | 1.70226E−28 | 5.95434E−26 | 0.775919732 | 3.109295607 |
| 6 | TPI1 | 11.5807333 | 4.033297539 | 9.22191E−27 | 2.91852E−24 | 0.414715719 | 2.377492189 |
| 6 | RPL37A | 11.56650734 | 2.709009171 | 8.70268E−28 | 3.00457E−25 | 0.62541806 | 2.805609226 |
| 6 | ATP5J | 11.54715443 | 3.779857874 | 8.87651E−27 | 2.87772E−24 | 0.431438127 | 2.485822916 |
| 6 | SLC25A3 | 11.49835873 | 3.65532136 | 1.13528E−26 | 3.50933E−24 | 0.441471572 | 2.445956945 |
| 6 | TNNI2 | 11.46724033 | 4.194161415 | 3.5535E−26 | 1.02681E−23 | 0.421404682 | 3.161053419 |
| 6 | VDAC1 | 11.41171265 | 3.62885046 | 2.40879E−26 | 7.19499E−24 | 0.43812709 | 2.429177999 |
| 6 | EEF2 | 11.31046677 | 2.959679604 | 1.54479E−26 | 4.72032E−24 | 0.531772575 | 2.566192627 |
| 6 | RPL23A | 11.30903625 | 2.782762051 | 1.0736E−26 | 3.35772E−24 | 0.575250836 | 2.552354336 |
| 6 | RPL41 | 11.26671219 | 2.478553534 | 8.86275E−27 | 2.87772E−24 | 0.7090301 | 2.967594624 |
| 6 | CHCHD2 | 11.25250435 | 2.741092443 | 1.99753E−26 | 6.03434E−24 | 0.565217391 | 2.526337862 |
| 6 | HSPA8 | 11.22584152 | 4.464906693 | 3.15714E−25 | 8.30985E−23 | 0.37458194 | 2.426956415 |
| 6 | RPS6 | 11.21953678 | 2.815506697 | 2.6334E−26 | 7.77848E−24 | 0.565217391 | 2.685528994 |
| 6 | RPL32 | 11.18045044 | 2.657191515 | 2.8178E−26 | 8.23169E−24 | 0.608695652 | 2.728359699 |
| 6 | HSP90AB1 | 11.17395687 | 3.872803688 | 2.68793E−25 | 7.21776E−23 | 0.408026756 | 2.442408085 |
| 6 | RPL35 | 11.13821602 | 2.843528986 | 6.16641E−26 | 1.74391E−23 | 0.545150502 | 2.586093426 |
| 6 | ATP5G3 | 11.11828899 | 3.60660696 | 3.01505E−25 | 8.01521E−23 | 0.424749164 | 2.429944515 |
| 6 | LDHA | 11.11703205 | 3.356002092 | 2.21407E−25 | 6.00602E−23 | 0.454849498 | 2.540354013 |
| 6 | BDNF-AS | 11.08764267 | 2.647432327 | 6.96026E−26 | 1.9477E−23 | 0.602006689 | 2.768410921 |
| 6 | RPL13A | 11.08376503 | 2.490677834 | 5.24074E−26 | 1.49806E−23 | 0.668896321 | 2.90099597 |
| 6 | RPL7 | 11.00989151 | 2.744934082 | 1.71755E−25 | 4.75618E−23 | 0.565217391 | 2.720552683 |
| 6 | BAIAP2L1 | 11.00104809 | 2.716681004 | 1.83196E−25 | 5.02071E−23 | 0.56187291 | 2.533944845 |
| 6 | RPS27A | 10.85273838 | 2.649956465 | 5.96224E−25 | 1.55392E−22 | 0.581939799 | 2.679792404 |
| 6 | RPS25 | 10.8289957 | 2.511637926 | 6.43929E−25 | 1.66196E−22 | 0.62541806 | 2.831284046 |
| 6 | RPL21 | 10.78038883 | 2.599620819 | 1.12704E−24 | 2.88088E−22 | 0.581939799 | 2.624423027 |
| 6 | RPLP0 | 10.65667915 | 2.655169964 | 3.86682E−24 | 9.79006E−22 | 0.558528428 | 2.620710135 |
| 6 | EIF4A2 | 10.63757133 | 2.865361452 | 6.91092E−24 | 1.70111E−21 | 0.498327759 | 2.554018736 |
| 6 | CFL2 | 10.63415909 | 3.470540285 | 1.78101E−23 | 4.3437E−21 | 0.408026756 | 2.408584356 |
| 6 | RPS16 | 10.62243271 | 2.708756447 | 5.91121E−24 | 1.46863E−21 | 0.54180602 | 2.729650259 |
| 6 | SMPX | 10.61978245 | 4.297241211 | 4.51261E−23 | 1.05231E−20 | 0.35451505 | 2.2745893 |
| 6 | XPO5 | 10.61269855 | 2.501939058 | 4.43124E−24 | 1.11132E−21 | 0.612040134 | 2.686529875 |
| 6 | CYCS | 10.57091427 | 3.639368534 | 3.48038E−23 | 8.26092E−21 | 0.397993311 | 2.366087437 |
| 6 | TOMM7 | 10.48166561 | 3.006508112 | 3.3534E−23 | 8.03123E−21 | 0.45819398 | 2.433679342 |
| 6 | RPL4 | 10.46050549 | 3.092139721 | 4.50891E−23 | 1.05231E−20 | 0.441471572 | 2.515724421 |
| 6 | SOD1 | 10.45531178 | 3.855461359 | 1.17812E−22 | 2.69993E−20 | 0.367892977 | 2.330346823 |
| 6 | MDH2 | 10.36954308 | 4.070189953 | 2.9704E−22 | 6.58043E−20 | 0.351170569 | 2.480656385 |
| 6 | NDUFB10 | 10.29617405 | 3.482399464 | 2.94375E−22 | 6.57618E−20 | 0.391304348 | 2.366122723 |
| 6 | TNNT3 | 10.24332619 | 3.027426481 | 2.70397E−22 | 6.09173E−20 | 0.494983278 | 3.335701704 |
| 6 | SOD2 | 10.23558331 | 2.292965889 | 1.13644E−22 | 2.62706E−20 | 0.675585284 | 3.014807701 |
| 6 | RPS5 | 10.21756268 | 2.774952888 | 2.31413E−22 | 5.25802E−20 | 0.488294314 | 2.438194513 |
| 6 | ATP5A1 | 10.19924355 | 4.087312698 | 1.16026E−21 | 2.42869E−19 | 0.337792642 | 2.373940229 |
| 6 | NDUFB2 | 10.19239521 | 3.76978159 | 9.85315E−22 | 2.09549E−19 | 0.357859532 | 2.425098419 |
| 6 | MYL6B | 10.19212723 | 3.316159725 | 6.59044E−22 | 1.43607E−19 | 0.397993311 | 2.490686655 |
| 6 | FHL3 | 10.16584396 | 3.125020981 | 6.87511E−22 | 1.48592E−19 | 0.414715719 | 2.446197748 |
| 6 | FAU | 10.14793205 | 2.654789925 | 3.64796E−22 | 8.01467E−20 | 0.508361204 | 2.544695616 |
| 6 | SKP1 | 10.12356949 | 2.888655424 | 7.25558E−22 | 1.5555E−19 | 0.454849498 | 2.622248173 |
| 6 | C8orf22 | 10.07701111 | 3.034464359 | 1.302E−21 | 2.70409E−19 | 0.434782609 | 2.928212881 |
| 6 | NDUFB9 | 10.00150967 | 2.594003916 | 1.34558E−21 | 2.77293E−19 | 0.505016722 | 2.473326445 |
| 6 | RPS24 | 9.999866486 | 2.532093048 | 1.15077E−21 | 2.42794E−19 | 0.531772575 | 2.653804779 |
| 6 | AK1 | 9.999616623 | 3.716363192 | 4.81813E−21 | 9.70342E−19 | 0.35451505 | 2.449182034 |
| 6 | ECH1 | 9.961932182 | 4.239552498 | 8.89731E−21 | 1.70163E−18 | 0.327759197 | 2.327424765 |
| 6 | NDUFA1 | 9.920082092 | 3.48846364 | 7.06311E−21 | 1.38063E−18 | 0.361204013 | 2.434457302 |
| 6 | OOEP | 9.916172981 | 2.611107588 | 2.62951E−21 | 5.37714E−19 | 0.498327759 | 2.57228899 |
| 6 | UQCRFS1 | 9.903003693 | 3.680698633 | 9.63014E−21 | 1.82863E−18 | 0.351170569 | 2.315984249 |
| 6 | RPS13 | 9.886984825 | 2.566643715 | 3.17213E−21 | 6.43724E−19 | 0.511705686 | 2.625119925 |
| 6 | MYF6 | 9.852994919 | 2.980843544 | 7.94942E−21 | 1.53136E−18 | 0.434782609 | 2.587153673 |
| 6 | PKM | 9.825388908 | 3.201445341 | 1.18309E−20 | 2.19938E−18 | 0.391304348 | 2.395657778 |
| 6 | RPL29 | 9.805163383 | 2.579823971 | 6.5665E−21 | 1.30272E−18 | 0.501672241 | 2.603920221 |
| 6 | ANKRD2 | 9.79777813 | 2.59807992 | 5.72271E−21 | 1.14385E−18 | 0.545150502 | 2.964773178 |
| 6 | APOO | 9.783823967 | 2.466961622 | 6.85261E−21 | 1.34941E−18 | 0.52173913 | 2.548393488 |
| 6 | IDH2 | 9.780405998 | 2.922806501 | 1.37008E−20 | 2.51187E−18 | 0.421404682 | 2.471904278 |
| 6 | RPS9 | 9.760458946 | 2.431060076 | 7.7572E−21 | 1.50524E−18 | 0.535117057 | 2.48966527 |
| 6 | ZNF90 | 9.750902176 | 2.685995102 | 1.19708E−20 | 2.20994E−18 | 0.468227425 | 2.695208549 |
| 6 | POLR2L | 9.746346474 | 3.354137421 | 2.39999E−20 | 4.22525E−18 | 0.364548495 | 2.33130312 |
| 6 | RPS3 | 9.734286308 | 2.599705696 | 1.18144E−20 | 2.19938E−18 | 0.491638796 | 2.565977812 |
| 6 | RPS14 | 9.725390434 | 2.405074358 | 1.02622E−20 | 1.93483E−18 | 0.548494983 | 2.552279949 |
| 6 | RPL30 | 9.702306747 | 2.461541653 | 1.38175E−20 | 2.51591E−18 | 0.518394649 | 2.605905771 |
| 6 | ADCK3 | 9.698839188 | 2.361030817 | 1.41481E−20 | 2.5586E−18 | 0.538461538 | 2.539010048 |
| 6 | ATP5G2 | 9.690031052 | 2.877333164 | 2.56174E−20 | 4.42217E−18 | 0.428093645 | 2.467015982 |
| 6 | LINC01405 | 9.687637329 | 3.477339745 | 4.47508E−20 | 7.48211E−18 | 0.35451505 | 2.427163839 |
| 6 | NDRG2 | 9.66271019 | 2.617923021 | 2.44193E−20 | 4.27081E−18 | 0.471571906 | 2.443942308 |
| 6 | RPL27 | 9.655572891 | 2.723209381 | 2.75735E−20 | 4.72911E−18 | 0.454849498 | 2.545369864 |
| 6 | RPL27A | 9.652393341 | 2.382420301 | 1.92088E−20 | 3.42716E−18 | 0.545150502 | 2.629798651 |
| 6 | RPLP2 | 9.593766212 | 2.086982965 | 2.46513E−20 | 4.2832E−18 | 0.658862876 | 2.661885738 |
| 6 | TXNRD1 | 9.576096535 | 2.160620213 | 3.01047E−20 | 5.13015E−18 | 0.618729097 | 2.673116922 |
| 6 | NEB | 9.540171623 | 2.003501415 | 4.25307E−20 | 7.15592E−18 | 0.869565217 | 3.666754484 |
| 6 | C14orf2 | 9.531763077 | 3.060477972 | 1.10498E−19 | 1.83593E−17 | 0.381270903 | 2.381160498 |
| 6 | HINT1 | 9.473603249 | 2.943382978 | 1.57908E−19 | 2.60734E−17 | 0.397993311 | 2.410817146 |
| 6 | CYC1 | 9.462348938 | 3.585278034 | 3.02048E−19 | 4.92617E−17 | 0.327759197 | 2.35316968 |
| 6 | UBB | 9.38723278 | 3.153939962 | 3.75488E−19 | 6.04968E−17 | 0.361204013 | 2.308714867 |
| 6 | RPS3A | 9.343842506 | 2.666898727 | 3.422E−19 | 5.54698E−17 | 0.43812709 | 2.411174536 |
| 6 | RPL10A | 9.250132561 | 2.303757191 | 5.08268E−19 | 8.09089E−17 | 0.525083612 | 2.500653505 |
| 6 | NDUFB4 | 9.24742794 | 3.305349588 | 1.32706E−18 | 2.08749E−16 | 0.344481605 | 2.41409874 |
| 6 | HSPB6 | 9.21066761 | 3.943265915 | 2.62663E−18 | 3.94499E−16 | 0.297658863 | 2.220843554 |
| 6 | YBX1 | 9.185613632 | 2.613735199 | 1.17137E−18 | 1.85355E−16 | 0.43812709 | 2.41982317 |
| 6 | RPL38 | 9.155163765 | 2.506020784 | 1.37368E−18 | 2.14811E−16 | 0.45819398 | 2.615750313 |
| 6 | ATP5E | 9.148055077 | 2.877767563 | 1.94531E−18 | 2.98925E−16 | 0.387959866 | 2.313272953 |
| 6 | MIR1-1HG | 9.144955635 | 3.410049915 | 3.15134E−18 | 4.68018E−16 | 0.331103679 | 2.191933632 |
| 6 | RPS15A | 9.128898621 | 2.48785305 | 1.66911E−18 | 2.59483E−16 | 0.468227425 | 2.683866024 |
| 6 | UBA52 | 9.127540588 | 2.627494812 | 1.89042E−18 | 2.9218E−16 | 0.431438127 | 2.435302496 |
| 6 | KDM5B | 9.11771965 | 2.641044378 | 2.14894E−18 | 3.26443E−16 | 0.418060201 | 2.396849871 |
| 6 | RPL7A | 9.10686779 | 2.534318686 | 2.09167E−18 | 3.19569E−16 | 0.448160535 | 2.539223433 |
| 6 | RPS19 | 9.064502716 | 2.36310792 | 2.42064E−18 | 3.65626E−16 | 0.494983278 | 2.615932703 |
| 6 | SRP14 | 9.0343256 | 2.85013032 | 4.7019E−18 | 6.90582E−16 | 0.377926421 | 2.307368517 |
| 6 | PRDX1 | 9.032553673 | 3.382690907 | 7.37652E−18 | 1.05999E−15 | 0.321070234 | 2.338950396 |
| 6 | NDUFB11 | 9.021365166 | 3.4148736 | 8.16692E−18 | 1.16725E−15 | 0.321070234 | 2.32764554 |
| 6 | RPS11 | 9.001734734 | 2.534461737 | 4.79605E−18 | 7.0054E−16 | 0.43812709 | 2.461723804 |
| 6 | RPS8 | 8.994083405 | 2.427566528 | 4.68195E−18 | 6.90582E−16 | 0.468227425 | 2.575674057 |
| 6 | RPS18 | 8.980336189 | 2.394661188 | 4.97993E−18 | 7.23424E−16 | 0.481605351 | 2.65426898 |
| 6 | RPL35A | 8.952508926 | 2.410307884 | 6.23174E−18 | 9.0035E−16 | 0.471571906 | 2.558992386 |
| 6 | ATP5G1 | 8.942605019 | 3.408504248 | 1.46842E−17 | 2.0438E−15 | 0.317725753 | 2.331415415 |
| 6 | LGALS1 | 8.929722786 | 2.895905256 | 1.12383E−17 | 1.57562E−15 | 0.371237458 | 2.327907801 |
| 6 | MYH2 | 8.922102928 | 5.78375864 | 3.98185E−17 | 5.34613E−15 | 0.240802676 | 2.939482689 |
| 6 | RAD23A | 8.915341377 | 2.70595336 | 1.12612E−17 | 1.57562E−15 | 0.387959866 | 2.3273139 |
| 6 | GNB2L1 | 8.904453278 | 2.396733284 | 9.17068E−18 | 1.30371E−15 | 0.468227425 | 2.476001024 |
| 6 | EEF1A2 | 8.894926071 | 4.036234856 | 3.12181E−17 | 4.23419E−15 | 0.277591973 | 2.435409784 |
| 6 | NDUFS7 | 8.826827049 | 3.415465593 | 3.57073E−17 | 4.8185E−15 | 0.311036789 | 2.24753499 |
| 6 | RPL6 | 8.795046806 | 2.430459261 | 2.24817E−17 | 3.08069E−15 | 0.454849498 | 2.478968143 |
| 6 | RPS27 | 8.792427063 | 2.171960831 | 1.77242E−17 | 2.44135E−15 | 0.555183946 | 2.627758265 |
| 6 | GLUL | 8.788108826 | 2.048192978 | 1.7414E−17 | 2.41112E−15 | 0.62541806 | 2.930974722 |
| 6 | RPL15 | 8.730522156 | 2.142110109 | 2.96821E−17 | 4.04651E−15 | 0.531772575 | 2.578978062 |
| 6 | PSMA7 | 8.686385155 | 2.872271299 | 7.36398E−17 | 9.83739E−15 | 0.344481605 | 2.263373852 |
| 6 | RPS20 | 8.638057709 | 2.367312193 | 7.45475E−17 | 9.90885E−15 | 0.448160535 | 2.491621017 |
| 6 | EEF1D | 8.636657715 | 2.62100482 | 9.02056E−17 | 1.18714E−14 | 0.384615385 | 2.380946159 |
| 6 | STAC3 | 8.609723091 | 2.241464376 | 8.89864E−17 | 1.17692E−14 | 0.468227425 | 2.371187925 |
| 6 | RPL24 | 8.588702202 | 2.446411133 | 1.14626E−16 | 1.50109E−14 | 0.418060201 | 2.393344641 |
| 6 | NDUFAB1 | 8.585981369 | 3.288400173 | 2.08073E−16 | 2.71148E−14 | 0.301003344 | 2.330874443 |
| 6 | LINC00116 | 8.494636536 | 3.572379827 | 4.53924E−16 | 5.77374E−14 | 0.284280936 | 2.212577105 |
| 6 | NDUFB3 | 8.487772942 | 3.78593874 | 5.18257E−16 | 6.56064E−14 | 0.267558528 | 2.276000023 |
| 6 | XIRP2 | 8.464289665 | 2.926943779 | 4.2838E−16 | 5.50148E−14 | 0.331103679 | 2.528687239 |
| 6 | PGAM2 | 8.450993538 | 3.256779671 | 5.34395E−16 | 6.73287E−14 | 0.294314381 | 2.335208654 |
| 6 | TPM1 | 8.43659687 | 2.417126179 | 3.77965E−16 | 4.87758E−14 | 0.461538462 | 2.917251348 |
| 6 | PARK7 | 8.41064167 | 3.129706621 | 6.81842E−16 | 8.55004E−14 | 0.304347826 | 2.28066349 |
| 6 | RPS4X | 8.390590668 | 2.223051786 | 4.3717E−16 | 5.58737E−14 | 0.474916388 | 2.624942303 |
| 6 | RRAD | 8.362243652 | 2.925583124 | 8.77866E−16 | 1.09564E−13 | 0.324414716 | 2.305049419 |
| 6 | RPL23 | 8.298952103 | 2.525326967 | 1.06519E−15 | 1.32323E−13 | 0.381270903 | 2.334743738 |
| 6 | AKR1C3 | 8.298778534 | 4.325381756 | 2.46403E−15 | 2.99104E−13 | 0.240802676 | 2.399517775 |
| 6 | HSP90AA1 | 8.260081291 | 2.857938051 | 1.8418E−15 | 2.25633E−13 | 0.327759197 | 2.49747777 |
| 6 | RPL34 | 8.233561516 | 2.106270075 | 1.3158E−15 | 1.62694E−13 | 0.498327759 | 2.626796007 |
| 6 | MRPL41 | 8.19377327 | 3.487635612 | 3.90672E−15 | 4.72074E−13 | 0.27090301 | 2.303924322 |
| 6 | ACADVL | 8.193453789 | 2.256564617 | 2.08209E−15 | 2.539E−13 | 0.424749164 | 2.337881088 |
| 6 | JSRP1 | 8.165268898 | 3.420452833 | 4.64162E−15 | 5.58338E−13 | 0.267558528 | 2.225161552 |
| 6 | G0S2 | 8.094220161 | 3.773112059 | 8.64383E−15 | 1.02128E−12 | 0.257525084 | 2.263516188 |
| 6 | SBDS | 8.081910133 | 3.048756599 | 7.08964E−15 | 8.41388E−13 | 0.301003344 | 2.24751687 |
| 6 | NDUFV2 | 8.078515053 | 3.018662214 | 7.01192E−15 | 8.35897E−13 | 0.294314381 | 2.182322025 |
| 6 | MYH1 | 8.065725327 | 4.478069305 | 1.36659E−14 | 1.5727E−12 | 0.234113712 | 3.026050329 |
| 6 | EEF1B2 | 8.043294907 | 2.955038071 | 8.91544E−15 | 1.04871E−12 | 0.304347826 | 2.314278841 |
| 6 | NDUFA6 | 8.036384583 | 3.46351099 | 1.15459E−14 | 1.34621E−12 | 0.260869565 | 2.231997967 |
| 6 | CNBP | 8.031626701 | 2.210984707 | 6.76196E−15 | 8.0973E−13 | 0.424749164 | 2.436335087 |
| 6 | EIF3K | 8.005523682 | 2.908904552 | 1.13144E−14 | 1.32504E−12 | 0.301003344 | 2.197974205 |
| 6 | UQCRH | 7.996405602 | 2.819694996 | 1.17231E−14 | 1.3609E−12 | 0.314381271 | 2.225724936 |
| 6 | SH3BGR | 7.969733238 | 3.668721914 | 2.06724E−14 | 2.36877E−12 | 0.25083612 | 2.243388414 |
| 6 | GAMT | 7.922488213 | 3.528636932 | 2.64214E−14 | 3.01454E−12 | 0.25083612 | 2.296684504 |
| 6 | HSPB8 | 7.855412483 | 2.427560329 | 2.74958E−14 | 3.12371E−12 | 0.361204013 | 2.318192482 |
| 6 | PEBP1 | 7.824744225 | 2.527966499 | 3.51596E−14 | 3.97738E−12 | 0.344481605 | 2.29316926 |
| 6 | VDAC2 | 7.800396442 | 2.960738897 | 4.91395E−14 | 5.51192E−12 | 0.287625418 | 2.20093894 |
| 6 | RPL18 | 7.754321098 | 2.201371908 | 4.78351E−14 | 5.38834E−12 | 0.408026756 | 2.377022266 |
| 6 | NDUFS5 | 7.717818737 | 2.590960741 | 7.5077E−14 | 8.38592E−12 | 0.324414716 | 2.279595137 |
| 6 | RPS12 | 7.659885406 | 2.10131073 | 8.91231E−14 | 9.87187E−12 | 0.428093645 | 2.392581463 |
| 6 | RPS7 | 7.65297842 | 2.300752163 | 1.04121E−13 | 1.14853E−11 | 0.371237458 | 2.431871176 |
| 6 | NDUFB7 | 7.647305965 | 3.163480759 | 1.52251E−13 | 1.6725E−11 | 0.260869565 | 2.138025045 |
| 6 | RPL14 | 7.559394836 | 2.032038212 | 1.75949E−13 | 1.90915E−11 | 0.441471572 | 2.483638048 |
| 6 | SERF2 | 7.556188107 | 1.935291886 | 1.74818E−13 | 1.90466E−11 | 0.471571906 | 2.479226351 |
| 6 | PPP1R1A | 7.545084476 | 4.023424149 | 3.87641E−13 | 4.15527E−11 | 0.214046823 | 2.160367012 |
| 6 | RPL28 | 7.541971207 | 2.101621151 | 2.04228E−13 | 2.207E−11 | 0.421404682 | 2.523748398 |
| 6 | GYG1 | 7.479449272 | 2.408244371 | 3.76073E−13 | 4.04758E−11 | 0.327759197 | 2.269204378 |
| 6 | UQCRC1 | 7.454180241 | 3.530325651 | 6.39532E−13 | 6.71989E−11 | 0.227424749 | 2.164545774 |
| 6 | ATP5L | 7.430192947 | 2.280918598 | 4.87847E−13 | 5.18757E−11 | 0.35451505 | 2.292150021 |
| 6 | RPL36 | 7.416443348 | 2.12958312 | 4.87065E−13 | 5.18757E−11 | 0.391304348 | 2.440320015 |
| 6 | ECHS1 | 7.389153957 | 4.440092564 | 1.16578E−12 | 1.20588E−10 | 0.197324415 | 2.17389226 |
| 6 | RPS29 | 7.37937212 | 2.205579281 | 6.75267E−13 | 7.06744E−11 | 0.367892977 | 2.473978043 |
| 6 | TCEB2 | 7.376532078 | 2.871729851 | 8.6979E−13 | 9.06764E−11 | 0.267558528 | 2.301090002 |
| 6 | HFE2 | 7.319180965 | 4.704819679 | 1.92637E−12 | 1.95461E−10 | 0.18729097 | 2.198435068 |
| 6 | GSTP1 | 7.306927681 | 2.746891737 | 1.31587E−12 | 1.35062E−10 | 0.277591973 | 2.188621998 |
| 6 | USMG5 | 7.293880939 | 2.461455345 | 1.28475E−12 | 1.32379E−10 | 0.314381271 | 2.197336674 |
| 6 | RPL5 | 7.264683247 | 2.070310593 | 1.38968E−12 | 1.42089E−10 | 0.387959866 | 2.366861105 |
| 6 | CASQ1 | 7.263728142 | 2.996763468 | 1.96085E−12 | 1.98202E−10 | 0.247491639 | 2.112336159 |
| 6 | AURKAIP1 | 7.236795425 | 3.55136919 | 2.65011E−12 | 2.62875E−10 | 0.214046823 | 2.245258808 |
| 6 | DHRS7 | 7.230448723 | 2.601550579 | 2.19176E−12 | 2.20703E−10 | 0.280936455 | 2.399691343 |
| 6 | LDB3 | 7.2232337 | 1.767409563 | 1.63771E−12 | 1.66808E−10 | 0.511705686 | 2.485309362 |
| 6 | PSMB5 | 7.213437557 | 2.714327097 | 2.52626E−12 | 2.51528E−10 | 0.267558528 | 2.301030159 |
| 6 | SCN1B | 7.188050747 | 2.818411589 | 3.0213E−12 | 2.97475E−10 | 0.264214047 | 2.19672513 |
| 6 | MAMDC2 | 7.169417381 | 1.763499141 | 2.32773E−12 | 2.33511E−10 | 0.508361204 | 2.43639493 |
| 6 | CST3 | 7.160818577 | 1.778345346 | 2.48815E−12 | 2.48666E−10 | 0.498327759 | 2.468938589 |
| 6 | PPP1R27 | 7.141262054 | 1.699460506 | 2.76664E−12 | 2.73414E−10 | 0.548494983 | 2.598356724 |
| 6 | CD59 | 7.114686966 | 1.990451336 | 3.70775E−12 | 3.62378E−10 | 0.394648829 | 2.37080574 |
| 6 | PSMB6 | 7.101378918 | 3.597841978 | 6.47058E−12 | 6.23239E−10 | 0.210702341 | 2.124040604 |
| 6 | DHFR | 7.10077095 | 2.210852385 | 4.40272E−12 | 4.28725E−10 | 0.344481605 | 2.463074446 |
| 6 | RAN | 7.082396984 | 3.504845142 | 7.07473E−12 | 6.7897E−10 | 0.210702341 | 2.143104553 |
| 6 | GPX4 | 7.059715271 | 2.387669563 | 6.02741E−12 | 5.82664E−10 | 0.301003344 | 2.278684616 |
| 6 | H2AFZ | 7.052471161 | 2.756967068 | 7.17548E−12 | 6.86162E−10 | 0.257525084 | 2.288018942 |
| 6 | SGCA | 7.031479836 | 2.660333395 | 7.99659E−12 | 7.56518E−10 | 0.264214047 | 2.169057131 |
| 6 | UBL5 | 7.031469822 | 2.862494946 | 8.40129E−12 | 7.91986E−10 | 0.247491639 | 2.2106812 |
| 6 | BTF3 | 7.013391018 | 2.278787613 | 7.80741E−12 | 7.41258E−10 | 0.317725753 | 2.234415054 |
| 6 | ATP2A2 | 6.995616913 | 1.613937378 | 7.27863E−12 | 6.93531E−10 | 0.568561873 | 2.679160118 |
| 6 | RPSA | 6.985838413 | 2.095212936 | 8.93477E−12 | 8.393E−10 | 0.35451505 | 2.377820492 |
| 6 | PRDX5 | 6.961462975 | 3.354679823 | 1.48548E−11 | 1.36644E−09 | 0.214046823 | 2.135206223 |
| 6 | RPL3L | 6.954126835 | 2.832454443 | 1.3854E−11 | 1.2788E−09 | 0.247491639 | 2.06870842 |
| 6 | RPL11 | 6.951191902 | 1.745062351 | 9.93939E−12 | 9.30383E−10 | 0.505016722 | 2.645953894 |
| 6 | TRDN | 6.922763824 | 1.537417054 | 1.14873E−11 | 1.07151E−09 | 0.692307692 | 3.02549696 |
| 6 | ADSSL1 | 6.911113262 | 3.22949481 | 2.03E−11 | 1.84183E−09 | 0.214046823 | 2.265688419 |
| 6 | TBC1D3P1-DHX40P1 | 6.882483482 | 2.195499897 | 1.8108E−11 | 1.64857E−09 | 0.321070234 | 2.439217329 |
| 6 | PINK1 | 6.872205734 | 3.185737133 | 2.50058E−11 | 2.2195E−09 | 0.217391304 | 2.068469524 |
| 6 | TXNIP | 6.869400501 | 1.552834153 | 1.63003E−11 | 1.48909E−09 | 0.638795987 | 2.78920126 |
| 6 | ISCU | 6.861443043 | 2.906537771 | 2.50471E−11 | 2.2195E−09 | 0.230769231 | 2.175162077 |
| 6 | RPS26 | 6.83658886 | 2.932400227 | 2.93421E−11 | 2.56589E−09 | 0.230769231 | 2.1206429 |
| 6 | UCP3 | 6.832147121 | 2.348556042 | 2.68659E−11 | 2.36491E−09 | 0.294314381 | 2.258117437 |
| 6 | MRPL33 | 6.831234932 | 2.130322933 | 2.50462E−11 | 2.2195E−09 | 0.327759197 | 2.320598364 |
| 6 | CD63 | 6.830426216 | 1.993582964 | 2.41769E−11 | 2.16404E−09 | 0.364548495 | 2.342012644 |
| 6 | ATPIF1 | 6.828724384 | 2.393219948 | 2.71971E−11 | 2.38617E−09 | 0.284280936 | 2.243522167 |
| 6 | ATP1B1 | 6.827972412 | 1.847535491 | 2.35177E−11 | 2.11524E−09 | 0.411371237 | 2.413181305 |
| 6 | UQCRHL | 6.820166588 | 3.526758909 | 3.74644E−11 | 3.24415E−09 | 0.193979933 | 2.136164427 |
| 6 | ATP5H | 6.820083141 | 2.594004869 | 2.99817E−11 | 2.61323E−09 | 0.257525084 | 2.130202055 |
| 6 | RPS17 | 6.813317776 | 1.94209075 | 2.58627E−11 | 2.28417E−09 | 0.387959866 | 2.399419069 |
| 6 | SIK3 | 6.812782764 | 1.449554682 | 2.34854E−11 | 2.11524E−09 | 0.672240803 | 2.760569096 |
| 6 | APOBEC2 | 6.751244545 | 2.786760569 | 5.00848E−11 | 4.3229E−09 | 0.230769231 | 2.264403343 |
| 6 | FEM1A | 6.709998608 | 2.857193232 | 6.44588E−11 | 5.50988E−09 | 0.224080268 | 2.072779894 |
| 6 | NPM1 | 6.69671917 | 2.710310936 | 6.74239E−11 | 5.74486E−09 | 0.247491639 | 2.169639111 |
| 6 | NHSL2 | 6.695146084 | 2.344657421 | 6.26438E−11 | 5.37201E−09 | 0.280936455 | 2.25737977 |
| 6 | TMEM70 | 6.657902241 | 3.143110991 | 9.37177E−11 | 7.93437E−09 | 0.204013378 | 2.259824514 |
| 6 | MRLN | 6.636392117 | 2.436299324 | 9.29115E−11 | 7.89124E−09 | 0.264214047 | 2.143034458 |
| 6 | ATP5C1 | 6.605370522 | 2.751166344 | 1.20319E−10 | 1.00901E−08 | 0.230769231 | 2.228946924 |
| 6 | ADRM1 | 6.575949669 | 3.568546295 | 1.6842E−10 | 1.37763E−08 | 0.183946488 | 2.206176043 |
| 6 | MYBPC1 | 6.572628498 | 1.555399656 | 1.12089E−10 | 9.42967E−09 | 0.765886288 | 3.305385828 |
| 6 | RP11-189B4.7 | 6.560814381 | 2.874093771 | 1.6053E−10 | 1.32532E−08 | 0.214046823 | 2.190091848 |
| 6 | ATP5F1 | 6.558166027 | 2.747328997 | 1.61106E−10 | 1.32596E−08 | 0.230769231 | 2.131269693 |
| 6 | BNIP3 | 6.554860592 | 2.688631773 | 1.62712E−10 | 1.33504E−08 | 0.230769231 | 2.230587721 |
| 6 | BAG3 | 6.550708771 | 3.196539402 | 1.84996E−10 | 1.50396E−08 | 0.197324415 | 2.163541794 |
| 6 | RP11-608021.1 | 6.530601025 | 1.741539478 | 1.47547E−10 | 1.22574E−08 | 0.424749164 | 2.488014698 |
| 6 | RPL36AL | 6.521360397 | 2.38768959 | 1.83695E−10 | 1.49796E−08 | 0.267558528 | 2.19942379 |
| 6 | PPDPF | 6.508202553 | 2.279397011 | 1.93511E−10 | 1.56838E−08 | 0.284280936 | 2.301501274 |
| 6 | NDUFS6 | 6.460418224 | 3.047722101 | 3.11713E−10 | 2.47361E−08 | 0.197324415 | 2.307617903 |
| 6 | FAM96B | 6.458079338 | 2.382074118 | 2.73029E−10 | 2.18914E−08 | 0.257525084 | 2.129049778 |
| 6 | PLEKHA6 | 6.451708794 | 1.860609293 | 2.4962E−10 | 2.01088E−08 | 0.367892977 | 2.340998411 |
| 6 | PRDX3 | 6.448242664 | 2.910031796 | 3.17736E−10 | 2.50644E−08 | 0.20735786 | 2.091418982 |
| 6 | MT-ND5 | 6.44479084 | 2.308079481 | 2.86401E−10 | 2.28639E−08 | 0.267558528 | 2.178037405 |
| 6 | PSMD8 | 6.432804108 | 2.150671721 | 3.03817E−10 | 2.41816E−08 | 0.287625418 | 2.2509377 |
| 6 | NDUFS2 | 6.424179554 | 2.4206357 | 3.43504E−10 | 2.70169E−08 | 0.254180602 | 2.140202045 |
| 6 | PLEKHA7 | 6.412182331 | 1.829835773 | 3.16976E−10 | 2.50644E−08 | 0.377926421 | 2.494570732 |
| 6 | MYEOV2 | 6.409226418 | 3.071406364 | 4.1625E−10 | 3.26419E−08 | 0.193979933 | 2.17652154 |
| 6 | NDUFS8 | 6.376704693 | 2.482207537 | 4.57724E−10 | 3.57886E−08 | 0.237458194 | 2.202676773 |
| 6 | FIS1 | 6.362212658 | 2.757818699 | 5.21756E−10 | 4.05566E−08 | 0.214046823 | 2.198486328 |
| 6 | MT1E | 6.354530811 | 2.028826237 | 4.63606E−10 | 3.61423E−08 | 0.324414716 | 2.601835728 |
| 6 | FXYD1 | 6.334512234 | 2.681018829 | 6.10137E−10 | 4.71508E−08 | 0.220735786 | 2.125230074 |
| 6 | AC072062.1 | 6.301343441 | 1.845905423 | 6.28576E−10 | 4.8435E−08 | 0.351170569 | 2.435872555 |
| 6 | COX7A2 | 6.30117321 | 2.385832548 | 6.96902E−10 | 5.33903E−08 | 0.247491639 | 2.094741583 |
| 6 | AKR1C1 | 6.296294689 | 2.870828152 | 8.1199E−10 | 6.14984E−08 | 0.20735786 | 2.381248474 |
| 6 | ATP5I | 6.289283752 | 2.392958641 | 7.43647E−10 | 5.6645E−08 | 0.244147157 | 2.15017581 |
| 6 | NFE2L1 | 6.28512764 | 2.286773443 | 7.61464E−10 | 5.78365E−08 | 0.257525084 | 2.193778753 |
| 6 | DUSP26 | 6.231910706 | 3.717075348 | 1.3016E−09 | 9.66532E−08 | 0.160535117 | 2.042312145 |
| 6 | PACSIN3 | 6.224504948 | 3.66722846 | 1.34804E−09 | 9.95449E−08 | 0.163879599 | 2.051064014 |
| 6 | PNRC1 | 6.213566303 | 1.783882141 | 1.04887E−09 | 7.85441E−08 | 0.364548495 | 2.340139866 |
| 6 | SRP9 | 6.19944191 | 2.37295413 | 1.26526E−09 | 9.44821E−08 | 0.244147157 | 2.093940735 |
| 6 | EIF4G2 | 6.199000835 | 2.756415367 | 1.38039E−09 | 1.01234E−07 | 0.204013378 | 2.176791191 |
| 6 | DUSP13 | 6.194509983 | 2.501157522 | 1.34689E−09 | 9.95449E−08 | 0.224080268 | 2.036283016 |
| 6 | ST13 | 6.177297115 | 2.598191977 | 1.51682E−09 | 1.09574E−07 | 0.217391304 | 2.200725794 |
| 6 | PGK1 | 6.174462318 | 2.450554848 | 1.51091E−09 | 1.09445E−07 | 0.227424749 | 2.215140104 |
| 6 | NMRK2 | 6.162436962 | 2.165223598 | 1.55857E−09 | 1.12285E−07 | 0.264214047 | 2.312809467 |
| 6 | LAMTOR5 | 6.15945816 | 2.322036028 | 1.5995E−09 | 1.14612E−07 | 0.247491639 | 2.206938267 |
| 6 | ZNF703 | 6.148017406 | 3.119174957 | 1.95921E−09 | 1.3889E−07 | 0.180602007 | 2.109464884 |
| 6 | ATP5EP2 | 6.147247314 | 2.66084075 | 1.79977E−09 | 1.28616E−07 | 0.20735786 | 2.125856876 |
| 6 | UQCRC2 | 6.135326862 | 2.31607008 | 1.82377E−09 | 1.29982E−07 | 0.244147157 | 2.084858656 |
| 6 | ANAPC11 | 6.114979744 | 2.792022705 | 2.19832E−09 | 1.55013E−07 | 0.204013378 | 1.931788921 |
| 6 | PDHA1 | 6.099985123 | 3.185259819 | 2.58028E−09 | 1.80511E−07 | 0.177257525 | 2.00345993 |
| 6 | SYNM | 6.086053371 | 3.795656919 | 3.00773E−09 | 2.08767E−07 | 0.150501672 | 2.115434647 |
| 6 | SLPI | 6.025917053 | 2.67373395 | 3.73412E−09 | 2.56506E−07 | 0.204013378 | 2.594487906 |
| 6 | ASXL2 | 6.023042679 | 1.901546478 | 3.29798E−09 | 2.27724E−07 | 0.304347826 | 2.243610859 |
| 6 | MRFAP1 | 6.017114639 | 2.54519105 | 3.78847E−09 | 2.59569E−07 | 0.210702341 | 2.18260932 |
| 6 | PFKM | 5.985378265 | 1.749054432 | 3.99441E−09 | 2.72975E−07 | 0.341137124 | 2.242970228 |
| 6 | IGFBP5 | 5.975106716 | 1.771970749 | 4.16562E−09 | 2.8322E−07 | 0.351170569 | 2.31854558 |
| 6 | PGM1 | 5.962012768 | 1.616532803 | 4.46369E−09 | 3.01941E−07 | 0.394648829 | 2.305981159 |
| 6 | PTMA | 5.961841583 | 1.644770861 | 4.43003E−09 | 3.00428E−07 | 0.401337793 | 2.460915089 |
| 6 | MPC2 | 5.945738792 | 2.938713074 | 6.02458E−09 | 4.0342E−07 | 0.177257525 | 2.089549541 |
| 6 | POMP | 5.94461298 | 2.26130414 | 5.48281E−09 | 3.69E−07 | 0.234113712 | 2.181485891 |
| 6 | ADI1 | 5.927765369 | 2.624666691 | 6.33322E−09 | 4.21961E−07 | 0.204013378 | 2.052067995 |
| 6 | PDLIM7 | 5.919290066 | 2.03382349 | 6.13694E−09 | 4.0991E−07 | 0.264214047 | 2.194314957 |
| 6 | FITM1 | 5.910078526 | 3.346367598 | 7.59178E−09 | 4.99554E−07 | 0.157190635 | 1.99018538 |
| 6 | CEBPB | 5.909508228 | 1.607381225 | 5.9774E−09 | 4.01271E−07 | 0.401337793 | 2.357259274 |
| 6 | EID1 | 5.903178215 | 2.362650156 | 6.94548E−09 | 4.60445E−07 | 0.224080268 | 2.091333866 |
| 6 | RPL37 | 5.902497768 | 1.870691895 | 6.50062E−09 | 4.32031E−07 | 0.304347826 | 2.283178806 |
| 6 | NDUFA12 | 5.885449409 | 2.199890137 | 7.49551E−09 | 4.94443E−07 | 0.240802676 | 2.123981714 |
| 6 | PCBP1 | 5.881803036 | 2.298487186 | 7.84097E−09 | 5.14677E−07 | 0.230769231 | 2.183685541 |
| 6 | PDLIM1 | 5.87678194 | 1.723639846 | 7.39305E−09 | 4.88898E−07 | 0.341137124 | 2.296848059 |
| 6 | FUNDC2 | 5.867947102 | 2.701520443 | 9.07082E−09 | 5.93937E−07 | 0.193979933 | 2.141511917 |
| 6 | C20orf24 | 5.858021736 | 3.278042078 | 1.0279E−08 | 6.68112E−07 | 0.153846154 | 2.179184437 |
| 6 | TUFM | 5.850725651 | 2.669922829 | 9.89446E−09 | 6.44692E−07 | 0.193979933 | 2.037167788 |
| 6 | ACADM | 5.83611393 | 2.487055302 | 1.05013E−08 | 6.80897E−07 | 0.200668896 | 2.131530285 |
| 6 | FH | 5.835201263 | 4.432899475 | 1.28195E−08 | 8.11413E−07 | 0.130434783 | 2.206704617 |
| 6 | NDUFA2 | 5.831182957 | 2.861986876 | 1.12779E−08 | 7.27159E−07 | 0.177257525 | 2.147917747 |
| 6 | GGACT | 5.818742275 | 2.406543255 | 1.12969E−08 | 7.27159E−07 | 0.214046823 | 1.996012449 |
| 6 | EMC6 | 5.814451218 | 2.955450535 | 1.25491E−08 | 7.96197E−07 | 0.173913043 | 2.105345964 |
| 6 | SSU72 | 5.808634281 | 2.459972382 | 1.20701E−08 | 7.7229E−07 | 0.204013378 | 2.083108664 |
| 6 | MRPL20 | 5.804640293 | 2.505429983 | 1.23864E−08 | 7.87753E−07 | 0.200668896 | 2.045611143 |
| 6 | PSMB1 | 5.803037643 | 2.246137142 | 1.20852E−08 | 7.7229E−07 | 0.230769231 | 2.109088659 |
| 6 | DAPK1 | 5.794298172 | 1.899397492 | 1.20367E−08 | 7.7229E−07 | 0.2909699 | 2.34252882 |
| 6 | MAF1 | 5.771891594 | 2.552605391 | 1.51451E−08 | 9.49571E−07 | 0.193979933 | 2.23315382 |
| 6 | HNRNPA1 | 5.75701189 | 1.945718765 | 1.49988E−08 | 9.42619E−07 | 0.27090301 | 2.336597919 |
| 6 | BANF1 | 5.75558567 | 2.722991467 | 1.67427E−08 | 1.04481E−06 | 0.18729097 | 2.017773867 |
| 6 | UBXN1 | 5.739196777 | 2.535008907 | 1.81451E−08 | 1.1244E−06 | 0.190635452 | 2.143242121 |
| 6 | GBAS | 5.714336872 | 1.50443542 | 1.78775E−08 | 1.11041E−06 | 0.404682274 | 2.255956411 |
| 6 | EIF5A | 5.687508583 | 2.283896923 | 2.30629E−08 | 1.41923E−06 | 0.214046823 | 2.211754322 |
| 6 | ARPP19 | 5.652518749 | 1.521298647 | 2.54537E−08 | 1.55197E−06 | 0.384615385 | 2.287395477 |
| 6 | GPD1 | 5.624283314 | 3.036879063 | 3.57801E−08 | 2.15199E−06 | 0.157190635 | 2.152986288 |
| 6 | HSPA2 | 5.611179352 | 3.990832329 | 4.18072E−08 | 2.4643E−06 | 0.127090301 | 2.343994856 |
| 6 | GADD45GIP1 | 5.608391762 | 3.136398315 | 3.92097E−08 | 2.33188E−06 | 0.150501672 | 2.190649033 |
| 6 | PSMA4 | 5.608319759 | 3.10548687 | 3.90344E−08 | 2.33143E−06 | 0.153846154 | 2.087281227 |
| 6 | CKMT2 | 5.605062008 | 1.508739233 | 3.26715E−08 | 1.96948E−06 | 0.394648829 | 2.33184886 |
| 6 | MAP7D1 | 5.599760532 | 2.769647598 | 3.95479E−08 | 2.34674E−06 | 0.16722408 | 2.113175154 |
| 6 | PHYH | 5.585939884 | 2.809625149 | 4.27033E−08 | 2.50601E−06 | 0.170568562 | 2.060195208 |
| 6 | STK25 | 5.577954292 | 2.655919552 | 4.39959E−08 | 2.57618E−06 | 0.177257525 | 2.111237288 |
| 6 | RPL31 | 5.576155663 | 1.268334031 | 3.75521E−08 | 2.24839E−06 | 0.54180602 | 2.439086199 |
| 6 | CSDE1 | 5.57545805 | 1.561449409 | 3.91144E−08 | 2.33143E−06 | 0.364548495 | 2.319823503 |
| 6 | RASD1 | 5.571712494 | 2.082929134 | 4.15309E−08 | 2.45346E−06 | 0.240802676 | 2.399752378 |
| 6 | PSMC5 | 5.56353569 | 3.188259125 | 5.01473E−08 | 2.89179E−06 | 0.147157191 | 2.176799774 |
| 6 | PSAP | 5.558187962 | 1.588436604 | 4.256E−08 | 2.50313E−06 | 0.357859532 | 2.335814238 |
| 6 | PFDN5 | 5.552555084 | 1.797766566 | 4.51538E−08 | 2.63239E−06 | 0.284280936 | 2.187562227 |
| 6 | NDUFB1 | 5.550048351 | 2.03710103 | 4.70781E−08 | 2.72663E−06 | 0.237458194 | 2.130825758 |
| 6 | C1QBP | 5.549823284 | 3.701573849 | 5.61785E−08 | 3.20483E−06 | 0.133779264 | 2.128165007 |
| 6 | HNRNPA2B1 | 5.543821812 | 1.483270884 | 4.56273E−08 | 2.65417E−06 | 0.387959866 | 2.284466505 |
| 6 | KIF5B | 5.53824091 | 2.355196476 | 5.20648E−08 | 2.99587E−06 | 0.204013378 | 2.041833878 |
| 6 | PSMB3 | 5.53663969 | 2.71405673 | 5.49374E−08 | 3.14754E−06 | 0.170568562 | 2.037930965 |
| 6 | C19orf70 | 5.522607327 | 2.927016973 | 6.01909E−08 | 3.42637E−06 | 0.157190635 | 2.043163776 |
| 6 | ACO2 | 5.51417017 | 1.705826521 | 5.51936E−08 | 3.15541E−06 | 0.301003344 | 2.154806137 |
| 6 | PABPC4 | 5.484155655 | 2.50506115 | 7.10094E−08 | 4.00788E−06 | 0.180602007 | 2.096660376 |
| 6 | CCDC28B | 5.470391273 | 3.415068388 | 8.3254E−08 | 4.63988E−06 | 0.137123746 | 1.879402876 |
| 6 | SELK | 5.465830803 | 2.551641226 | 7.74215E−08 | 4.35131E−06 | 0.177257525 | 1.976877213 |
| 6 | KEAP1 | 5.464803696 | 2.946998358 | 8.17094E−08 | 4.56337E−06 | 0.153846154 | 1.973635554 |
| 6 | ETFB | 5.450778008 | 3.023011684 | 8.92345E−08 | 4.9421E−06 | 0.147157191 | 2.115751266 |
| 6 | SUB1 | 5.44700098 | 1.793137312 | 7.94444E−08 | 4.44621E−06 | 0.277591973 | 2.176582813 |
| 6 | MYBPC2 | 5.43390131 | 2.356826067 | 9.23029E−08 | 5.08029E−06 | 0.18729097 | 2.440089941 |
| 6 | FAM177B | 5.431649685 | 3.398240089 | 1.0157E−07 | 5.56731E−06 | 0.133779264 | 2.11294651 |
| 6 | TRIM54 | 5.42350769 | 1.62296474 | 8.89539E−08 | 4.93685E−06 | 0.317725753 | 2.148635149 |
| 6 | SLC25A11 | 5.41541338 | 3.526627541 | 1.1113E−07 | 6.0475E−06 | 0.127090301 | 2.101318598 |
| 6 | MRPL14 | 5.400605202 | 2.698200941 | 1.1229E−07 | 6.09205E−06 | 0.160535117 | 2.09807682 |
| 6 | RP11-244M2.1 | 5.376281738 | 2.207093477 | 1.21448E−07 | 6.56212E−06 | 0.20735786 | 2.12028718 |
| 6 | AKR1C2 | 5.375475407 | 2.074311256 | 1.204E−07 | 6.51874E−06 | 0.220735786 | 2.320838451 |
| 6 | NDUFA3 | 5.373517513 | 3.219352961 | 1.35316E−07 | 7.22181E−06 | 0.137123746 | 2.169434071 |
| 6 | EIF5 | 5.371281624 | 2.074109316 | 1.22494E−07 | 6.60522E−06 | 0.220735786 | 2.037100554 |
| 6 | CDC37 | 5.362427235 | 2.251095295 | 1.31751E−07 | 7.07411E−06 | 0.197324415 | 2.143185377 |
| 6 | PFN1 | 5.359222889 | 2.191026926 | 1.31988E−07 | 7.07411E−06 | 0.204013378 | 2.199781656 |
| 6 | COX17 | 5.35556221 | 2.453938484 | 1.38303E−07 | 7.35332E−06 | 0.177257525 | 2.086509705 |
| 6 | MGST3 | 5.351976395 | 1.933246255 | 1.34186E−07 | 7.17748E−06 | 0.237458194 | 2.257138491 |
| 6 | EEF1A1 | 5.338895798 | 1.469492435 | 1.35558E−07 | 7.22181E−06 | 0.414715719 | 2.767102718 |
| 6 | SMDT1 | 5.33812952 | 2.719304323 | 1.55449E−07 | 8.21563E−06 | 0.157190635 | 2.103857994 |
| 6 | RASGEF1B | 5.327791691 | 1.260126472 | 1.42305E−07 | 7.55096E−06 | 0.501672241 | 2.499474049 |
| 6 | SNU13 | 5.318223 | 2.215666056 | 1.63533E−07 | 8.60866E−06 | 0.200668896 | 2.047688246 |
| 6 | XIRP1 | 5.317071915 | 3.719923258 | 1.88219E−07 | 9.77266E−06 | 0.123745819 | 2.085065126 |
| 6 | NCL | 5.316083431 | 2.948515892 | 1.76812E−07 | 9.23454E−06 | 0.147157191 | 1.945894599 |
| 6 | RPL22 | 5.314373016 | 2.50930953 | 1.73461E−07 | 9.07734E−06 | 0.16722408 | 2.262530804 |
| 6 | C14orf166 | 5.309322357 | 2.945468187 | 1.83527E−07 | 9.5477E−06 | 0.147157191 | 2.021707535 |
| 6 | NDUFB5 | 5.301839352 | 2.809886456 | 1.90063E−07 | 9.82477E−06 | 0.153846154 | 2.162264347 |
| 6 | SMTNL2 | 5.285618305 | 1.918104768 | 1.90331E−07 | 9.82477E−06 | 0.237458194 | 2.136087418 |
| 6 | CMBL | 5.276971817 | 2.934702396 | 2.19796E−07 | 1.12583E−05 | 0.140468227 | 2.151745319 |
| 6 | NDUFA5 | 5.269839764 | 2.214372396 | 2.10672E−07 | 1.08327E−05 | 0.197324415 | 2.03007555 |
| 6 | TMEM261 | 5.266799927 | 3.137479305 | 2.33888E−07 | 1.18432E−05 | 0.133779264 | 2.243900776 |
| 6 | MRPL51 | 5.26422739 | 2.840142965 | 2.30171E−07 | 1.16995E−05 | 0.147157191 | 2.061555624 |
| 6 | EIF3F | 5.256771564 | 2.164299965 | 2.23912E−07 | 1.14251E−05 | 0.200668896 | 2.080815554 |
| 6 | CSRP3 | 5.252486229 | 4.344708443 | 2.68417E−07 | 1.34634E−05 | 0.110367893 | 2.09969306 |
| 6 | PSMC1 | 5.24920702 | 2.060019255 | 2.30766E−07 | 1.17074E−05 | 0.20735786 | 2.037430048 |
| 6 | ATP2A1 | 5.223749161 | 1.662118673 | 2.55532E−07 | 1.28901E−05 | 0.287625418 | 2.648332596 |
| 6 | HNRNPK | 5.218812943 | 1.773732424 | 2.617E−07 | 1.31762E−05 | 0.254180602 | 2.14149785 |
| 6 | EIF3G | 5.217940807 | 2.501131535 | 2.82955E−07 | 1.40337E−05 | 0.170568562 | 2.034852505 |
| 6 | MKNK2 | 5.209130764 | 1.712717414 | 2.75848E−07 | 1.37577E−05 | 0.267558528 | 2.247205019 |
| 6 | EIF5B | 5.19635725 | 2.348413467 | 3.11751E−07 | 1.53759E−05 | 0.173913043 | 2.207693815 |
| 6 | GHITM | 5.195266724 | 2.884361982 | 3.28347E−07 | 1.61048E−05 | 0.140468227 | 2.183633804 |
| 6 | CMYA5 | 5.194921017 | 1.144853354 | 2.81666E−07 | 1.39959E−05 | 0.605351171 | 2.62312007 |
| 6 | CCT7 | 5.183633327 | 3.285080194 | 3.56213E−07 | 1.73754E−05 | 0.130434783 | 1.979016423 |
| 6 | TUBA4A | 5.178539753 | 2.426283121 | 3.41474E−07 | 1.6687E−05 | 0.170568562 | 1.971544266 |
| 6 | EDF1 | 5.177988052 | 1.935243726 | 3.26609E−07 | 1.60491E−05 | 0.224080268 | 2.109323978 |
| 6 | ABRA | 5.166537762 | 2.47181344 | 3.67661E−07 | 1.79009E−05 | 0.163879599 | 2.090537071 |
| 6 | BAG1 | 5.163764954 | 2.532054424 | 3.73168E−07 | 1.80698E−05 | 0.157190635 | 2.15823102 |
| 6 | MRPS18A | 5.152301788 | 2.875415087 | 4.07848E−07 | 1.95708E−05 | 0.140468227 | 2.080387354 |
| 6 | KLHL40 | 5.142549992 | 3.402488708 | 4.42283E−07 | 2.1071E−05 | 0.120401338 | 2.044775724 |
| 6 | MYL12B | 5.13049984 | 2.2896173 | 4.29349E−07 | 2.04916E−05 | 0.177257525 | 2.102497101 |
| 6 | HADHB | 5.127391815 | 1.227674127 | 3.99851E−07 | 1.92566E−05 | 0.484949833 | 2.414978981 |
| 6 | CCNG1 | 5.125065327 | 2.406084776 | 4.48156E−07 | 2.13037E−05 | 0.163879599 | 2.067242384 |
| 6 | MDH1 | 5.114988804 | 1.830878258 | 4.50852E−07 | 2.13264E−05 | 0.237458194 | 2.13937521 |
| 6 | SNX3 | 5.110743523 | 1.896098852 | 4.61958E−07 | 2.18129E−05 | 0.227424749 | 2.224426746 |
| 6 | NDUFA8 | 5.10172987 | 3.061941862 | 5.29063E−07 | 2.49373E−05 | 0.130434783 | 2.048140526 |
| 6 | CHMP2A | 5.08532095 | 2.975672722 | 5.68806E−07 | 2.65749E−05 | 0.133779264 | 2.042643547 |
| 6 | HCFC1R1 | 5.084489822 | 2.607536554 | 5.58102E−07 | 2.61208E−05 | 0.150501672 | 2.088150978 |
| 6 | ATF4 | 5.071686745 | 1.852736115 | 5.55143E−07 | 2.60281E−05 | 0.227424749 | 2.103831768 |
| 6 | RPS28 | 5.069897652 | 1.388700485 | 5.39611E−07 | 2.53894E−05 | 0.381270903 | 2.473649263 |
| 6 | SNRPG | 5.065374851 | 2.284965515 | 5.94937E−07 | 2.7747E−05 | 0.173913043 | 2.105860233 |
| 6 | NDUFS3 | 5.049787045 | 2.675616503 | 6.65074E−07 | 3.09638E−05 | 0.140468227 | 2.094953537 |
| 6 | ANKRD9 | 5.038779736 | 2.338721275 | 6.82964E−07 | 3.17411E−05 | 0.16722408 | 1.961720347 |
| 6 | SYNGR2 | 5.03120327 | 2.146867752 | 6.9842E−07 | 3.23464E−05 | 0.183946488 | 2.049886465 |
| 6 | IDI2 | 5.024289131 | 2.716320515 | 7.65147E−07 | 3.51915E−05 | 0.143812709 | 2.114146233 |
| 6 | SQSTM1 | 5.011182308 | 1.404871106 | 7.28314E−07 | 3.36722E−05 | 0.351170569 | 2.276377678 |
| 6 | PSMC3 | 5.005663395 | 2.923407316 | 8.40678E−07 | 3.82681E−05 | 0.130434783 | 1.969446063 |
| 6 | CLTB | 5.004262447 | 1.871542454 | 7.83674E−07 | 3.59193E−05 | 0.214046823 | 2.184718847 |
| 6 | AC007325.4 | 4.9988451 | 3.088625669 | 8.77457E−07 | 3.9806E−05 | 0.127090301 | 2.047916889 |
| 6 | RTN2 | 4.996490955 | 2.103905201 | 8.30599E−07 | 3.78742E−05 | 0.183946488 | 2.110096455 |
| 6 | FKBP3 | 4.970762253 | 2.197218895 | 9.4256E−07 | 4.26866E−05 | 0.173913043 | 1.965816259 |
| 6 | MT-ND4L | 4.93316555 | 1.688633084 | 1.09292E−06 | 4.92444E−05 | 0.25083612 | 2.248774052 |
| 6 | MYOZ2 | 4.927094936 | 2.416986227 | 1.19164E−06 | 5.3511E−05 | 0.153846154 | 2.034335613 |
| 6 | THOC7 | 4.921144009 | 3.102139711 | 1.27965E−06 | 5.70775E−05 | 0.120401338 | 1.942938209 |
| 6 | GOT2 | 4.916740894 | 2.761715412 | 1.29465E−06 | 5.76499E−05 | 0.133779264 | 2.219871998 |
| 6 | LDHB | 4.910601139 | 2.526643515 | 1.30409E−06 | 5.79731E−05 | 0.143812709 | 2.195652246 |
| 6 | GABARAPL2 | 4.891491413 | 1.978885055 | 1.36899E−06 | 6.04538E−05 | 0.197324415 | 2.131793261 |
| 6 | ACTN2 | 4.887901306 | 1.193114042 | 1.31755E−06 | 5.83763E−05 | 0.47826087 | 2.507802486 |
| 6 | PDAP1 | 4.886259556 | 2.333023548 | 1.44132E−06 | 6.3228E−05 | 0.157190635 | 2.056086302 |
| 6 | SAP18 | 4.872737408 | 2.139447451 | 1.51207E−06 | 6.6222E−05 | 0.173913043 | 2.115651369 |
| 6 | SERBP1 | 4.857795715 | 1.838281274 | 1.598E−06 | 6.95276E−05 | 0.210702341 | 2.107850313 |
| 6 | HRC | 4.856356621 | 3.992540121 | 1.8182E−06 | 7.8212E−05 | 0.100334448 | 1.83497262 |
| 6 | VBP1 | 4.855873108 | 3.138667107 | 1.765E−06 | 7.62939E−05 | 0.117056856 | 2.190115452 |
| 6 | RAC1 | 4.854376316 | 1.577231288 | 1.58809E−06 | 6.93231E−05 | 0.274247492 | 2.13755393 |
| 6 | THSD4-AS1 | 4.846907139 | 2.40792799 | 1.74825E−06 | 7.56931E−05 | 0.153846154 | 1.948049307 |
| 6 | SLC26A3 | 4.845502853 | 1.22357142 | 1.62658E−06 | 7.06554E−05 | 0.418060201 | 2.440496206 |
| 6 | PRMT1 | 4.829930305 | 2.699828386 | 1.92377E−06 | 8.23534E−05 | 0.137123746 | 1.914654613 |
| 6 | AKIRIN2 | 4.81284523 | 2.278613091 | 2.03459E−06 | 8.68177E−05 | 0.160535117 | 1.939352393 |
| 6 | CYB5R1 | 4.806802273 | 1.580387831 | 2.01606E−06 | 8.61653E−05 | 0.257525084 | 2.193804026 |
| 6 | POLR3GL | 4.803567886 | 3.137506962 | 2.24758E−06 | 9.46904E−05 | 0.117056856 | 1.911339045 |
| 6 | RBM8A | 4.795472622 | 1.912260056 | 2.17138E−06 | 9.19173E−05 | 0.193979933 | 2.140673399 |
| 6 | RPL12 | 4.792969227 | 1.593225479 | 2.13941E−06 | 9.0853E−05 | 0.260869565 | 2.242785454 |
| 6 | CIRBP | 4.786728859 | 1.359882474 | 2.16797E−06 | 9.19173E−05 | 0.344481605 | 2.279529572 |
| 6 | HBB | 4.783227921 | 2.459084749 | 2.40337E−06 | 0.000101065 | 0.143812709 | 2.545548916 |
| 6 | RBM38 | 4.779273033 | 3.044258595 | 2.52105E−06 | 0.000105543 | 0.117056856 | 2.037162066 |
| 6 | PDHB | 4.769976139 | 3.12617898 | 2.62566E−06 | 0.000108893 | 0.113712375 | 1.987374544 |
| 6 | UTP11L | 4.76715517 | 2.307806492 | 2.55996E−06 | 0.000106668 | 0.147157191 | 2.131465435 |
| 6 | PTGES2 | 4.76324749 | 2.789224625 | 2.65232E−06 | 0.000109828 | 0.123745819 | 1.947252035 |
| 6 | NDUFAF3 | 4.762890816 | 2.831311703 | 2.66966E−06 | 0.000110373 | 0.123745819 | 1.959034085 |
| 6 | KLF10 | 4.759530544 | 2.679884672 | 2.69798E−06 | 0.000111371 | 0.130434783 | 2.089118242 |
| 6 | ZMYM4 | 4.751392365 | 1.273067594 | 2.56638E−06 | 0.000106768 | 0.37458194 | 2.370170116 |
| 6 | FABP4 | 4.751252174 | 2.419424057 | 2.73251E−06 | 0.000112622 | 0.147157191 | 2.402862549 |
| 6 | PREB | 4.733300686 | 2.595375299 | 3.03364E−06 | 0.000124071 | 0.130434783 | 1.985854387 |
| 6 | NDUFC1 | 4.725479126 | 2.38892293 | 3.12809E−06 | 0.000127541 | 0.143812709 | 2.181867123 |
| 6 | CUTA | 4.694243431 | 2.535085201 | 3.61813E−06 | 0.000146399 | 0.133779264 | 2.024043798 |
| 6 | CKB | 4.684345245 | 2.849484682 | 3.88553E−06 | 0.000156656 | 0.113712375 | 2.339785576 |
| 6 | LINGO1 | 4.680550098 | 0.969091654 | 3.54688E−06 | 0.000143735 | 0.682274247 | 2.916880608 |
| 6 | MRPS18B | 4.678149223 | 2.516927719 | 3.90876E−06 | 0.000156759 | 0.133779264 | 2.061320305 |
| 6 | NAA38 | 4.668952465 | 2.064262867 | 3.98696E−06 | 0.000159382 | 0.163879599 | 2.330352306 |
| 6 | FABP5 | 4.665528297 | 2.512733936 | 4.13847E−06 | 0.000164205 | 0.137123746 | 2.540095091 |

In some cases, the nucleic specific genes may be one or more from Table 2B. In some cases, the nucleic specific genes may be one or more from cluster 1 of Table 2B. In some cases, the nucleic specific genes may be one or more from cluster 3 of Table 2B. In some cases, the nucleic specific genes may be one or more from cluster 4 of Table 2B. In some cases, the nucleic specific genes may be one or more from cluster 5 of Table 2B. In some cases, the nucleic specific genes may be one or more from cluster 7 of Table 2B.

TABLE 2B
Single Nuclei CST Preparation (Top ranked genes for each cluster)

| Cluster 1 | Cluster 3 | Cluster 4 | Cluster 5 | Cluster 7 |
|---|---|---|---|---|
| Fibroblast | Endothelial | Macrophage | Smooth muscle | Adipocyte |
| gene | gene | gene | gene | gene |
| DCN | ST6GALNAC3 | RBPJ | ACTA2 | CPM |
| LAMA2 | DOCK9 | RBM47 | MYH11 | PPARG |
| NEGR1 | ANO2 | MRC1 | RBPMS | PLIN1 |
| APOD | LDB2 | FMN1 | CALD1 | ACACB |
| GSN | PECAM1 | TBXAS1 | CACNA1C | ADIPOQ |
| LAMC1 | MECOM | FRMD4B | LPP | SLC19A3 |
| GPX3 | B2M | CPM | SORBS2 | MGST1 |
| NOVA1 | VWF | SRGAP2 | TAGLN | FZD4 |
| COL6A3 | PTPRB | F13A1 | KCNAB1 | RP11- |
| | | | | 563P16.1 |
| ABCA6 | TIMP3 | HDAC9 | MYL9 | ADH1B |
| CFD | PLEKHG1 | CD163 | PDGFRB | TENM3 |
| PID1 | FLT1 | DPYD | NR2F2-AS1 | TMEM132C |
| COL4A1 | PTPRM | FCHSD2 | FLNA | GSN |
| TGFBR3 | EPAS1 | GNAQ | DSTN | TNFAIP8 |
| COL4A2 | NEDD9 | SRGAP2B | JUNB | ACSL1 |
| LINC01088 | TACC1 | TRPS1 | TPM1 | RP11- |
| | | | | 665G4.1 |
| EBF1 | SASH1 | ELMO1 | PRKG1 | RP11- |
| | | | | 427J23.1 |
| GREB1L | RP3-510L9.1 | STARD13 | RGS6 | LPL |
| PTPRG | PLCB4 | ZEB2 | LMOD1 | FRMD4A |
| GALNT15 | KLF2 | MS4A6A | RP11- | PARD3B |
| | | | 759A24.3 | |
| ABCA8 | TMTC1 | CELF2 | SPARCL1 | SVEP1 |
| SCN7A | DOCK4 | MERTK | CARMN | FOXO1 |
| NID1 | PITPNC1 | C10orf11 | ACTB | MARC1 |
| ARHGAP24 | CLDN5 | SLC1A3 | IGFBP7 | GPAM |
| EGFR | ARL15 | SLC8A1 | PDE3A | SLC39A11 |
| VIT | EGFL7 | SMAP2 | ADIRF | FP325317.1 |
| LAMB1 | SPARCL1 | SYK | ACTN1 | AQP7 |
| IGFBP7 | HLA-E | AGFG1 | MYL6 | PDE8B |
| CBLB | SEC14L1 | LDLRAD4 | PPP1R14A | LIPE-AS1 |
| AC007319.1 | HLA-B | SPTLC2 | ITGA8 | EBF1 |
| RBMS3 | ELMO1 | PIK3R5 | RP11- | PDE3B |
| | | | 394O4.5 | |
| MGST1 | MAGI1 | CSGALNACT1 | MYLK | TCF7L2 |
| LHFP | EMCN | RGL1 | RHOB | AGPAT2 |
| CXCL14 | SNTG2 | STAB1 | PLN | PNPLA2 |
| MAML2 | APP | IQGAP2 | MCAM | CIDEC |
| MAN1A1 | BACE2 | RAB31 | CSRP1 | ECHDC2 |
| PLXDC2 | SRGN | DOCK2 | CACNB2 | ADRBK2 |
| MAP1B | PPFIBP1 | FGD2 | ADAMTS9-AS2 | PTGER3 |
| SH3PXD2B | TCF4 | SLC9A9 | NOTCH3 | SLC1A3 |
| ADH1B | ASAP1 | RNASE1 | TACC1 | MLXIPL |
| SVEP1 | TANC1 | FAM49B | CNN1 | MAST4 |
| TGFBR2 | ERG | TBC1D14 | NTRK3 | MAGI2 |
| SRPX2 | AC129778.2 | MSR1 | TPM4 | BNIP3L |
| TSC22D3 | PLPP1 | RNF149 | CLMN | PIK3R1 |
| DCLK1 | FLNB | PDGFC | ADGRL3 | MT1M |
| RORA | SYNE1 | VSIG4 | RASAL2 | SPON1 |
| MFAP5 | TIE1 | FOXP1 | DLC1 | ANGPTL4 |
| FTL | SLC9A3R2 | MAN1A1 | ACTG2 | LAMB1 |
| FBN1 | ID1 | SRGAP2C | FHL5 | GPD1 |
| CFH | TMSB10 | SLCO2B1 | ARHGEF7 | WWOX |
| AKT3 | C10orf11 | DOCK8 | CDH6 | PLIN4 |
| SMOC2 | TJP1 | COLEC12 | FRY | TPRG1 |
| COL6A2 | ARHGAP31 | PSD3 | TNC | SLC7A6 |
| CYP4B1 | DUSP1 | LYVE1 | NBEAL1 | PTPRS |
| AOX1 | PRKCH | RNF144B | TINAGL1 | FAM213A |
| FSTL1 | ABLIM3 | CCDC141 | CAV2 | NHS |
| ARHGAP10 | SYNE2 | MTSS1 | TNS1 | LIPE |
| MEG3 | RASGRF2 | ARHGAP15 | ID3 | MDFIC |
| FBXL7 | MYH9 | FLI1 | MYO1D | PTPRF |
| TACC1 | MSN | C1QA | HEYL | RP11- |
| | | | | 444D3.1 |
| LRRTM4 | UTRN | CHST11 | CAV1 | GPC6 |
| EBF2 | BMS1P14 | PICALM | CSDC2 | AKAP12 |
| COL5A2 | WWTR1 | TET3 | EBF1 | MMD |
| FBLN2 | AKT3 | MAFB | HES4 | AGFG1 |
| DLC1 | RASAL2 | MGAT5 | ZNF331 | LEP |
| LTBP4 | RIN2 | DISC1 | CLIC4 | PHLDB1 |
| OPHN1 | HLA-C | ANKS1A | HIP1 | ATP10A |
| C1S | TPO | SRGAP1 | RNF152 | EYA2 |
| SAMHD1 | HIF3A | GAS7 | C11orf96 | PCOLCE2 |
| COL6A1 | ADGRL4 | RIN3 | ATP10A | PTEN |
| SASH1 | CTTNBP2NL | CAMK1D | TOB1 | SOX5 |
| PLPP3 | RAPGEF5 | P2RY14 | CPE | FABP4 |
| VIM | CADPS2 | TG | INPP4B | PDZRN3 |
| ABCA9 | CNTNAP3B | MS4A4E | EPS8 | ITSN1 |
| UST | ANXA3 | ANKRD44 | UBA2 | BCL2 |
| RP11- | POSTN | RP11- | ATP1B3 | RP11- |
| 296E23.1 | | 452H21.1 | | 407P15.2 |
| S100A6 | ADAMTS9 | IRAK3 | LRRC16A | DHRS3 |
| CEBPD | CD36 | SH3TC1 | A2M | C19orf12 |
| SERPING1 | EPB41L4A | SAT1 | SPARC | ATP2B4 |
| SRPX | HLA-A | C1QC | PPP1R12A | PRELID2 |
| C1R | STOM | RP11- | MSRB3 | CPAMD8 |
| | | 624C23.1 | | |
| LRP1 | BTNL9 | CD14 | STK38L | FAM126B |
| AXL | PELI1 | ATP8B4 | PLS3 | ADRA1A |
| COL12A1 | CLIC4 | MCTP1 | CCDC107 | LRP1B |
| CELF2 | ARHGAP29 | ITSN1 | MYH9 | ADCY5 |
| SPRED2 | LRCH1 | PTPRC | ELN | PLXNA4 |
| CRISPLD2 | ST6GAL1 | CLEC7A | SELM | GPX3 |
| TXNIP | TPM4 | GAB2 | SGIP1 | GABRE |
| GLUL | ABLIM1 | ARHGAP18 | NTRK2 | DPT |
| TFPI | RP11- | C20orf194 | AC005358.3 | STAT5A |
| | 588H23.3 | | | |
| TNXB | ID3 | LPAR6 | TPM2 | SAT1 |
| ZFP36L2 | ADGRF5 | CD74 | RCAN2 | AGMO |
| FBLN5 | NKTR | MS4A4A | WTIP | CALCRL |
| NCOA7 | MCTP1 | HCLS1 | SLIT3 | C1QTNF1 |
| S100A13 | TMSB4X | CPVL | APBB2 | PHLDA3 |
| PARD3B | DOCK1 | KYNU | ADAMTS1 | PDGFD |
| RP1-15D23.2 | LDLRAD4 | RUNX1 | SSBP3 | SYNE3 |
| NAV2 | AQP1 | PELI1 | PLCE1 | CLMP |
| MMP2 | MMRN1 | CSF1R | NCALD | PPP1R12B |
| C3 | ITGA6 | ELMO1-AS1 | MRVI1 | SLIT3 |
| IGF1 | CRIP2 | MYO5A | SMTN | AR |
| PRELP | EVA1C | C2CD5 | TMSB4X | PLIN5 |
| MAMDC2 | MKL2 | ARHGAP26 | AXL | RP1-193H18.3 |
| DOCK5 | EFNB2 | MGAT1 | ITGA1 | RP11- |
| | | | | 507B12.2 |
| FBLN1 | CSGALNACT1 | AKAP13 | SNCG | ABHD5 |
| TMSB4X | CYYR1 | ADRBK2 | CEBPD | APCDD1 |
| CTGF | CPNE8 | LYN | RGS5 | RBPMS |
| SEPP1 | TEK | SRGN | CCDC3 | NTRK2 |
| PCDH9 | CD74 | MKRN3 | ESYT2 | ITIH5 |
| LPAR1 | RALGAPA2 | C1QB | WDR45 | PRKAR2B |
| RNF144A | EPHA4 | FOXN3 | CPM | ZBTB7C |
| BMPER | LNX1 | ETV6 | PDK3 | SORBS1 |
| RP11- | SDPR | STX7 | TGM2 | GHR |
| 39M21.1 | | | | |
| ANTXR2 | ADIPOR2 | SLA | SORBS1 | AC002066.1 |
| ABCA9-AS1 | PREX2 | VAV3 | JAG1 | CIDEA |
| CYBRD1 | IFI27 | USP15 | ZFP36L1 | EFNA5 |
| DUSP1 | TINAGL1 | RCSD1 | TIMP3 | MAGI1 |
| BMP5 | PLS3 | DOCK11 | BCAM | TRHDE |
| SLC9A9 | CAV2 | BCAT1 | SLMAP | TLN2 |
| NOX4 | KLF6 | PIP4K2A | C12orf75 | COL4A2 |
| RP11- | ESYT2 | MARCH1 | ETV6 | GPT2 |
| 444D3.1 | | | | |
| LTBP2 | HYAL2 | LRRC16A | GJA4 | USP33 |
| COL1A2 | CDH13 | TLR2 | TTLL7 | SLC16A7 |
| SPARCL1 | C10orf10 | PTPRJ | RYR2 | MAOA |
| ADH1C | ENTPD1-AS1 | RP11- | CRISPLD2 | PRKD1 |
| | | 1080G15.1 | | |
| SH3D19 | IGFBP4 | FCHO2 | VIM | MYCBP2 |
| TCF7L2 | HIP1 | PRKCB | DENND3 | CEBPA |
| ITM2B | PKHD1L1 | ARHGAP24 | RP5-857K21.4 | TACC1 |
| MGP | F8 | CERS6 | MYOCD | CD36 |
| PTGDS | TM4SF1 | RNF130 | SH3GL2 | PLA2G16 |
| ADGRD1 | GPM6A | PTK2B | MAP3K5 | FGD4 |
| DDR2 | PODXL | AOAH | LINC01088 | PALM2-AKAP2 |
| PKD2 | FABP5 | APBB1IP | NPNT | PTPRG |
| BICC1 | TSHZ2 | SEPP1 | UTRN | XIST |
| PTPRS | CD9 | ZSWIM6 | CNTN4 | SEMA3A |
| AGTR1 | CLU | SNX29 | ZFAND5 | HSPB7 |
| RPL11 | SLC2A3 | SKAP2 | ACTN4 | DOCK11 |
| FMO2 | ARHGAP26 | CCND3 | ITIH5 | BMP1 |
| STXBP6 | DGKH | RTN1 | AEBP1 | CHKA |
| USP53 | PPP1R13B | CYBA | LINC01568 | MYO1C |
| CILP | NFIB | CMIP | HSPB1 | RASSF4 |
| FIBIN | CTA-276F8.2 | PLTP | COL18A1 | ELL2 |
| CXCL12 | LRRC1 | PARP8 | PAWR | CYB5A |
| ARL15 | KALRN | ACAP2 | ADCY5 | ADIPOR2 |
| RP11- | CDK17 | ALOX5 | LTBP1 | RP11- |
| 608O21.1 | | | | 1101K5.1 |
| S100A4 | FLI1 | CCDC88A | COL4A2 | CLSTN3 |
| SLC12A1 | ITPRIP | PREX1 | EDIL3 | CCDC107 |
| SCARA5 | CD59 | MAML3 | LINC01197 | RBMS3 |
| LPP | PICALM | DENND4C | AC008440.5 | ACER3 |
| CTDSPL | MYOF | ROCK1 | HES1 | ADIRF |
| SHISA6 | CXorf36 | ARRB2 | RASL12 | AOX1 |
| EEF1A1 | ACTN4 | MED12L | MTHFD2 | ESYT1 |
| KCNT2 | ENG | REL | RERG | AP000304.12 |
| CYP4Z1 | SPC25 | SIPA1L1 | FRMD4A | ACO1 |
| DIO3OS | RP11- | CEP170 | PFN1 | FTL |
| | 638I2.8 | | | |
| TIMP3 | RBMS2 | RP11- | KALRN | NRIP1 |
| | | 16C1.3 | | |
| MTSS1 | CLEC14A | PTPRE | PRKCDBP | FASN |
| LRRC16A | FRYL | FYB | RASSF1 | EMP1 |
| FTH1 | LIMS2 | RHBDF2 | ZBTB7C | RBP4 |
| PODN | ABCG1 | FCGR2A | WFDC1 | IRS2 |
| PLAGL1 | DACH1 | FAR2 | DUSP1 | FKBP5 |
| EFHD1 | KIAA0355 | NAIP | SIK3 | NNT |
| IGFBP6 | ITGA9 | RBMS1 | ENTPD1-AS1 | SRPX |
| RPS27 | STOX2 | FOXN2 | MALAT1 | EHBP1 |
| PLEKHH2 | MYCT1 | EMB | WSB1 | TMEM120A |
| GFRA1 | RPGR | NOTCH2 | PTMA | PARVA |
| SPRY1 | NEBL | TNFRSF1B | SOD3 | GYG2 |
| PI16 | EMP1 | TCF12 | CCNI | PDXK |
| ZBTB20 | RP4-678D15.1 | C1orf162 | BGN | ELOVL5 |
| PROCR | ITGA1 | MEF2C | ECE1 | SMIM3 |
| H3F3B | ABR | ST6GAL1 | CD9 | AASS |
| SDK1 | TMTC2 | SLC8A1-AS1 | GADD45B | NCOA7 |
| RPS2 | ADIRF | WDFY2 | SEPW1 | TNRC6A |
| DYNC2H1 | C8orf4 | INPP5D | BTG2 | SLC26A3 |
| CYR61 | TSPAN14 | STK17B | NR2F2 | ASPH |
| EIF1 | HLA-DRB1 | GNG2 | TLN1 | NME3 |
| CCNG2 | PPP1R16B | ADAM28 | FBLIM1 | NHLRC3 |
| RPL13 | A2M | POU2F2 | DGKH | GPBAR1 |
| SOX5 | DOCK6 | CTSB | FOS | HIVEP3 |
| WASF3 | PPARG | CCDC91 | NFASC | CTIF |
| PDGFRA | RP4-613B23.1 | CEBPD | CSRP2 | C2CD2 |
| RP11- | CD93 | CSF3R | SDK1 | MT1A |
| 210L7.3 | | | | |
| HS3ST1 | FBXL7 | MVB12B | NR4A1 | FOXO3 |
| RPL41 | PIK3C2A | GTDC1 | ID4 | GLUL |
| SESTD1 | RPS6KA2 | VOPP1 | COL6A2 | C14orf180 |
| HIF3A | ICA1 | NRP1 | PIP5K1B | RAB2A |
| MEDAG | LIMCH1 | RAP1A | RPL15 | DDX5 |
| LRP1B | FABP4 | TFEC | ACTG1 | RGS3 |
| GRK5 | FGD5 | HCK | EEF1A1 | NGF |
| SLIT2 | RUNX1T1 | AP000304.12 | VASN | SCP2 |
| CPNE4 | MLLT4 | NPL | MYO1B | STAT2 |
| MYOC | HLA-DRB5 | SNCA | NDE1 | FIGN |
| ECE1 | MYO1E | ATG7 | CKB | CLMN |
| EYA2 | ELMO1-AS1 | HIF1A | LINC00702 | DDX17 |
| MME | ENTPD1 | PHACTR2 | PHACTR1 | EEA1 |
| MAMDC2-AS1 | CCND3 | FGD4 | ELL2 | CACHD1 |
| IGFBP3 | GMDS | TYROBP | SPECC1 | RTN1 |
| B2M | CALCRL | DENND1A | PPDPF | JMJD1C |
| PLEKHA5 | CDH5 | JAML | PDZRN3 | PHLPP1 |
| DDIT4 | MGLL | RNF150 | ARHGAP10 | COL4A1 |
| NCAM2 | PTGIS | SLC11A1 | COBLL1 | ST3GAL6 |
| RP4-678D15.1 | FAM196A | MAML2 | RPL41 | PSMA1 |
| SLC44A1 | CMTM8 | CSGALNACT2 | SPINT2 | GRINA |
| RBMS3-AS3 | WNK1 | SCN9A | ISYNA1 | SESTD1 |
| CYTH3 | CAV1 | LAPTM5 | LGALS1 | RREB1 |
| NUPR1 | FCHSD2 | NHSL1 | PRDM16 | ADCY6 |
| TIMP2 | SMAD1 | DLEU2 | TBC1D1 | PER3 |
| NLGN4Y | MEF2A | IL1R2 | PRMT9 | TMEM164 |
| RPS15 | ICAM2 | SH3BP5 | RPL10 | RP11- |
| | | | | 736K20.5 |
| RPL3 | ESAM | CHKA | ADCY3 | CTD-2282P23.2 |
| UAP1 | IFI44L | GRB2 | AOC3 | MACF1 |
| CDO1 | MMRN2 | MPPED2 | RP11- | THSD7A |
| | | | 665G4.1 | |
| PLA2G2A | MGP | PLXDC2 | ILK | DUSP4 |
| PRICKLE2 | SPTBN1 | ATG16L2 | PHLDB2 | YWHAZ |
| ADAM33 | IRAK3 | AP000476.1 | AC097724.3 | SLC2A4RG |
| RP11- | HSPG2 | PDE4B | RBPMS2 | ELMOD3 |
| 160H12.2 | | | | |
| LMO3 | SLCO4A1 | AGTPBP1 | S100A6 | PTPRM |
| PQLC2L | NRG3 | FNBP1 | SORT1 | ZFP36L2 |
| DDX5 | CCDC85A | DIP2B | SOX5 | SMOC1 |
| RPL13A | AKAP12 | SIRPB2 | RP11- | LINC01301 |
| | | | 315E17.1 | |
| FGFR1 | LYST | LCP2 | CSPG4 | AFAP1L1 |
| MMP14 | RP11- | ASAP1 | RPS27 | JAK2 |
| | 362K2.2 | | | |
| XPO5 | PKN3 | RP11- | RPL7 | SHOC2 |
| | | 1143G9.4 | | |
| SPATA6 | SH3BGRL2 | ITGAM | HEY2 | STARD13 |
| RP11- | TGM2 | ENTPD1-AS1 | LBH | ADAMTS9-AS2 |
| 289H16.1 | | | | |
| NLGN1 | ITGA5 | ALCAM | SMIM12 | SH3KBP1 |
| ITIH5 | NOTCH4 | GSAP | PTPRG | MARK1 |
| RPS28 | CPAMD8 | STK4 | SPEG | SLC7A6OS |
| PIK3R1 | RP5-1101C3.1 | BMP2K | RPS27A | PILRB |
| APBB2 | TSPAN9 | ARHGAP6 | CIRBP | TUSC5 |
| IFITM3 | CDC42BPA | STK10 | CRIM1 | TNKS1BP1 |
| FNDC3B | NUAK1 | CDK6 | RPL21 | C10orf11 |
| CD99 | GFOD1 | TMEM2 | GRK5 | IRAK3 |
| ADAMTS5 | SULF1 | KIAA1551 | SYNPO2 | ACADS |
| RPL34 | PLXDC2 | CD163L1 | AC002066.1 | PXDN |
| RUNX1T1 | CCNY | SIGLEC1 | ITPR1 | ARHGAP20 |
| F3 | ST8SIA6 | TGFBI | TGFB1I1 | MGEA5 |
| WISP2 | RAPGEF1 | CHN2 | PNRC1 | MPST |
| GLI3 | IFITM3 | PLXND1 | EEF2 | RETSAT |
| RBMS3-AS2 | SERPINE1 | GNG12-AS1 | RPL23A | AK4 |
| C10orf10 | RGCC | SPI1 | GUCY1A3 | COL5A2 |
| KCND2 | CNKSR3 | EPB41L3 | PPP1CB | OXR1 |
| SDC2 | HEG1 | RP11- | SLC39A11 | LENG8 |
| | | 277P12.20 | | |
| CIRBP | HMCN1 | MAP3K8 | SYTL2 | FTH1 |
| COL15A1 | CMIP | GPR34 | RPS14 | SPTBN1 |
| GAS1 | S1PR1 | RP11- | AC140912.1 | TNS1 |
| | | 347P5.1 | | |
| CYP4X1 | RAPGEF4 | KIF13B | GPRC5C | FBXO11 |
| ALDH1A2 | PGM5 | NUMB | TMEM184B | TIMP4 |
| CST3 | RNF125 | SUSD6 | ARHGAP17 | RASGEF1B |
| WIPF1 | PLSCR4 | MANBA | RPL3 | DCXR |
| RPLP0 | RHOJ | CXCR4 | RPL5 | DOCK1 |
| RND3 | RPH3AL | GPR183 | PICALM | ITGA7 |
| EFEMP1 | RAMP3 | TPK1 | RP11- | ASH2L |
| | | | 466A19.3 | |
| FYN | RHOB | CD53 | EDNRA | KCNIP2-AS1 |
| STK24 | STK38L | CPNE8 | EPAS1 | VEGFB |
| TRIM4 | KLF4 | RNF175 | RBMS3 | SLC25A1 |
| RPL28 | FOXP1 | GNB1 | KCNMB1 | VEGFA |
| ARMC9 | PLXNA2 | FCER1G | SEPT7 | WASH1 |
| LUM | EDN1 | MAP2K1 | SH3BGRL | CAT |
| BMP4 | CCDC68 | MEF2A | SLC38A11 | IMMP2L |
| HTRA3 | ADD1 | KCNE1 | DYNLT1 | EIF4EBP1 |
| OLFML3 | UVRAG | MYO1F | H3F3B | VKORC1L1 |
| AMPH | FRY | ADCY7 | PALLD | FHIT |
| DPT | BTG1 | TMSB4X | EGFLAM | DLC1 |
| WDFY3 | CD34 | RNASE6 | C1QTNF1 | HK2 |
| ADD3 | PTMA | ZNF562 | C9orf3 | ADAMTS15 |
| PCDH9-AS2 | HECW2 | SH3KBP1 | ABHD2 | ZBED3-AS1 |
| MAGI2 | RP3-523E19.2 | ADAP2 | RAD51B | ITPK1 |
| S100A10 | WARS | LGMN | ANKRD37 | EPB41L4B |
| PHLDB1 | GIMAP8 | ARSB | DTX3 | GNAI1 |
| PDE1A | PLVAP | ZCCHC2 | PDLIM7 | ABCD2 |
| SERPINF1 | CXCL2 | RAB20 | ARHGEF17 | LONRF1 |
| NFIA | SH2D3C | CD86 | EIF4A2 | TRIO |
| VCAN | RASIP1 | HLA-DRA | ACKR3 | C1QTNF7 |
| AC018890.6 | GNG11 | ITGB2 | MAML3 | TMBIM1 |
| KAZN | BST2 | AP2A2 | SMURF1 | CDYL |
| RP11- | DNAJC18 | RCAN1 | PLEKHO1 | RP11- |
| 197K3.1 | | | | 286B14.1 |
| WWOX | MCF2L | AC010127.3 | MT1E | SELENBP1 |
| LAPTM4A | APBB2 | CR1 | CBX6 | AGBL4 |
| ADAMTSL3 | ZBTB46 | EPS8 | ARL6IP4 | C1RL |
| CPB1 | TLL1 | MAF | NT5DC3 | CH507-528H12.1 |
| MXRA8 | SNHG7 | IKZF1 | PCED1B | SOS1 |
| MEG8 | GNAI2 | HNMT | SERF2 | SLTM |
| EMP2 | LHFP | ST8SIA4 | TBX2 | APBB2 |
| CALCRL | RP11- | RNF13 | FRYL | TTLL5 |
| | 35G9.5 | | | |
| SYTL4 | CEP112 | DIRC3 | NBL1 | PRDX6 |
| TC2N | SLCO2A1 | HLA-DRB1 | ZNF90 | SNTB1 |
| TLN2 | YES1 | ITPR2 | ATF4 | FSTL3 |
| ANGPTL1 | TMEM204 | CMTM7 | FTH1 | PC |
| HMCN2 | NOSTRIN | VCAN | RPS18 | STK39 |
| TBRG1 | LIFR | FCGR2B | TRABD2B | YPEL5 |
| UGGT2 | GUK1 | NCKAP1L | SLC25A6 | FGF2 |
| ECHDC2 | TANC2 | IFI16 | UBB | SCRN2 |
| LAMC2 | MYO5C | H2AFY | APOO | RP11- |
| | | | | 756H20.1 |
| PDGFD | ELN | MIR181A1HG | SCN3A | PHF3 |
| MKL1 | FGF2 | C3AR1 | ESAM | PQLC1 |
| DENND2A | CA8 | LCP1 | FOSL2 | TWIST2 |
| SLC2A13 | ID2 | EDA | UNC5C | TJP2 |
| SLC39A14 | IFITM2 | LRRK1 | EVA1C | THRB |
| ZFAND5 | EPB41L2 | SNX2 | H2AFJ | PCED1B |
| DOCK1 | CAPZA2 | CSF2RA | ZNF141 | CAV2 |
| VGLL3 | LMBR1 | FCGRT | RPL6 | CDC37L1 |
| LIFR | ITPR2 | BLVRB | RPS4X | IL1RAP |
| KCNH1 | TUSC3 | RASA2 | AC013463.2 | MAPK10 |
| MED13L | JUNB | LINC00278 | DTNA | EPB41L3 |
| LARP6 | RHOC | MPEG1 | CREB3L2 | FBXL5 |
| TRPC1 | RAPGEF2 | INTS10 | KCNA5 | ABL2 |
| S100A11 | NDRG1 | RP11- | TSC22D1 | TAPT1-AS1 |
| | | 815J21.4 | | |
| C1RL | SYN3 | PAPSS2 | CTD-2009A10.1 | HSDL2 |
| TRHDE | CD2AP | SND1 | WBSCR17 | AOC3 |
| ERRFI1 | ZNF264 | WLS | MICAL3 | MAP3K5 |
| PLAC9 | PLCG2 | CCR1 | RASSF3 | DGAT2 |
| TSHZ2 | RNASE1 | C22orf34 | PRUNE2 | CSPG4 |
| PLXDC1 | MYRIP | PIK3AP1 | FIGN | KATNAL1 |
| CALD1 | THBD | NCF4 | TCEAL4 | HSPG2 |
| CYP1B1-AS1 | TCF4-AS1 | TYMP | ITGB1 | LIMA1 |
| RAMP2-AS1 | ARL4A | SHTN1 | TANC1 | API5 |
| ZEB1 | PALMD | TEC | RASGRP2 | GSDMB |
| RIMS3 | EEF1A1 | NCOA3 | TASP1 | NCK1 |
| MTCH1 | GNAQ | LYST | MYL12A | F3 |
| CPQ | PRKX | CTD-2282P23.2 | GARNL3 | RP1-41C23.1 |
| SPSB1 | ADAM15 | MDFIC | PLCB4 | CD81 |
| ARHGAP21 | HIPK3 | IL10RA | COL4A1 | DHODH |
| TEX26-AS1 | BMP6 | PAN3 | PLPP3 | BMS1P14 |
| PCSK5 | PCAT19 | SH3BGRL | KANK2 | INTS10 |
| FAM196A | HLA-DRA | ADGRG6 | FABP4 | CTD-2337A12.1 |
| SMIM3 | BMX | TBC1D22A | ZFP36 | ATXN3 |
| COL3A1 | GNB1 | TBC1D5 | FAM46B | RP11- |
| | | | | 573D15.3 |
| SRSF5 | RELN | RP6-159A1.4 | CRIP2 | MT-ND4 |
| FAM162B | CRIM1 | CTSZ | SLC27A4 | CKB |
| RPL14 | RPL3 | FER | ADARB1 | MRPL41 |
| APP | SPTAN1 | IL13RA1 | MIR22HG | JAG1 |
| DANT2 | PTPRE | DAPK1 | YWHAH | PRKCD |
| TENM1 | CH17-80A12.1 | FOS | BAIAP2L1 | TTC38 |
| SMIM14 | RP11- | EXT1 | RP11- | PPP3CA |
| | 314N13.10 | | 394O4.6 | |
| ADH5 | GIMAP4 | SPIDR | PLCL1 | FMNL2 |
| DSE | LY6E | PEAK1 | AKT3 | FGF1 |
| NFKBIA | BMPR2 | FTL | RPL27A | CCDC69 |
| SLC5A9 | PTK2 | FOLR2 | PTRF | IL17RE |
| PGF | RP5-1121H13.3 | EMP1 | NDUFA4 | TNIP1 |
| EMX2OS | CNTNAP3 | IGSF21 | FAU | MAP3K8 |
| RPS17 | ARFGEF2 | CYTIP | MARK1 | ACVR1C |
| ISLR | PLAT | CHPT1 | ACTR3 | HEXDC |
| SIK3 | LRRC32 | CD68 | RPL14 | ASS1 |
| RBPMS | ACKR1 | TLR1 | TMEM181 | IGF1 |
| SLC25A43 | SWAP70 | PTEN | NDUFA4L2 | ALDH7A1 |
| RPS4X | S100A10 | WDR36 | MAF1 | FAM13A |
| PDGFRL | LRRFIP1 | CAB39 | SNTB1 | ZBTB44 |
| MROH9 | NOD1 | AKR1A1 | FER | HRSP12 |
| RPL35A | CYYR1-AS1 | TTYH3 | DOCK8 | MBD2 |
| PTGR1 | C22orf34 | DPYD-AS1 | LDOC1 | MRPL34 |
| THBS4 | RFTN1 | CTSC | ARPC5 | ARHGAP26 |
| CEP126 | NAV1 | CTSS | RPL36AL | TRIM35 |
| IPO9-AS1 | FRMD4B | ABR | RP11- | CDKN1C |
| | | | 290H9.2 | |
| FER | SHE | SNX10 | LGI4 | LINC00623 |
| RPS27A | ARAP3 | CROCC | CNN2 | RSPO3 |
| PCOLCE2 | MAP2 | MIS18BP1 | MT2A | TMEM72-AS1 |
| RPS15A | LAMA5 | CD44 | LAMA5 | MT1E |
| SRSF7 | TAGLN2 | WIPF1 | IGFBP2 | ANXA6 |
| TIPARP | ACTN1 | PLEKHA2 | DPY19L2 | HIPK3 |
| PTPN9 | SEMA6A-AS1 | TAOK3 | TJP1 | DCAKD |
| WDFY2 | ANKS1A | HLA-DRB5 | MYADM | MSL2 |
| WBP5 | GALNT18 | CMKLR1 | SRSF3 | CDK5RAP1 |
| KCTD3 | RASSF1 | FGR | CAMK2G | CERS6 |
| PRR16 | MGAT5 | CH507-513H4.1 | RPS6 | HEBP2 |
| PAK3 | PIK3C2B | PDE3B | CACNA1H | PAN3 |
| CCDC85A | FNBP1L | COLGALT1 | ROCK1 | CYB5B |
| PTBP2 | TPST1 | SP100 | SSBP2 | RNF19B |
| PMP22 | SHROOM4 | PLA2G4A | ATP5E | AIFM2 |
| RPL15 | STX12 | RP11- | CYCS | RP11- |
| | | 289H16.1 | | 962G15.1 |
| STEAP4 | RUNDC3B | RNF145 | SUSD5 | LRP1 |
| RP11- | MMP28 | ZNF665 | RPL36 | ETFB |
| 553L6.5 | | | | |
| SLC25A6 | SHANK3 | ATF6 | DST | LOXL2 |
| PDZRN4 | RP11- | PAPD4 | FAM155A | ADRA2A |
| | 767I20.1 | | | |
| SDCBP | MGAT4A | MAP3K1 | COX7A2 | MAMLD1 |
| YPEL2 | CD151 | WASF2 | DIRC3 | LGALS12 |
| PRKD1 | MTHFD1L | LYZ | CFL2 | RGS12 |
| TMEM204 | RPL36 | LST1 | RPS16 | ARSD |
| SELM | RP11- | LIMD1 | TBX18 | PGM3 |
| | 526F3.1 | | | |
| PDK3 | NRP1 | EPSTI1 | BTF3 | EPS8 |
| GPX4 | HEY1 | NLK | MYO1E | DDR2 |
| RP1-193H18.3 | APOLD1 | CHSY1 | AC018816.3 | SRRM2 |
| RPL10 | CLEC1A | DRAM2 | EIF5A | CENPO |
| CYP4A22-AS1 | RPS6 | RASSF3 | KCNJ8 | MATN2 |
| RECK | ADCY4 | LRRK2 | RPLP1 | CAV1 |
| RGMA | SMAD6 | SAMSN1 | B2M | INTS6 |
| BOC | MGST2 | MEGF9 | CD151 | BCKDHB |
| CLSTN2 | PELI2 | FPR1 | NPY1R | MYL2 |
| RHOBTB3 | SPRY1 | CLEC4E | PTMS | OXCT1 |
| ACVRL1 | ABCB1 | CARD8 | UBL5 | JAK1 |
| RPL18 | CCL21 | SPRED1 | TGFBI | C16orf13 |
| CH507-513H4.1 | MTUS1 | SNX13 | TUBB6 | GOLGA8B |
| SPRY2 | KIAA1671 | GLIPR1 | SLFNL1 | TFDP2 |
| NIPAL2 | WSB1 | TIAM1 | EFHD1 | SIK2 |
| GPNMB | S100A16 | SCRG1 | ARHGEF10L | TSPAN15 |
| RP11- | REEP3 | CD84 | BRSK2 | PPP1R15B |
| 196H14.2 | | | | |
| ITGA5 | RNF115 | HSPA6 | CBR4 | G0S2 |
| PCYOX1 | ASAP2 | TNFRSF11A | PACSIN2 | TSPYL2 |
| PDGFRB | ADAMTS6 | SERINC5 | IGFBP5 | CARMN |
| ODF2L | COL8A1 | KLHL2 | CNN3 | ZFAND6 |
| RAP2C-AS1 | NDRG4 | ZDHHC20 | ATP5J | ITPKB |
| MAP1LC3A | EFNA5 | PPP3CA | RPS23 | ZC3H3 |
| FAP | JAM2 | CTTNBP2NL | HIPK2 | ECHDC3 |
| MMP3 | CHN1 | UNC93B1 | ITM2C | PER1 |
| TRERF1 | UBC | THEMIS2 | DGKG | ATF4 |
| CAB39L | RP1-90G24.10 | ZNRF2 | PITPNB | MCCC1 |
| IQCH | SAMD12 | PRMT2 | RP11- | AZGP1 |
| | | | 444D3.1 | |
| ACADL | FDPS | FGL2 | PDZRN4 | MARC2 |
| CFTR | PITPNM2 | RP11- | STEAP1B | TSKU |
| | | 196H14.2 | | |
| ATF7IP2 | TMEM88 | ATRN | LIMS2 | SPARC |
| SPG20 | PKP4 | NPC2 | CRTC3 | ZKSCAN1 |
| RERG | GRK5 | ATM | NHS | NAT8L |
| CCNI | GPIHBP1 | RB1 | SLC6A16 | STK24 |
| FBXO42 | RAPGEF3 | NBPF19 | PEAR1 | ZSWIM8 |
| NAA11 | TSPAN13 | CEPT1 | GJC1 | SLC5A6 |
| NAV3 | CSNK1E | DENND4B | CTD-2269F5.1 | IDH1 |
| EVC2 | PTMS | PARP4 | PLEKHG3 | MYEOV |
| AC006994.2 | GRASP | TRPM2 | WDR1 | AACS |
| MUSK | ZNF366 | MAP3K3 | RPL10A | TNK2 |
| AC010084.1 | STK38 | PPP1R21 | TCEB2 | APMAP |
| PTEN | NFKBIA | IL27 | MAP2 | FIG4 |
| CLMP | XAF1 | DERA | EIF1 | CPSF7 |
| KIAA0040 | ITPKB | GCA | PTH1R | RABEP1 |
| ARL4D | ITSN2 | ALOX5AP | CERS6 | TRPS1 |
| MT1G | ATP10D | SCAMP2 | FEZ2 | RP11- |
| | | | | 175K6.1 |
| FAU | ADAMTS9-AS2 | CYTH4 | GUCY1A2 | BOK |
| DPYSL2 | TMEM184B | KCTD12 | GLDN | PRR5 |
| LSP1 | GRAPL | KCNAB2 | NMNAT2 | APOL6 |
| TMA7 | STC1 | C5AR1 | COL4A5 | ARHGAP21 |
| RP5-965F6.2 | EFNA1 | PTPN18 | CCNH | TYRO3 |
| IMMP2L | PDCD4 | GABRB2 | EPN2 | C6orf106 |
| PTGFR | RPL34 | VPS41 | EMD | PABPN1 |
| MFAP4 | FAM107A | TNRC18 | PBXIP1 | RP11- |
| | | | | 353M9.1 |
| CTSF | AC007319.1 | ATP2B1 | RP11- | SLC38A10 |
| | | | 499P20.2 | |
| STARD9 | PLEKHA7 | SOAT1 | RPL35A | GSK3B |
| TASP1 | RAI14 | TNFAIP2 | YBX1 | APBB1IP |
| GNB2L1 | NOS1AP | SLC2A13 | RPL35 | CECR5 |
| ADGRB3 | VPS37B | RP11- | HMGN2 | C6 |
| | | 286E11.1 | | |
| ANOS1 | TBCD | HAVCR2 | RPL34 | HNRNPU-AS1 |
| SLC1A5 | ART4 | RP11- | PABPC1 | DOCK8 |
| | | 556E13.1 | | |
| LINC00882 | ITM2B | SYNE3 | RP4-593C16.4 | RP5-965F6.2 |
| H2AFJ | H3F3B | STAT6 | MSC-AS1 | ARID1A |
| RP11- | CX3CL1 | OXR1 | RPL19 | RP11- |
| 82L20.1 | | | | 745L13.2 |
| PLD1 | PHACTR2 | ENTPD1 | ASAP1 | PFKFB3 |
| HMGCLL1 | CHSY1 | BAZ2B | C9orf16 | DMTF1 |
| RASSF4 | IFI6 | NABP1 | ATP8B1 | PFKFB1 |
| PSAP | AC007743.1 | HERPUD1 | RPL8 | AF127936.9 |
| USP33 | AC007193.10 | TMEM106A | IFITM3 | NUPR1 |
| CTTNBP2 | RP11- | UBXN2B | SYNM | RALY |
| | 382A20.3 | | | |
| RPS13 | DLL4 | DNM1 | EOGT | DHX8 |
| ADAMTS9-AS2 | ZFP36 | AGO4 | KIF5B | NR3C1 |
| RPS18 | PARP14 | IL18R1 | SEPT4 | STIM2 |
| RNF19B | TFF3 | ATP11A | GUK1 | MMP19 |
| LINC00968 | PPP1R14A | WDFY4 | SELK | PCDH9 |
| RPL7A | C6orf141 | NCOA2 | PPFIA2 | MIR193BHG |
| MPZL1 | TJP2 | EMILIN2 | LHFP | SPATA5 |
| B4GALT1 | BAIAP2L1 | ADPGK | OST4 | AHNAK |
| SERF2 | YWHAH | MOB1B | CBL | SPEN |
| EFHC1 | ADAMTS9-AS1 | IFNGR1 | RP11- | AGO3 |
| | | | 391J2.3 | |
| ARHGAP6 | KLF3 | EPS15 | PHLDA3 | RP11- |
| | | | | 479O9.4 |
| PARVA | SOX17 | ZNF846 | RP11- | RHOT1 |
| | | | 1000B6.3 | |
| RPL10A | ZDHHC14 | ARHGAP25 | DDX5 | SH3GLB1 |
| APOL3 | CNOT6L | SLCO3A1 | SLC8A1 | ADH5 |
| VIMP | ESR2 | ST6GALNAC3 | LRP10 | TGFBR3 |
| BDNF-AS | ENPP2 | NFATC2 | XIST | SEC31B |
| TXNRD1 | MPZL2 | E2F3 | NR1H2 | LAMA4 |
| RBMS1 | INPP5D | PPP1R12A | DGKB | LINGO1 |
| RTN4 | RARB | ME1 | DKK3 | BCAP31 |
| CYP1B1 | KAT6A | CBL | ARHGAP26 | TBC1D16 |
| PAPPA | IFI16 | EIF2B5 | NR3C2 | PPP2R2A |
| CATSPERB | RBMS3 | MLKL | ENTPD3 | NAT9 |
| PTPN13 | DENND3 | CD4 | RAPGEF2 | ABCA1 |
| HSPG2 | FZD6 | RFWD2 | NKD1 | KLHL25 |
| XYLT1 | NLRC5 | RP11- | ELOVL5 | KIRREL |
| | | 426C22.5 | | |
| OSR2 | GIMAP7 | TBC1D9 | RPL38 | BRD2 |
| RPS25 | CDC42EP3 | PAG1 | DDAH2 | KLB |
| COL5A1 | KDR | GRAMD3 | KLF12 | PCSK5 |
| RPS23 | GJA5 | BLNK | SAP18 | MAP3K6 |
| IGF1R | RPS14 | PRSS36 | CYP7B1 | AKR1A1 |
| WSB1 | TMEM255B | LRRFIP1 | RP11- | PEX11A |
| | | | 15B24.5 | |
| CSTB | MALL | AFF3 | ID2 | ARMC8 |
| CNTN4 | GNA14-AS1 | RCBTB2 | RP11- | DUS1L |
| | | | 156K13.1 | |
| XG | SRP14 | TRMT1 | TEAD3 | WDFY2 |
| AP3S1 | SPARC | TRIM14 | LINC00924 | TMEM135 |
| EEF2 | RPS27A | TNFAIP8 | PLEKHA6 | TESK1 |
| PCSK6 | WDR45 | ARHGAP31 | IGFBP4 | FZD7 |
| FN1 | ITPKB-AS1 | CPPED1 | RPS8 | BANK1 |
| EPB41L2 | AFAP1L1 | ZNF710 | GPR20 | ARHGEF28 |
| LONRF1 | RPL10 | FGD3 | RPS3 | THSD7B |
| EIF4A2 | PTRF | CLEC2D | GAPDH | PRRC2B |
| TANK | RB1 | FCGBP | MRPL33 | RP11- |
| | | | | 147C23.1 |
| SPTBN1 | RPL27A | ARPC3 | RPS7 | MSH3 |
| EBF3 | ATF7IP2 | RREB1 | MAGI1 | PEX5 |
| PXDN | DDX5 | SLC12A6 | ABTB1 | TKT |
| PLEKHA7 | RPL29 | PAQR3 | DAPK3 | GPX1 |
| ELF1 | SMARCA2 | ARPC1B | CLIC1 | PECR |
| RBBP6 | DHRS3 | DLEU1 | NDUFS5 | INTS3 |
| SLC22A15 | SOCS2 | TNFSF8 | DEC1 | PDHX |
| INHBA | CTA-243E7.1 | GPX1 | CYFIP2 | ACSS3 |
| AKAP12 | PARD6G | ARID1A | MGP | RP11- |
| | | | | 103J8.1 |

In some cases, the nucleic specific genes may be one or more from Table 3A. In some cases, the nucleic specific genes may be one or more from cluster 0 of Table 3A. In some cases, the nucleic specific genes may be one or more from cluster 1 of Table 3A. In some cases, the nucleic specific genes may be one or more from cluster 2 of Table 3A.

TABLE 3A
Single Nuclei EZ Preparation (Myocyte clusters)

| | | | | | | | |
|---|---|---|---|---|---|---|---|
| cluster | gene | tstat | log2FC | pval | pval_adj | percent_exp | mean_exp |

Cluster 0 Myocyte

| | | | | | | | |
|---|---|---|---|---|---|---|---|
| 0 | PDE4D | 101.4143143 | 4.786413193 | 0 | 0 | 0.98912792 | 4.504366398 |
| 0 | GBE1 | 95.69503784 | 4.423283577 | 0 | 0 | 0.938237335 | 3.402583838 |
| 0 | DMD | 83.36749268 | 4.035079479 | 0 | 0 | 0.983344899 | 3.900079966 |
| 0 | MLIP | 81.49430847 | 3.826128483 | 0 | 0 | 0.953735832 | 3.334388733 |
| 0 | EYA4 | 79.35470581 | 3.792492628 | 0 | 0 | 0.895442979 | 2.989447832 |
| 0 | NEAT1 | 78.65914154 | 4.309886456 | 0 | 0 | 0.994679621 | 5.017502308 |
| 0 | ACACB | 78.27854156 | 3.705726862 | 0 | 0 | 0.930372427 | 3.217884302 |
| 0 | UBE2E2 | 75.7383728 | 3.709742546 | 0 | 0 | 0.866527874 | 2.932776928 |
| 0 | TBC1D8 | 73.55910492 | 3.714147806 | 0 | 0 | 0.85426787 | 2.932938337 |
| 0 | KIAA1217 | 71.66703796 | 3.674530745 | 0 | 0 | 0.860744853 | 3.024675369 |
| 0 | RYR1 | 69.62826538 | 3.295396566 | 0 | 0 | 0.919731668 | 3.027299643 |
| 0 | ZBTB16 | 69.43442535 | 3.193599224 | 0 | 0 | 0.958593569 | 3.304039717 |
| 0 | ABLIM2 | 66.81189728 | 3.430619717 | 0 | 0 | 0.840157298 | 2.853957891 |
| 0 | KCNMA1 | 66.66548157 | 4.140600204 | 0 | 0 | 0.699976868 | 2.758461237 |
| 0 | FKBP5 | 65.69849396 | 3.086441517 | 0 | 0 | 0.981494333 | 4.015912056 |
| 0 | ZFAND3 | 65.14512634 | 3.197586775 | 0 | 0 | 0.851260699 | 2.810356379 |
| 0 | MALAT1 | 65.02056885 | 2.816757441 | 0 | 0 | 0.999306037 | 7.213562965 |
| 0 | CTNNA3 | 63.68095398 | 3.684278011 | 0 | 0 | 0.720333102 | 2.673369169 |
| 0 | GTF2IRD1 | 61.8019104 | 3.52835989 | 0 | 0 | 0.733055748 | 2.667934179 |
| 0 | MAST2 | 61.40848923 | 3.414988041 | 0 | 0 | 0.738144807 | 2.620412827 |
| 0 | THRB | 60.24114609 | 3.381398201 | 0 | 0 | 0.74184594 | 2.713703394 |
| 0 | PDE7A | 60.21441269 | 3.414543152 | 0 | 0 | 0.719639139 | 2.568400145 |
| 0 | PARD3 | 60.15167618 | 3.209443092 | 0 | 0 | 0.811473514 | 2.880681753 |
| 0 | FBXO32 | 59.80839539 | 3.169672966 | 0 | 0 | 0.869303724 | 3.287328243 |
| 0 | CAP2 | 59.78777313 | 3.140753031 | 0 | 0 | 0.811936155 | 2.811924934 |
| 0 | NCOA1 | 58.31863403 | 3.054593563 | 0 | 0 | 0.798519547 | 2.737102509 |
| 0 | TRIM63 | 58.1307869 | 2.742858887 | 0 | 0 | 0.940781864 | 3.285308123 |
| 0 | CUX1 | 57.5647049 | 3.066031456 | 0 | 0 | 0.767522554 | 2.606505394 |
| 0 | SVIL | 57.30783844 | 2.878723383 | 0 | 0 | 0.911635438 | 3.109068394 |
| 0 | RALGAPA2 | 56.95275116 | 3.429069519 | 0 | 0 | 0.674994217 | 2.546990633 |
| 0 | PPARGC1A | 56.82494736 | 3.767965555 | 0 | 0 | 0.618320611 | 2.591074228 |
| 0 | PPM1L | 56.44360352 | 3.275944948 | 0 | 0 | 0.707610456 | 2.692656517 |
| 0 | GPHN | 56.43142319 | 3.078798294 | 0 | 0 | 0.765440666 | 2.66832304 |
| 0 | GPATCH8 | 56.22784805 | 3.015026093 | 0 | 0 | 0.777238029 | 2.704372168 |
| 0 | RBFOX1 | 55.55692291 | 3.130341291 | 0 | 0 | 0.771223687 | 2.857392073 |
| 0 | CLIP1 | 54.85471344 | 2.929381132 | 0 | 0 | 0.776312746 | 2.670673847 |
| 0 | CACNA2D1 | 54.73996735 | 2.970013142 | 0 | 0 | 0.765209345 | 2.71243453 |
| 0 | SSH2 | 54.57065582 | 2.861040592 | 0 | 0 | 0.825352764 | 2.890378714 |
| 0 | PPP1R12B | 54.27468872 | 3.098406553 | 0 | 0 | 0.699514226 | 2.554077148 |
| 0 | LINC-PINT | 54.22130585 | 2.91142416 | 0 | 0 | 0.841313902 | 3.016038418 |
| 0 | FOXO1 | 54.02421188 | 2.824852705 | 0 | 0 | 0.845246357 | 2.981667519 |
| 0 | ARPP21 | 53.97344971 | 3.331341982 | 0 | 0 | 0.654406662 | 2.581500053 |
| 0 | WDPCP | 53.43887711 | 3.162247181 | 0 | 0 | 0.749710849 | 2.903521061 |
| 0 | ZBTB20 | 53.19958115 | 2.689676523 | 0 | 0 | 0.874161462 | 2.923128843 |
| 0 | AKAP6 | 53.07445145 | 3.286131859 | 0 | 0 | 0.643997224 | 2.577456236 |
| 0 | MYOM3 | 52.98542404 | 3.314714432 | 0 | 0 | 0.64006477 | 2.5847435 |
| 0 | RERE | 52.97116852 | 2.772049427 | 0 | 0 | 0.776544067 | 2.626306057 |
| 0 | TMEM135 | 52.78007889 | 3.58013463 | 0 | 0 | 0.582928522 | 2.556276321 |
| 0 | FNDC3B | 52.7542572 | 2.831931353 | 0 | 0 | 0.790423317 | 2.7819345 |
| 0 | SLC16A10 | 52.65286636 | 3.084773779 | 0 | 0 | 0.699282905 | 2.667057753 |

Cluster 1 Myocyte

| | | | | | | | |
|---|---|---|---|---|---|---|---|
| 1 | MB | 91.84922028 | 3.319354057 | 0 | 0 | 0.996064099 | 5.042815685 |
| 1 | ACTA1 | 87.19593048 | 3.442605972 | 0 | 0 | 0.997469778 | 6.198307514 |
| 1 | TNNC1 | 85.91455841 | 3.39825654 | 0 | 0 | 0.990441383 | 4.939857006 |
| 1 | TCAP | 84.70059967 | 3.511332273 | 0 | 0 | 0.98734889 | 4.728960991 |
| 1 | TPM2 | 84.41035461 | 3.145828247 | 0 | 0 | 0.991284791 | 4.835346699 |
| 1 | CKM | 82.5300827 | 3.457877636 | 0 | 0 | 0.991003655 | 5.212295532 |
| 1 | MT-CO2 | 81.69557953 | 3.764410734 | 0 | 0 | 0.981163902 | 4.841016769 |
| 1 | MT-CO1 | 81.35375977 | 3.810287714 | 0 | 0 | 0.997188642 | 6.000914097 |
| 1 | MT-ATP6 | 81.00028992 | 3.7050457 | 0 | 0 | 0.972729829 | 4.49339056 |
| 1 | TNNI1 | 80.73355865 | 3.733238459 | 0 | 0 | 0.961203261 | 4.30051899 |
| 1 | MT-CO3 | 80.26324463 | 3.831389904 | 0 | 0 | 0.989879112 | 5.497419834 |
| 1 | MYL2 | 79.96720123 | 3.77920866 | 0 | 0 | 0.989035704 | 5.701789379 |
| 1 | GAPDH | 79.13586426 | 3.451181889 | 0 | 0 | 0.975260051 | 4.403001308 |
| 1 | MT-ND1 | 78.02526093 | 3.545531034 | 0 | 0 | 0.970480742 | 4.514090061 |
| 1 | MT-ND4 | 77.99588776 | 3.781897068 | 0 | 0 | 0.982569581 | 5.205623627 |
| 1 | TPT1 | 77.73480988 | 3.310798168 | 0 | 0 | 0.979195952 | 4.580207348 |
| 1 | MT-CYB | 77.23577118 | 3.725570917 | 0 | 0 | 0.970761878 | 4.74882555 |
| 1 | MT-ND2 | 75.27470398 | 3.687479973 | 0 | 0 | 0.965139162 | 4.76717186 |
| 1 | MYH7 | 73.28559113 | 3.714814901 | 0 | 0 | 0.931965139 | 4.232325077 |
| 1 | DES | 73.05266571 | 3.185059786 | 0 | 0 | 0.958110768 | 4.174062729 |
| 1 | RPLP1 | 71.21856689 | 3.139355898 | 0 | 0 | 0.958954175 | 4.182795048 |
| 1 | CA3 | 70.17639923 | 3.539601564 | 0 | 0 | 0.907506326 | 3.951590776 |
| 1 | TNNT1 | 69.30379486 | 2.447407722 | 0 | 0 | 0.991847062 | 4.913854599 |
| 1 | MYL1 | 64.50784302 | 3.218435287 | 0 | 0 | 0.906100647 | 3.897249937 |
| 1 | RP11-451G4.2 | 63.17667389 | 3.148574114 | 0 | 0 | 0.916221535 | 3.976822615 |
| 1 | MT-ND3 | 62.04638672 | 3.398599863 | 0 | 0 | 0.857464155 | 3.798155308 |
| 1 | MT2A | 61.83117294 | 2.981716156 | 0 | 0 | 0.933651954 | 4.018405437 |
| 1 | ALDOA | 60.7022438 | 3.103455544 | 0 | 0 | 0.885296598 | 3.783791065 |
| 1 | COX6A2 | 59.77955627 | 3.307111025 | 0 | 0 | 0.842001687 | 3.732350826 |
| 1 | RPS23 | 51.55150223 | 2.985025883 | 0 | 0 | 0.796738825 | 3.64713335 |
| 1 | MYL3 | 51.2010994 | 3.03745842 | 0 | 0 | 0.776778184 | 3.649103403 |
| 1 | FTH1 | 50.32514954 | 2.916522264 | 0 | 0 | 0.800955862 | 3.658000946 |
| 1 | TPM3 | 50.2421875 | 2.278628349 | 0 | 0 | 0.935338769 | 4.016551018 |
| 1 | RPL32 | 49.77017975 | 2.911195517 | 0 | 0 | 0.789148159 | 3.637048244 |
| 1 | RPS25 | 48.21488953 | 2.862089396 | 0 | 0 | 0.771998875 | 3.610204935 |
| 1 | TXNRD1 | 47.80765915 | 2.749428034 | 0 | 0 | 0.792521788 | 3.621031523 |
| 1 | UQCRB | 47.59354782 | 2.875733376 | 0 | 0 | 0.751757099 | 3.555393457 |
| 1 | COX7C | 47.34233856 | 2.949123859 | 0 | 0 | 0.721394434 | 3.494252682 |
| 1 | RPL13 | 46.49856949 | 2.743798256 | 0 | 0 | 0.774529098 | 3.605832815 |
| 1 | RPL10 | 46.34063721 | 2.709569693 | 0 | 0 | 0.775653641 | 3.581132889 |
| 1 | RPL29 | 45.20239258 | 2.806707859 | 0 | 0 | 0.724205791 | 3.530909538 |
| 1 | RPL7 | 44.03061295 | 2.859272718 | 0 | 0 | 0.677818386 | 3.453494072 |
| 1 | COX5B | 43.40749741 | 2.822931528 | 0 | 0 | 0.669384313 | 3.429303408 |
| 1 | COX7A1 | 43.3430481 | 2.832176685 | 0 | 0 | 0.663761597 | 3.452292442 |
| 1 | RPS13 | 43.29623413 | 2.824866295 | 0 | 0 | 0.666572955 | 3.421344995 |
| 1 | RPS15 | 42.74211884 | 2.715245962 | 0 | 0 | 0.699465842 | 3.502584457 |
| 1 | NDUFA4 | 42.12366104 | 2.821094513 | 0 | 0 | 0.642114141 | 3.431546688 |
| 1 | SLC25A4 | 41.95499802 | 2.676285744 | 0 | 0 | 0.689626089 | 3.470487356 |
| 1 | RPL8 | 41.50556564 | 2.734069824 | 0 | 0 | 0.653078437 | 3.411704302 |
| 1 | RPS24 | 40.15060806 | 2.582562447 | 0 | 0 | 0.670789992 | 3.432752132 |

Cluster 2 Myocyte

| | | | | | | | |
|---|---|---|---|---|---|---|---|
| 2 | NEAT1 | 30.68847084 | 2.97032547 | 6.7291E−173 | 1.3211E−169 | 0.969559585 | 5.193494797 |
| 2 | MALAT1 | 29.86100388 | 2.165190935 | 3.08E−164 | 4.9132E−161 | 0.998056995 | 7.363081455 |
| 2 | BCL6 | 28.84972 | 2.643959761 | 3.5688E−162 | 5.3581E−159 | 0.79857513 | 3.516818762 |
| 2 | NEXN | 25.86568642 | 2.396879673 | 1.1484E−133 | 1.1273E−130 | 0.768134715 | 3.350649357 |
| 2 | DMD | 23.82266235 | 2.23607254 | 1.4894E−114 | 1.2672E−111 | 0.885362694 | 4.077739716 |
| 2 | NEDD4 | 22.65476227 | 2.330491304 | 6.8869E−105 | 4.8826E−102 | 0.633419689 | 3.08161974 |
| 2 | TNNT3 | 22.17012978 | 2.612362862 | 3.1814E−100 | 2.19456E−97 | 0.533031088 | 3.090705872 |
| 2 | ART3 | 21.92366219 | 2.750362873 | 2.47695E−97 | 1.66366E−94 | 0.436528497 | 2.633883715 |
| 2 | SESN1 | 21.51962852 | 2.05092597 | 1.07288E−95 | 6.84581E−93 | 0.687176166 | 3.069423199 |
| 2 | SVIL | 21.33188057 | 1.890286565 | 3.21984E−94 | 2.00439E−91 | 0.829015544 | 3.434760571 |
| 2 | MYLK4 | 21.2521019 | 4.285891056 | 1.49334E−89 | 8.10947E−87 | 0.284974093 | 2.896261215 |
| 2 | MLIP | 20.72967148 | 1.892499208 | 2.52149E−89 | 1.34075E−86 | 0.829015544 | 3.481071234 |
| 2 | TRDN | 20.10221863 | 1.922663927 | 1.47307E−84 | 7.09381E−82 | 0.775906736 | 3.616367579 |
| 2 | BACH1 | 19.75351524 | 1.886599779 | 7.56104E−82 | 3.50873E−79 | 0.687823834 | 3.028022766 |
| 2 | RP11-434D9.1 | 19.48142815 | 2.392071724 | 5.70798E−79 | 2.60151E−76 | 0.457901554 | 2.973782778 |
| 2 | KLHL29 | 19.16649437 | 3.202505589 | 1.58526E−75 | 6.52591E−73 | 0.295336788 | 2.49774313 |
| 2 | SORBS1 | 19.04637718 | 2.158126354 | 4.56413E−76 | 1.90968E−73 | 0.486398964 | 2.775076628 |
| 2 | FAM134B | 18.58670998 | 2.033220291 | 8.15734E−73 | 3.25312E−70 | 0.503238342 | 2.738197803 |
| 2 | MYPN | 17.87212181 | 1.704197764 | 4.96686E−68 | 1.78548E−65 | 0.663860104 | 2.93957448 |
| 2 | OSBPL9 | 17.54953766 | 1.780570745 | 1.08848E−65 | 3.56171E−63 | 0.587435233 | 2.886753321 |
| 2 | GGT7 | 17.36830521 | 2.868407965 | 2.55872E−63 | 7.86823E−61 | 0.275906736 | 2.598615646 |
| 2 | FTX | 17.24509621 | 1.70461905 | 1.22483E−63 | 3.90768E−61 | 0.619170984 | 2.887447596 |
| 2 | NPAS2 | 17.17085075 | 2.140250921 | 1.16247E−62 | 3.49056E−60 | 0.396373057 | 2.729994297 |
| 2 | PDLIM3 | 16.91735458 | 1.641801 | 1.82046E−61 | 5.40275E−59 | 0.736398964 | 3.240107536 |
| 2 | MBNL1 | 16.7145195 | 1.493893862 | 4.21299E−60 | 1.23521E−57 | 0.755829016 | 3.063050747 |
| 2 | KIF1B | 16.51938057 | 1.570191026 | 8.56389E−59 | 2.37583E−56 | 0.660621762 | 2.889220476 |
| 2 | ARID5B | 16.32891655 | 1.635024786 | 1.73787E−57 | 4.71868E−55 | 0.585492228 | 2.759566545 |
| 2 | PDLIM5 | 16.3124733 | 1.407245874 | 2.01955E−57 | 5.42578E−55 | 0.827720207 | 3.399428129 |
| 2 | GATS | 16.25320053 | 2.076553345 | 1.23024E−56 | 3.17166E−54 | 0.360751295 | 2.434517145 |
| 2 | PDE4DIP | 16.23176003 | 1.414978981 | 6.62181E−57 | 1.72458E−54 | 0.781088083 | 3.117293596 |
| 2 | MAFG | 15.98687458 | 2.874735832 | 2.14157E−54 | 5.20565E−52 | 0.235103627 | 2.385196447 |
| 2 | UACA | 15.860672 | 1.86092937 | 2.89735E−54 | 6.97634E−52 | 0.414507772 | 2.609840393 |
| 2 | ATRNL1 | 15.68160343 | 3.555809259 | 3.34637E−52 | 7.55835E−50 | 0.191062176 | 2.368801117 |
| 2 | SLC7A2 | 15.42689133 | 1.858652711 | 1.53812E−51 | 3.4137E−49 | 0.392487047 | 2.528899431 |
| 2 | ZBTB16 | 15.17070293 | 1.293138027 | 4.65753E−50 | 9.90617E−48 | 0.858160622 | 3.18776083 |
| 2 | SLC20A2 | 15.09835911 | 1.66727972 | 1.24938E−49 | 2.63536E−47 | 0.462435233 | 2.604684114 |
| 2 | EIF4G3 | 14.98401928 | 1.660827398 | 6.28584E−49 | 1.30434E−46 | 0.462435233 | 2.704310179 |
| 2 | RORA | 14.85598564 | 1.592671156 | 3.2594E−48 | 6.55037E−46 | 0.582253886 | 3.27860117 |
| 2 | PPM1B | 14.79623985 | 1.517491817 | 7.6393E−48 | 1.51146E−45 | 0.529792746 | 2.713263273 |
| 2 | FKBP5 | 14.70949936 | 1.26316154 | 3.45996E−47 | 6.69004E−45 | 0.920336788 | 3.785018921 |
| 2 | AFF1 | 14.66799545 | 1.550988436 | 4.48372E−47 | 8.60436E−45 | 0.509067358 | 2.73216486 |
| 2 | ATP2A1 | 14.64425564 | 2.245908499 | 1.29347E−46 | 2.40972E−44 | 0.276554404 | 2.675018072 |
| 2 | EGF | 14.56193733 | 1.941646338 | 3.04741E−46 | 5.55565E−44 | 0.325777202 | 2.538100243 |
| 2 | PHKA1 | 14.51071644 | 1.909703732 | 6.04699E−46 | 1.09459E−43 | 0.329015544 | 2.457320929 |
| 2 | MYO18B | 14.41104317 | 1.46609652 | 1.38997E−45 | 2.49833E−43 | 0.582901554 | 2.886646271 |
| 2 | CD70 | 14.35990715 | 2.467304468 | 8.90057E−45 | 1.55595E−42 | 0.224740933 | 2.478243351 |
| 2 | ABCA10 | 14.35300732 | 1.801764131 | 4.5978E−45 | 8.09307E−43 | 0.351036269 | 2.466420889 |
| 2 | KCNQ5 | 14.33800411 | 1.553141236 | 3.56279E−45 | 6.3148E−43 | 0.60880829 | 3.292612553 |
| 2 | PDE4D | 14.27385807 | 1.438064694 | 1.33485E−44 | 2.30199E−42 | 0.888601036 | 3.920683622 |
| 2 | AGBL1 | 14.19988346 | 1.853292465 | 3.34284E−44 | 5.72613E−42 | 0.382124352 | 3.202352762 |

In some cases, the nucleic specific genes may be one or more from Table 3B. In some cases, the nucleic specific genes may be one or more from cluster 3 of Table 3B. In some cases, the nucleic specific genes may be one or more from cluster 4 of Table 3B. In some cases, the nucleic specific genes may be one or more from cluster 5 of Table 3B. In some cases, the nucleic specific genes may be one or more from cluster 6 of Table 3B.

TABLE 3B
Single Nuclei EZ Preparation (Top ranked genes for each cluster)

| Cluster 3 | Cluster 4 | Cluster 5 | Cluster 6 |
|---|---|---|---|
| Fibroblast | Endothelial | Satellite cell | Unclassified |
| gene | gene | gene | gene |
| LAMA2 | TIMP3 | MEG3 | ANK3 |
| DCN | SPARCL1 | LAMA2 | DOCK9 |
| NEGR1 | TACC1 | PLXDC2 | LAMA2 |
| APOD | FLT1 | CADM2 | PLCB4 |
| GSN | VWF | PTPRG | FGF13 |
| COL4A1 | ST6GALNAC3 | SPARCL1 | XPR1 |
| CFD | ANO2 | TRHDE | CDH13 |
| GPX3 | NEDD9 | TGFBR3 | MUSK |
| COL4A2 | B2M | FBXL7 | NAV2 |
| PTPRG | PECAM1 | TLN2 | CBLB |
| EBF1 | PITPNC1 | PAX7 | LMBR1 |
| LAMB1 | LDB2 | LPP | COL25A1 |
| LAMC1 | RASAL2 | RBMS3 | RBMS3 |
| LINC01088 | HLA-C | MUSK | PSD3 |
| PID1 | EPAS1 | AKT3 | LINC01091 |
| GREB1L | HLA-B | GPM6B | TMEM233 |
| TGFBR3 | SASH1 | CNKSR3 | FOCAD |
| AKT3 | AKT3 | SNED1 | FRY |
| NID1 | ADGRF5 | ABCA8 | GGT7 |
| COL6A3 | ELMO1 | RORA | RP11- |
| | | | 1080G15.1 |
| NOVA1 | TMTC1 | MARCH1 | AC108938.5 |
| IGFBP7 | CALD1 | NAV2 | UTRN |
| RBMS3 | TMSB4X | GPC6 | CHRNA1 |
| MAN1A1 | APBB2 | EGFR | DTNA |
| MAML2 | PLEKHG1 | PON2 | SCN5A |
| TACC1 | ENG | DYNC1I1 | NPAS2 |
| TGFBR2 | SYNE1 | ARHGAP24 | INPP4B |
| EGFR | DOCK9 | ELL2 | PRKAR1A |
| SCN7A | ID3 | ECHDC2 | MEF2C |
| C1R | ESYT2 | APOE | DGKD |
| ARHGAP24 | ACTB | HMCN2 | RAB11FIP3 |
| CBLB | TINAGL1 | DOCK9 | SLC8A3 |
| COL6A2 | TCF4 | MEG8 | LIMS1 |
| TSC22D3 | EBF1 | CDH4 | ITGA10 |
| LHFP | GSN | PRKD1 | HS6ST2 |
| CYP4B1 | UTRN | DANT2 | ANKRD10 |
| SVEP1 | ASAP1 | PITPNC1 | PDLIM3 |
| C1S | IFITM3 | ADAMTS9-AS2 | TLN2 |
| ARL15 | A2M | RNF150 | GALNT18 |
| RORA | HLA-E | PLEKHH2 | CAP2 |
| PLPP3 | IGFBP7 | ECE1 | AC016723.4 |
| LTBP4 | RGS5 | NAV1 | FAM20A |
| FBXL7 | FABP4 | PTCHD1-AS | SLC38A1 |
| AOX1 | PTPRB | CELF2 | MTSS1 |
| MAP1B | DLC1 | FBN1 | KCNQ5 |
| GALNT15 | MYH9 | CHN1 | RP11- |
| | | | 526F3.1 |
| ABCA6 | APP | BCL2 | RP11- |
| | | | 768F21.1 |
| SH3PXD2B | RBPMS | ZBTB20 | SLCO5A1 |
| ARHGAP10 | EMCN | CEBPD | CECR2 |
| SERPING1 | SPTBN1 | APBB2 | RAPGEF4 |

In some cases, the nucleic specific genes may be one or more from Table 4A. In some cases, the nucleic specific genes may be one or more from cluster 0 of Table 4A. In some cases, the nucleic specific genes may be one or more from cluster 3 of Table 4A.

TABLE 4A
Single Nuclei NST Preparation (Myocyte clusters)

| | | | | | | | |
|---|---|---|---|---|---|---|---|
| cluster | gene | tstat | log2FC | pval | pval_adj | percent_exp | mean_exp |

Cluster 0 Myocyte

| | | | | | | | |
|---|---|---|---|---|---|---|---|
| 0 | PDE4D | 86.28809357 | 3.382457495 | 0 | 0 | 0.952200729 | 4.040971756 |
| 0 | MLIP | 79.96066284 | 3.024142981 | 0 | 0 | 0.869638352 | 3.134570837 |
| 0 | DMD | 79.86351013 | 2.917862654 | 0 | 0 | 0.958228203 | 3.826997995 |
| 0 | SVIL | 78.10348511 | 2.949514389 | 0 | 0 | 0.907765629 | 3.380862236 |
| 0 | RP11-362A9.3 | 77.63995361 | 3.264942884 | 0 | 0 | 0.836697505 | 3.370554924 |
| 0 | NEB | 76.71774292 | 3.005819798 | 0 | 0 | 0.925567704 | 3.613859415 |
| 0 | TTN | 72.05146027 | 2.679608822 | 0 | 0 | 0.956405943 | 3.644062519 |
| 0 | EYA4 | 67.12184143 | 2.723618746 | 0 | 0 | 0.802074572 | 3.0225389 |
| 0 | NEXN | 66.27083588 | 2.647407293 | 0 | 0 | 0.836697505 | 3.133488178 |
| 0 | FBXO32 | 65.4458313 | 2.730417252 | 0 | 0 | 0.823521166 | 3.281497717 |
| 0 | TBC1D8 | 65.1006546 | 2.608644962 | 0 | 0 | 0.802915615 | 3.010251284 |
| 0 | NEAT1 | 64.90393829 | 1.862595558 | 0 | 0 | 0.991729745 | 4.966375828 |
| 0 | TRIM63 | 64.20433044 | 2.652400732 | 0 | 0 | 0.799551444 | 3.087059259 |
| 0 | PDLIM5 | 63.81215286 | 2.474675894 | 0 | 0 | 0.868376787 | 3.225019693 |
| 0 | MYPN | 63.602314 | 2.682075024 | 0 | 0 | 0.731987665 | 2.829251766 |
| 0 | ACACB | 61.73292923 | 2.504084826 | 0 | 0 | 0.796327446 | 2.996240377 |
| 0 | RYR1 | 61.31725311 | 2.546749353 | 0 | 0 | 0.741799832 | 2.806555033 |
| 0 | EMC10 | 60.68687439 | 2.951526403 | 0 | 0 | 0.736052705 | 3.452820063 |
| 0 | RP11-434D9.1 | 59.96511841 | 2.784704447 | 0 | 0 | 0.755817213 | 3.364788532 |
| 0 | TTN-AS1 | 58.13369751 | 2.355644464 | 0 | 0 | 0.770955985 | 2.889874935 |
| 0 | MYBPC1 | 57.38003159 | 2.775486708 | 0 | 0 | 0.783851976 | 3.787090778 |
| 0 | PDLIM3 | 57.05669403 | 2.655153513 | 0 | 0 | 0.756237735 | 3.327852249 |
| 0 | CAP2 | 57.04840469 | 2.441671371 | 0 | 0 | 0.726380712 | 2.875377893 |
| 0 | TRDN | 55.95820999 | 2.411050797 | 0 | 0 | 0.770955985 | 3.097038269 |
| 0 | PDE4DIP | 53.98460007 | 2.046288013 | 0 | 0 | 0.839921503 | 3.022218466 |
| 0 | MPV17 | 53.85848618 | 2.395528555 | 0 | 0 | 0.64255677 | 2.637868166 |
| 0 | FAM184B | 53.33772278 | 2.320257664 | 0 | 0 | 0.693159518 | 2.809761286 |
| 0 | ABLIM2 | 52.99470901 | 2.299114704 | 0 | 0 | 0.666526493 | 2.65839982 |
| 0 | GPATCH8 | 52.69500732 | 2.05796504 | 0 | 0 | 0.803476311 | 2.959880114 |
| 0 | MYO18B | 52.17118835 | 2.451497078 | 0 | 0 | 0.634707037 | 2.850847721 |
| 0 | SLC16A10 | 49.95625687 | 2.293143749 | 0 | 0 | 0.611017662 | 2.635055065 |
| 0 | BACH1 | 48.98518753 | 2.102327347 | 0 | 0 | 0.687132044 | 2.731636047 |
| 0 | RBFOX1 | 48.92731476 | 2.194832802 | 0 | 0 | 0.661900757 | 2.810971975 |
| 0 | TP63 | 48.91043472 | 2.308315277 | 0 | 0 | 0.605270535 | 2.755381346 |
| 0 | PEX5L | 48.71851349 | 2.391325235 | 0 | 0 | 0.614662181 | 2.973362923 |
| 0 | TACC2 | 48.57481003 | 2.209881783 | 0 | 0 | 0.627417998 | 2.715618372 |
| 0 | CLIP1 | 48.39741898 | 2.027094603 | 0 | 0 | 0.701569947 | 2.722061157 |
| 0 | KIF1B | 48.32849121 | 2.012714148 | 0 | 0 | 0.724137931 | 2.798211336 |
| 0 | RP11-532N4.2 | 48.17814636 | 2.468132257 | 0 | 0 | 0.572329689 | 3.03913641 |
| 0 | FAM134B | 48.16740799 | 2.299618959 | 0 | 0 | 0.613120269 | 2.840901613 |
| 0 | RP11-64D24.2 | 47.43904114 | 2.009727001 | 0 | 0 | 0.710681245 | 2.843584776 |
| 0 | PCNT | 45.9152298 | 2.124797344 | 0 | 0 | 0.576394729 | 2.522727489 |
| 0 | RIF1 | 45.34344864 | 2.075732231 | 0 | 0 | 0.615082703 | 2.710366011 |
| 0 | KCNQ5 | 44.73711014 | 2.136064291 | 0 | 0 | 0.613120269 | 2.910439014 |
| 0 | WDPCP | 44.68787766 | 2.064140797 | 0 | 0 | 0.666246145 | 2.992846966 |
| 0 | ALK | 44.52365875 | 2.202203274 | 0 | 0 | 0.54079058 | 2.664259911 |
| 0 | PDK4 | 44.49716568 | 2.233272552 | 0 | 0 | 0.781749369 | 3.879845619 |
| 0 | GRIK1 | 44.17916107 | 2.135849476 | 0 | 0 | 0.566302215 | 2.691514254 |
| 0 | CMYA5 | 43.92105865 | 1.923693657 | 0 | 0 | 0.643257639 | 2.623984337 |
| 0 | GBE1 | 42.98879623 | 1.87792027 | 0 | 0 | 0.723156714 | 3.030931234 |

Cluster 3 Myocyte

| | | | | | | | |
|---|---|---|---|---|---|---|---|
| 3 | MB | 45.36845016 | 5.039746761 | 7.2129E−278 | 1.9303E−273 | 0.960339943 | 4.138577938 |
| 3 | CA3 | 34.81185532 | 4.451338768 | 2.3391E−190 | 3.13E−186 | 0.889518414 | 3.875718117 |
| 3 | DES | 34.66391754 | 4.242812634 | 2.7831E−189 | 2.4827E−185 | 0.902266289 | 3.73320508 |
| 3 | TCAP | 34.25564575 | 4.496630192 | 1.3127E−183 | 8.7826E−180 | 0.872521246 | 3.747440577 |
| 3 | CKM | 33.64890671 | 3.682870388 | 4.0434E−180 | 2.1642E−176 | 0.970254958 | 4.142796516 |
| 3 | ACTA1 | 32.93518829 | 3.441322327 | 7.8474E−173 | 3.0002E−169 | 0.992917847 | 4.957188606 |
| 3 | MT-CO1 | 32.65647507 | 3.743468285 | 7.5662E−174 | 3.3748E−170 | 0.95184136 | 4.075603485 |
| 3 | ALDOA | 32.47722626 | 4.033285618 | 2.3301E−171 | 7.7947E−168 | 0.862606232 | 3.267829418 |
| 3 | TPM2 | 31.9103241 | 3.56080389 | 1.8361E−168 | 5.4599E−165 | 0.934844193 | 3.60007596 |
| 3 | TNNC1 | 31.80289841 | 4.126588345 | 3.6129E−166 | 9.6689E−163 | 0.875354108 | 3.836868286 |
| 3 | GAPDH | 31.20012474 | 3.576480389 | 6.7803E−163 | 1.6496E−159 | 0.915014164 | 3.413009405 |
| 3 | MYH7 | 31.13933754 | 5.795368195 | 5.1036E−143 | 9.1055E−140 | 0.695467422 | 3.568230867 |
| 3 | TNNI1 | 30.21241951 | 4.523618698 | 9.8125E−148 | 2.02E−144 | 0.779036827 | 3.676169634 |
| 3 | MT-CO3 | 29.60902214 | 3.326941252 | 2.3206E−149 | 5.1754E−146 | 0.946175637 | 3.726438999 |
| 3 | MT-CO2 | 29.28540611 | 3.578066349 | 1.9286E−147 | 3.6867E−144 | 0.879603399 | 3.354665041 |
| 3 | TNNT1 | 28.32418633 | 3.537322283 | 7.3986E−140 | 1.2375E−136 | 0.898016997 | 3.855657578 |
| 3 | MYL2 | 27.39080238 | 3.495162249 | 8.5424E−133 | 1.3448E−129 | 0.924929178 | 4.221122265 |
| 3 | CRYAB | 26.0314579 | 3.786319017 | 1.5072E−118 | 2.2409E−115 | 0.733711048 | 3.084027767 |
| 3 | COX6A2 | 25.55956268 | 3.735337734 | 1.9636E−115 | 2.6274E−112 | 0.723796034 | 3.037820339 |
| 3 | RPLP1 | 25.49644279 | 3.219713211 | 5.0387E−118 | 7.0972E−115 | 0.837110482 | 3.191402674 |
| 3 | NDUFA4 | 25.3147583 | 4.416327953 | 4.608E−109 | 5.3617E−106 | 0.624645892 | 2.846125126 |
| 3 | MT-CYB | 25.22691154 | 3.470152378 | 1.1962E−114 | 1.5245E−111 | 0.769121813 | 3.063998938 |
| 3 | MT2A | 24.72338104 | 3.14894557 | 2.2997E−112 | 2.7975E−109 | 0.890934844 | 3.538580418 |
| 3 | MYL3 | 24.51583672 | 4.114356041 | 5.7997E−105 | 6.4671E−102 | 0.640226629 | 3.249326468 |
| 3 | HSPB1 | 24.34414291 | 3.878652334 | 1.0053E−104 | 1.0761E−101 | 0.661473088 | 2.986209631 |
| 3 | MT-ND4 | 23.76493835 | 3.260474205 | 1.9768E−104 | 2.0347E−101 | 0.780453258 | 3.15635252 |
| 3 | FHL1 | 23.62839127 | 3.173719883 | 1.3066E−103 | 1.2951E−100 | 0.781869688 | 3.124896526 |
| 3 | MYL1 | 23.35695076 | 3.130089045 | 1.2634E−101 | 1.20758E−98 | 0.788951841 | 3.146692514 |
| 3 | FABP3 | 22.99947357 | 3.891649485 | 1.114E−94 | 1.02803E−91 | 0.603399433 | 2.787765265 |
| 3 | SLC25A4 | 22.54064941 | 3.710465431 | 2.43586E−92 | 2.17295E−89 | 0.616147309 | 2.861616373 |
| 3 | SLN | 22.09153748 | 4.073943615 | 1.05854E−87 | 9.13828E−85 | 0.556657224 | 2.720176458 |
| 3 | ENO3 | 20.91774178 | 3.252687216 | 1.5236E−82 | 1.19925E−79 | 0.630311615 | 2.73875308 |
| 3 | CHCHD10 | 20.87193298 | 3.395799637 | 7.97979E−82 | 6.10158E−79 | 0.593484419 | 2.675555706 |
| 3 | FTH1 | 20.84409904 | 2.932204485 | 5.76145E−84 | 4.81837E−81 | 0.747875354 | 3.12355113 |
| 3 | EIF1 | 20.70482826 | 2.984888077 | 1.46006E−82 | 1.18406E−79 | 0.69121813 | 2.87906599 |
| 3 | MYOZ1 | 20.34199142 | 3.192083359 | 6.82911E−79 | 5.07669E−76 | 0.611898017 | 2.650874376 |
| 3 | COX5B | 20.31842613 | 3.766685486 | 4.25406E−77 | 2.99598E−74 | 0.526912181 | 2.548796892 |
| 3 | COX7C | 20.07178688 | 3.53760314 | 3.79696E−76 | 2.60549E−73 | 0.54815864 | 2.666780949 |
| 3 | TPT1 | 19.91294098 | 2.628203392 | 8.49652E−78 | 6.14551E−75 | 0.787535411 | 3.121062756 |
| 3 | PYGM | 19.78445625 | 3.657372952 | 1.11867E−73 | 6.80406E−71 | 0.52266289 | 2.540686607 |
| 3 | RPS2 | 19.75072098 | 3.109641314 | 1.15093E−75 | 7.51248E−73 | 0.61898017 | 2.728892803 |
| 3 | MT-ATP6 | 19.72942162 | 2.911108732 | 8.87436E−76 | 5.93739E−73 | 0.678470255 | 2.917506218 |
| 3 | COX4I1 | 19.69806671 | 3.463656664 | 1.05081E−73 | 6.53994E−71 | 0.535410765 | 2.531657457 |
| 3 | TNNC2 | 19.55847359 | 3.327177048 | 2.47581E−73 | 1.44039E−70 | 0.635977337 | 3.476270914 |
| 3 | RPL13 | 19.42108345 | 2.868082047 | 5.50097E−74 | 3.50516E−71 | 0.66713881 | 2.846966743 |
| 3 | MT-ND1 | 19.38569832 | 2.820511103 | 1.28058E−73 | 7.61577E−71 | 0.682719547 | 2.914809942 |
| 3 | COX7A1 | 19.15505981 | 3.50350976 | 3.81575E−70 | 2.08402E−67 | 0.512747875 | 2.570352316 |
| 3 | MT-ND2 | 19.09560776 | 2.833659649 | 1.08526E−71 | 6.05077E−69 | 0.664305949 | 2.84440589 |
| 3 | RPL19 | 18.92830276 | 3.090246916 | 8.12166E−70 | 4.34704E−67 | 0.569405099 | 2.659833908 |
| 3 | S100A1 | 18.79019737 | 3.678087234 | 2.18413E−67 | 1.08244E−64 | 0.480169972 | 2.624881983 |

In some cases, the nucleic specific genes may be one or more from Table 4B. In some cases, the nucleic specific genes may be one or more from cluster 1 of Table 4B. In some cases, the nucleic specific genes may be one or more from cluster 2 of Table 4B. In some cases, the nucleic specific genes may be one or more from cluster 4 of Table 4B. In some cases, the nucleic specific genes may be one or more from cluster 5 of Table 4B. In some cases, the nucleic specific genes may be one or more from cluster 6 of Table 4B.

TABLE 4B
Single Nuclei NST Preparation (Top ranked genes for each cluster)

| Cluster 1 | Cluster 2 | Cluster 4 | Cluster 5 | Cluster 6 |
|---|---|---|---|---|
| Fibroblast | Endothelial | Macrophage | Smooth muscle | Satellite cell |
| gene | gene | gene | gene | gene |
| DCN | ST6GALNAC3 | RBPJ | ACTA2 | MEG3 |
| LAMA2 | ANO2 | MRC1 | PDGFRB | LAMA2 |
| NEGR1 | LDB2 | RBM47 | KCNAB1 | CADM2 |
| LAMC1 | FLT1 | FRMD4B | RBPMS | PAX7 |
| NOVA1 | RP3- | ZEB2 | CACNA1C | TLN2 |
| | 510L9.1 | | | |
| ABCA6 | TACC1 | TBXAS1 | MYH11 | ABCA8 |
| GSN | MECOM | FMN1 | NR2F2- | MARCH1 |
| | | | AS1 | |
| PID1 | TIMP3 | CPM | CALD1 | TRHDE |
| COL6A3 | DOCK9 | CD163 | DLC1 | PON2 |
| EBF1 | PLEKHG1 | F13A1 | SORBS2 | NLGN4X |
| APOD | VWF | DPYD | LPP | MUSK |
| LINC01088 | PTPRB | HDAC9 | RP11- | PTPRG |
| | | | 759A24.3 | |
| COL4A1 | PTPRM | ELMO1 | CLMN | DYNC1I1 |
| COL4A2 | PITPNC1 | MS4A6A | PDE3A | CNKSR3 |
| PTPRG | NEDD9 | SRGAP2 | RGS6 | ECHDC2 |
| GPX3 | SASH1 | C10orf11 | PRKG1 | PLXDC2 |
| GREB1L | PECAM1 | CELF2 | CPM | TGFBR3 |
| TGFBR3 | TCF4 | GNAQ | CACNB2 | CDH4 |
| ABCA8 | EPAS1 | FCHSD2 | LMOD1 | RBMS3 |
| ARHGAP24 | TMTC1 | FGD2 | CARMN | NAV2 |
| NID1 | SPARCL1 | DOCK8 | RP11- | AC005237.4 |
| | | | 394O4.5 | |
| SCN7A | ELMO1 | CSGALNACT1 | EPS8 | DANT2 |
| GALNT15 | ARL15 | SYK | IGFBP7 | PLEKHH2 |
| MAML2 | AKT3 | STARD13 | TAGLN | IPO9-AS1 |
| CFD | SEC14L1 | SLC8A1 | ADAMTS9- | MEG8 |
| | | | AS2 | |
| VIT | RASGRF2 | RGL1 | EBF1 | RNF150 |
| MAN1A1 | MAGI1 | MERTK | MYL9 | TPRG1 |
| AC007319.1 | PLCB4 | PIK3R5 | TACC1 | PTCHD1-AS |
| CBLB | RIN2 | RAB31 | TPM1 | GPM6B |
| TSC22D3 | EMCN | IQGAP2 | JUNB | FBXL7 |
| MGST1 | ABLIM3 | SPTLC2 | NBEAL1 | SPARCL1 |
| IGFBP7 | TANC1 | FOXP1 | SPARCL1 | CALCR |
| ADH1B | DOCK4 | PTPRC | HES4 | LPP |
| MAP1B | MCTP1 | AGFG1 | ITGA8 | PRKD1 |
| LAMB1 | UTRN | SLC1A3 | SLIT3 | ELL2 |
| EGFR | LRCH1 | RNF149 | INPP4B | AGFG1 |
| TGFBR2 | TPO | TRPS1 | APBB2 | COL25A1 |
| DCLK1 | RAPGEF5 | LDLRAD4 | HEYL | NAV1 |
| SVEP1 | SNTG2 | SMAP2 | ARHGAP10 | ECE1 |
| RBMS3 | HIF3A | SAT1 | ACTN1 | PNMT |
| SRPX2 | BTNL9 | MSR1 | RNF152 | BCL2 |
| SH3PXD2B | RASAL2 | FAM49B | ZNF331 | APOE |
| FBXL7 | PICALM | SRGAP2B | PTPRG | GPC6 |
| TACC1 | C10orf11 | LRRC16A | ARHGEF7 | ADAMTS9- |
| | | | | AS2 |
| COL6A2 | CADPS2 | ANKRD44 | NOTCH3 | SRPX2 |
| CXCL14 | APP | RP11- | RAD51B | SNED1 |
| | | 452H21.1 | | |
| FBN1 | ABLIM1 | MAN1A1 | RASAL2 | KANK1 |
| FTL | ASAP1 | RP11- | ETV6 | RP11- |
| | | 624C23.1 | | 556I14.1 |
| MFAP5 | AC129778.2 | RIN3 | ADGRL3 | CHN1 |
| AKT3 | SYNE1 | CPVL | FLNA | WDFY2 |

In some embodiments, the regulatory sequence is not the CMV, CK8, MHCK7, MCK, MB, ACTA1, TPM2, TPM3 or CKM promoter.

### Vectors

The composition herein may comprise one or more vectors. For example, the nucleotide sequence may be a part of a vector. The vector may be used for delivering the nucleotide sequence and other components in the composition to cells and/or control the expression of the polynucleotide. A vector refers to a nucleic acid molecule capable of transporting another nucleic acid to which it has been linked. A vector may be a replicon, such as a plasmid, phage, or cosmid, into which another DNA segment may be inserted so as to bring about the replication of the inserted segment. Generally, a vector is capable of replication when associated with the proper control elements. Examples of vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques.

Certain vectors may be capable of directing the expression of genes to which they are operatively-linked. Such vectors are referred to herein as “expression vectors.” Common expression vectors of utility in recombinant DNA techniques are often in the form of plasmids. A vector may be a recombinant expression vector that comprises a nucleic acid of the invention in a form suitable for expression of the nucleic acid in a host cell, which means that the recombinant expression vectors include one or more regulatory elements, which may be selected on the basis of the host cells to be used for expression, that is, operatively-linked to the nucleic acid sequence to be expressed. As used herein, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

A vector may be a viral vector, wherein virally-derived DNA or RNA sequences are present in the vector for packaging into a virus. Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome.

In some embodiments, vectors herein are lentiviral vectors. For example, the vectors may be packaged in lentiviruses. The vectors may be delivered into cells that are transduced by the lentiviruses. Within the cells, the vectors or portions thereof may be integrated into the genome of the cells. A lentiviral vector may be a vector derived from at least a portion of a lentivirus genome, including a self-inactivating lentiviral vector. Lentiviral vectors are a type of retrovirus that can infect both dividing and nondividing cells because their preintegration complex (virus “shell”) can get through the intact membrane of the nucleus of the target cell. Examples of lentivirus vectors that may be used in the clinic include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The lentiviral vectors may include sequences form the 5′ and 3′ LTRs of a lentivirus. In some examples, the vectors include the R and U5 sequences from the 5′ LTR of a lentivirus and an inactivated or self-inactivating 3′ LTR from a lentivirus. The LTR sequences may be LTR sequences from any lentivirus from any species. For example, they may be LTR sequences from HIV, SIV, FIV or BIV. The vectors may contain deletions of the regulatory elements in the downstream long-terminal-repeat sequence, eliminating transcription of the packaging signal that is required for vector mobilization. As such, the vector region may include an inactivated or self-inactivating 3′ LTR. The 3′ LTR may be made self-inactivating. For example, the U3 element of the 3′ LTR may contain a deletion of its enhancer sequence, such as the TATA box, Sp1 and NF-kappa B sites. As a result of the self-inactivating 3′ LTR, the provirus that is integrated into the host cell genome will comprise an inactivated 5′ LTR. Optionally, the U3 sequence from the lentiviral 5′ LTR may be replaced with a promoter sequence in the viral construct. This may increase the titer of virus recovered from the packaging cell line. An enhancer sequence may also be included. In certain aspects, the barcoded trans-splicing viral construct is a non-integrating lentiviral construct, where the construct does not integrate by virtue of having a defective (e.g., by site-specific mutation) or absent integrase gene.

In some embodiments, the vector is a adeno-associated virus (AAV) vector. The term “adeno-associated virus vector” or “AAV vector” refers to a vector comprising a viral genome based a serotype of Adeno-Associated Virus genome, and optionally additional nucleotide sequences (functional genes, transgenes, promoters, enhancers and any other desired gene sequences) that are inserted into the vector through cloning or any other method known in the art of recombinant genetic engineering, which is capable of transducing (infecting) cells and expressing these additional nucleotide sequences in the transduced cells. In some embodiments, the viral vector is a recombinant AAV (rAAV). Examples of rAAVs include rAAV6, rAAV1, rAAV7, rAAV8, and rAAV 9. In some examples, the rAAV is rAAV6. In certain embodiments, AAV can be used to target specific tissue. Eleven serotypes of AAV have thus far been identified, with the best characterized and most commonly used being AAV2. These serotypes differ in their tropism, or the types of cells they infect, making AAV a very useful system for preferentially transducing specific cell types. The chart below gives a summary of the tropism of AAV serotypes, indicating the optimal serotype(s) for transduction of a given organ.

| Tissue | Optimal Serotype |
|---|---|
| CNS | AAV1, AAV2, AAV4, AAV5, AAV8, |
| | AAV9 |
| Heart | AAV1, AAV8, AAV9 |
| Kidney | AAV2 |
| Liver | AAV7, AAV8, AAV9 |
| Lung | AAV4, AAV5, AAV6, AAV9 |
| Pancreas | AAV8 |
| Photoreceptor Cells | AAV2, AAV5, AAV8 |
| RPE (Retinal Pigment | AAV1, AAV2, AAV4, AAV5, AAV8 |
| Epithelium) | |
| Skeletal Muscle | AAV1, AAV6, AAV7, AAV8, AAV9 |

In certain embodiments, synthetic AAVs can be used to target specific tissues (see, e.g., Colella et al., Emerging Issues in AAV-Mediated In Vivo Gene Therapy, Mol Ther Methods Clin Dev. 2018 Mar. 16; 8: 87-104). Researchers have further refined the tropism of AAV through pseudotyping, or the mixing of a capsid and genome from different viral serotypes (see, e.g., Designer gene delivery vectors: molecular engineering and evolution of adeno-associated viral vectors for enhanced gene transfer. Kwon I, Schaffer D V. Pharm Res. 2008 March; 25(3):489-99; Adeno-associated virus serotypes: vector toolkit for human gene therapy. Wu Z, Asokan A, Samulski R J. Mol. Ther. 2006 September; 14(3):316-27. Epub 2006 Jul. 7; Recombinant AAV viral vectors pseudotyped with viral capsids from serotypes 1, 2, and 5 display differential efficiency and cell tropism after delivery to different regions of the central nervous system. Burger C, Gorbatyuk O S, Velardo M J, Peden C S, Williams P, Zolotukhin S, Reier P J, Mandel R J, Muzyczka N. Mol. Ther. 2004 August; 10(2):302-17; and From virus evolution to vector revolution: use of naturally occurring serotypes of adeno-associated virus (AAV) as novel vectors for human gene therapy. Grimm D, Kay M A. Curr Gene Ther. 2003 August; 3(4):281-304). These serotypes are denoted using a slash, so that AAV2/5 indicates a virus containing the genome of serotype 2 packaged in the capsid from serotype 5. Use of these pseudotyped viruses can improve transduction efficiency, as well as alter tropism. For example, AAV2/5 targets neurons that are not efficiently transduced by AAV2/2, and is distributed more widely in the brain, indicating improved transduction efficiency. Many of these hybrid viruses have been well characterized and may be preferred over standard viruses for in vivo applications.

Scientists have also experimented with hybrid capsids derived from multiple different serotypes, which also alter viral tropism. One common example is AAV-DJ, which contains a hybrid capsid derived from eight serotypes. AAV-DJ displays a higher transduction efficiency in vitro than any wild type serotype; in vivo, it displays very high infectivity across a broad range of cell types. The mutant AAV-DJ8 displays the properties of AAV-DJ, but with enhanced brain uptake.

One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is, operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety. Thus, the embodiments disclosed herein may also comprise transgenic cells comprising the CRISPR effector system. In certain example embodiments, the transgenic cell may function as an individual discrete volume. In other words samples comprising a masking construct may be delivered to a cell, for example in a suitable delivery vesicle, and if the target is present in the delivery vesicle, the CRISPR effector is activated and a detectable signal generated.

### Dosage of Vectors

In some embodiments, the vector, e.g., plasmid or viral vector is delivered to the tissue of interest by, for example, an intramuscular injection, while other times the delivery is via intravenous, transdermal, intranasal, oral, mucosal, or other delivery methods. Such delivery may be either via a single dose, or multiple doses. One skilled in the art understands that the actual dosage to be delivered herein may vary greatly depending upon a variety of factors, such as the vector choice, the target cell, organism, or tissue, the general condition of the subject to be treated, the degree of transformation/modification sought, the administration route, the administration mode, the type of transformation/modification sought, etc.

Such a dosage may further contain, for example, a carrier (water, saline, ethanol, glycerol, lactose, sucrose, calcium phosphate, gelatin, dextran, agar, pectin, peanut oil, sesame oil, etc.), a diluent, a pharmaceutically-acceptable carrier (e.g., phosphate-buffered saline), a pharmaceutically-acceptable excipient, and/or other compounds known in the art. The dosage may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, surfactants, antioxidants, anticaking agents, fillers, chelating agents, coating agents, chemical stabilizers, etc. may also be present, especially if the dosage form is a reconstitutable form. Suitable exemplary ingredients include microcrystalline cellulose, carboxymethylcellulose sodium, polysorbate 80, phenylethyl alcohol, chlorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

In an embodiment herein the vector is an adenovirus vector, which may be at a single booster dose containing at least 1×10⁵ particles (also referred to as particle units, pu) of adenoviral vector. In an embodiment herein, the dose preferably is at least about 1×10⁶ particles (for example, about 1×10⁶-1×10¹² particles), more preferably at least about 1×10⁷ particles, more preferably at least about 1×10⁸ particles (e.g., about 1×10⁸-1×10¹¹ particles or about 1×10⁸-1×10¹² particles), and most preferably at least about 1×100 particles (e.g., about 1×10⁹-1×10¹⁰ particles or about 1×10⁹-1×10¹² particles), or even at least about 1×10¹⁰ particles (e.g., about 1×10¹⁰-1×10¹² particles) of the adenoviral vector. Alternatively, the dose comprises no more than about 1 x 10¹⁴ particles, preferably no more than about 1×10¹³ particles, even more preferably no more than about 1×10¹² particles, even more preferably no more than about 1×10¹¹ particles, and most preferably no more than about 1×10¹⁰ particles (e.g., no more than about 1×10⁹ articles). Thus, the dose may contain a single dose of adenoviral vector with, for example, about 1×10⁶ particle units (pu), about 2×10⁶ pu, about 4×10⁶ pu, about 1×10⁷ pu, about 2×10⁷ pu, about 4×10⁷ pu, about 1×10⁸ pu, about 2×10⁸ pu, about 4×10⁸ pu, about 1×10⁹ pu, about 2×10⁹ pu, about 4 x 10⁹ pu, about 1×10¹⁰ pu, about 2×10¹⁰ pu, about 4×10¹⁰ pu, about 1×10¹¹ pu, about 2×10¹¹ pu, about 4×10¹¹ pu, about 1×10¹² pu, about 2×10¹² pu, or about 4×10¹² pu of adenoviral vector. See, for example, the adenoviral vectors in U.S. Pat. No. 8,454,972 B2 to Nabel, et. al., granted on Jun. 4, 2013; incorporated by reference herein, and the dosages at col 29, lines 36-58 thereof. In an embodiment herein, the adenovirus is delivered via multiple doses.

In an embodiment herein, the vector is an AAV vector. A therapeutically effective dosage for in vivo delivery of the AAV to a human is believed to be in the range of from about 20 to about 50 ml of saline solution containing from about 1×10¹⁰ to about 1×10¹⁰ functional AAV/ml solution. The dosage may be adjusted to balance the therapeutic benefit against any side effects. In an embodiment herein, the AAV dose is generally in the range of concentrations of from about 1×10⁵ to 1×10⁵⁰ genomes AAV, from about 1×10⁸ to 1×10²⁰ genomes AAV, from about 1×10¹⁰ to about 1×10¹⁶ genomes, or about 1×10¹¹ to about 1×10¹⁶ genomes AAV. A human dosage may be about 1×10¹³ genomes AAV. Such concentrations may be delivered in from about 0.001 ml to about 100 ml, about 0.05 to about 50 ml, or about 10 to about 25 ml of a carrier solution. Other effective dosages can be readily established by one of ordinary skill in the art through routine trials establishing dose response curves. See, for example, U.S. Pat. No. 8,404,658 B2 to Hajjar, et al., granted on Mar. 26, 2013, at col. 27, lines 45-60.

In an embodiment, herein the vector is a plasmid. In such plasmid compositions, the dosage should be a sufficient amount of plasmid to elicit a response. For instance, suitable quantities of plasmid DNA in plasmid compositions can be from about 0.1 to about 2 mg, or from about 1 μg to about 10 μg per 70 kg individual. Plasmids of the invention will generally comprise (i) a promoter; (ii) a sequence encoding a CRISPR-Cas protein, operably linked to said promoter; (iii) a selectable marker; (iv) an origin of replication; and (v) a transcription terminator downstream of and operably linked to (ii). The plasmid can also encode the RNA components of a CRISPR complex, but one or more of these may instead be encoded on a different vector.

The doses herein are based on an average 70 kg individual. The frequency of administration is within the ambit of the medical or veterinary practitioner (e.g., physician, veterinarian), or scientist skilled in the art. It is also noted that mice used in experiments are typically about 20 g and from mice experiments one can scale up to a 70 kg individual.

The dosage used for the compositions provided herein include dosages for repeated administration or repeat dosing. In particular embodiments, the administration is repeated within a period of several weeks, months, or years. Suitable assays can be performed to obtain an optimal dosage regime. Repeated administration can allow the use of lower dosage, which can positively affect off-target modifications.

### Nucleoproteins

The composition may be a nucleoprotein complex. A nucleoprotein may be a protein that can associate (e.g., bind, form a complex with) with a nucleic acid or nucleic acid sequence. The term also encompasses a complex comprising a protein that can associate with a nucleic acid or nucleic acid sequence and a nucleic acid or nucleic acid sequence (e.g., a histone associated with a DNA sequence). Thus, as the term is employed herein, a “nucleoprotein” comprises a complex comprising a protein that can associate with a nucleic acid as well just as a protein that is capable of associating with a nucleic acid or nucleic acid sequence, with no nucleic acid or nucleic acid sequence bound to the protein. In some embodiments, the composition comprises a nucleotide sequence and one or more proteins. The proteins may deliver the nucleotide sequence to cells or specific nuclei in multinucleated cells.

In some embodiments, the nucleoproteins may be ribonucleoproteins (RNP). RNPs have the advantage that they lead to rapid editing effects because this process avoids the need for transcription. An important advantage is that both RNP delivery is transient, reducing off-target effects and toxicity issues. Efficient genome editing in different cell types has been observed by Kim et al. (2014, Genome Res. 24(6):1012-9), Paix et al. (2015, Genetics 204(1):47-54), Chu et al. (2016, BMC Biotechnol. 16:4), and Wang et al. (2013, Cell. 9; 153(4):910-8). In particular embodiments, the ribonucleoprotein is delivered by way of a polypeptide-based shuttle agent as described in International Patent Publication No. WO 2016/161516. WO2016161516 describes efficient transduction of polypeptide cargos using synthetic peptides comprising an endosome leakage domain (ELD) operably linked to a cell penetrating domain (CPD), to a histidine-rich domain and a CPD.

### Genetic Modulating Agents

The nucleotide sequences may be or encode one or more genetic modulating agents. The genetic modulating agents may manipulate nucleic acids (e.g., genomic DNA or mRNA) in specific nuclei. In certain embodiments, mutations in genomic DNA is repaired. In certain embodiments, exon skipping or alternative splicing is induced in mRNA. In certain embodiments, mRNA is edited to alter splicing or repair a mutation. In certain embodiments, diseases caused by a dominant mutation in a gene is targeted by deactivating or silencing the mutated gene. In certain embodiments, a gene is repaired or deactivated only in nuclei where the gene is expressed.

### Gene Editing Systems

In some embodiments, the nucleotide sequence may be or encode one or more components of a gene editing system. Examples of gene editing systems include CRISPR-Cas systems, zinc finger nuclease systems, TALEN systems, and meganuclease systems.

In some cases, the gene editing system may edit a splice site of an mRNA (e.g., an mRNA transcribed from a mutated endogenous gene related to a disease). For example, the gene editing system may edit a splice site of dystrophin mRNA (e.g., in a muscle cell). The splice site may be within about 100, 80, 60, 50, 40, 30, 20, 10, 5, 4, 3, 2, 1 base(s) either upstream (e.g., 5′ side) or downstream (e.g., 3′ side) of an exon of an mRNA. The splice site may be within about 100, 80, 60, 50, 40, 30, 20, 10, 5, 4, 3, 2, 1 base(s) from exon 43, 44, 50, 51, 52, or 55 in dystrophin mRNA. In certain cases, there is no base between the splice site and exon 43, 44, 50, 51, 52, or 55 in dystrophin mRNA.

In certain cases, the gene editing system comprises or can generate a homologous template for repairing a gene (e.g., an endogenous gene in the specific nuclei). In one example, the endogenous gene is dystrophin gene. In some embodiments, the composition may comprise a means for generating a homologous donor template. Such means may be a nucleotide sequence that is or encodes a homologous donor template. In certain cases, the composition comprises a nucleotide sequence that is or encodes a homologous donor template. In some examples, an endogenous gene may be cut by the gene editing system herein. The donor template may insert to the cutting site (e.g., to repair the endogenous gene).

### CRISPR-Cas Modification

In some embodiments, a polynucleotide of the present invention described elsewhere herein can be modified using a CRISPR-Cas and/or Cas-based system (e.g., genomic DNA or mRNA, preferably, for a disease gene). The nucleotide sequence may be or encode one or more components of a CRISPR-Cas system. For example, the nucleotide sequences may be or encode guide RNAs. The nucleotide sequences may also encode CRISPR proteins, variants thereof, or fragments thereof.

In general, a CRISPR-Cas or CRISPR system as used herein and in other documents, such as International Patent Publication No. WO 2014/093622 (PCT/US2013/074667), refers collectively to transcripts and other elements involved in the expression of or directing the activity of CRISPR-associated (“Cas”) genes, including sequences encoding a Cas gene, a tracr (transactivating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

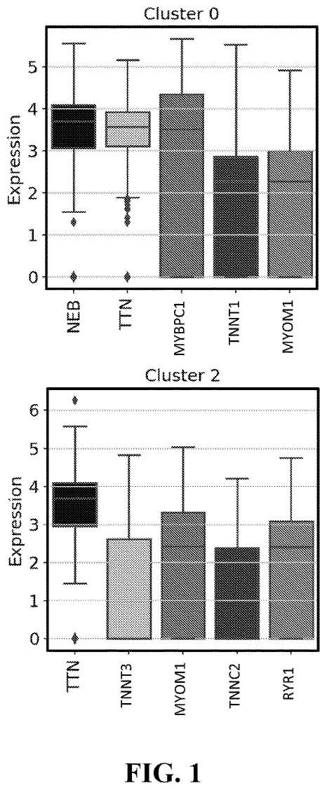

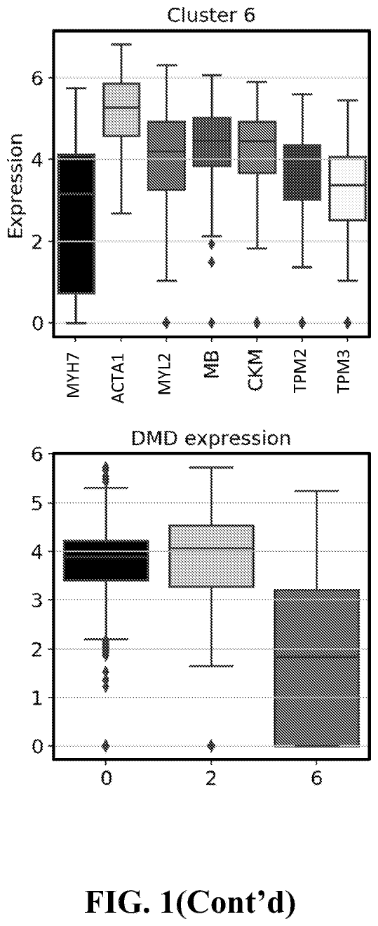

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

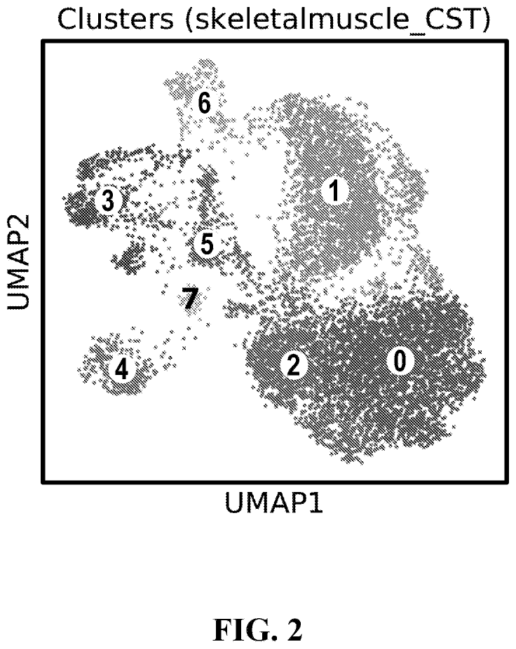

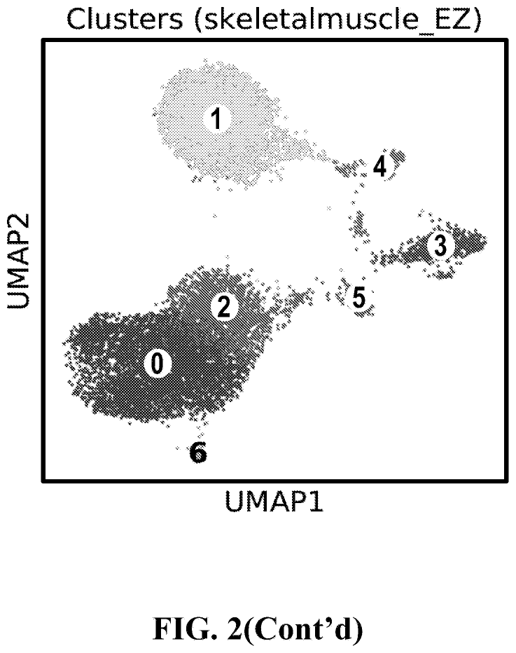

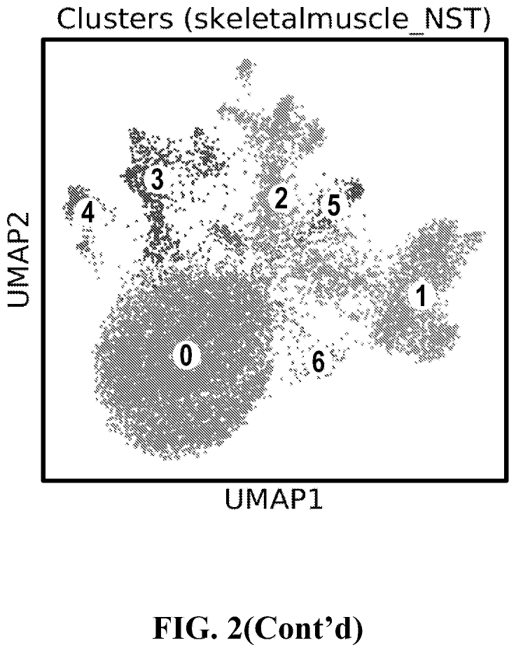

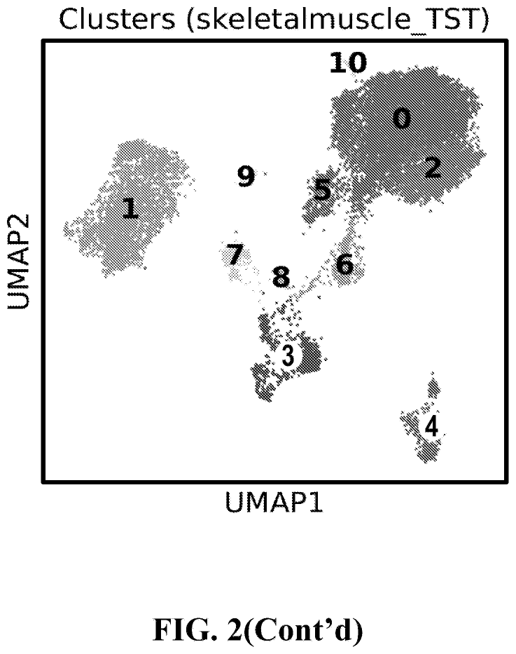

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Patent Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and International Patent Publication No. WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in International Patent Publication Nos. WO 2018/213708 and WO 2018/213726, and International Patent Application Nos. PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference. An example FnCas9 system that may be adapted for RNA base editing purposes is described in International Patent Publication No. WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system (See e.g. Anzalone et al. 2019. Nature. 576: 149-157). Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

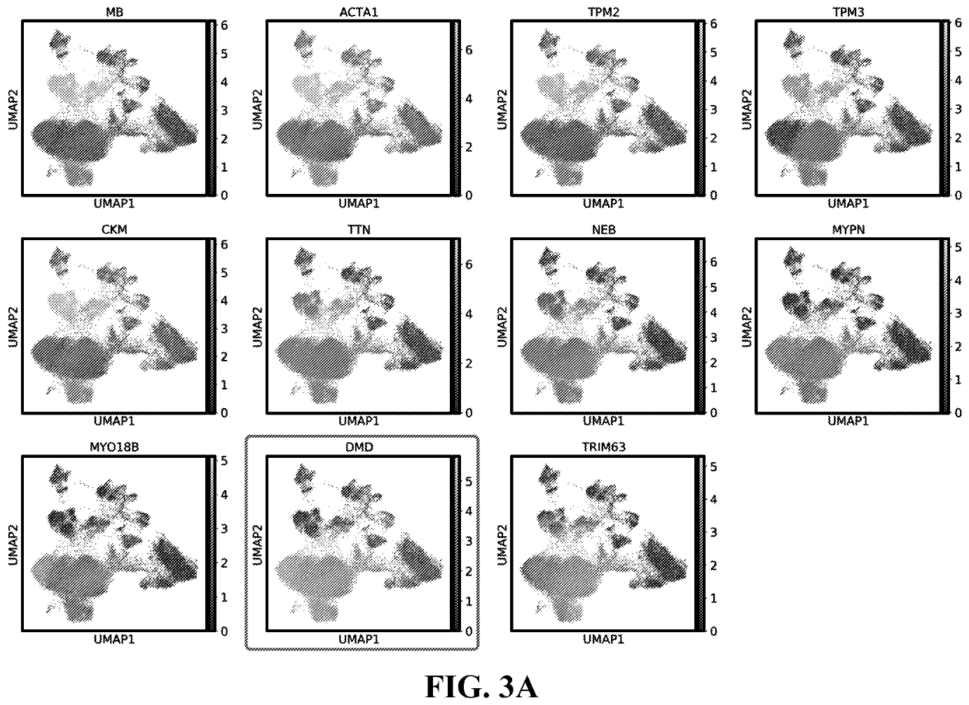

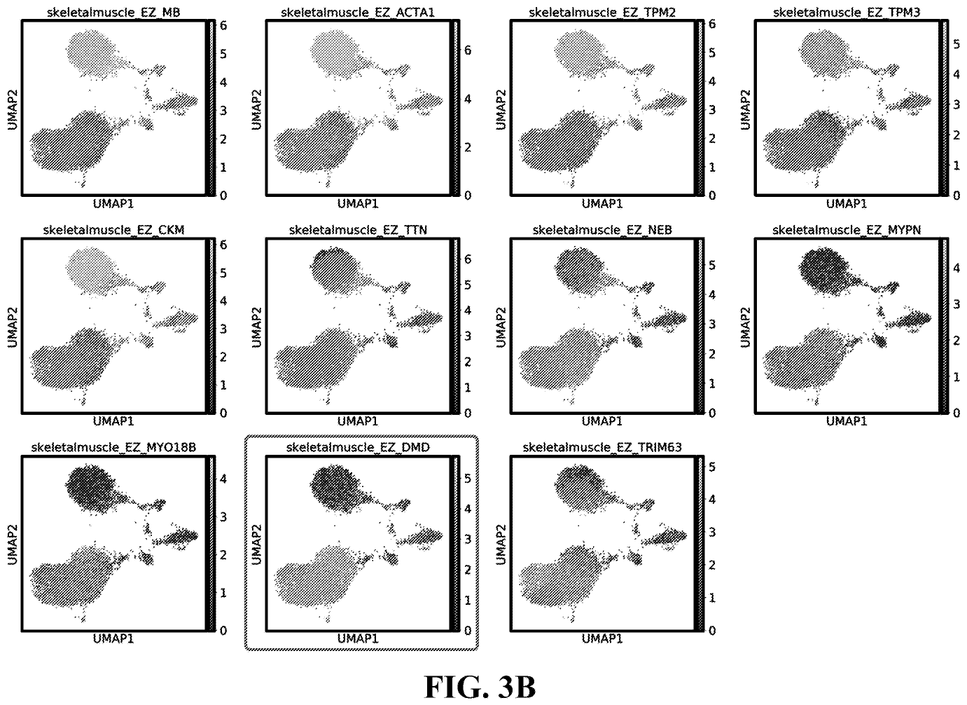

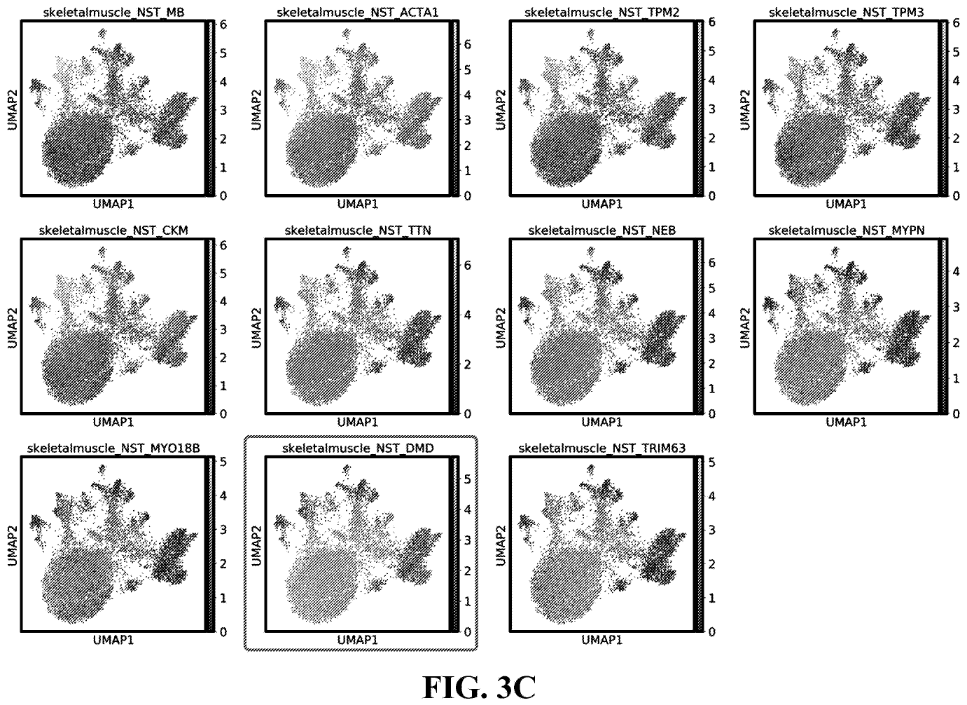

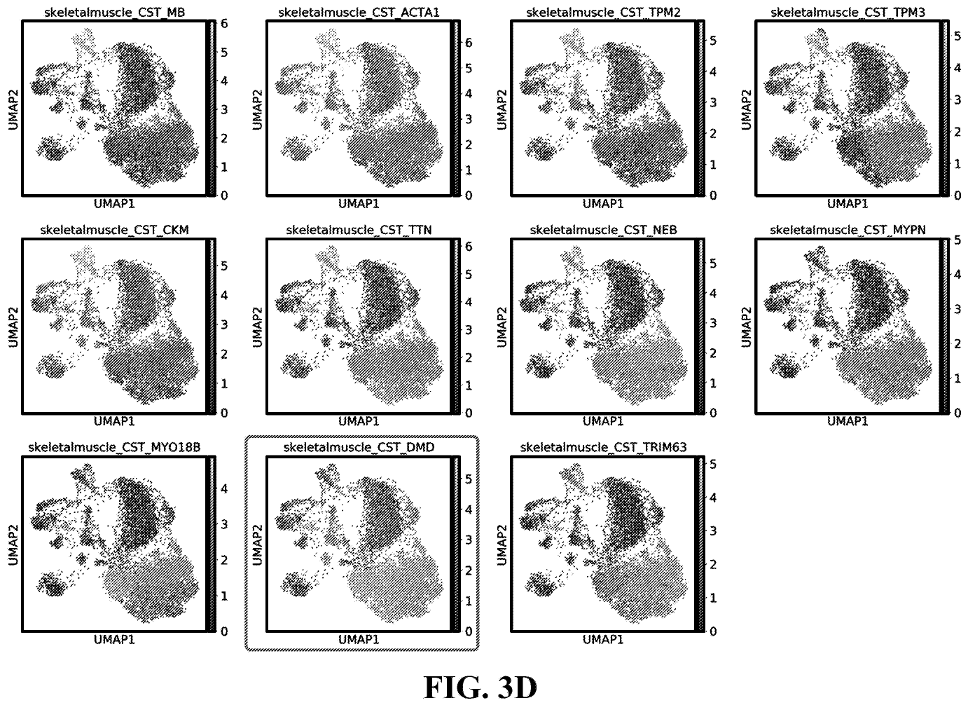

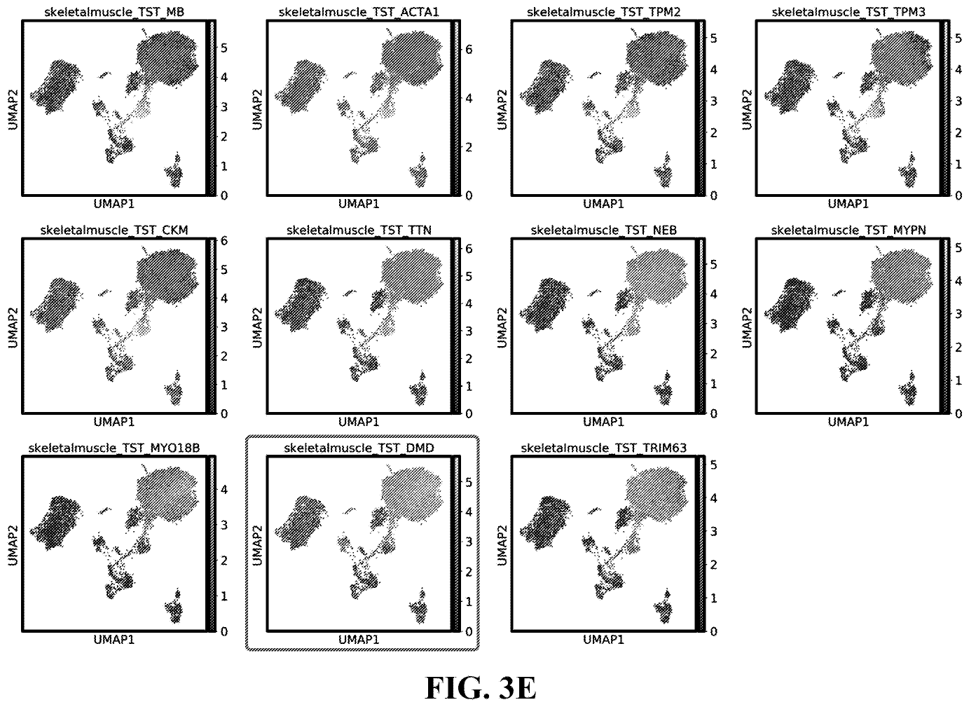

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) (including | TTTV |
| LbCpf1 and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAsl3a) have a specific discrimination against G at the 3′end of the target RNA. The presence of a C at the corresponding crRNA repeat site can indicate that nucleotide pairing at this position is rejected. However, some Cas13 proteins (e.g., LwaCAsl3a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

M D P I R S R T P S P A R E L L S G P Q P D G V Q
P T A D R G V S P P A G G P L D G L P A R R T M S
R T R L P S P P A P S P A F S A D S F S D L L R Q
F D P S L F N T S L F D S L P P F G A H H T E A A
T G E W D E V Q S G L R A A D A P P P T M R V A V
T A A R P P R A K P A P R R R A A Q P S D A S P A
A Q V D L R T L G Y S Q Q Q Q E K I K P K V R S T
V A Q H H E A L V G H G F T H A H I V A L S Q H P
A A L G T V A V K Y Q D

An exemplary amino acid sequence of a C-terminal capping region is:

(SEQ ID NO: 2)
R P A L E S I V A Q L S R P D P A L A A L T N D H
L V A L A C L G G R P A L D A V K K G L P H A P A
L I K R T N R R I P E R T S H R V A D H A Q V V R
V L G F F Q C H S H P A Q A F D D A M T Q F G M S
R H G L L Q L F R R V G V T E L E A R S G T L P P
A S Q R W D R I L Q A S G M K R A K P S P T S T Q
T P D Q A S L H A F A D S L E R D L D A P S P M H
E G D Q T R A S

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated herein by reference.

### Sequences Related to Nucleus Targeting and Transportation

In some embodiments, one or more components (e.g., the Cas protein and/or deaminase, Zn Finger protein, TALE, or meganuclease) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO:3) or PKKKRKVEAS (SEQ ID NO:4); the NLS from nucleoplasmin (e.g., the nucleoplasmin bipartite NLS with the sequence KRPAATKKAGQAKKKK (SEQ ID NO:5)); the c-myc NLS having the amino acid sequence PAAKRVKLD (SEQ ID NO:6) or RQRRNELKRSP (SEQ ID NO:7); the hRNPA1 M9 NLS having the sequence NQSSNFGPMKGGNFGGRSSGPYGGGGQYFAKPRNQGGY (SEQ ID NO:8); the sequence RMRIZFKNKGKDTAELRRRRVEVSVELRKAKKDEQILKRRNV (SEQ ID NO:9) of the IBB domain from importin-alpha; the sequences VSRKRPRP (SEQ ID NO:10) and PPKKARED (SEQ ID NO:11) of the myoma T protein; the sequence PQPKKKPL (SEQ ID NO:12) of human p53; the sequence SALIKKKKKMAP (SEQ ID NO:13) of mouse c-abl IV; the sequences DRLRR (SEQ ID NO:14) and PKQKKRK (SEQ ID NO:15) of the influenza virus NS1; the sequence RKLKKKIKKL (SEQ ID NO:16) of the Hepatitis virus delta antigen; the sequence REKKKFLKRR (SEQ ID NO:17) of the mouse Mx1 protein; the sequence KRKGDEVDGVDEVAKKKSKK (SEQ ID NO:18) of the human poly(ADP-ribose) polymerase; and the sequence RKCLQAGMNLEARKTKK (SEQ ID NO:19) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to a nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may be designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In certain embodiments, a template nucleic acid for correcting a mutation may be designed for use with a homology-independent targeted integration system. Suzuki et al. describe in vivo genome editing via CRISPR/Cas9 mediated homology-independent targeted integration (2016, Nature 540:144-149). Schmid-Burgk, et al. describe use of the CRISPR-Cas9 system to introduce a double-strand break (DSB) at a user-defined genomic location and insertion of a universal donor DNA (Nat Commun. 2016 Jul. 28; 7:12338). Gao, et al. describe “Plug-and-Play Protein Modification Using Homology-Independent Universal Genome Engineering” (Neuron. 2019 Aug. 21; 103(4):583-597).

### RNAi

In some embodiments, the genetic modulating agents may be interfering RNAs. In certain embodiments, diseases caused by a dominant mutation in a gene is targeted by silencing the mutated gene using RNAi. In some cases, the nucleotide sequence may comprise coding sequence for one or more interfering RNAs. In certain examples, the nucleotide sequence may be interfering RNA (RNAi). As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

In certain embodiments, a modulating agent may comprise silencing one or more endogenous genes. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Other Examples of Modulating Agents

The composition may comprise one or more other types of genetic modulating agents. The nucleotide sequences may be or comprise encoding sequences of the one or more genetic modulating agents. Agents useful in the methods as disclosed herein are proteins and/or peptides or fragment thereof, which inhibit the gene expression of a target gene or gene product, or the function of a target protein. Such agents include, for example but are not limited to protein variants, mutated proteins, therapeutic proteins, truncated proteins and protein fragments. Protein agents can also be selected from a group comprising mutated proteins, genetically engineered proteins, peptides, synthetic peptides, recombinant proteins, chimeric proteins, antibodies, midibodies, minibodies, triabodies, humanized proteins, humanized antibodies, chimeric antibodies, modified proteins and fragments thereof. As disclosed herein, a protein which inhibits the function of a target protein may be a soluble dominant negative form of the target protein or a functional fragment or variant thereof which inhibits wild-type full length target protein function.

In certain embodiments, the agents may be small molecules, antibodies, therapeutic antibody, antibody fragment, antibody-like protein scaffold, aptamer, protein, genetic modifying agent or small molecule. The chemical entity or biological product is preferably, but not necessarily a low molecular weight compound, but may also be a larger compound, or any organic or inorganic molecule effective in the given situation, including modified and unmodified nucleic acids such as antisense nucleic acids, RNAi, such as siRNA or shRNA, CRISPR-Cas systems, peptides, peptidomimetics, receptors, ligands, and antibodies, aptamers, polypeptides, nucleic acid analogues or variants thereof. Examples include an oligomer of nucleic acids, amino acids, or carbohydrates including without limitation proteins, oligonucleotides, ribozymes, DNAzymes, glycoproteins, siRNAs, lipoproteins, aptamers, and modifications and combinations thereof. Agents can be selected from a group comprising: chemicals; small molecules; nucleic acid sequences; nucleic acid analogues; proteins; peptides; aptamers; antibodies; or fragments thereof. A nucleic acid sequence can be RNA or DNA, and can be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), modified RNA (mod-RNA), single guide RNA etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides, CRISPR guide RNA, for example that target a CRISPR enzyme to a specific DNA target sequence etc. A protein and/or peptide or fragment thereof can be any protein of interest, for example, but are not limited to mutated proteins; therapeutic proteins and truncated proteins, wherein the protein is normally absent or expressed at lower levels in the cell. Proteins can also be selected from a group comprising mutated proteins, genetically engineered proteins, peptides, synthetic peptides, recombinant proteins, chimeric proteins, antibodies, minibodies, humanized proteins, humanized antibodies, chimeric antibodies, modified proteins and fragments thereof. Alternatively, the agent can be intracellular within the cell as a result of introduction of a nucleic acid sequence into the cell and its transcription resulting in the production of the nucleic acid and/or protein modulator of a gene within the cell. In some embodiments, the agent is any chemical, entity or moiety, including without limitation synthetic and naturally-occurring non-proteinaceous entities. In certain embodiments, the agent is a small molecule having a chemical moiety. Agents can be known to have a desired activity and/or property or can be selected from a library of diverse compounds.

### Exogenous Genes

In some embodiments, the nucleotide sequences comprise exogenous genes or functional fragments thereof. When expressed in specific nuclei or delivered to specific nuclei in multinucleated cells, the exogenous gene may express a product (e.g., protein or nucleic acid) that manipulates the function of the cells or treats a disease related to the function of the cells. For example, the exogenous gene may encode dystrophin or a functional fragment thereof (see, e.g., Duan, Systemic AAV Micro-dystrophin Gene Therapy for Duchenne Muscular Dystrophy, Molecular Therapy, Volume 26, Issue 10, 3 Oct. 2018, Pages 2337-235). The exogenous gene may be delivered to specific nuclei in muscle cells for treating muscular dystrophy.

### Exon Skipping

In some embodiments, the nucleotide sequences may encode nucleic acids capable of inducing exon skipping. Such encoded nucleic acids may be antisense oligonucleotides or antisense nucleotide systems. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using antisense oligonucleotides (AON) that are complementary to splice donor sequences within a pre-mRNA.

In some embodiments, the nucleotide sequences encode antisense oligonucleotides or antisense nucleotide systems capable of inducing exon skipping in dystrophin mRNA. For example, a non-sense or frameshift mutation within exon x of a dystrophin gene yields a carboxy-terminally truncated, non-functional dystrophin protein. The expression of that mature mRNA transcript may yield a functional dystrophin protein that is deleted in the amino acids encoded by exon x but that includes dystrophin amino acids both N-terminal and C-terminal to those deleted amino acids.

The nucleotide sequences may encode antisense oligonucleotides or antisense nucleotide systems capable of inducing exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof. Nucleotide sequences may encode antisense oligonucleotides or antisense nucleotide systems capable of inducing exon skipping at exon 43, 44, 50, 51, 52, 55, or any combination thereof.

In certain embodiments, the antisense oligonucleotide is linked to a snRNA, such as U7 snRNA (see, e.g., Imbert et al., Viral Vector-Mediated Antisense Therapy for Genetic Diseases, Genes 2017 February; 8(2): 51 doi:10.3390/genes8020051).

Viral vectors have been developed to deliver antisense oligonucleotides (see, e.g., Imbert et al., Viral Vector-Mediated Antisense Therapy for Genetic Diseases, Genes 2017 February; 8(2): 51 doi:10.3390/genes8020051; and Phillips, Antisense Inhibition and Adeno-Associated Viral Vector Delivery for Reducing Hypertension, Hypertension. 1997; 29:177-187). In certain embodiments, viral vectors include, but are not limited to the adeno-associated virus (AAV) vector, retroviruses and adenovirus.

### Homologous Recombination (HR)

In certain embodiments, donor vectors for homologous recombination are used to repair mutations in specific nuclei that express the mutated gene. In certain embodiments, —IR is used to repair a mutation in specific nuclei by using specific regulatory sequences specific to the nuclei. Precise genome editing can be accomplished using designer nucleases (e.g. ZFNs and TALENs) or RNA-guided nucleases (e.g. CRISPR/Cas9), which create site-specific double-strand breaks that stimulate homologous recombination (HR) when supplied with a homologous donor DNA template. For example, a nuclease system (e.g., CRISPR, TALEN, Zinc finger nuclease, or meganuclease) can be expressed using a nuclei specific regulatory sequence.

In certain embodiments, the donor DNA template is supplied by a vector. Viral vectors derived from the non-pathogenic, single-stranded DNA virus, adeno-associated virus (AAV), can transduce both dividing and non-dividing cells and have recently been effectively used as donor vectors for HR both in vitro and in vivo. In certain embodiments, AAV is used to supply the donor construct (see, e.g., Bak and Porteus, CRISPR-mediated Integration of Large Gene Cassettes using AAV Donor Vectors, Cell Rep. 2017 Jul. 18; 20(3): 750-756; Sather et al., Efficient modification of CCR5 in primary human hematopoietic cells using a megaTAl nuclease and AAV donor template, Sci Transl Med. 2015 Sep. 30; 7(307):307ra156; Wang et al., Homology-dri ven genome editing in hematopoietic stem and progenitor cells using ZFN mRNA and AAV6 donors, Nat Biotechnol. 2015 December; 33(12):1256-1263; and Yang et al., A dual AAV system enables the Cas9-mediated correction of a metabolic liver disease in newborn mice, Nat Biotechnol. 2016 March; 34(3):334-8).

### Methods of Using the Compositions

In another aspect, the present disclosure provides methods for treating diseases. The methods may comprise administering the compositions herein to a subject, e.g., a subject in need thereof. In some embodiments, the composition may be for use in treating a disease (e.g., muscular dystrophy) in a subject in need thereof. The composition may be capable of altering expression of dystrophin in the specific nuclei.

In some embodiments, the composition is capable of altering expression of one or more genes causing the disease in specific nuclei where the gene is normally expressed in a healthy subject. In some cases, the gene is altered exclusively in nuclei where it expressed in a normal subject.

### Cells

The methods herein may comprise delivering the compositions to cells in a subject. The cells may be multinucleated cells. A multinucleated cell may be a cell that has a plurality of cell nuclei. In such cases, the composition may target specific nuclei in the cells. Selective targeting specific nuclei in the cells may allow for accurately targeting genes related to the diseases and reduce side effects. Examples of multinucleated cells include skeletal muscle cells, cardiac muscle cells, placenta cells, syncytia, giant cells, osteoclasts, megakaryocytes, certain cancer cells (e.g., breast cancer cells, multiple myeloma cells, and certain metastatic cancer cells), macrophage-derived multinucleate cells, and hepatocytes. There is wide agreement that cell fusion is a physiological process in cells in mammalian bone, muscle and placenta. In other organs, such as the cerebellum, cell fusion is controversial. The liver contains a considerable number of polyploid cells; about 70% of hepatocytes are polyploid, arising either from genome duplication without division (endoreplication) or from cell fusion (see, e.g., Lizier et al., Cell fusion in the liver, revisited, World J Hepatol. 2018 Feb. 27; 10(2): 213-221).

In certain embodiments, cells from a subject are modified ex vivo and introduced back to the subject. In certain embodiments, pluripotent cells (e.g., stem cells) are obtained from a subject and a composition of the present invention is introduced to the cells. The pluripotent cells may be transferred back to the subject to be differentiated into the target multinucleated cell. Not being bound by a theory, the composition will only be expressed in the target nuclei upon differentiation into a target cell type.

### Diseases

The diseases that can be treated using the compositions and methods herein include those related to multinucleated cells (e.g., dysfunction of multinucleated cells). In certain embodiments, a gene mutation in a gene expressed in specific nuclei is targeted in a disease affecting multinucleated cells. In some cases, the disease is muscular dystrophy. In some examples, the muscular dystrophy is Duchenne's muscular dystrophy (DMD). In some examples, the muscular dystrophy is Becker muscular dystrophy (BMD). Other examples of muscular dystrophy include myotonic (Steinert's disease) dystrophy, congenital dystrophy, facioscapulohumeral (FSHD) dystrophy, limb-girdle dystrophy, and oculopharyngeal muscular dystrophy. In some examples, the muscular dystrophy results from one or more mutations of dystrophin.

In some examples, the disease is a monogenic disease associated with multinucleated cells (see, e.g., Kaplan and Hamroun, The 2016 version of the gene table of monogenic neuromuscular disorders (nuclear genome). Neuromuscul Disord. 2015 December; 25(12):991-1020. doi: 10.1016/j.nmd.2015.10.010. Epub 2015 Dec. 2; Bonne, Rivier and Hamroun, The 2018 version of the gene table of monogenic neuromuscular disorders (nuclear genome). Neuromuscul Disord. 2017 December; 27(12):1152-1183; and www.musclegenetable.fr). a monogenic disease may be a disease or condition that results from inactivation or malfunctioning of a single gene (e.g., due to mutation or deletion) occurring in cells, e.g., all cells, in a subject. In some cases, the monogenic disease is autosomal dominant. In some cases, the monogenic disease is autosomal recessive. In some cases, the monogenic disease is X-linked recessive. In some cases, the monogenic disease is X-linked dominant.

In certain embodiments, the compositions of the present invention are used to repair or inactivate a disease gene in specific nuclei for the condition selected from: Congenital Muscular Dystrophies (ACTA1, B3GALNT2, B4GAT1, CHKB, COL12A1, COL6A1, COL6A2, COL6A3, DAG1, DNM2, DPM1, DPM2, FKRP, FKTN, GMPPB, GOLGA2, GOSR2, INPP5K, ISPD, ITGA7, LAMA2, LARGE1, LMNA, MSTO1, POMGNT1, POMGNT2, POMK, POMT1, POMT2, RXYLT1, RYR1, SELENON, TCAP, TRAPPC11, TRIP4), Congenital Myasthenic Syndromes (AGRN, ALG14, ALG2, CHAT, CHRNA1, CHRNB1, CHRND, CHRNE, CHRNG, COL13A1, COLQ, DOK7, DPAGT1, GFPT1, GMPPB, LAMA5, LAMB2, LRP4, MUSK, MYO9A, PLEC, PREPL, RAPSN, RPH3A, SCN4A, SLC18A3, SLC25A1, SLC5A7, SNAP25, SYT2, UNC13A, VAMP1), Congenital Myopathies (ACTA1, ACTN2, BINI, CACNAlH, CACNA1S, CCDC78, CFL2, CNTN1, DNM2, FXR1, HACD1, HNRNPA1, HRAS, KBTBD13, KLHL40, KLHL41, LMOD3, MAP3K20, MEGF10, MTM1, MYBPC3, MYH2, MYH7, MYL1, MYL2, MYMK, MYO18B, MYPN, NEB, PAX7, PYROXD1, RYR1, RYR3, SCN4A, SELENON, SPEG, SPTBN4, STAC3, TNNT1, TNPO3, TPM2, TPM3, TRIM32, TTN), Distal Myopathies (ADSSL1, ANO5, CAV3, DNAJB6, DNM2, DYSF, FLNC, GNE, HSPB8, KLHL9, LDB3, MATR3, MYH7, MYOT, NEB, SQSTM1, TIAl, TTN, VCP), Hereditary Ataxias (ADCK3, AFG3L2, ANO10, APTX, ATG5, ATM, ATXN1, ATXN10, ATXN2, ATXN3, ATXN7, ATXN8OS, BEAN1, CACNA1A, CACNAlG, CACNB4, CCDC88C, COA7, CWF19L1, EEF2, ELOVL4, ELOVL5, FAT2, FGF14, FXN, GDAP2, GRID2, GRM1, IFRD1, ITPR1, KCNC3, KCND3, MME, MRE11A, MSTO1, NOP56, PCNA, PDYN, PEX7, PHYH, PLD3, POLG, PPP2R2B, PRKCG, PUMI, RFC1, RNF216, RUBCN, SACS, SCYL1, SETX, SILl, SLC1A3, SLC9A1, SNX14, SPTBN2, STUBI, SYNE1, SYT14, TBP, TDP1, TDP2, TGM6, TK2, TMEM240, TPP1, TRPC3, TTBK2, TTPA, TWNK, UBA5, VPS13D, VWA3B, WDR73, WWOX, XRCC1), Hereditary Cardiomyopathies (AARS2, ABCC9, ACTC1, ACTN2, AKAP9, ALPK3, ANK2, ANKRD1, BAG3, CACNAlC, CACNB2, CALM1, CALM2, CALR3, CASQ2, CAV3, COX15, CRYAB, CSRP3, CTNNA3, DES, DMD, DOLK, DSC2, DSG2, DSP, DTNA, EYA4, FKTN, FLNA, FLNC, GAA, GATAD1, GJA5, GPD1L, HACD1, HCN4, ILK, JPH2, JUP, KCNA5, KCND3, KCNE1, KCNE2, KCNE3, KCNH2, KCNJ2, KCNJ5, KCNQ1, LAMA4, LDB3, LMNA, MIB1, MRPL3, MRPL44, MTO1, MURC, MYBPC3, MYH6, MYH7, MYL2, MYL3, MYL4, MYLK2, MYOZ2, MYPN, NDUFAF1, NEXN, NPPA, NUP155, PKP2, PLN, PRDM16, PRKAG2, PSEN1, PSEN2, RAF1, RBM20, RYR2, SCN1B, SCN2B, SCN3B, SCN4B, SCN5A, SC02, SDHA, SGCD, SNTA1, SYNE1, TAZ, TCAP, TECRL, TGFB3, TMEM43, TMPO, TNNC1, TNNI3, TNNT2, TPM1, TRDN, TSFM, TTN, VCL), Hereditary Motor and Sensory Neuropathies (HMSN) (78 kb Chro8 insertion, AARS, AHNAK2, AIFM1, ARHGEF10, ATL1, ATL3, ATP1A1, BAG3, Clorfl94, CCT5, CLTCL1, COX6A1, CTDP1, DCAF8, DGAT2, DHTKD1, DNAJB2, DNM2, DNMT1, DST, DYNCIHI, EGR2, ELP1, FBLN5, FGD4, FIG4, FLVCR1, GAN1, GARS, GDAP1, GJB1, GJB3, GNB4, HARS, HINT1, HK1, HOXD10, HSPB1, HSPB8, IGHMBP2, INF2, KARS, KIF1A, KIF1B, KIF5A, LITAF, LMNA, LRSAM1, MARS, MFN2, MME, MORC2, MPV17, MPZ, MTMR2, NAGLU, NDRG1, NEFH, NEFL, NGF, NOTCH2NLC, NTRK1, PDK3, PLEKHG5, PMP2, PMP22, PNKP, PRDM12, PRPS1, PRX, RAB7A, RETREGI, SACS, SBF1, SBF2, SCN11A, SCN9A, SC02, SEPT9, SGPL1, SH3TC2, SLC12A6, SPG11, SPTLC1, SPTLC2, SURF1, TFG, TRIM2, TRPV4, VCP, VRK1, WNK1, YARS, ZFHX2), Hereditary Paraplegias (AFG3L2, ALDH18A1, ALDH3A2, ALS2, AMPD2, AP4B1, AP4E1, AP4M1, AP4S1, AP5Z1, ARL6IP1, ATL1, ATP13A2, B4GALNT1, BSCL2, C12orf65, C19orf12, CAPN1, CHP1, CPT1C, CYP2U1, CYP7B1, DDHD1, DDHD2, ENTPD1, ERLIN1, ERLIN2, FA2H, FARS2, GBA2, GJC2, HACE1, HSPD1, IBA57, KIAA0196, KIDINS220, KIF1A, KIF1C, KIF5A, KLC2, LlCAM, MAG, MARS2, MTPAP, NIPA1, NKX6-2, NT5C2, PLP1, PNPLA6, REEP1, REEP2, RTN2, SACS, SLC33A1, SPAST, SPG11, SPG20, SPG21, SPG7, TECPR2, TFG, UBAP1, UCHL1, VAMP1, VPS37A, ZFYVE26, ZFYVE27), Ion Channel Muscle Diseases (ATP1A2, CACNA1A, CACNAlS, CLCN1, KCNA1, KCNE3, KCNJ18, SCN4A), Malignan Hyperthermia (CACNAlS, RYR1, METABOLIC MYOPATHIES (ABHDS, ACAD9, ACADVL, AGL, CPT2, ENO3, ETFA, ETFB, ETFDH, FLAD1, GAA, GBE1, GYG1, GYS1, LDHA, LPIN1, PFKM, PGAM2, PGK1, PGM1, PHKA1, PNPLA2, PNPLA8, PRKAG2, PYGM, RBCK1, SLC16A1, SLC22A5, SLC25A20), Motor Neuron Diseases (AARS, ALS2, ANG, ANXA11, AR, ASAH1, ASCC1, ATP7A, ATXN2, BICD2, BSCL2, C9orf72, CHCHD10, CHMP2B, DCTN1, DNAJB2, DYNCIHI, ERBB3, ERBB4, EXOSC3, EXOSC8, FBXO38, FIG. 4, FUS, GARS, GLE1, HEXB, HINT1, HNRNPA1, HSPB1, HSPB3, HSPB8, IGHMBP2, KIF26B, KIF5A, MAPT, MATR3, MYH14, NEFH, NEK1, OPTN, PFN1, PIP5K1C, PLEKHG5, PRPH, PRUNE1, RBM7, REEP1, SETX, SIGMARI, SLC25A46, SLC52A2, SLC52A3, SLC5A7, SMN1, SOD1, SPG11, SPTAN1, SQSTM1, SYT2, TARDBP, TBK1, TRIP4, TRPV4, TUBA4A, UBA1, UBQLN2, VAPB, VCP, VRK1, WARS), Muscular Dystrophies (ANO5, BVES, CAPN3, CAV3, COL6A1, COL6A2, COL6A3, DAG1, DES, DMD, DNAJB6, DPM3, DUX4, DYSF, EMD, FHL1, FKRP, FKTN, GAA, GMPPB, HNRNPDL, ISPD, KBTBD13, LAMA2, LIMS2, LMNA, MYOT, PLEC, POGLUT1, POMGNT1, POMGNT2, POMT1, POMT2, PTRF, PYROXD1, SGCA, SGCB, SGCD, SGCG, SMCHD1, SYNE1, SYNE2, TCAP, TMEM43, TNPO3, TOR1AIP1, TRAPPC11, TRIM32, TTN, VCP), Myotonic Syndromes (ATP2A1, CAV3, CLCN1, CNBP, DMPK, HSPG2), Other Myopathies (ACVR1, BAG3, CASQ1, CAV3, CLN3, CRYAB, DES, FHL1, FLNC, HNRNPA2B1, ISCU, KY, LAMP2, LDB3, LRP12, MB, MSTN, MYOT, ORAIl, PABPN1, PLEC, PYROXD1, RYR1, SELENON, STIM1, TRIM54, TRIM63, TTN, VCP, VMA21), and Other Neuromuscular Disorders (ADCK3, AIFM1, ASCC1, BICD2, CHCHD10, CNTNAP1, COL25A1, COQ2, COQ4, COQ6, COQ7, COQ9, COX6A2, DGUOK, DNA2, DOK7, ECEL1, ELP1, FAM111B, FASTKD2, FBXL4, FDX2, KIF21A, MET, MGME1, MRPS25, MSTO1, MUSK, MYBPC1, MYH3, MYH8, NUP88, OPAl, PHOX2A, PIEZO2, POLG, POLG2, PTRH2, PUS1, RAPSN, RNASEH1, RRM2B, RYR1, SCN4A, SGCE, SLC25A4, SLC25A42, SUCLA2, SUCLG1, SYNE1, TIMM22, TK2, TMEM65, TNNI2, TNNT3, TOP3A, TOR1A, TPM2, TTR, TUBB3, TWNK, TYMP, YARS2).

### Pharmaceutical Compositions

The composition may be a pharmaceutical composition. The pharmaceutical compositions may comprise a nucleotide sequence and one or more other components making it suitable for pharmaceutical uses. For example, a “pharmaceutical composition” may comprise an excipient, such as a pharmaceutically acceptable carrier that is conventional in the art and that is suitable for administration to cells or to a subject.

In yet other embodiments, the methods of the disclosure include administering to a subject in need thereof an effective amount (e.g., therapeutically effective amount or prophylactically effective amount) of the treatments provided herein. Such treatment may be supplemented with other known treatments, such as surgery on the subject. In certain embodiments, the surgery is strictureplasty, resection (e.g., bowel resection, colon resection), colectomy, surgery for abscesses and fistulas, proctocolectomy, restorative proctocolectomy, vaginal surgery, cataract surgery, or a combination thereof.

The term “pharmaceutically acceptable” as used throughout this specification is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline or phosphate buffered saline), solubilisers, colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives, stabilisers, antioxidants, tonicity controlling agents, absorption delaying agents, and the like. The use of such media and agents for pharmaceutical active components is well known in the art. Such materials should be non-toxic and should not interfere with the activity of the cells or active components.

The precise nature of the carrier or excipient or other material will depend on the route of administration. For example, the composition may be in the form of a parenterally acceptable aqueous solution, which is pyrogen-free and has suitable pH, isotonicity and stability. For general principles in medicinal formulation, the reader is referred to Cell Therapy: Stem Cell Transplantation, Gene Therapy, and Cellular Immunotherapy, by G. Morstyn & W. Sheridan eds., Cambridge University Press, 1996; and Hematopoietic Stem Cell Therapy, E. D. Ball, J. Lister & P. Law, Churchill Livingstone, 2000.

The pharmaceutical composition can be applied parenterally, rectally, orally or topically. Preferably, the pharmaceutical composition may be used for intravenous, intramuscular, subcutaneous, peritoneal, peridural, rectal, nasal, pulmonary, mucosal, or oral application. In a preferred embodiment, the pharmaceutical composition according to the invention is intended to be used as an infuse. The skilled person will understand that compositions which are to be administered orally or topically will usually not comprise cells, although it may be envisioned for oral compositions to also comprise cells, for example when gastro-intestinal tract indications are treated. Each of the cells or active components as discussed herein may be administered by the same route or may be administered by a different route. By means of example, and without limitation, cells may be administered parenterally and other active components may be administered orally. In some cases, the composition or pharmaceutical composition may by intramuscular injection. In some cases, the composition or pharmaceutical composition may by intravascular injection.

Liquid pharmaceutical compositions may generally include a liquid carrier such as water or a pharmaceutically acceptable aqueous solution. For example, physiological saline solution, tissue or cell culture media, dextrose or other saccharide solution or glycols such as ethylene glycol, propylene glycol or polyethylene glycol may be included.

The composition may include one or more cell protective molecules, cell regenerative molecules, growth factors, anti-apoptotic factors or factors that regulate gene expression in the cells. Such substances may render the cells independent of their environment.

Such pharmaceutical compositions may contain further components ensuring the viability of the cells therein. For example, the compositions may comprise a suitable buffer system (e.g., phosphate or carbonate buffer system) to achieve desirable pH, more usually near neutral pH, and may comprise sufficient salt to ensure isoosmotic conditions for the cells to prevent osmotic stress. For example, suitable solution for these purposes may be phosphate-buffered saline (PBS), sodium chloride solution, Ringer's Injection or Lactated Ringer's Injection, as known in the art. Further, the composition may comprise a carrier protein, e.g., albumin (e.g., bovine or human albumin), which may increase the viability of the cells.

Further suitably pharmaceutically acceptable carriers or additives are well known to those skilled in the art and for instance may be selected from proteins such as collagen or gelatine, carbohydrates such as starch, polysaccharides, sugars (dextrose, glucose and sucrose), cellulose derivatives like sodium or calcium carboxymethylcellulose, hydroxypropyl cellulose or hydroxypropylmethyl cellulose, pregeletanized starches, pectin agar, carrageenan, clays, hydrophilic gums (acacia gum, guar gum, arabic gum and xanthan gum), alginic acid, alginates, hyaluronic acid, polyglycolic and polylactic acid, dextran, pectins, synthetic polymers such as water-soluble acrylic polymer or polyvinylpyrrolidone, proteoglycans, calcium phosphate and the like.

If desired, cell preparation can be administered on a support, scaffold, matrix or material to provide improved tissue regeneration. For example, the material can be a granular ceramic, or a biopolymer such as gelatine, collagen, or fibrinogen. Porous matrices can be synthesized according to standard techniques (e.g., Mikos et al., Biomaterials 14: 323, 1993; Mikos et al., Polymer 35:1068, 1994; Cook et al., J. Biomed. Mater. Res. 35:513, 1997). Such support, scaffold, matrix or material may be biodegradable or non-biodegradable. Hence, the cells may be transferred to and/or cultured on suitable substrate, such as porous or non-porous substrate, to provide for implants.

The pharmaceutical compositions may comprise one or more pharmaceutically acceptable salts. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. The term “pharmaceutically acceptable salt” further includes all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. It will be understood that, as used herein, references to specific agents (e.g., neuromedin U receptor agonists or antagonists), also include the pharmaceutically acceptable salts thereof.

Methods of administrating the pharmacological compositions, including agents, cells, agonists, antagonists, antibodies or fragments thereof, to an individual include, but are not limited to, intradermal, intrathecal, intramuscular, intraperitoneal, intravenous, subcutaneous, intranasal, epidural, by inhalation, and oral routes. The compositions can be administered by any convenient route, for example by infusion or bolus injection, by absorption through epithelial or mucocutaneous linings (for example, oral mucosa, rectal and intestinal mucosa, and the like), ocular, and the like and can be administered together with other biologically-active agents. Administration can be systemic or local. In addition, it may be advantageous to administer the composition into the central nervous system by any suitable route, including intraventricular and intrathecal injection. Pulmonary administration may also be employed by use of an inhaler or nebulizer, and formulation with an aerosolizing agent. It may also be desirable to administer the agent locally to the area in need of treatment; this may be achieved by, for example, and not by way of limitation, local infusion during surgery, topical application, by injection, by means of a catheter, by means of a suppository, or by means of an implant.

Therapy or treatment according to the invention may be performed alone or in conjunction with another therapy, and may be provided at home, the doctor's office, a clinic, a hospital's outpatient department, or a hospital. Treatment generally begins at a hospital so that the doctor can observe the therapy's effects closely and make any adjustments that are needed. The duration of the therapy depends on the age and condition of the patient, the stage of the cancer, and how the patient responds to the treatment. Additionally, a person having a greater risk of developing an inflammatory response (e.g., a person who is genetically predisposed or predisposed to allergies or a person having a disease characterized by episodes of inflammation) may receive prophylactic treatment to inhibit or delay symptoms of the disease.

### Delivery of Modulating Agents and Pharmaceutical Compositions

Various delivery systems are known and can be used to administer the agents and pharmacological compositions including, but not limited to, encapsulation in liposomes, microparticles, microcapsules; minicells; polymers; capsules; tablets; and the like. In one embodiment, the agent may be delivered in a vesicle, in particular a liposome. In a liposome, the agent is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. Nos. 4,837,028 and 4,737,323. In yet another embodiment, the pharmacological compositions can be delivered in a controlled release system including, but not limited to: a delivery pump (See, for example, Saudek, et al., New Engl. J. Med. 321: 574 (1989) and a semi-permeable polymeric material (See, for example, Howard, et al., J. Neurosurg. 71: 105 (1989)). Additionally, the controlled release system can be placed in proximity of the therapeutic target (e.g., a tumor), thus requiring only a fraction of the systemic dose. See, for example, Goodson, In: Medical Applications of Controlled Release, 1984. (CRC Press, Boca Raton, Fla.).

Delivery of Modulating Agents that are Polynucleotides

In cases in which the modulating agents are polynucleotides, they may be delivered to cell using suitable methods. In some embodiments, the polynucleotides may be packaged in viruses or particles, or conjugated to a vehicle for delivering into cells.

In some embodiments, the methods include packaging the polynucleotides in viruses and transducing cell with the viruses. Transduction or transducing herein refers to the delivery of a polynucleotide molecule to a recipient cell either in vivo or in vitro, by infecting the cells with a virus carrying that polynucleotide molecule. The virus may be a replication-defective viral vector. In some examples, the viruses may be virus (e.g. retroviruses, replication defective retroviruses, adenoviruses, replication defective adenoviruses, and adeno-associated viruses (AAVs)), and lentiviruses.

In some examples, the viruses are lentiviruses. Lentiviruses are complex retroviruses that have the ability to infect and express their genes in both mitotic and post-mitotic cells. Examples of lentiviruses include human immunodeficiency virus (HIV) (e.g., strain 1 and strain 2), simian immunodeficiency virus (SIV), feline immunodeficiency virus (FIV), BLV, EIAV, CEV, and visna virus. Lentiviruses may be used for nondividing or terminally differentiated cells such as neurons, macrophages, hematopoietic stem cells, retinal photoreceptors, and muscle and liver cells, cell types for which previous gene therapy methods could not be used. A vector containing such a lentivirus core (e.g. gag gene) can transduce both dividing and non-dividing cells.

In certain embodiments, the viruses are adeno-associated viruses (AAVs). AAVs are naturally occurring defective viruses that require helper viruses to produce infectious particles (Muzyczka, N., Curr. Topics in Microbiol. Immunol. 158:97 (1992)). It is also one of the few viruses that can integrate its DNA into nondividing cells. Vectors containing as little as 300 base pairs of AAV can be packaged and can integrate, but space for exogenous DNA is limited to about 4.5 kb. In some cases, an AAV vector may include all the sequences necessary for DNA replication, encapsidation, and host-cell integration. The recombinant AAV vector can be transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells.

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. Delivery can be to cells (e.g. in vitro or ex vivo administration) or target tissues (e.g. in vivo administration). Physical methods of introducing polynucleotides may also be used. Examples of such methods include injection of a solution containing the polynucleotides, bombardment by particles covered by the polynucleotides, soaking a cell, tissue sample or organism in a solution of the polynucleotides, or electroporation of cell membranes in the presence of the polynucleotides.

Examples of delivery methods and vehicles include viruses, nanoparticles, exosomes, nanoclews, liposomes, lipids (e.g., LNPs), supercharged proteins, cell permeabilizing peptides, and implantable devices. The nucleic acids, proteins and other molecules, as well as cells described herein may be delivered to cells, tissues, organs, or subjects using methods described in paragraphs [00117] to [00278] of Feng Zhang et al., (WO2016106236A1), which is incorporated by reference herein in its entirety.

### Single Nuclei Sequencing

In certain embodiments, single nuclei sequencing of tissues is used to identify single nuclei that express a gene associated with a disease as described herein. In certain embodiments, the expression of the gene is determined in single nuclei obtained from a healthy tissue sample, such that normal expression of the gene is determined. In certain embodiments, the tissue is a frozen tissue sample.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO 2017/164936 on Sep. 28, 2017; International Patent Application No. PCT/US2018/060860, published as WO 2019/094984 on May 16, 2019; International Patent Application No. PCT/US2019/055894, published as WO 2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

Previously, Applicants developed single nucleus RNA sequencing as a method to profile the expression of single cells. The outer membrane of the nucleus is continuous with the rough endoplasmic reticulum (RER). The RER is a site of RNA translation. Preserving a portion of it with the nucleus improves RNA recovery and single cell expression profiling. Applicants can isolate nuclei using different buffer compositions. The compositions of nuclei isolation solutions that worked best preserve a portion of the nuclear outer membrane/RER along with ribosomes as determined by electron microscopy.

Applicants performed single nucleus RNA profiling of cells from tissues using four cell lysis compositions (Nuclei EZ lysis buffer, SIGMA, CST, NST and TST).

Tween-20, and Nonidet P-40/IGEPAL CA-630 are both non-ionic detergents. CHAPS is a zwitterionic detergent. In certain embodiments, another zwitterionic detergents is used. Applicants used fixed salts concertation for the tests, although Applicants did try hypotonic solutions. The salts concentration was based on cellular concentrations of salts and what has been previously reported. Applicants used 146 mM NaCl, 1 mM CaCl₂), and 21 mM MgCl2. The NaCl concentration can likely be varied up to 300 mM, or completely eliminated, and replaced with another salt such as KCl (as has been done in various biochemistry preparations as needed). Similar, CaCl₂) can likely be replaced with other calcium containing salts and concentrations can be increased to 20 mM or more. The same is true for varying MgCl2 or adding in other salts.

Applicants examined these nuclei preparations with electron microscopy and found that they preserved a portion of the outer nuclear envelope/RER with the nuclei. As a comparison, Applicants tested the commercial Nuclei EZ lysis buffer from Sigma, which did not preserve the nuclear envelope. CST with 0.49% CHAPS was the top extraction solution with the highest score and lowest contamination. The nuclei have a nuclear membrane (not double membrane in all places), the membrane contiguous with RER and has ribosomes, and mitochondrial contamination was reduced.

Applicants found that the CST buffer has a lower intron/exon ratio compared to nuclei-only preps with EZ lysis reagent supporting more spliced RNA. The Intron/Exon ratio for each were as follows: CST=1.27904; EZ frozen=1.642955; and EZ chop=2.081659.

Buffers used to extract nuclei are described below:

| | | | | Detergent | | |
|---|---|---|---|---|---|---|
| | | Buffer | | concentration | | Additives |
| Composition | Buffer | conc. | Detergent | (%) | Salt conc. | concentration |
| ST | Tris | 10 mM | | | 146 mM NaCl, | |
| | | | | | 1 mM CaCl2, | |
| | | | | | 21 mM MgCl2 | |
| CST | Tris | 10 mM | CHAPS | 0.49 | 146 mM NaCl, | 0.01% BSA |
| | | | | | 1 mM CaCl2, | |
| | | | | | 21 mM MgCl2 | |
| TST | Tris | 10 mM | Tween-20 | 0.03 | 146 mM NaCl, | 0.01% BSA |
| | | | | | 1 mM CaCl2, | |
| | | | | | 21 mM MgCl2 | |
| NSTnPo | Tricine | 20 mM | NP40 | 0.2 | 146 mM NaCl, | 0.15 mM spermine |
| | | | | | 1 mM CaCl2, | 0.5 mM spermidine |
| | | | | | 21 mM MgCl2 | 0.01% BSA |
| NST | Tris | 10 mM | NP40 | 0.2 | 146 mM NaCl, | 0.01% BSA |
| | | | | | 1 mM CaCl2, | |
| | | | | | 21 mM MgCl2 | |

In certain embodiments, dimension reduction is used to cluster nuclei from single cells based on differentially expressed genes. In certain embodiments, the dimension reduction technique may be, but is not limited to, Uniform Manifold Approximation and Projection (UMAP) or t-SNE (see, e.g., Becht et al., Evaluation of UMAP as an alternative to t-SNE for single-cell data, bioRxiv 298430; doi.org/10.1101/298430; and Becht et al., Dimensionality reduction for visualizing single-cell data using UMAP, Nature Biotechnology volume 37, pages 38-44 (2019)).

### EXAMPLES

### Example 1

This example demonstrates the feasibility of applying single-nucleus RNA-seq methods to frozen, banked donor tissue in order to generate single-cell atlases across the human body. Applicants applied four different nuclei extraction methods to eight human tissue types. For each extraction method and tissue type, tissue from three donors was assayed. Extracted nuclei transcriptomes were then profiled using droplet-based methods. For skeletal muscle nuclei, applicants observed nuclei heterogeneity. One example was the expression of the Duchenne muscular dystrophy disease gene, DMD, in a subset of nuclei. The data are shown in Tables 1A, 1B, 2A, 2B, 3A, 3B, 4A, and 4B, and FIGS. 1, 2, 3A, 3B, 3C, 3D, and 3E.

The following nuclei extractions were performed and subsequently processed for profiling:

Nuclei were extracted using the Nuclei EZ Prep (NUC101-1KT, Sigma-Aldrich) as described (N. Habib et al., Massively parallel single-nucleus RNA-seq with DroNc-seq. Nat. Methods. 14, 955-958 (2017)), with the following modifications. The tissues were dounce homogenized with a 7 mL Dounce Tissue Grinder (VWR 22877-280) (20 times pestle A, 20 times pestle B) and buffer volumes were increased to 5 mL for homogenization.

Fresh-frozen tissues were disaggregated in 1 mL of custom nuclear extraction buffer (CST, NST, TST) with mild chopping by Tungsten Carbide Straight 11.5 cm Fine Scissors (14558-11, Fine Science Tools, Foster City, Calif.) for 10 minutes on ice. Large debris were removed with a 40 m strainer (Falcon). An additional 1 mL of buffer was used to wash the filter before proceeding to fluorescence-activated cell sorting (FACS). For droplet-based RNA-Seq, nuclei were isolated as described above, but with the addition of 3 ml of ST (Salts and Tris) to extracted nuclei. Nuclei were then pelleted at 500 g for 5 mins at 4° C. Supernatant was discarded and the nuclei pellet was resuspended in 100-500 μL of ST buffer (Salts and Tris) before filtering through a 40 μm strainer-capped round bottom tube (Falcon).

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A composition for specifically targeting a nuclei type in a multinucleated cell or population of multinucleated cells, wherein the composition comprises one or more nucleotide sequences operably linked to one or more regulatory sequences specifically active in the nuclei.

2. The composition of claim 1, wherein the multinucleated cell is selected from the group consisting of a skeletal muscle cell, cardiac muscle cell, liver cell, and placenta cell.

3. The composition of claim 1 or 2, wherein the one or more regulatory sequences comprises a promoter active in the nuclei.

4. The composition of any of claims 1 to 3, wherein the one or more regulatory sequences comprises an enhancer active in the nuclei.

5. The composition of any of claims 1 to 4, further comprising one or more viral vectors selected from the group consisting of adeno-associated virus (AAV) vectors, lentivirus vectors, and adenovirus vectors.

6. The composition of claim 5, wherein the viral vector is a recombinant AAV (rAAV) vector.

7. The composition of claim 6, wherein the rAAV is selected from the group consisting of rAAV6, rAAV1, rAAV7, rAAV8, and rAAV 9.

8. The composition of claim 7, wherein the rAAV is rAAV6.

9. The composition of any of claims 1 to 4, wherein the composition is a nucleoprotein complex.

10. The composition of any of claims 1 to 9, wherein the one or more nucleotide sequences encodes one or more components of: a CRISPR system, a zinc finger nuclease system, a TALE system, a TALEN system, a meganuclease, antisense oligonucleotide, or RNAi system.

11. The composition of any of claims 1 to 10, further comprising a homologous donor template.

12. The composition of claim 10, wherein the CRISPR system is a Class 1 or Class 2 CRISPR system.

13. The composition of claim 12, wherein the Class 2 system comprises a Type II Cas polypeptide.

14. The composition of claim 13, wherein the Type II Cas is a Cas9.

15. The composition of claim 12, wherein the Class 2 system comprises a Type V Cas polypeptide.

16. The composition of claim 15, wherein the Type V Cas is Cas12a, Cas12b, Cas12c, Cas12d (CasY), Cas12e(CasX), or Cas14.

17. The composition of claim 12, wherein the Class 2 system comprises a Type VI Cas polypeptide.

18. The composition of claim 17, wherein the Type VI Cas is Cas13a, Cas13b, Cas13c or Cas13d.

19. The composition of any of claims 12 to 18, wherein the CRISPR system comprises an inactivated CRISPR enzyme (dCas) fused or otherwise linked to a nucleotide deaminase.

20. The composition of claim 19, wherein the nucleotide deaminase is a cytidine deaminase or an adenosine deaminase.

21. The composition of claim 19, wherein the dCas is a dCas9, dCas12, or dCas13.

22. The composition of claim 19, wherein the nucleotide deaminase is capable of DNA editing.

23. The composition of claim 19, wherein the nucleotide deaminase is capable of altering mRNA splicing by editing mRNA.

24. The composition of any of claims 1 to 10, further comprising an AAV replication system capable of generating antisense DNA molecules or homologous donor templates.

25. The composition of any of claims 1 to 24, wherein the one or more regulatory sequences are derived from endogenous regulatory sequences that control expression of one or more myocyte nuclei specific genes selected from the group consisting of:
a) PDK4, PDE4D, DMD, NEB, TTN, SVIL, RP11-434D9.1, MLIP, PDLIM5 and EYA4; or
b) TTN, NEB, MLIP, SVIL, DMD, NEXN, RP11-362A9.3, PDE4D, FBXO32 and TRDN; or
c) PDE4D, NEB, TRIM63, MLIP, EYA4, FBXO32, TBC1D8, TTN, GBE1 and DMD; or
d) RP11-434D9.1, ART3, EMC10, SVIL, NEXN, BCL6, DMD, MYO18B, SESN1 and TTN; or
e) GBE1, PDE4D, DMD, MLIP, EYA4, NEAT1, ACACB, UBE2E2, TBC1D8 and KIAA1217; or
f) NEAT1, MALAT1, BCL6, NEXN, DMD, NEDD4, TNNT3, ART3, SESN1 and SVIL; or
g) MLIP, PDE4D, DMD, SVIL, RP11-362A9.3, NEB, TTN, EYA4, NEXN and FBXO32; or
h) genes in Table 1A cluster 2; or
i) genes in Table 1A cluster 0; or
j) genes in Table 2A cluster 2; or
k) genes in Table 2A cluster 0; or
l) genes in Table 3A cluster 2; or
m) genes in Table 3A cluster 0; or
n) genes in Table 4A cluster 0,
whereby the composition is specific to nuclei expressing the one or more markers.

26. The composition of claim 25, wherein the one or more regulatory sequences comprises a promoter specific to the one or more genes.

27. The composition of claim 25, wherein the one or more regulatory sequences comprises an enhancer specific to the one or more genes.

28. The composition of any of claims 25 to 27 for use in treating muscular dystrophy in a subject in need thereof, wherein the composition is capable of altering expression of dystrophin in the specific nuclei.

29. The composition of claim 28, wherein the muscular dystrophy is Duchenne's muscular dystrophy (DMD).

30. The composition of claim 28, wherein the muscular dystrophy is Becker muscular dystrophy (BMD).

31. The composition of any of claims 28 to 30, wherein the nucleotide sequence encodes an antisense oligonucleotide or antisense nucleotide system capable of inducing exon skipping in dystrophin mRNA.

32. The composition of claim 31, wherein the antisense oligonucleotide or antisense nucleotide system induces exon skipping at exon 43, 44, 50, 51, 52 or 55.

33. The composition of any of claims 28 to 30, wherein the nucleotide sequence encodes an exogenous dystrophin gene or functional fragment.

34. The composition of any of claims 28 to 30, wherein the CRISPR system, zinc finger nuclease system, or TALE system edits a splice site in dystrophin mRNA.

35. The composition of claim 34, wherein the splice site is within 50 bases from exon 43, 44, 50, 51, 52, or 55 in dystrophin mRNA.

36. The composition of any of claims 28 to 30, wherein the CRISPR system, zinc finger nuclease system, TALEN system, or a meganuclease specifically cuts the endogenous dystrophin gene.

37. The composition of claim 36, further comprising a means for generating a homologous donor template specific for repairing the endogenous dystrophin gene.

38. The composition of any of claims 1 to 25 for use in treating a monogenic disease associated with multinucleated cells in a subject in need thereof, wherein the composition is capable of altering expression of the gene causing the disease in the specific nuclei where the gene is normally expressed in a healthy subject.

39. The composition of claim 38, wherein the disease is autosomal dominant, autosomal recessive, X-linked recessive, or X-linked dominant.

40. The composition according to any of the preceding claims, wherein the gene is altered exclusively in nuclei where it is expressed in a normal subject.

41. The composition according to any of the preceding claims, wherein the regulatory sequence is not the CMV, CK8, MHCK7, MCK, MB, ACTA1, TPM2, TPM3 or CKM promoter.

42. A method of treating muscular dystrophy in a subject in need thereof comprising administering a composition according to any one of claims 28 to 37 to the subject.

43. The method of claim 42, wherein the composition is administered by intramuscular or intravascular injection.

44. A method of treating a monogenic disease associated with multinucleated cells in a subject in need thereof comprising administering a composition according to any one of claims 38 or 41 to the subject.

45. The method of claim 44, wherein the composition is administered by intramuscular or intravascular injection.
